(19) United States
(12) Patent Application Publication
(10) Pub. No.: US 2013/0236486 A1
(43) Pub. Date: Sep. 12, 2013

(54) IMMUNOGENIC VACCINE

(71) Applicants: Geert-Jan Boons, Athens, GA (US); Margaretha Wolfert, Athens, GA (US); Sandra J. Gendler, Scottsdale, AZ (US); Vani Lakshminarayanan, Scottsdale, AZ (US); Peter A. Cohen, Scottsdale, AZ (US)

(72) Inventors: Geert-Jan Boons, Athens, GA (US); Margaretha Wolfert, Athens, GA (US); Sandra J. Gendler, Scottsdale, AZ (US); Vani Lakshminarayanan, Scottsdale, AZ (US); Peter A. Cohen, Scottsdale, AZ (US)

(73) Assignees: MAYO FOUNDATION FOR MEDICAL EDUCATION AND RESEARCH, Rochester, MN (US); UNIVERSITY OF GEORGIA RESEARCH FOUNDATION,INC, Athens, GA (US)

(21) Appl. No.: 13/703,434

(22) PCT Filed: Jun. 10, 2011
(86) PCT No.: PCT/US11/40037

§ 371 Date: May 20, 2013

Related U.S. Application Data
(63) Continuation-in-part of application No. 13/002,180, filed on Aug. 5, 2011.
(60) Provisional application No. 61/354,076, filed on Jun. 11, 2010.

(51) Int. Cl. C07K 14/705; C12N 9/96

(52) U.S. Cl. CPC C07K 14/705; C12N 9/96
USPC 424/186.1; 530/359; 424/185.1; 435/188

(57) ABSTRACT

A glycolipopeptide comprising a carbohydrate component, a lipid component, and a MUC1 peptide component that induces both a humoral and a cellular immune response for use as a therapeutic or prophylactic vaccine.

This application claims the benefit of U.S. Provisional Application Ser. No. 61/354,076, filed Jun. 12, 2010, and U.S. patent application Ser. No. 13/002,180, filed Dec. 30, 2010, each of which is incorporated herein by reference in its entirety.

### STATEMENT OF GOVERNMENT RIGHTS

This invention was made with government support under Grant No. R01 CA088986, awarded by that National Cancer Institute of the National Institutes of Health. The U.S. Government has certain rights in this invention.

### BACKGROUND

A large number of tumor-associated carbohydrate antigens (TACA) are expressed on human cancer cells in the form of glycolipids and glycoproteins. A common feature of oncogenic transformed cells is the over-expression of oligosaccharides, such as Globo-H, Lewis^{Y}, and Tn antigens. Numerous studies have shown that this abnormal glycosylation can promote metastasis and hence it is strongly correlated with poor survival rates of cancer patients.

The differential expression that is characteristic of these tumor-associated carbohydrate antigens renders them attractive targets for immunotherapy and the development of cancer vaccines. Recently, several elegant studies have attempted to capitalize on the differential expression of tumor-associated carbohydrates for the development of cancer vaccines (e.g., Raghupathi, 1996, Cancer Immunol; 43:152-157; Musselli et al., 2001, J Cancer Res Clin Oncol; 127:R20-R26; Sabbatini et al., 2000, Int J Cancer; 87:79-85; Lo-Man et al., 2004, Cancer Res; 64:4987-4994; Kagan et al., 2005, Immunol Immunother; 54:424-430).

Carbohydrate antigens are also abundant on the surface the human immunodeficiency virus (HIV), the causative agent of acquired immune deficiency syndrome (AIDS). Hepatitis C virus (HCV) is also known to contain carbohydrate antigens.

For most immunogens, including carbohydrates, antibody production depends on the cooperative interaction of two types of lymphocytes, B-cells and helper T-cells. Carbohydrates alone, however, cannot activate helper T-cells and therefore are characterized by poor immunogenicity. The formation of low affinity IgM antibodies and the absence of IgG antibodies manifest this limited immunogenicity. It has proven difficult to overcome the immunotolerance that characterizes these antigens.

In an effort to activate helper T cells, researchers have conjugated carbohydrate antigens to a foreign carrier protein, e.g. keyhole limpet hemocyanin (KLH) or detoxified tetanus toxoid (TT). The carrier protein enhances the presentation of the carbohydrate to the immune system and supplies T-epitopes (typically peptide fragments of 12-15 amino acids) that can activate T-helper cells.

However, conjugation of carbohydrates to a carrier protein poses several new problems. The conjugation chemistry is difficult to control, resulting in conjugates with ambiguities in composition and structure that may affect the reproducibility of an immune response. In addition, the foreign carrier protein may elicit a strong B-cell response, which in turn may lead to the suppression of an antibody response against the carbohydrate epitope. The latter is particularly a problem when self-antigens are employed such as tumor-associated carbohydrates. Also, linkers employed for conjugating carbohydrates to proteins can themselves be immunogenic, leading to epitope suppression. See also McGeary et al., for a review of lipid and carbohydrate based adjuvant/carriers in vaccines (J. Peptide Sci. 9 (7): 405-418, 2003).

Not surprisingly, several clinical trials with carbohydrate-protein conjugate cancer vaccines failed to induce sufficiently strong helper T-cell responses in all patients. Therefore, alternative strategies need to be developed for the presentation of tumor associated carbohydrate epitopes that will result in a more efficient class switch to IgG antibodies. These strategies may prove useful as well for the development of vaccines based on other carbohydrate epitopes, particularly those from pathogenic viruses such as HIV and HCV.

### SUMMARY OF THE INVENTION

The present invention includes a method of generating antibody-dependent cell-mediated cytotoxicity (ADCC) in a subject, the method including immunizing the subject with a glycolipopeptide including at least one glycosylated MUC1 glycopeptide component including a B-cell epitope; at least one peptide component including a MHC class II restricted helper T-cell epitope; and at least one lipid component. In some aspects, the ADCC is natural killer (NK) cell mediated. In some aspects, the ADCC lyses tumor cells. In some aspects, the tumor cells are breast cancer cells or epithelial cancer cells. In some aspects, the ADCC lyses cells expressing a MUC1 peptide sequence. In some aspects, the MUC1 peptide is aberrantly glycosylated.

The present invention includes a method of treating cancer in a subject, the method including immunizing the subject with a glycolipopeptide including: at least one glycosylated MUC1 glycopeptide component including a B-cell epitope; at least one peptide component including a MHC class II restricted helper T-cell epitope; and at least one lipid component.

The present invention includes a method of reducing the tumor burden in a subject, the method including immunizing the subject with a glycolipopeptide including: at least one glycosylated MUC1 glycopeptide component including a B-cell epitope; at least one peptide component including a MHC class II restricted helper T-cell epitope; and at least one lipid component.

The present invention includes a method of preventing tumor recurrence in a subject, the method including immunizing the subject with a glycolipopeptide including: at least one glycosylated MUC1 glycopeptide component including a B-cell epitope; at least one peptide component including a MHC class II restricted helper T-cell epitope; and at least one lipid component.

The present invention includes a method of preventing cancer in a subject, the method including immunizing the subject with a glycolipopeptide including: at least one glycosylated MUC1 glycopeptide component including a B-cell epitope; at least one peptide component including a MHC class II restricted helper T-cell epitope; and at least one lipid component.

In some aspects of the methods of the present invention, the cancer or tumor is breast cancer or epithelial cancer.

In some aspects of the methods of the present invention, the cancer or tumor expresses aberrantly glycosylated MUC1.

The present invention includes a method of generating a cytotoxic T cell response directed at MUC1 expressing cells in a subject, the method including immunizing the subject with a glycolipopeptide including: at least one glycosylated MUC1 glycopeptide component including a B-cell epitope; at least one peptide component including a MHC class II restricted helper T-cell epitope; and at least one lipid component. In some aspects, the MUC1 expressing cells are tumor cells.

The present invention includes a method of promoting anti-MUC1 antibody class switching in a subject, the method including immunizing the subject with a glycolipopeptide including: at least one glycosylated MUC1 glycopeptide component including a B-cell epitope; at least one peptide component including a MHC class II restricted helper T-cell epitope; and at least one lipid component.

The present invention includes a method of immunizing a subject, the method including immunizing the subject with a glycolipopeptide including: at least one glycosylated MUC1 glycopeptide component including a B-cell epitope; at least one peptide component including a MHC class II restricted helper T-cell epitope; and at least one lipid component; wherein antibodies of the IgG subtype that specifically bind to a MUC1 protein expressed on a tumor cell are induced in the subject.

In some aspects of the methods of the present invention, the glycosylated MUC1 glycopeptide component including a B-cell epitope includes glycosylation at one or more serine and/or threonine residues.

In some aspects of the methods of the present invention, the glycosylated MUC1 glycopeptide component including a B-cell epitope includes glycosylation with a sugar residue selected from the group consisting of GalNAc, GlcNAc, Gal, NANA, NGNA, fucose, mannose, and glucose.

In some aspects of the methods of the present invention, the glycolipopeptide includes one of those shown in FIG. 19.

In some aspects of the methods of the present invention, the glycosylated MUC1 glycopeptide component including a B-cell epitope is a class I MHC restricted epitope.

In some aspects of the methods of the present invention, the glycosylated MUC1 glycopeptide component including a B-cell epitope and/or the peptide component including a MHC class II restricted helper T-cell epitope includes a human MUC1 peptide sequence.

In some aspects of the methods of the present invention, the glycosylated MUC1 glycopeptide component including a B-cell epitope and/or the peptide component including a MHC class II restricted helper T-cell epitope includes an amino acid sequence that is homologous to an endogenous MUC1 sequence of the subject.

In some aspects of the methods of the present invention, the glycosylated MUC1 glycopeptide component including a B-cell epitope and/or the peptide component including a MHC class II restricted helper T-cell epitope includes about 5 to 30 amino acids of a MUC1 protein sequence, the MUC1 protein sequence including an extracellular region of the MUC1 protein and including one or more serine or threonine residues that are glycosylated.

In some aspects of the methods of the present invention, the MUC1 glycopeptide component including a B-cell peptide epitope includes an amino acid sequence with at least about 50% sequence identity to SAPDTRPAP (SEQ ID NO:20), TSAPDTRPAP (SEQ ID NO:21), SAPDTRPL (SEQ ID NO:22), or TSAPDTRPL (SEQ ID NO:23). In some aspects, the amino acid sequence includes glycosylation at one or more serine and/or threonine residues. In some aspects of the methods of the present invention, the MUC1 glycopeptide component including a B-cell peptide epitope includes SAPDTRPAP (SEQ ID NO:20), TSAPDTRPAP (SEQ ID NO:21), SAPDTRPL (SEQ ID NO:22), or TSAPDTRPL (SEQ ID NO:23). In some aspects, the amino acid sequence includes glycosylation at one or more serine and/or threonine residues.

In some aspects of the methods of the present invention, the lipid component includes one or more lipid chains, one or more cysteine residues and one or more lysine residues.

In some aspects of the methods of the present invention, the lipid component includes a Toll-like receptor (TLR) ligand. In some aspects, the Toll-like receptor (TLR) ligand includes a TLR2 ligand. In some aspects, the TLR2 ligand includes Pam3CysSK4.

In some aspects of the methods of the present invention, the lipid component includes the TLR9 agonist Pam3CysSK4. In some aspects of the methods of the present invention, the lipid component includes a lipidic adjuvant. In some aspects, the lipidic adjuvant includes a lipidated amino acid (LAA).

In some aspects of the methods of the present invention, the peptide component including a MHC class II restricted helper T-cell epitope includes the polio viruses sequence KLFAVWKITYKDT (SEQ ID NO:3).

In some aspects of the methods of the present invention, the peptide component including a MHC class II restricted helper T-cell epitope includes the T cell pan DR epitope PADRE sequence AKFVAAWTLKAAA (SEQ ID NO:24) or FVAAWTLKAAA (SEQ ID NO:25).

In some aspects of the methods of the present invention, the peptide component including a MEW class II restricted helper T-cell epitope includes a MUC1-derived WIC class II restricted helper T-cell peptide epitope. In some aspects, the MUC1-derived B-cell peptide epitope and the MUC1-derived MHC class II restricted helper T-cell peptide epitope includes a contiguous amino acid sequence. In some aspects, the contiguous amino acid sequence is glycosylated at one or more threonine and/or serine residues.

In some aspects of the methods of the present invention, the MUC1-derived B-cell peptide epitope and the MUC1-derived MHC class II restricted helper T-cell peptide epitope includes a contiguous amino acid sequence. In some aspects, the contiguous amino acid sequence includes a sequence with at least about 50% sequence identity to the amino acid sequence APGSTAPPAHGVTSA (SEQ ID NO:26), APGSTAPPAHGVTSAPDTRPL (SEQ ID NO:27), APGSTAPPAHGVTSAPDTRPL (SEQ ID NO:28), or APGSTAPPAHGVTSAPDTRPL (SEQ ID NO:29). In some aspects, the contiguous amino acid sequence includes the amino acid sequence APGSTAPPAHGVTSA (SEQ ID NO:26), APGSTAPPAHGVTSAPDTRPL (SEQ ID NO:27), APGSTAPPAHGVTSAPDTRPL (SEQ ID NO:28), or APGSTAPPAHGVTSAPDTRPL (SEQ ID NO:29). In some aspects, the contiguous amino acid sequence is glycosylated at one or more threonine and/or serine residues. In some aspects of the methods of the present invention, the glycolipopeptide is administered as a liposome. In some aspects, the lipid component of the glycolipopeptide facilitates liposome formation.

In some aspects of the methods of the present invention, the method includes further administering an immune modulator. In some aspects, a composition including the glycolipopeptide and the immune modulator is administered. In some aspects, the immune modulator is covalently linked to the glycolipopeptide. In some aspects, the immune modulator includes a TLR agonist. In some aspects, the TLR agonist includes a TLR9 agonist. In some aspects, the TLR9 agonist includes CpG. In some aspects, the immune modulator is a TLR9 agonist, a COX-2 inhibitor, GM-CSF, an inhibitor of indoleamine 2, 3-dioxygenase (IDO), a chemotherapy agent, or a combination thereof.

The present invention includes a glycolipopeptide including: at least one glycosylated MUC1 glycopeptide component including a B-cell epitope; at least one peptide component including a MUC1-derived MHC class II restricted helper T-cell epitope; and at least one lipid component. In some aspects of the glycolipopeptides of the present invention, the glycosylated MUC1 glycopeptide component including a B-cell epitope includes glycosylation at one or more serine and/or threonine residues.

In some aspects of the glycolipopeptides of the present invention, the glycosylated MUC1 glycopeptide component including a B-cell epitope includes glycosylation with a sugar residue includes GAlNAc, GlcNAc, Gal, NANA, NGNA, fucose, mannose, or glucose.

In some aspects of the glycolipopeptides of the present invention, the glycosylated MUC1 glycopeptide component including a B-cell epitope is a class I MHC restricted epitope.

In some aspects of the glycolipopeptides of the present invention, the glycosylated MUC1 glycopeptide component including a B-cell epitope and/or the peptide component including a MHC class II restricted helper T-cell epitope includes a human MUC1 peptide sequence.

In some aspects of the glycolipopeptides of the present invention, the glycolipopeptide including a B-cell epitope and/or the peptide component including a MHC class II restricted helper T-cell epitope includes about 5 to 30 amino acids of a MUC1 protein sequence, the MUC1 protein sequence including an extracellular region of the MUC1 protein and including one or more serine or threonine residues that are glycosylated.

In some aspects of the glycolipopeptides of the present invention, the glycosylated MUC1 glycopeptide component including a B-cell epitope includes an amino acid sequence with at least about 50% sequence identity to SAPDTRPAP (SEQ ID NO:20), TSAPDTRPAP (SEQ ID NO:21), SAPDTRPL (SEQ ID NO:22, or TSAPDTRPL (SEQ ID NO:23). In some aspects, the glycosylated MUC1 glycopeptide component including a B-cell epitope includes glycosylation at one or more serine and/or threonine residues.

In some aspects of the glycolipopeptides of the present invention, the glycosylated MUC1 glycopeptide component including a B-cell epitope includes SAPDTRPAP (SEQ ID NO:20), TSAPDTRPAP (SEQ ID NO:21), SAPDTRPL (SEQ ID NO:22), or TSAPDTRPL (SEQ ID NO:23). In some aspects, the glycosylated MUC1 glycopeptide component including a B-cell epitope includes glycosylation at one or more serine and/or threonine residues.

In some aspects of the glycolipopeptides of the present invention, the lipid component includes one or more lipid chains, one or more cysteine residues and one or more lysine residues.

In some aspects of the glycolipopeptides of the present invention, the lipid component includes a Toll-like receptor (TLR) ligand. In some aspects, the Toll-like receptor (TLR) ligand includes a TLR2 ligand. In some aspects, the TLR2 ligand includes Pam₃CysSK₄.

In some aspects of the glycolipopeptides of the present invention, the lipid component includes a lipidic adjuvant. In some aspects, the lipidic adjuvant includes a lipidated amino acid (LAA).

In some aspects of the glycolipopeptides of the present invention, the MUC1-derived B-cell peptide epitope and the MUC1-derived MHC class II restricted helper T-cell peptide epitope includes a contiguous amino acid sequence.

In some aspects of the glycolipopeptides of the present invention, the contiguous amino acid sequence includes a sequence with at least 50% sequence identity to the amino acid sequence APGSTAPPAHGVTSA (SEQ ID NO:26), APGSTAPPAHGVTSAPDTRPL (SEQ ID NO:27), APGSTAPPAHGVTSAPDTRPL (SEQ ID NO:28), or APGSTAPPAHGVTSAPDTRPL (SEQ ID NO:29). In some aspects, the contiguous amino acid sequence is glycosylated at one or more threonine and/or serine residues.

In some aspects of the glycolipopeptides of the present invention, the contiguous amino acid sequence includes the amino acid sequence APGSTAPPAHGVTSA (SEQ ID NO:26), APGSTAPPAHGVTSAPDTRPL (SEQ ID NO:27), APGSTAPPAHGVTSAPDTRPL (SEQ ID NO:28), or APGSTAPPAHGVTSAPDTRPL (SEQ ID NO:29). In some aspects, the contiguous amino acid sequence is glycosylated at one or more threonine and/or serine residues.

In some aspects of the glycolipopeptides of the present invention, the glycolipopeptide includes any of those shown in FIG. 19. In some aspects, the amino acid sequence is glycosylated at one or more threonine and/or serine residues.

In some aspects, the glycolipopeptides further includes a covalently linked immune modulator. In some aspects, immune modulator includes a TLR9 agonist, a COX-2 inhibitor, GM-CSF, an inhibitor of indoleamine 2, 3-dioxygenase (IDO), a chemotherapy agent, or a combination thereof.

The present invention includes pharmaceutical compositions including: a glycolipopeptide as described herein and a pharmaceutically acceptable carrier.

The present invention includes a composition including liposomes including a glycolipopeptide as described herein. In some aspects, the lipid component of the glycolipopeptide facilitates liposome formation. In some aspects, a composition further includes an immune modulator. In some aspects, the immune modulator includes a TLR agonist. In some aspects, TLR agonist includes a TLR9 agonist. In some aspects, the TLR9 agonist includes CpG.

In some aspects, the immune modulator includes a TLR9 agonist, a COX-2 inhibitor, GM-CSF, an inhibitor of indoleamine 2, 3-dioxygenase (MO), a chemotherapy agent, or a combination thereof.

The present invention includes an immunogenic vaccine including a glycolipopeptide as described herein or a composition as described herein.

The present invention includes the use of a glycolipopeptide as described herein or a composition as described herein for the manufacture of a medicament to treat or prevent an infection, disease or disorder.

The present invention includes a kit including: a glycolipopeptide as described herein; packaging; and instructions for use.

Unless otherwise specified, “a,” “an,” “the,” and “at least one” are used interchangeably and mean one or more than one.

### BRIEF DESCRIPTION OF THE DRAWINGS

FIG. 1 shows an exemplary glycolipopeptide of the invention.

FIG. 2 shows flow cytometry analysis for specific anti-MUC-1 antibodies. Reactivity was tested on MCF-7 (A) and SK-MEL-28 (B) cells. Fluorescence intensity of serum (1:50 diluted) was assessed before (serum control; open peak) and after immunization with 3 (filled peak).

FIG. 3 shows TNF-α production by murine macrophages after stimulation with LPS and synthetic compounds. Murine RAW γNO(−) cells were incubated for 5.5 hours with increasing concentrations of E. coli LPS (▪), 1 (), Pam₂CysSK₄ (▾), 2 (♦), Pam₃CysSK₄ (▴), or 3 (□) as indicated.

FIG. 4 shows the effect of TLR ligand on cellular uptake.

FIG. 5 shows the chemical structures of synthetic antigens.

FIG. 6 shows TNF-α and IFN-β production by murine macrophages after stimulation with synthetic compounds 21-24, E. coli LPS, and E. coli lipid A. Murine 264.7 RAW γNO(−) cells were incubated for 5.5 h with increasing concentrations of 21-24, E. coli LPS, or E. coli lipid A as indicated. TNF-α (A) and IFN-β (B) in cell supernatants were measured using ELISAs. Data represent mean values±SD (n=3).

FIG. 7 shows cell recognition analysis for specific anti-MUC1 antibodies. Reactivity of sera was tested on MCF7 cells. Serial dilutions of serum samples after 4 immunizations with 21 (A), 22/23 (B), or 22/24 (C) were incubated with MCF7 cells. After incubation with FITC-labeled anti-mouse IgG antibody, the fluorescence intensity was assessed in cell lysates. No fluorescence over background was observed with pre-immunization sera and incubation of the serum samples with control SK-MEL-28 cells (shown in FIG. 9). AU indicates arbitrary fluorescence units.

FIG. 8 shows ELISA anti-MUC1 and anti-T-epitope antibody titers after 4 immunizations with 21, 22, 22/23, 22/24 and 25/26. ELISA plates were coated with BSA-MI-MUC-1 conjugate (A-F) or neutravidin-biotin-T-epitope (G) and titers were determined by linear regression analysis, plotting dilution vs. absorbance. Titers were defined as the highest dilution yielding an optical density of 0.1 or greater over that of normal control mouse sera. Each data point represents the titer for an individual mouse after 4 immunizations and the horizontal lines indicate the mean for the group of five mice.

FIG. 9 shows cell recognition analysis for specific anti-MUC-1 antibodies. Reactivity of sera was tested on MCF7 and SK-MEL-28 cells. Serum samples (1:30 diluted) after 4 immunizations with 21, 22/23, or 22/24 were incubated with MCF7 and SK-MEL-28 cells. After incubation with FITC-labeled anti-mouse IgG antibody, the fluorescence intensity was assessed in cell lysates. Also shown are media, conjugate, and mouse (normal control mouse sera) controls. Data represent mean values±SD. AU indicates arbitrary fluorescence units.

FIG. 10 shows compound 22.

FIG. 11 shows compound 23.

FIG. 12 shows compound 25.

FIG. 13 shows compound 26.

FIG. 14 shows compound 27.

FIG. 15 shows the structure of fully synthetic three-component immunogens.

FIG. 16. Chemical structures of synthetic antigens 1, 2, 3, 4, and 5.

FIG. 17. MMT tumor burden of MUC1.Tg mice is reduced by three component vaccine. MUC1.Tg mice were immunized with empty liposomes (EL) as control or with liposomes containing 1, 2, 3, 4+5 or 5 (25 μg containing 3 μg of carbohydrate). Chemical structures of synthetic antigens 1, 2, 3, 4, and 5 are as shown in FIG. 16. Three bi-weekly immunizations were given prior to a tumor challenge with MUC1-expressing MMT tumor cells (1 10⁶ cells) followed by one boost one week after. The animals were sacrificed 7 days after the last injection and tumor wet weight was determined. Data are presented as percentage of control (mice vaccinated with empty liposomes). Each data point represents an individual mouse and the horizontal lines indicate the mean for the group of mice.

FIGS. 18A and 18B. Induction of antibody-dependent cell-mediated cytotoxicity (ADCC). Tumor cells, Yac-MUC1 (FIG. 18A) and C57mg.MUC1 (FIG. 18B), were labeled with chromium for 2 h and then incubated with serum (1:50 diluted) obtained from mice immunized with empty liposomes (EL) or liposomes containing 1, 2, 3, 4+5 or 5 with or without (NT) tumor induction as indicated for 30 min at 37° C. Chemical structures of synthetic antigens 1, 2, 3, 4, and 5 are as shown in FIG. 16. The tumor cells were then incubated with effector cells (NK cells KY-1 clone) for 4 h. Effector to target ratio is 50:1. Spontaneous release was below 15% of complete release. Each data point represents an individual mouse and the horizontal lines indicate the mean for the group of mice.

FIGS. 19A to 19C. Cellular responses. FIG. 19A assays IFN-γ producing CD8⁺ T-cells in MUC1.Tg mice. CD8⁺ T-cells isolated from lymph nodes of mice immunized with empty liposomes (EL) or liposomes containing 1, 2, 3, 4+5 or 5 with or without (NT) tumor induction as indicated were analyzed for MUC1-specific IFN-γ spot formation. Chemical structures of synthetic antigens 1, 2, 3, 4, and 5 are as shown in FIG. 16. Each data point represents an individual mouse and the horizontal lines indicate the mean for the group of mice. FIG. 19B assays induction of CD8⁺ cytolytic T-cells in MUC1.Tg mice. CD8⁺ T-cells were isolated from lymph nodes of mice immunized with empty liposomes (EL) or liposomes containing 1, 2, 3, 4+5 or 5 with or without (NT) tumor induction as indicated and subjected to a ⁵¹Cr-release assay without any in vitro stimulation. DCs pulsed with MUC1(Tn) peptide 6 (SAPDT(Tn)RPAP) (SEQ ID NO:26) for 1 (NT), 1, 3, 4+5 and 5, MUC1 peptide 7 (SAPDTRPAP) (SEQ ID NO:20) for 2 or empty liposomes for EL were used as targets. Effector to target ratio was 100:1 as CTLs were not stimulated in vitro. Spontaneous release was below 15% of complete release. Each data point represents an individual mouse and the horizontal lines indicate the mean for the group of mice. FIG. 19C assays epitope requirements of CD8⁺ T-cells. Mice were immunized with liposomes containing 1 or 2. Lymph node derived T-cells expressing low levels of CD62L were obtained by cell sorting and cultured for 14 days in the presence of DCs pulsed with glycopeptide 6 for 1 or peptide 7 for 2. The resulting cells were analyzed for the presence of CD8⁺IFNγ⁺ T-cells after exposure to DCs pulsed with (glyco)peptides 6-9. Peptide 6 is SEQ ID NO:26, peptide 7 is SEQ ID NO:20, peptide 8 is SEQ ID NO:27, and peptide 9 is SEQ ID NO:29.

FIGS. 20A to 20H. ELISA anti-MUC1 and anti-helper T-epitope antibody titers after three (FIG. 20A) or four (FIGS. 20B-H) immunizations with 1, 2, 3, 4+5 or 5 with or without (NT) tumor induction as indicated. Chemical structures of synthetic antigens 1, 2, 3, 4, and 5 are as shown in FIG. 16. ELISA plates were coated with BSA-MI-MUC1(Tn) conjugate (FIGS. 20A-G) or neutravidin-biotin-helper T-epitope (FIG. 20H) and titers were determined by linear regression analysis, plotting dilution vs. absorbance. Titers were defined as the highest dilution yielding an optical density of 0.1 or greater over that of normal control mouse sera. Each data point represents the titer for an individual mouse after four immunizations and the horizontal lines indicate the mean for the group of mice.

FIGS. 21A and 21B. Competitive inhibition of antibody binding to BSA-MI-MUC1(Tn) conjugate by MUC1(Tn) 6, unglycosylated MUC1 7 and Tn monomer.

Sequences of compounds 6 and 7 are as shown in FIG. 19. ELISA plates were coated with BSA-MI-MUC1(Tn) conjugate. Serum samples after immunizations with 1 (FIG. 21A) and 2 (FIG. 21B), diluted to obtain in the absence of an inhibitor an OD of approximately 1 in the ELISA, were first mixed with 6, 7 or Tn monomer (0-500 μM final concentration) and then applied to the coated microtiter plate. Optical density values were normalized for the optical density values obtained with serum alone (0 μM inhibitor, 100%). The data are reported as the means±s.e.m of groups of mice (n=7).

FIGS. 22A to 22J. Cytokine production by dendritic cells after stimulation with liposome preparations loaded with compound 1, 2 or 3, or E. coli LPS for 24 h. Chemical structures of synthetic antigens 1, 2, or 3 are as shown in FIG. 16. Primary dendritic mouse cells were incubated for 24 h with increasing concentrations of liposome preparations loaded with compound 1, 2 or 3, or E. coli LPS as indicated. TNF-α (FIG. 22A), IFN-β (FIG. 22B), RANTES (FIG. 22C), IL-6 (FIG. 22D), extracellular IL-10 (FIG. 22E and FIG. 22F), IL-10 (FIG. 22G), IP-1β (FIG. 22H), IL-12 p70 (FIG. 22I) and IL-12/23 p40 (FIG. 22J) in cell supernatants were measured using ELISAs. For estimation of IL-1β secretion after ATP treatment, cells were incubated with ATP (5 mM) for 30 min subsequent to the 24 h incubation with inducers. The data are reported as the means±SD of triplicate treatments.

FIG. 23. Tumor weight in grams (gm) in MUC1.Tg mice immunized with preparations of Compound 2 (Pam₃CysSK₄-T helper ep. (Polio)-MUC1 (unglycosylated)); Compound 1 (Pam₃CysSK₄-T helper ep. (polio)-MUC1(Tn)); Compound 1 plus CpG (CpG oligodeoxynucleotides (CpG ODN))); Compound 5 (Pam₃CysSK₄) plus Compound 4 (T helper ep. (Polio)-MUC1(Tn)); Compound 5; Compound 3 (Pam₃CysSK₄-T helper ep. (Polio)); Compound 3 plus CpG; EL (empty liposomes) plus CpG; or EL. Chemical structures of synthetic antigens 1, 2, 3, 4, and 5 are as shown in FIGS. 16 and 26.

FIG. 24. Cytolytic activity of CD8+ cells obtained from MUC1.Tg mice immunized with preparations of Compound 2 (Pam₃CysSK₄-T helper ep. (Polio)-MUC1 (unglycosylated)); Compound 1 (Pam₃CysSK₄-T helper ep. (polio)-MUC1(Tn)); Compound 1 plus CpG (CpG oligodeoxynucleotides (CpG ODN))); Compound 5 (Pam₃CysSK₄) plus Compound 4 (T helper ep. (Polio)-MUC1(Tn)); Compound 5; Compound 3 (Pam₃CysSK₄-T helper ep. (Polio)); Compound 3 plus CpG; EL (empty liposomes) plus CpG; or EL. Chemical structures of synthetic antigens 1, 2, 3, 4, and 5 are as shown in FIGS. 16 and 26.

FIG. 25. Determination of IFN-γ production by CD8+ T cells obtained from MUC1.Tg mice immunized with preparations of Compound 2 (Pam₃CysSK₄-T helper ep. (Polio)-MUC1 (unglycosylated)); Compound 1 (Pam₃CysSK₄-T helper ep. (polio)-MUC1(Tn)); Compound 1 plus CpG (CpG oligodeoxynucleotides (CpG ODN))); Compound 5 (Pam₃CysSK₄) plus Compound 4 (T helper ep. (Polio)-MUC1(Tn)); Compound 5; Compound 3 (Pam₃CysSK₄-T helper ep. (Polio)); Compound 3 plus CpG; EL (empty liposomes) plus CpG; or EL. Chemical structures of synthetic antigens 1, 2, 3, 4, and 5 are as shown in FIGS. 16 and 26.

FIG. 26. Structure of Compound 1 (Pam₃CysSK₄-T-helper-MUC1), Compound 2 ((Pam₃CysSK₄-T-helper), LAA-T-helper-MUC1, and LAA-T-helper.

FIG. 27. Immunization protocol.

FIG. 28. Three-component vaccine reduced tumor burden. MUC1.Tg mice were immunized with liposomes containing Compound 1 (Pam₃CysSK₄-T-helper-MUC1), Compound 2 ((Pam₃CysSK₄-T-helper), LAA-T-helper-MUC1, or LAA-T-helper (25 μg containing 3 μg of carbohydrate) or with empty liposomes as control. Three bi-weekly immunizations were given prior to a tumor challenge with MUC1-expressing MMT tumor cells (1×10⁶ cells) followed by one boost one week after. The animals were sacrificed 7 days after the last injection and tumor wet weight was determined. Data are presented as percentage of control (mice vaccinated with empty liposomes). Each data point represents an individual mouse and the horizontal lines indicate the mean for the group of mice.

FIG. 29. MUC1-specific cytotoxic CD8 T cells were induced by vaccine. CD8+ T cells were isolated from lymph nodes of mice immunized with empty liposomes or liposomes containing Compound 1 (Pam₃CysSK₄-T-helper-MUC1), Compound 2 ((Pam₃CysSK₄-T-helper), LAA-T-helper-MUC1, or LAA-T-helper with tumor induction as indicated and subjected to a 51Cr-release assay without any in vitro stimulation. DCs pulsed with Tn-MUC1 peptide (SAPDT(Tn)RPAP) (SEQ ID NO:26) or empty liposomes were used as targets. Effector to target ratio is 100:1 as CTLs were not stimulated in vitro. Spontaneous release was below 15% of complete release. Each data point represents an individual. Chemical structures of synthetic antigens 1, 2, 3, 4, and 5 are as shown in FIGS. 16 and 26.

FIG. 30. Three-component vaccine elicited strong antibody titers. ELISA anti-MUC1 and anti-T-epitope antibody titers after four immunizations. Anti-MUC1 and anti-T-epitope antibody titers are presented as median values for groups of four to seven mice. ELISA plates were coated with BSA-MI-MUC1(Tn) conjugate for anti-MUC1 antibody titers or neutravidin-biotin-T-epitope for anti-T-helper epitope antibody titers. Titers were determined by linear regression analysis, with plotting of dilution versus absorbance. Titers are defined as the highest dilution yielding an optical density of 0.1 or greater relative to normal control mouse sera.

FIG. 31. Antibodies were effective at antibody-dependent cellular cytotoxicity (ADCC). C57 mg.MUC1 mammary tumor cells were labeled with chromium for two hours and then incubated with control serum (MUC1.Tg) or serum (1:50 diluted) obtained from MMT tumor bearing mice immunized with empty liposomes or liposomes containing Compound 1 (Pam₃CysSK₄-T-helper-MUC1), Compound 2 ((Pam₃CysSK₄-T-helper), LAA-T-helper-MUC1, and LAA-T-helper as indicated for 30 minutes (min) at 37° C. The tumor cells were then incubated with effector cells (KY-1 cells-NK clone) for four hours. Effector to target ratio is 50:1. Spontaneous release was below 15% of complete release. Each data point represents an individual mouse and the horizontal lines indicate the mean for the group of mice. Chemical structures of synthetic antigens 1, 2, 3, 4, and 5 are as shown in FIGS. 16 and 26.

FIG. 32. Lead sequences of human MUC1 with Rankpep findings highlighted. Initial sequences of tandem repeat underlined. The dashed line shows 15mers showing RANKPEP score for binding to I-A^{b}. 9mers showing RANKPEP score for binding to H2-D^{b} (dddd) or H2-K^{b} (kkkk) or promiscuous binding to both (bbbb) are designated.

FIGS. 33A and 33B. Mice were immunized with the peptides described in FIG. 33A and lymph node-derived T-cells expressing low levels of CD62L were obtained by cell sorting and cultured for 14 days in the presence of DCs pulsed with the immunizing peptide. The resulting cells were analyzed by intracellular cytokine for the presence of CD4⁺IFNγ⁺ and CD8⁺IFNγ⁺ T-cells after exposure of the DCs pulsed with the peptides listed on the y-axis (FIG. 33B).

FIG. 34. Synthetic constructs utilizing human MUC1 T-helper sequences.

FIG. 35 shows the structures of fully synthetic three-component immunogens 52 and 53 and the reagents 63-65 for their preparation.

FIG. 36 shows ELISA anti-GSTPVS(β-O-GlcNAc) SANM (68) antibody titers after 4 immunizations with 52 and 53. ELISA plates were coated with BSA-MI-GSTPVS(β-O-GlcNAc) SANM (BSA-MI-66) conjugate and (a) IgG total, (b) IgG1, (c) IgG2a, (d) IgG2b, (e) IgG3 and (f) IgM titers were determined by linear regression analysis, plotting dilution vs. absorbance. Titers were defined as the highest dilution yielding an optical density of 0.1 or greater over that of normal control mouse sera. Each data point represents the titer for an individual mouse after 4 immunizations and the horizontal lines indicate the mean for the group of five mice.

FIG. 37 shows compound 52.

FIG. 38 shows compound 53.

FIG. 39 shows compound 63.

FIG. 40 shows compound 64.

FIG. 41 shows compound 65.

FIG. 42 shows compound 66.

FIG. 43 shows compound 67; SEQ ID NO: 12.

FIG. 44 shows compound 68.

FIG. 45 shows compound 69; SEQ ID NO: 11.

FIG. 46 shows compound 70.

### DESCRIPTION OF ILLUSTRATIVE EMBODIMENTS

The glycolipopeptide of the invention includes at least one B-epitope, at least one T-epitope, and a lipid component. In a preferred embodiment, the glycolipopeptide consists essentially of three main components: at least one carbohydrate component that contains a B-epitope; at least one peptide component that contains a helper T-epitope; and at least one lipid component. Exemplary carbohydrate, peptide and lipid components are described herein and also, for example, in references cited herein, including Koganty et al., US Patent Publication 20060069238, published Mar. 30, 2006; see also Koganty et al., 1996, Drug Disc Today; 1 (5):190-198. The three components are covalently linked, either directly or indirectly, to form a single glycolipopeptide molecule. Indirect linkage involves the use of an optional linker component “L” to link two or more of the main components together. The three main components can be linked together (directly or indirectly) in any order. For example, the lipid and carbohydrate component can each be covalently linked to the peptide component to form the glycolipopeptide. Alternatively, the lipid component and the peptide component can each be covalently linked to the carbohydrate component. Likewise, the carbohydrate component and the peptide component can each be covalently linked to the lipid component. Or, all three components can be linked such that each of the three components is covalently linked to each of the other two components. Intermolecular crosslinking is also possible, as described in more detail below.

In a preferred embodiment, the glycolipopeptide of the invention contains one carbohydrate component, one peptide component, and one lipid component. In another embodiment, the glycolipopeptide contains a plurality of carbohydrate components, which may be the same, or may be different. Likewise, in another embodiment, the glycolipopeptide contains a plurality of peptide components, which may be the same, or may be different. Further, in another embodiment, the glycolipopeptide contains a plurality of lipid components, which may be the same, or may be different. Thus, various embodiments of the glycolipopeptide of the invention may contain one or more carbohydrate components, one or more peptide components, and/or one or more lipid components. For example, the concept of “multiple antigenic glycopeptides” (Bay et al., U.S. Pat. No. 6,676,946, Jan. 13, 2004, Bay et al.; WO 98/43677, published Oct. 8, 1998, Bay et al.) can be adapted for use in the present invention. High antigen density can be achieved using a core, for example a poly-lysine core, to which extended peptidic “arms” (the peptide component of the glycolipopeptide of the invention) are attached, which peptidic arms display the carbohydrate antigen components of the glycolipopeptide in clustered presentation. The lipid component of the glycolipopeptide can likewise extend from the lysine core, particularly in embodiments wherein the peptide component is attached to the lysine core via a nonterminal amino acid. High antigen density can also be achieved by using a liposome as a delivery vehicle, as exemplified in Examples 2 and 3. Additionally or alternatively, the glycolipopeptides can be optionally cross-linked to form a multi-molecular complex, thereby increasing the antigen density.

The various carbohydrate, peptide and lipid components of the glycolipopeptide can be structurally derived from or based on, and/or can mimic, those found in naturally occurring biological molecules. The glycolipopeptide components preferably contain molecular structures or parts of structures (including epitopes) that are identical to or similar to those found in a living organism. Typically, while the components of the glycolipopeptide are derived from, are structurally based on, and/or mimic naturally occurring structures, they are prepared synthetically, using chemical or in vitro enzymatic methods, for example. In some embodiments, epitopes that are formed in the naturally occurring antigen from molecular elements that are close in space but distant from each other in terms of chemical bonding can be formed in the glycolipopeptide of the invention by a different chemical structure (with different bonding order or pattern) that forms the same or a similar epitope.

The three component glycolipopeptide of the invention can be viewed as cassette, wherein the carbohydrate component, the peptide component, and the lipid component are each independently selected for inclusion in the glycolipopeptide. Any combination (i.e., mixing and matching) of carbohydrate component, peptide component and lipid component as described herein to form a glycolipopeptide is encompassed by the invention.

### Carbohydrate Component

The carbohydrate component of the glycolipopeptide can be any component that contains a carbohydrate. Examples of suitable carbohydrate components include oligosaccharides, polysaccharides and monosaccharides, and glycosylated biomolecules (glycoconjugates) such as glycoproteins, glycopeptides, glycolipids, glycosylated amino acids, DNA, or RNA. Glycosylated peptides (glycopeptides) and glycosylated amino acids, which contain one or more carbohydrate moieties as well as a peptide or amino acid, are particularly preferred as the carbohydrate component of the glycolipopeptide of the invention. An example of a glycopeptide is CD52, which is expressed on virtually all human lymphocytes and believed to play an important role in the human immune system. An example of a glycosylated amino acid is the Tn antigen. It should be understood that when the carbohydrate component is a glycopeptide, the peptide part of the glycopeptide optionally includes a T-epitope as well as a B-epitope and thus may serve as a peptide component of the glycolipopeptide. A glycopeptide that contains both a T-epitope and a B-epitope is sometimes referred to as possessing a “B-T” epitope or a “T-B” epitope. The B-epitope and the T-epitope present on the glycolipopeptide of the invention may or may not overlap. In preferred embodiments, a T-epitope, B-epitope, and/or T-B epitope is derived from a MUC1 polypeptide sequence, including, but not limited to a human MUC1 polypeptide sequence.

The carbohydrate component of the glycolipopeptide of the invention includes a carbohydrate that contains one or more saccharide monomers. For example, the carbohydrate can include a monosaccharide, a disaccharide or a trisaccharide; it can include an oligosaccharide or a polysaccharide. An oligosaccharide is a oligomeric saccharide that contains two or more saccharides and is characterized by a well-defined structure. A well-defined structure is characterized by the particular identity, order, linkage positions (including branch points), and linkage stereochemistry (α, β) of the monomers, and as a result has a defined molecular weight and composition. An oligosaccharide typically contains about 2 to about 20 or more saccharide monomers. A polysaccharide, on the other hand, is a polymeric saccharide that does not have a well defined structure; the identity, order, linkage positions (including brand points) and/or linkage stereochemistry can vary from molecule to molecule. Polysaccharides typically contain a larger number of monomeric components than oligosaccharides and thus have higher molecular weights. The term “glycan” as used herein is inclusive of both oligosaccharides and polysaccharides, and includes both branched and unbranched polymers. When the carbohydrate component contains a carbohydrate that has three or more saccharide monomers, the carbohydrate can be a linear chain or it can be a branched chain. In a preferred embodiment, the carbohydrate component contains less than about 15 saccharide monomers; more preferably in contains less than about 10 saccharide monomers.

The carbohydrate component of the glycolipopeptide includes a carbohydrate that contains a B-epitope. It should be understood that the carbohydrate may be coextensive with the B-epitope, or the carbohydrate may be inclusive of the B-epitope, or the carbohydrate may include only part of the B-epitope (i.e., the B-epitope may additionally encompass other parts of the glycolipopeptide such as the peptide component, the lipid component, and/or the linker component). An example of a glycopeptide that includes a B-epitope is the glycosylated peptide MUC-1 (also referred to herein as MUC1). Thus, a carbohydrate or carbohydrate component that “comprises” a B-epitope is to be understood to mean a carbohydrate or carbohydrate component that encompasses all or part of a B-epitope that is present on the glycolipopeptide.

The B-epitope can be a naturally occurring epitope or a non-naturally occurring epitope. Preferably, two or more saccharide monomers of the carbohydrate interact to form a conformational epitope that serves as the B-epitope. A B-epitope is an epitope recognized by a B cell. Any antigenic carbohydrate that contains a B-epitope can be used as the carbohydrate component, without limitation. In preferred embodiments, a B-epitope is derived from a MUC1 polypeptide sequence, including, but not limited to, a human MUC1 polypeptide sequence.

Non-naturally occurring carbohydrates that can be used as components of the glycolipopeptide of the invention include glycomimetics, which are molecules that mimic the shape and features of a sugar such as a monosaccharide, disaccharide or oligosaccharide (see, e.g., Barchi, 2000, Current Pharmaceutical Design; 6(4):485-501; Martinez-Grau et al., 1998, Chemical Society Reviews; 27(2):155-162; Schweizer, 2002, Angewandte Chemie-International Edition; 41(2):230-253). Glycomimetics can be engineered to supply the desired B-epitope and potentially provide greater metabolic stability.

In another embodiment, the carbohydrate component contains all or part of a self-antigen. Self-antigens are antigens that are normally present in an animal's body. They can be regarded as “self-molecules,” e.g., the molecules present in or on the animal's cells, or proteins like insulin that circulate in the animal's blood. An example of a self-antigen is a carbohydrate-containing component derived from a cancer cell of the animal, such as a tumor-associated carbohydrate antigen (TACA). Typically, such self-antigens exhibit low immunogenicity. Examples include tumor-related carbohydrate B-epitope such as Le^{y} antigen (a cancer related tetrasaccharide; e.g., Fucα(1,2)-Galβ(1,4)-[Fucα(1,3)]-GlcNAc); Globo-H antigen (e.g., L-Fucα(1,2)-Galβ(1,3)-GalNAcβ(1,3)-Galα(1,4)-Galβ(1,4)-Glu); T antigen (e.g., Galβ(1,3)-GalNAcα-O-Ser/Thr); STn antigen (sialyl Tn, e.g., NeuAcα(2,6)-GalNAcα-O-Ser/Thr); and Tn antigen (e.g., α-GalNAc-O-Ser/Thr). Another example of a self-antigen is a glycopeptide derived from the tandem repeat of the breast tumor-associated MUC-1 of human polymorphic epithelial mucin (PEM), an epithelial mucin (Baldus et al., Crit. Rev. Clin. Lab. Sci., 41(2):189-231 (2004)). A MUC-1 glycopeptide comprises at least one Tn and/or sialyl Tn (sialyl α-6 GalNAc, or “STn”) epitope; preferably linked to a threonine (T-Tn or T-STn).

In preferred embodiments, the carbohydrate component includes a glycosylated MUC1 glycopeptide that is glycosylated at one or more serine and/or threonine residues of a MUC1-derived amino acid peptide sequence. Such a MUC1-derived amino acid sequence, includes, but is not limited to, any of the MUC1 sequence described herein.

Structures of exemplary tumor-associated carbohydrate antigens (TACA) that can be used as a component of the glycolipopeptide include, without limitation, the structures shown in Schemes 1 and 2.

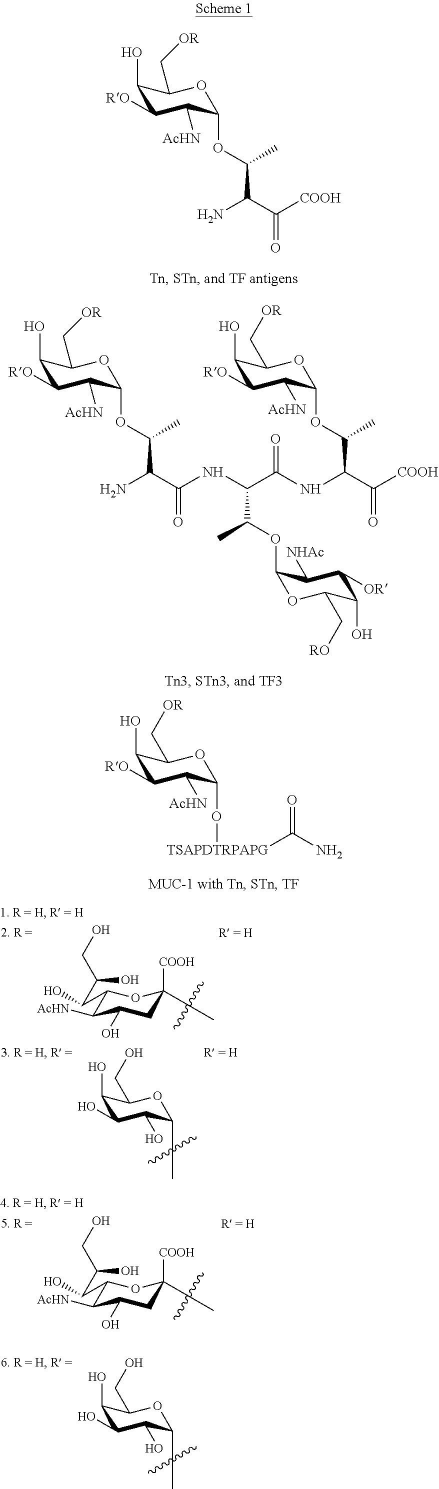

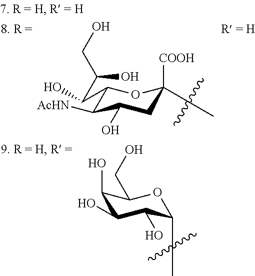

It should be noted that the Tn, STn, and TF structures shown in Scheme 1 (monomeric, trimeric, clustered) are all shown with a threonine residue. The corresponding serine analogues are also suitable structures. In the case of Tn3, STn3, TF3 and their respective clusters, all possible homo- and hetero-analogues with differences in the threonine/serine composition of the backbone are included.

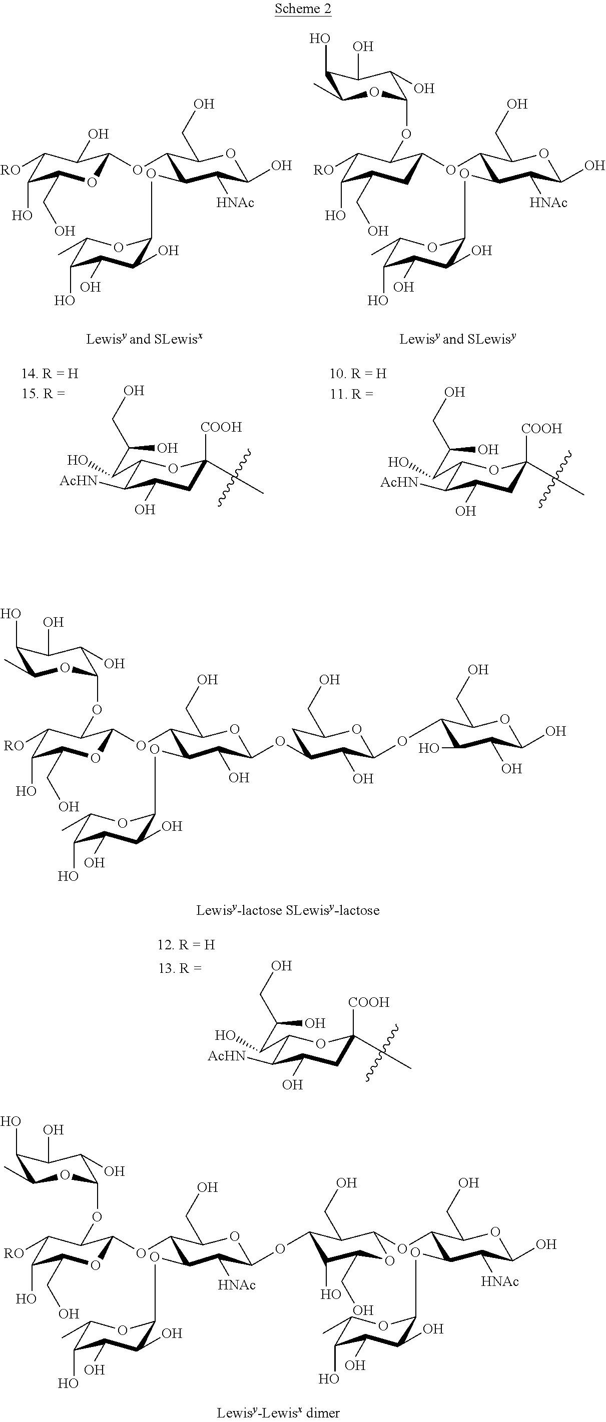

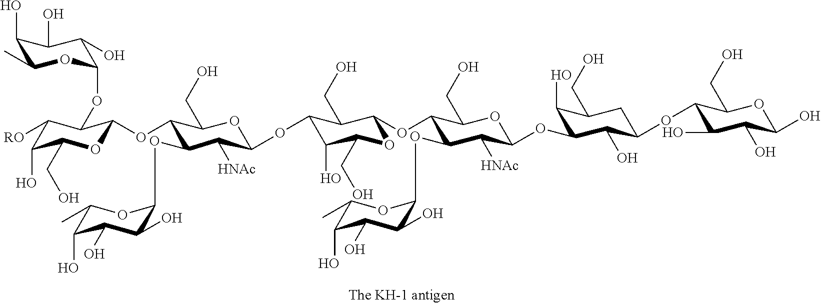

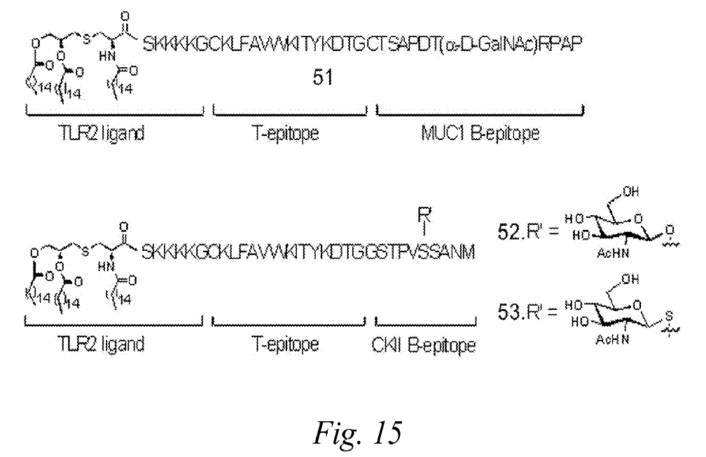

Another self-antigen for use in the carbohydrate component of the glycolipopeptide is a glycopeptide that includes an amino acid or peptide covalently linked to a monosaccharide. Preferably the monosaccharide is N-methylglucosamine (GlcNAc) or N-acetylgalactoseamine (GalNAc). A preferred glycopeptide self-antigen is a β-N-acetylglucosamine (β-O-GlcNAc) modified peptide. Preferably the monosaccharide is O-linked to a serine or a threonine of the polypeptide. Also suitable for use as a self-antigen are the related thiol (S-linked) and amine (N-linked) analogues. The monosaccharide is preferably linked to the peptide via a beta (β) linkage but it may be an alpha (α) linkage. In a particularly preferred embodiment, the carbohydrate component of the glycolipopeptide of the invention (which may be coextensive with the peptide component when formulated as a glycopeptide) contains a TPVSS (SEQ ID NO:10) amino acid sequence modified by O-GlcNAc. Examples of a carbohydrate that contains a β-GlcNAc modified glycopeptide as a B-epitope are shown as compounds 52 (O-linked) and 53 (S-linked) in FIG. 15.

In another embodiment, the carbohydrate component contains all or part of a carbohydrate antigen (typically a glycan) from a microorganism, preferably a pathogenic microorganism, such as a virus (e.g., a carbohydrate present on gp120, a glycoprotein derived from the HIV virus), a Gram-negative or Gram-positive bacterium (e.g., a carbohydrate derived from Haemophilus influenzae, Streptococcus pneumoniae, or Neisseria meningitides), a fungus (e.g., a 1,3-(3-linked glucan) a parasitic protozoan (e.g., a GPI-anchor found in protozoan parasites such as Leishmania and Trypanosoma brucei), or a helminth. Preferably, the microorganism is a pathogenic microorganism.

An exemplary glycan from viral pathogens, Man9 from HIV-1 gp120, is shown in Scheme 3.

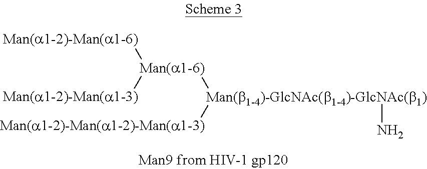

Exemplary HIV carbohydrate and glycopeptide antigens are described in Wang et al., Current Opinion in Drug Disc. & Develop., 9(2): 194-206 (2006), and include both naturally occurring HIV carbohydrates and glycopeptides, as well as synthetic carbohydrates and glycopeptides based on naturally occurring HIV carbohydrates and glycopeptides.

Exemplary HCV carbohydrate and glycopeptide antigens are described in Koppel et al. Cellular Microbiology 2005; 7(2):157-165 and Goffard et al. J. of Virology 2005; 79(13):8400-8409, and include both naturally occurring HCV carbohydrates and glycopeptides, as well as synthetic carbohydrates and glycopeptides based on naturally occurring HCV carbohydrates and glycopeptides.

Exemplary glycans from bacterial pathogens are shown in Scheme 4.

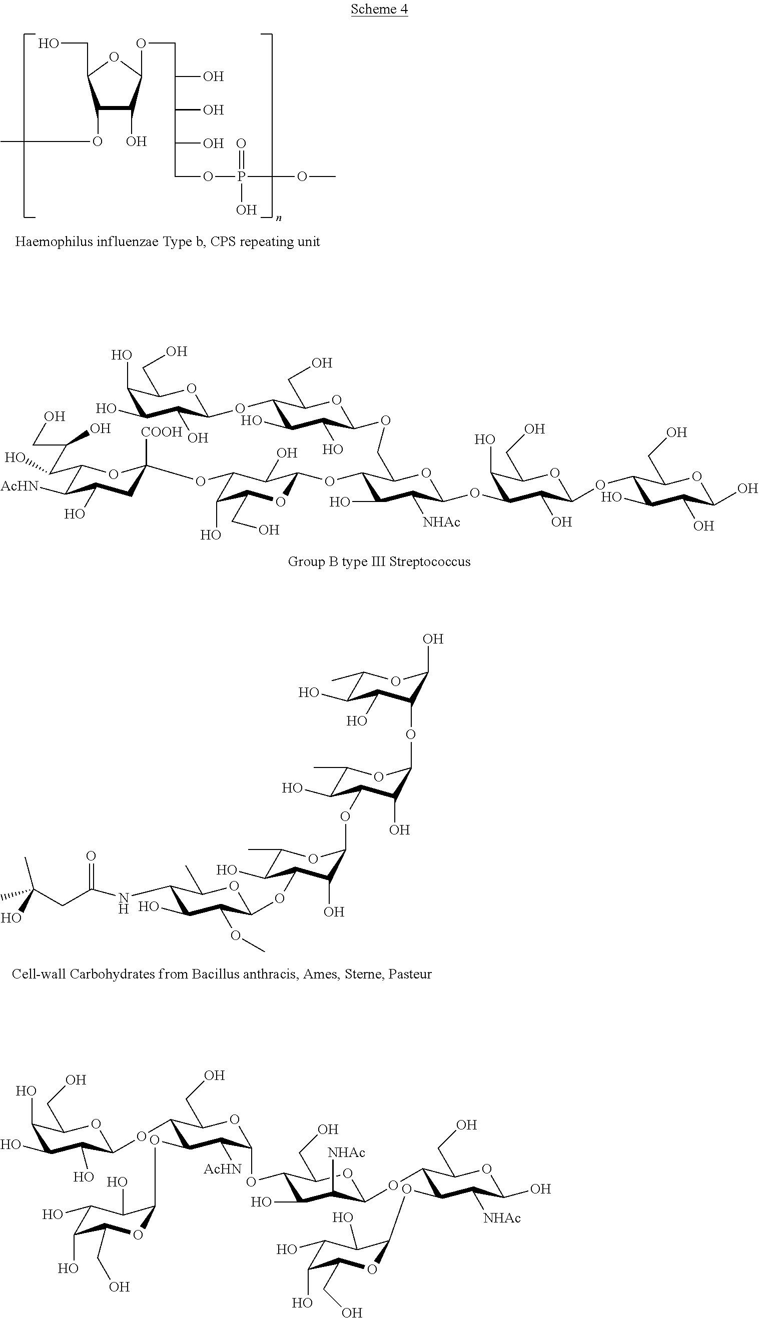

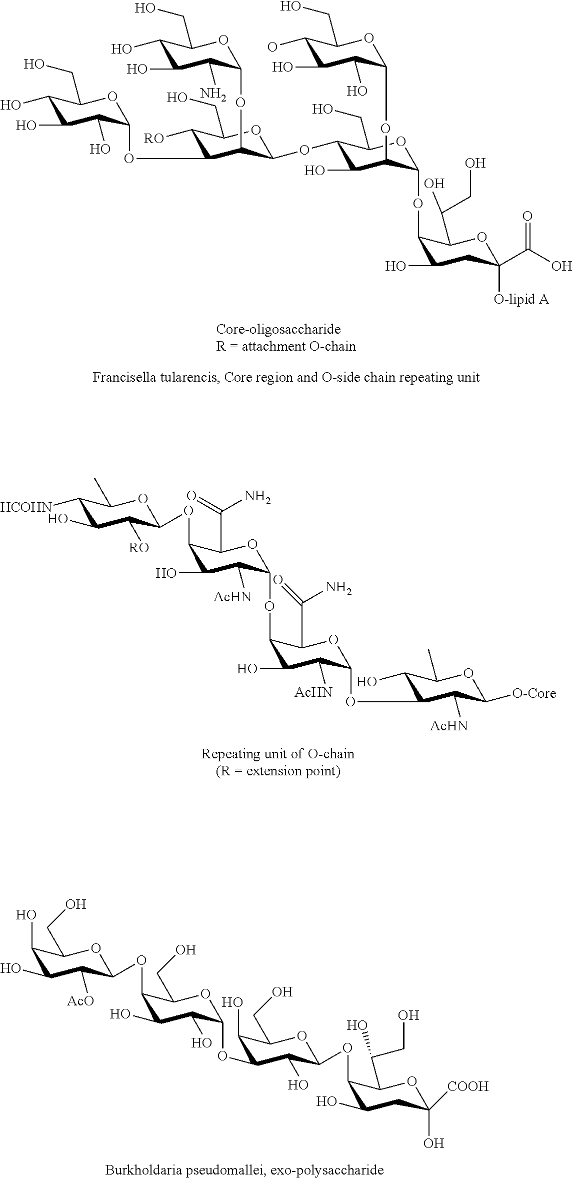

Exemplary glycans from protozoan pathogens are shown in Scheme 5.

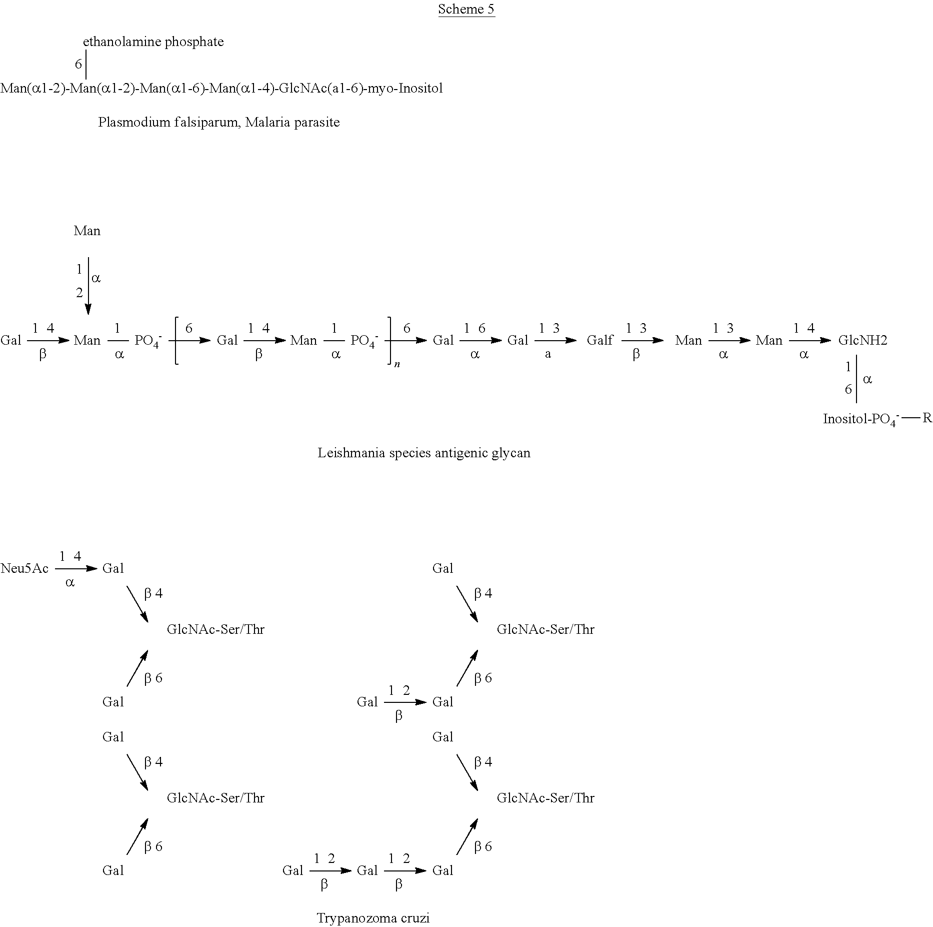

An exemplary glycan from a fungal pathogen is shown in Scheme 6.

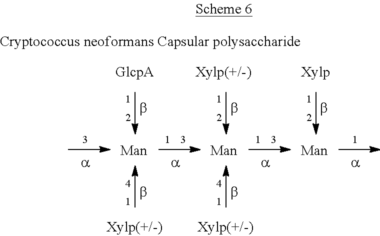

An exemplary glycan from helminth pathogen is shown in Scheme 7.

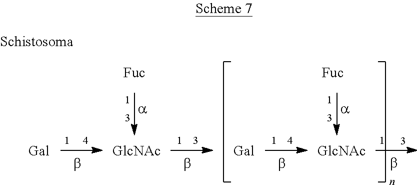

It will be appreciated by one of skill in the art that while numerous antigenic carbohydrate structures are known, many more exist, since only a small fraction of the antigenic or immunogenic carbohydrates have been identified thus far. Examples of the many carbohydrate antigens discovered thus far can be found in Kuberan et al., Curr. Org. Chem, 4, 653-677 (2000); Ouerfelli et al., Expert Rev. Vaccines 4(5):677-685 (2005); Hakomori et al., Chem. Biol. 4, 97-104 (1997); Hakomori, Acta Mat. 161, 79-90 (1998); Croce and Segal-Eiras, Drugs of Today 38(11):759-768 (2002); Mendonca-Previato et al., Curr Opin. Struct. Biol. 15(5):499-505 (2005); Jones, Anais da Academia Brasileira de Ciencias 77(2):293-324 (2005); Goldblatt, J. Med. Microbiol. 47(7):563-567 (1998); Diekman et al., Immunol. Rev., 171: 203-211, 1999; Nyame et al., Arch. Biochem. Biophys., 426 (2): 182-200, 2004; Pier, Expert Rev. Vaccines, 4 (5): 645-656, 2005; Vliegenthart, FEBS Lett., 580 (12): 2945-2950, Sp. Iss., 2006; Ada et al., Clin. Microbiol. Inf., 9 (2): 79-85, 2003; Fox et al., J. Microbiol. Meth., 54 (2): 143-152, 2003; Barber et al., J. Reprod. Immunol., 46 (2): 103-124, 2000; and Sorensen, Persp. Drug Disc. Design, 5: 154-160, 1996. Any antigenic carbohydrate derived from a mammal or from an infectious organism can be used as the carbohydrate component of the glycolipopeptide of the invention, without limitation.

### Peptide Component

The peptide component of the glycolipopeptide includes a T-epitope, preferably a helper T epitope. The peptide component can be any peptide-containing structure, and can contain naturally occurring and/or non-naturally occurring amino acids and/or amino acid analogs (e.g., D-amino acids). The peptide component may be from a microorganism, such as a virus, a bacterium, a fungus, and a protozoan. The T-epitope can therefore constitute all or part of a viral antigen. Alternatively or additionally, the T-epitope can be from a mammal, and optionally constitutes all or part of a self-antigen. For example, the T-epitope can be part of a glycopeptide that is overexpressed on a cancer cell. When the peptide component of the glycolipopeptide of the invention is a glycopeptide, the peptide component may also include all or part of the B-epitope, as described elsewhere herein. More generally, it should be understood that the peptide component of the glycolipopeptide may be coextensive with the T-epitope, or the peptide component may be inclusive of the T-epitope, or the peptide component may include only part of the T-epitope (i.e., the T-epitope may additionally encompass other parts of the glycolipopeptide such as the carbohydrate component, the lipid component, and/or the linker component). Thus, a peptide or peptide component that “comprises” a T-epitope is to be understood to mean a peptide or peptide component that encompasses all or part of a T-epitope that is present on the glycolipopeptide.

A peptide component may contain, for example, fewer than about 50 amino acids and/or amino acid analogs, fewer than about 40 amino acids and/or amino acid analogs, fewer than about 30 amino acids and/or amino acid analogs, or fewer than about 20 amino acids and/or amino acid analogs. A peptide component may contain, for example, about 9 to about 50 amino acids and/or amino acid analogs, about 9 to about 40 amino acids and/or amino acid analogs, about 9 to about 30 amino acids and/or amino acid analogs, or about 9 to about 20 amino acids and/or amino acid analogs. A peptide component may contain, for example, about 9, about 10, about 11, about 12, about 13, about 14, about 15, about 16, about 17, about 18, about 19, about 20, about 21, about 22, about 23, about 24, about 25, about 30, about 35, about 40, about 45, about 50, about 55, about 60, about 65, about 70, or about 80 amino acids and/or amino acid analogs, or any range of these cited sizes.

Examples of peptide components include the universal helper T peptide, QYIKANSKFIGITEL (“QYI”) (SEQ ID NO:1), the universal helper T peptide YAFKYARHANVGRNAFELFL (“YAF”) (SEQ ID NO:2), the murine helper T peptide KLFAVWKITYKDT (“KLF”) (SEQ ID NO:3) derived from polio virus, and pan-DR binding (PADRE) peptides (PCT WO 95/07707; Alexander et al., Immunity 1:751-761 (1994); Alexander et al., J. Immunol. 2000 Feb. 1; 164(3):1625-33; U.S. Pat. No. 6,413,935 (Sette et al., Jul. 2, 2002)).

Immunogenic peptide components for use in the glycolipopeptide of the invention include universal (degenerate or “promiscuous”) helper T-cell peptides, which are peptides that are immunogenic in individuals of many major histocompatibility complex (MHC) haplotypes. Numerous universal helper T-cell peptide structures are known; however, it should be understood that additional universal T-epitopes, including some with similar or even higher potency, will be identified in the future, and such peptides are well-suited for use as the peptide component the glycolipopeptide of the invention.

Exemplary T-cell peptides for use in the glycolipopeptide include, without limitation:

Synthetic, nonnatural PADRE peptide, DAla-Lys-Cha-Val-Ala-Ala-Trp-Thr-Leu-Lys-Ala-Ala-DAla, including all the analogues described by J Alexander et al. in Immunity, Vol. 1, 751-761, 1994;

Peptides derived from tetanus toxin, e.g., (TT830-843) QYIKANSKFIGITEL (SEQ ED NO:1), (TT1084-1099) VSIDKFRIFCKANPK (SEQ ID NO:4), (TT1174-1189) LKFIIKRYIPNNEIDS (SEQ ID NO:5), (TT1064-1079) IREDNNITLKLDRCNN (SEQ ID NO:6), and (TT947-967) FNNFTVSFWLRVPKVSASHLE (SEQ ID NO:7);

Peptides derived from polio virus, e.g., KLFAVWKITYKDT (SEQ ID NO:3);

Peptides derived from Neisseria meningitidis, e.g., YAFKYARHANVGRNAFELFL (SEQ ID NO:8); and

Peptides derived from P. falsiparum CSP, e.g., EKKIAKMEKASSVFNVNN (SEQ ID NO:9).

The peptide component of the glycolipopeptide contains a T-epitope. A T-epitope is an epitope recognized by a T cell. The T-epitope can elicit a CD4+ response, thereby stimulating the production of helper T cells; and/or it can elicit a CD8+ response, thereby stimulating the production of cytotoxic lymphocytes. Preferably, the T-epitope is an epitope that stimulates the production of helper T cells (i.e., a helper T-cell epitope or Th-epitope), which in turn makes possible a humoral response to the B-epitope supplied by the carbohydrate component of the glycolipopeptide of the invention.

It should be understood that the glycolipopeptide of the invention can contain multiple T-epitopes, which may be the same or different. Further, T-epitopes may be present on the carbohydrate component and/or the lipid component (e.g., in embodiments that include glycopeptides and/or glycolipids as the carbohydrate and/or lipid components) in addition to, or in place of, the peptide component.

In some embodiments, the B-epitopes and the T-epitopes are homologous; that is, they are derived from the same organism. For example, in a glycolipopeptide suitable for use as a vaccine against a microbial pathogen, the T-epitope in addition to the B-epitope may be epitopes that are present in the microbial pathogen. In another embodiment, the B-epitopes and the T-epitopes are heterologous; that is, they are not derived from the same organism. For example, a glycolipopeptide suitable for use as an anti-cancer vaccine may have a B-cell epitope from a cancer cell, but a T-cell epitope from a bacterium or virus.

In preferred embodiments of the immunogenic vaccine of the present invention, a T-epitope or a B-epitope may be derived from the MUC1 polypeptide. In some embodiments, both the T-epitopes and the B-epitopes are derived from the MUC1 polypeptide. MUC1 (MUC1 in humans and Muc1 in nonhuman species) is a heavily glycosylated type I transmembrane protein expressed in epithelial cells lining various mucosal surfaces as well as hematopoietic cells. Human MUC1 is composed of a cytoplasmic signaling peptide, a 28 amino acid transmembrane domain and an ectodomain composed of a variable number tandem repeats of twenty amino acids. Each repeat contains 5 potential O-glycosylation sites. MUC1 is associated with several adenocarcinomas at the mucosal sites and is over-expressed in more than 90% of breast carcinomas and associated with ovarian, lung, colon, and pancreatic carcinomas. Tumor associated MUC1 is aberrantly glycosylated, producing truncated carbohydrate structures.

A MUC1 peptide sequence may include human or mouse MUC1 sequences. A MUC1 peptide sequence may include MUC1 tandem repeat sequences. Such a MUC1 tandem repeat sequence may contain both a B-epitope and a helper T epitope.

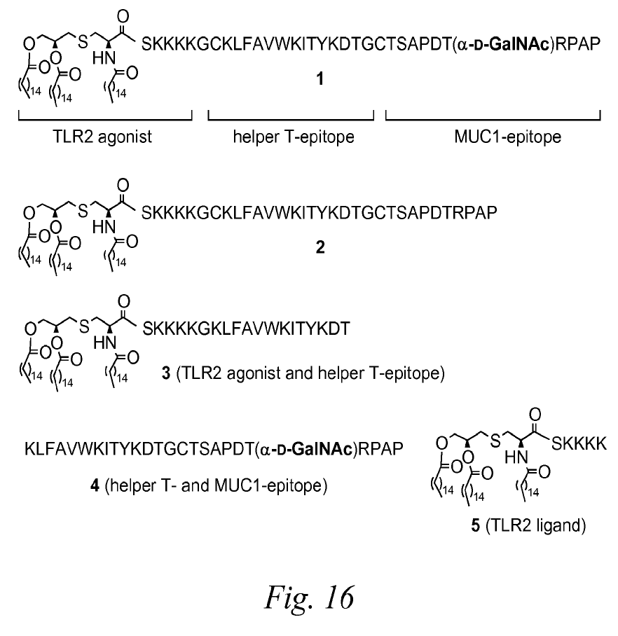

A MUC1 sequence may be homologous, thus a self-antigen. A MUC1 sequence may include one, two, three, four, five, six, or more amino acid changes from a human or mouse MUC1 peptide. A MUC1 sequence may be heteroclitic, including one, two, three, four, or more amino acid changes to enhance binding of the MUC1 peptide at a class I and/or class H major histocompatibility complex (MHC) protein. The human MHC is also referred to herein as the HLA complex. A MUC1 sequence may include sequences from the extracellular region of the MUC1 protein. A MUC1 sequence may include sequences that are responsible for class I MHC restriction. A MUC1 sequence may include sequences that are responsible for class II MHC restriction and/or binding. In some embodiments, such class I and class II restricted sequences may be a contiguous amino acid sequence in the immunogenic vaccine construct. MHC restricted sequences include, but are not limited to, any of those described herein, such as, for example, and any of those represented in FIGS. 16. 19, and 32 to 34.

A MUC1 sequence may include one or more serine or threonine residues that are glycosylated, for example, glycosylated at one, two, three, four, or more such residues. Such glycosylation may represent the glycosylation pattern of normal tissue or such glycosylation may reflect aberrant glycosylation. A MUC1 sequence may contain one or more B-epitopes and/or helper T epitopes.

A MUC1 sequence may include about 5 to about 30 amino acids of a MUC1 protein sequence. A MUC1 sequence may include fewer than about 50 amino acids and/or amino acid analogs, fewer than about 40 amino acids and/or amino acid analogs, fewer than about 30 amino acids and/or amino acid analogs, or fewer than about 20 amino acids and/or amino acid analogs of a MUC1 protein sequence. A MUC1 sequence may include, for example, about 9 to about 50 amino acids and/or amino acid analogs, about 9 to about 40 amino acids and/or amino acid analogs, about 9 to about 30 amino acids and/or amino acid analogs, or about 9 to about 20 amino acids and/or amino acid analogs. A peptide component may contain, for example, about 9, about 10, about 11, about 12, about 13, about 14, about 15, about 16, about 17, about 18, about 19, about 20, about 21, about 22, about 23, about 24, about 25, about 30, about 35, about 40, about 45, about 50, about 55, about 60, about 65, about 70, or about 80 amino acids and/or amino acid analogs, acids of a MUC1 protein sequence, or any range of these cited sizes.

A MUC1 sequence may include a sequence demonstrating about 50%, about 55%, about 60%, about 65%, about 70%, about 75%, about 80%, about 85%, about 90%, about 95%, or about 96%, about 97%, about 98%, or about 99% sequence identity to a human MUC1 sequence.

A MUC1 sequence may include any of the MUC1 sequence described herein, for example, including, but not limited to, any of those represented in FIGS. 16, 19, 33A, 33B, and 34. For example, a MUC1 sequence may include SAPDTRPAP (SEQ ID NO:20), TSAPDTRPAP (SEQ ID NO:21), SAPDTRPL (SEQ ID NO:22, TSAPDTRPL (SEQ ID NO:23, APGSTAPPAHGVTSA (SEQ ID NO:26), APGSTAPPAHGVTSAPDTRPL (SEQ ID NO:27), APGSTAPPAHGVTSAPDTRPL (SEQ ID NO:28), or APGSTAPPAHGVTSAPDTRPL (SEQ ID NO:29), SKKKKGAPGSTAPPAHGVTSAPDTRPX (SEQ ID NO:30) wherein X is L, A, or AP, SKKKKGSTAPPAHGVTSAPDTRPAP (SEQ ID NO:31), SKKKKGSLSYTNPAVAAATASNL (SEQ ID NO:32), SKKKKGCKLFAVWKITYKDTGTSAPDTRPAP (SEQ ID NO:33), SKKKKGCKLFAVWKITYKDT (SEQ ID NO:34), GGKLFAVWKITYKDTGTSAPDTRPAP (SEQ ID NO:35) or APGSTAPPAHGVTSAPDTRPAP (SEQ ID NO:28). Also included are MUC1 sequences that have about 50%, about 55%, about 60%, about 65%, about 70%, about 75%, about 80%, about 85%, about 90%, about 95%, or about 96%, about 97%, about 98%, or about 99% sequence identity to these sequences. Also included are MUC1 sequences that are glycosylated at any combination of one, two, three, four, or more serine or threonine residues.

### Lipid Component

It was originally postulated that a glycopeptide having just two main components, i.e., a carbohydrate component and a peptide component, would be effective to elicit an immune response in an animal. The helper T-cell epitope was expected to induce a T-cell dependent immune response, resulting in the production of IgG antibodies against a tumor-related carbohydrate B-epitope such as Le^{y} and Tn. However, in some applications, the two component vaccine was not found to be very effective. It was postulated that the B-cell and helper T-cell epitopes lack the ability to provide appropriate “danger signals” for dendritic cell (DC) maturation. To remedy this problem, a lipid component was included in the compound, resulting in the glycolipopeptide of the invention.

The lipid component can be any lipid-containing component, such as a lipopeptide, fatty acid, phospholipid, steroid, or a lipidated amino acids and glycolipids such as Lipid A derivatives. Preferably, the lipid component is non-antigenic; that is, it does not elicit antibodies directed against specific regions of the lipid component. However, the lipid component may and preferably does serve as an immunoadjuvant. The lipid component can serve as a carrier or delivery system for the multi-epitopic glycolipopeptide. It assists with incorporation of the glycolipopeptide into a vesicle or liposome to facilitate delivery of the glycolipopeptide to a target cell, and it enhances uptake by target cells, such as dendritic cells. Further, the lipid component stimulates the production of cytokines.

One class of preferred lipid components for use in the glycolipopeptide of the invention comprises molecular ligands of the various Toll-like receptors (TLRs). There are many known subclasses of Toll-like receptors (e.g., TLR1, TLR2, TRL3, TLR4, TLR5, TLR6, TLR7, TLR8, TLR9, TLR10, TLR11, TLR12, TLR13, TLR14, TLR15 and TLR16). See Roach et al., PNAS 2005, 102:9577-9582, for a review of the relationships between and evolution of Toll-like receptors; and Duin et al., TRENDS Immunol., 2006, 27:49-55, for a discussion of TLR signaling in vaccination.

TLRs are a family of pattern recognition receptors that are activated by specific components of microbes and certain host molecules. They constitute the first line of defense against many pathogens and play a crucial role in the function of the innate immune system. TLRs in mammals were first identified in 1997 and it has been estimated that most mammalian species have between ten and fifteen types of Toll-like receptors. Known TLRs include: TLR1 (TLR1 ligands include triacyl lipoproteins); TLR2 (TLR2 ligands include lipoproteins, gram positive peptidoglycan, lipoteichoic acids, fungi, and viral glycoproteins); TLR3 (TLR3 ligands include double-stranded RNA, as found in certain viruses, and poly I:C); TLR4 (TLR4 ligands include lipopolysaccharide and viral glycoproteins); TLR5 (TLR5 ligands include flagellin); TLR6 (TLR6 ligands include diacyl lipoproteins); TLR7 (TLR7 ligands include small synthetic immune modifiers (such as imiquimod, R-848, loxoribine, and bropirimine) and single-stranded RNA); TLR8 (TLR8 ligands include small synthetic compounds and single-stranded RNA); and TLR9 (TLR9 ligands include unmethylated CpG DNA motifs). See, for example, reviews by Akira, “Mammalian Toll-like receptors,” Curr Opin Immunol 2003; 15(1): 5-11 and Akira and Hemmi, “Recognition of pathogen-associated molecular patterns by TLR family,” Immunol Lett 2003; 85(2): 85-95.

Particularly preferred are lipid components that interact with TLR2 and TLR4. TLR2 is involved in the recognition of a wide array of microbial molecules from Gram-positive and Gram-negative bacteria, as well as mycoplasma and yeast. TLR2 ligands include lipoglycans, lipopolysaccharides, lipoteichoic acids and peptidoglycans. TLR4 recognizes Gram-negative lipopolysaccharide (LPS) and lipid A, its toxic moiety. TLR ligands are widely available commercially, for example from Apotech and InvivoGen. Preferably, the lipid component is a TLR ligand that facilitates uptake of the glycolipopeptide by antigen presenting cells (see Example 3).

Suitable lipids for use as the lipid component of the glycolipopeptide of the invention include PamCys-type lipid structures, such as those derived from Pam₃Cys (S—[(R)-2,3-dipalmitoyloxy-propyl]-N-palmitoyl-(R) cysteine) and Pam₂Cys (S—[(R)-2,3-dipalmitoyloxy-propyl]-(R)-cysteine), which lacks the N-palmitoyl group of Pam₃Cys. Pam₃Cys and Pam₂Cys are derived from the immunologically active N-terminal sequence of the principal lipoprotein of Escherichia coli. This class of lipids also includes Pam₃CysSK₄ (N-palmitoyl-S—[(R)-2,3-bis(palmitoyloxy)-propyl]-(R)-cysteinyl-(S)-seryl-(S)-lysine-(S)-lysine-(S)-lysine-(S)-lysyne) and Pam₂CysSK₄ (S—[(R)-2,3-bis(palmitoyloxy)-propyl]-(R)-cysteinyl-(S)-seryl-(S)-lysine-(S)-lysine-(S)-lysine-(S)-lysyne), which lacks the N-palmitoyl group of Pam₃CysSK4; it should be understood that the number of lysines in these structures can be 0, 1, 2, 3, 4, 5 or more (i.e., Kₙ where n=0, 1, 2, 3, 4, 5 or more). In some embodiments, a lipid component includes one or more lipid chains, one or more cysteine residues and one or more lysine residues.

Another preferred class of lipids includes Lipid A (LpA) type lipids, such as Lipid As derived from E. coli, S. typhimurium and Neisseria meningitidis. The Lipid As can be attached to the carbohydrate component (containing a B-epitope) of the glycolipopeptide and/or to the peptide component (containing a T-epitope) through a linker that is connected, for example, to the anomeric center or anomeric phosphate, the C-4′ phosphate or the C-6′ position. The phosphates can be modified, for example, to include one or more phosphate ethanolamine diesters. Exemplary Lipid A derivatives are described in, for example, Caroff et al., 2002, Microbes Infect; 4:915-926; Raetz et al., 2002, Annu Rev Biochem; 71:635-700; and Dixon et al., 2005, J Dent Res; 84: 584-595.

In some embodiments, the lipid component is a lipidated amino acid. In some embodiments, the lipid aspect of the lipid TLR2 agonist component is substituted with a different class of adjuvant compound, such as, for example, a TLR4 agonist, a TLR7 agonist, a TLR8 agonist, or a TLR9 agonist. In some embodiments, the agonist is the TLR9 agonist CpG.

Below, in Scheme 8, are exemplary immunogenic lipids for the incorporation into the glycolipopeptide of the invention. The first structure in the first row is Pam₃CysSKₙ; the second structure in the first row is Pam₂CysSKₙ; and the last 4 structures are Lipid A derivatives.

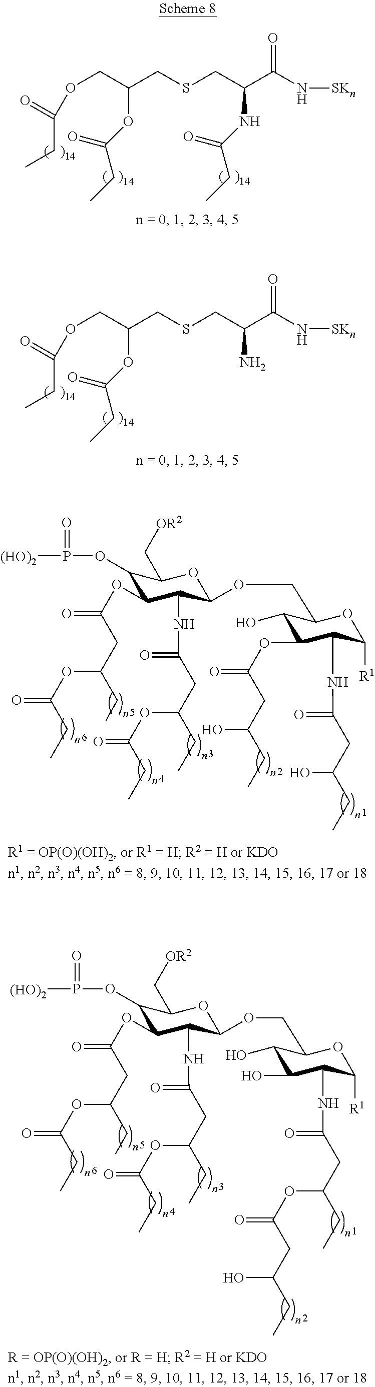

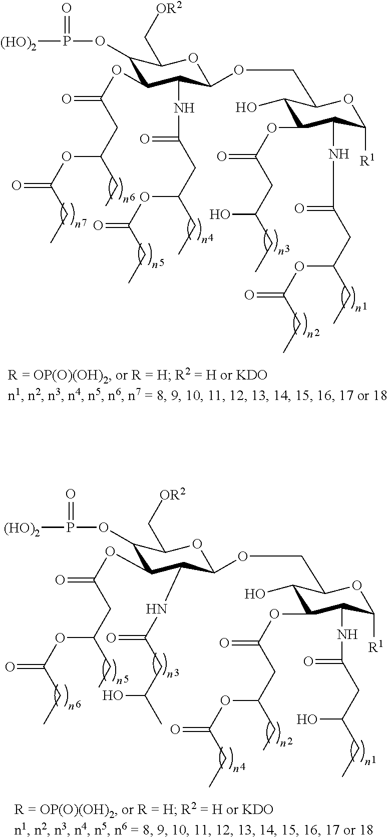

Lipids that are structurally based on Pam₃Cys are particularly preferred for use as the lipid component. Pam₃Cys is derived from the immunologically active N-terminal sequence of the principal lipoprotein of Escherichia coli. These lipopeptides are powerful immunoadjuvants. Recent studies have shown that Pam₃Cys exerts its activity through the interaction with Toll-like receptor-2 (TLR2).

Without being bound by theory, it is believed that interaction between the lipid component and a TLR results in the production of pro-inflammatory cytokines and chemokines, which, in turn, stimulates antigen-presenting cells (APCs), and thus, initiating helper T cell development and activation. Covalent attachment of the TLR ligand to the B- and T-epitopes ensures that cytokines are produced at the site where the vaccine interacts with immune cells. This leads to a high local concentration of cytokines facilitating maturation of relevant immune cells. The lipopeptide promotes selective targeting and uptake by antigen presenting cells and B-lymphocytes. Additionally, the lipopeptide facilitates the incorporation of the glycolipopeptide into liposomes. Liposomes have attracted interest as vectors in vaccine design due to their low intrinsic immunogenicity, thus, avoiding undesirable carrier-induced immune responses.

An immunogenic vaccine of the invention can be synthesized, for example, by chemoselective ligation, more particularly native chemical ligation (NCL), as described in WO 2007/146070 and US Patent Publication 2009/0196916A1. Briefly, one or more individual components of the vaccine are embedded or solubilized within a lipidic structure such as a lipid monolayer, lipid bilayer, a liposome, a micelle, a film, an emulsion, matrix, or a gel. The reactants used in the ligation reaction can include a carbohydrate component, a peptide component, a lipid component, or conjugates or combinations thereof. These reactants are designed or selected to include desired antigenic or immunogenic features, such as T-epitopes or B-epitopes of the immunogenic vaccine of the invention

### Optional Linker

One or more linkers (“L”) are optionally used for assembly of the three components of the glycolipopeptide. In one embodiment, the linker is a bifunctional linker that has functional groups in two different places, preferably at a first and second end, in order to covalently link two of the three components together. A bifunctional linker can be either homofunctional (i.e., containing two identical functional groups) or heterofunctional (i.e., containing two different functional groups). In another embodiment, the linker is trifunctional (hetero- or homo-) and can link all three components of the glycolipopeptide together. A suitable functional group has reactivity toward or comprises any of the following: amino, alcohol, carboxylic acid, sulfhydryl, alkene, alkyne, azide, thioester, ketone, aldehyde, or hydrazine. An amino acid, e.g., cysteine, can constitute a linker.

Bifunctional linkers are exemplified in Scheme 9.

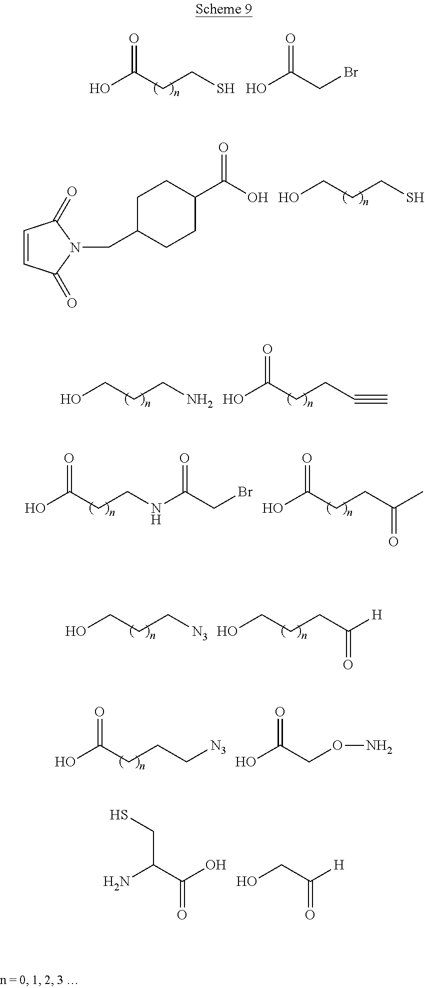

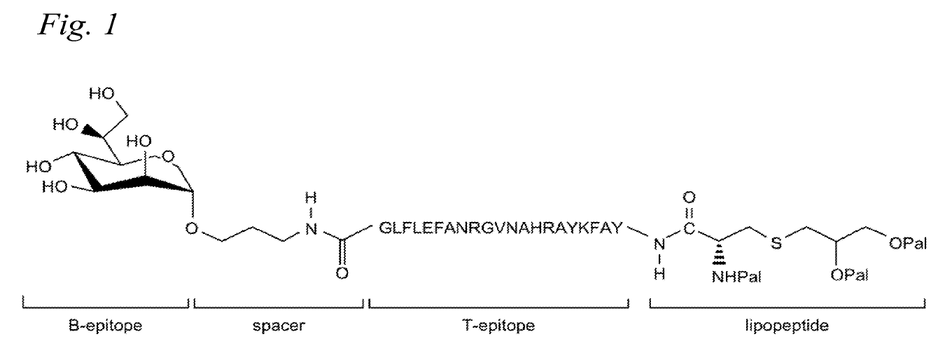

FIG. 1 shows an exemplary fully synthetic glycolipopeptide of the invention containing a carbohydrate-based B-epitope, a peptide T-epitope and a lipopeptide. The compound shown in FIG. 1 contains a L-glycero-D-manno-heptose sugar that acts as a B-epitope, the peptide sequence YAFKYARHANVGRNAFELFL (SEQ ID NO:2) that has been identified as a MI-IC class II restricted recognition site for human T-cells and is derived from an outer-membrane protein of Neisseria meningitidis, and the lipopeptide S-2-3[dipalmitoyloxy]-(R/S)-propyl-N-palmitoyl-R-Cysteine (Pam₃Cys). As noted elsewhere herein, lipopeptide Pam₃Cys and the related compound Pam₃CysSK₄ are highly potent B-cell and macrophage activators.

Methods of making the glycolipopeptide, as exemplified in the Examples, are also encompassed by the invention. Preferably, the method for making the glycolipopeptide utilizes chemical synthesis, resulting in a fully synthetic glycolipopeptide. In embodiments that make use of one or more linkers, the optional linker component is functionalized so as to facilitate covalent linkage of one of the main components to another of the main components. For example, the linker can be functionalized at each end with a thiol-reactive group, such as maleimide or bromoacetyl, and the components to be joined are modified to include reactive thiols. Other options for ligation chemistry include Native Chemical Ligation, the Staudinger Ligation and Huisgen ligation (also known as “Click Chemistry”). Example 2 illustrates how the carbohydrate component, in that case an oligosaccharide, and the peptide component can be functionalized with a thiol-containing linker. Preferably, the linker component, if used, is nonantigenic.

The glycolipopeptide of the invention is capable of generating an immune response in a mammal. The glycolipopeptide is antigenic, in that it can generate a humoral response, resulting in the activation of B cells and production of antibodies (immunoglobulins) such as IgM. Additionally, the glycolipopeptide is immunogenic, in that it can generate a cellular response; for example, it facilitates the activation of T cells, particularly helper T cells which are also instrumental in the generation of a more complex antibody response that includes the production of IgG. Ultimately, the immune response elicited in the animal includes the production of anti-carbohydrate antibodies.

In another embodiment of the present invention, the immunogenic vaccine is a two component vaccine comprising, covalently linked, at least one peptide component and at least one adjuvant component. The peptide component includes a T epitope, preferably a helper T epitope of MUC1 origin, including, but not limited to, any of those described herein. While this embodiment of the vaccine may not generate specific immunity against a particular B epitope, it exhibits antitumor properties. An example of a two component vaccine is Pam3CysSK4 covalently linked to a helper T epitope; see, for example, compound 3 in Example 8. In one embodiment, the adjuvant component of the immunogenic vaccine comprises a toll-like receptor (TLR) ligand. At least 15 different mammalian TLRs are known (e.g., TLR1, TLR2, TLR3, TLR4, TLR5, TLR6, TLR7, TLR8, TLR9, TLR10, TLR11, TLR12, TLR13, TLR14 and TLR15) and their ligands exhibit significant structural variation. Some TLR ligands are described herein, but it should be understood that such listings do not limit the invention in any way. In some embodiments, a two component immunogenic vaccine may be formulated for administration as a composition that further includes additional agents, such as, for example, immune modulators, adjuvants, TLR agonists and/or excipients. TLR ligands are well-known to the skilled artworker. They can be take the form of lipopeptides, glycolipids, lipoproteins, carbohydrates, small organic molecules, nucleic acids such as single or double stranded DNA or RNA, and many are known to function as immunostimulants. One example of an immunostimulatory TLR ligand is a TLR2 ligand, including, but not limited to, any of those described herein. Another example is a TLR9 ligand commonly referred to as “CpG.” This compound is an immunostimulatory oligodeoxynucleotide (ODN) containing a CpG motif. CpG motifs are recognized as a ligand by TLR9 (Rothenfusser et al., 2002, Human immunology 63 (12): 1111-1119). Preferably, CpG ODN is unmethylated. CpG ODNs are short, single stranded, DNA molecules that contain a cytosine (“C” nucleotide) followed by a guanine (“G” nucleotide). The “p” typically refers to the phosphodiester backbone of DNA. Optionally, the CpG motifs may be modified to contain a phosphorothioate (PS) backbone in order to protect the ODN from being degraded by nucleases such as DNAse (Dalpke et al., 2002, Immunology 106(1):102-12). CpG ODNs typically range in length from about 18 nucleotides to about 28 nucleotides in length. Optionally they contain a palindromic sequence. One example of a CpG for use in the invention is 5′-TCCATGACGTTCCTGACGTT-3′ (SEQ ID NO:36). CpG motifs present in vertebrate DNA are frequently methylated as a mechanism of transcriptional regulation (Sulewska et al., 2007, Folia Histochemica et Cytobiologica 45(3):149-158). Unmethylated CpG motifs have been shown to act as immunostimulants (Weiner et al., 1997, Proc. Natl. Acad. Sci. USA, 94:10833-10837). CpG has been used in studies to enhance tumor immunity (Nierkens et al., 2009, PLoS One. 4(12):e8368; Cooper et al., 2004, J. Clin. Immunol. 24(6):693-701; Leichman et al., 2005, J. Clin. Oncol. 2005 ASCO Annual Meeting Proceedings. 23(16S):7039).

A number of CpG ODNs are commercially available. For example, CPG ODNs can be purchased through InvivoGen (San Diego, Calif.) as a Type A, Type B, or Type C molecule. These classes are based on both structural differences and in their immunostimulatory activities (Krug et al., 2001. Eur J Immunol, 31(7): 2154-63; Marshall et al., 2005 DNA Cell Biol. 24(2):63-72; Martinson et al., 2006, Immunology 120:526-535).

In another embodiment, the adjuvant component of the immunogenic vaccine is a lipid component, as described herein (see also WO 2007/079448, US Patent Publication 2009/0041836 A1, and WO 2010/002478). Some TLR ligands, such as the ligand for TLR2, also constitute lipid components, but the lipid component of the immunogenic vaccine is not limited to a TLR ligand; i.e., the lipid component can be any suitable immunogenic or antigenic lipid that can act as an adjuvant, such as, for example, lipidated amino acid (“LAA”).

In another aspect, the glycolipopeptide of the invention is used to produce a polyclonal or monoclonal antibody that recognizes either or both of the carbohydrate component and the peptide component. The invention encompasses the method of making said antibodies, as well as the antibodies themselves and hybridomas that produce monoclonal antibodies of the invention.

The immunogenic glycolipopeptide of the invention for use in the production an antibody can contain any carbohydrate component described herein, without limitation. Preferably it contains, as its carbohydrate component, a glycopeptide. The glycopeptide includes a glycosylated peptide sequence that includes a carbohydrate moiety, such as a saccharide. The saccharide can be a monosaccharide, an oligosaccharide or a polysaccharide. Preferably, the carbohydrate component of the glycolipopeptide used to generate the antibodies contains a self-antigen as described above. Advantageously, even if carbohydrate component, e.g., the glycopeptide, is poorly antigenic (such as a self-antigen), covalent attachment of the carbohydrate component to the peptide component and the lipid component produces a remarkably immunogenic glycolipopeptide.

Antibodies of the invention that bind to the glycolipopeptide preferably bind to a B-epitope that includes the saccharide moiety and, in a preferred embodiment, at least part of the peptide that forms the glycopeptide. A preferred antibody binds to the glycopeptide used as the carbohydrate component, but does not bind to the deglycosylated peptide or to the saccharide residue alone.

When used to generate antibodies, the glycolipopeptide of the invention successfully generates high affinity IgG antibodies. This is especially surprising and unexpected for embodiments of the glycolipopeptide having a poorly antigenic carbohydrate component, such as a self-antigen. The polyclonal or monoclonal antibody is thus preferably an IgG isotype antibody. Without being bound by theory, it is believed that the glycolipopeptide of the invention is a superior antigen (compared to the non-lipidated glycopeptide) because it stimulates local production of cytokines, upregulates co-stimulatory proteins, enhances uptake by macrophages and dendritic cells and/or avoids epitope suppression.

Antibodies of the invention include but are not limited to those that recognize B-epitopes that contain O-GlcNAc, O-GalNAc, O-mannose, or other saccharide modifications. Other B-epitopes that may be recognized by the antibodies of the invention include those that contain fragments of glycosaminoglycans such as heparin, heparan sulfate, chondroitin sulfate, dermatan sulfate, keratan sulfate, hyaluronan, and generally any glycosaminoglycan. In the case of a glycosaminoglycan formed by repeating disaccharide units, the B-epitope may contain one or more disaccharide unit. B-epitopes recognized by the antibodies of the invention may contain pentose, hexose or other sugar moieties including acids, including but not limited to glucuronic acid, iduronic acid, hyaluronic acid, glucose, galactose, galactosamine, glucosamine and the like. The antibodies of the invention are preferably produced using, as an immunogen, the glycolipopeptide of the invention wherein the carbohydrate component contains the B-epitope of interest. Analogues of naturally occurring B-epitopes, such as those containing N-linked or S-linked structures or glycomimetics, can be used as the carbohydrate component, for example to make the glycolipopeptide immunogen more metabolically stable.

The antibodies produced using the glycolipopeptide of the invention advantageously include high affinity IgG antibodies that recognize a broad spectrum of glycoproteins. Thus, even though antibodies produced using the glycolipopeptide of the invention as an immunogen are specific for the glycopeptide used as the carbohydrate component, they may bind to a broad spectrum of glycoproteins. An antibody with relatively broad selectivity for glycosylated peptides or proteins containing a B-epitope component of interest is referred to herein as a “pan-specific” antibody. A polyclonal or monoclonal antibody of the invention may be either pan-specific or site-specific. An antibody that is pan-specific, as the term is used herein, is one that specifically recognizes a selected B-epitope, for example a B-epitope that contains O-GlcNAc, but that has a relatively broad selectivity for proteins and peptides containing the B-epitope. A pan-specific antibody is thus able to bind multiple different glycosylated proteins or peptides that contain the B-epitope of interest, although it does not necessarily bind all glycosylated proteins or peptides that contain the selected B-epitope.

Without intending to be being bound by theory, the different glycoproteins recognized by the pan-specific antibodies of the invention may share a substantially similar or identical (glyco)peptide sequence (i.e., primary sequence) or a substantially similar secondary or tertiary structure at the glycosylation site, thereby resulting in a broad spectrum of binding targets being recognized by the antibody. A secondary or tertiary epitope structure shared by the O-GlcNAc modified glycoproteins to which an antibody binds may advantageously be maintained in the glycolipopeptide immunogen, as evidenced by the successful production of IgG antibodies that recognize the broad spectrum of glycoproteins.

Preferably, the antibody of the invention binds to a plurality of glycosylated proteins or peptides having an epitope comprising O-GlcNAc, O-GalNAc, or other saccharide modifications, but does not detectably bind a protein or peptide that does not contain the saccharide. More preferably, the antibody binds to a protein or peptide having an epitope comprising O-GlcNAc, O-GalNAc, or other saccharide modifications, but does not detectably bind the same protein or peptide that does not contain O-GlcNAc, O-GalNAc, or other saccharide modifications.

An example of a preferred polyclonal or monoclonal antibody is one that binds to a glycopeptide that contains an O-GlcNAc monosaccharide residue. In a particularly preferred embodiment, the antibody has a relatively broad selectivity for O-GlcNAc modified proteins. For example, many proteins of interest have a TPVSS (SEQ ID NO:10) sequence modified by O-GlcNAc, and a preferred monoclonal antibody recognizes this and/or similar glycosylated peptide sequences. Examples of preferred monoclonal antibodies specific for O-GlcNAc modified sequences include the monoclonal antibodies produced by hybridoma cell lines 1F5.D6, 9D1.E4, 18B10.C7 and 5H11.H6. These monoclonal antibodies were produced using compounds 52 and/or 53 as an immunogen. Thus, in one embodiment, the antibody of the invention binds to the carbohydrate component of compound 52 or of compound 53. Hybridoma cell lines 1F5.D6, 9D1.E4 and 18B10.C7 were deposited with the American Type Culture Collection (ATCC), 10801 University Blvd., Manassas, Va., 20110-2209, USA, on Jul. 1, 2008, and assigned ATCC deposit numbers PTA-9339, PTA-9340, and PTA-9341, respectively. The invention encompasses the hybridoma cell lines as well as the monoclonal antibodies they produce.

Another example of a preferred polyclonal or monoclonal antibody is one that binds to a heparan sulfate fragment.

It is to be understood that any carbohydrate or glycopeptide of clinical significance or interest can be incorporated as the carbohydrate and/or peptide component of the glycolipopeptide of the invention and used to generate polyclonal and monoclonal antibodies according to the method of the invention. Such carbohydrates and peptides include those of medical and veterinary interest, as well as those with other commercial or research applications. It should be understood that the monoclonal and polyclonal antibodies of the invention are not limited to those that recognize any particular ligand but include, without limitation and by way of example only, antibodies against any type of tumor associated carbohydrate antigen (TACA) and against any saccharides derived from any microorganism.

To recapitulate, use of the glycolipopeptide of the invention to make monoclonal antibody of the invention is surprisingly effective in producing monoclonal IgG antibodies having high affinity for their carbohydrate or glycopeptide antigen, even when the antigens are poorly antigenic. This opens the door for the creation of antibodies useful to study, diagnose and treat immune-related diseases or diseases having autoimmune or inflammatory components including cancer, diabetes type II, allergies, asthma, Crohn's disease, Alzheimer's disease, muscular dystrophy, microbial infections and the like. Monoclonal antibodies of the invention that recognize O-GlcNAc-modified glycoproteins, for example, are far superior to commercially available antibodies such CTD110.6 (Covance Research Products, Inc.). The glycolipopeptide of the invention can be assembled using a modular synthesis, wherein the lipid, peptide and carbohydrate component are selected according to the desired application. Moreover, the glycolipopeptide of the invention is a remarkably effective antigen for use in producing pan-specific antibodies, particularly pan-specific monoclonal IgG antibodies that recognize glycosylated peptides and proteins that contain an O-linked monosaccharide such as O-GlcNAc.

The antibodies of the invention and those created by the method of the invention are important research tools for the identification and characterization of proteins, peptides and other biomolecules associated with various disease states. For example, the pan-specific antibodies of the invention can be used to pull down glycoproteins from complex biological samples. This method can be used to detect and identify proteins not heretofore known to be identified with a particular disorder or disease state, thereby identifying potential therapeutic or diagnostic targets. In one embodiment, an antibody of the invention can be contacted with a biological sample under conditions that enable the antibody to bind to a plurality of glycosylated proteins or glycosylated peptides and detecting antibody-protein binding. Optionally the method may include isolating the glycosylated proteins or glycosylated peptides. The method may further include identifying one or more of the proteins or peptides within the plurality of glycosylated proteins or glycosylated peptides. The identification of glycosylated proteins and peptides may provide an opportunity to explore the role of glycosylation and its biological implications in various biological processes. For example, glycosylation of proteins or peptides may be involved in a number of biological processes including, but not limited to, transcription, translation, signal transduction, the ubiquitin pathway, anterograde trafficking of intracellular vesicles and post-translational modifications (e.g. SUMOylation and phosphorylation). Methods for identifying a protein or peptide are well known in the art and may include, without limitation, techniques such as mass spectrometry and Edman degradation.

The pan-specific antibody of the invention may also be used to identify proteins or peptides having altered glycosylation in a disease state. O-GlcNAc modifications are associated with a variety of disease states. For example, an increase of O-GlcNAc modifications in skeletal muscle and pancreas glycopeptides correlates with development of Type II Diabetes while a reduction in O-GlcNAc modifications in neural glycopeptides correlates with the onset of Alzheimer's disease (Dias and Hart; Mol. BioSyst. 3:766-772 (2007)). Therefore, detection of changes in the levels of O-GlcNAc modifications may be used as a diagnostic or prognostic tool. Additionally, the glycosylation state of such proteins or peptides may be correlated with disease state. A method for identifying proteins or peptides having altered glycosylation that is correlated with disease state includes incubating an antibody of the present invention with a first biological sample of a known disease state and incubating the antibody with a second biological sample of a non-diseased state under conditions enabling the antibody to bind to a plurality of glycosylated proteins and peptides within the first sample and to a plurality of glycosylated proteins and peptides within the second sample, independently isolating the glycosylated proteins and glycosylated peptides from the samples, and identifying the glycosylated proteins and glycosylated peptides. The method may further include comparing the identified glycosylated proteins and glycosylated peptides in the first sample to the glycosylated proteins and glycosylated peptides in the second sample wherein a protein or peptide that demonstrates a change in glycosylation state between first and second samples is indicative of the glycosylated protein or a glycosylated peptide being associated with a disease state. Correlations between glycosylation and disease state include the disease state having increased or decreased glycosylation relative to the non-diseased state. In addition, the disease state may exhibit glycosylation while the non-disease state shows complete absence of glycosylation or conversely, the disease state may show complete absence of glycosylation while the non-disease exhibits the presence of glycosylation. In each example, the protein or peptide is considered to have differential or altered glycosylation in the disease state. Methods of using the antibody of the invention to detect the presence or overexpression glycosylation and to detect changes in the level of glycosylation have been previously described.

The antibodies of the invention are broadly useful in diagnostic or therapeutic applications as described in more detail elsewhere herein. Comparative analysis can be done on two or more different biological samples. For example, large scale immunoprecipitation can be performed on samples before and after a treatment intervention, or over time to monitor the progression of disease, or to compare normal samples with samples from patients suspected of suffering from a disease, infection or disorder characterized by changes in protein glycosylation.

In one embodiment, the present invention includes methods to diagnose the presence of a disease state in a subject. The method includes incubating a biological sample from the subject with an antibody of the present invention and detecting binding of the antibody to a protein or peptide having differential glycosylation in the disease state. Methods of detecting antibody binding have been previously described. In cases where glycosylation is completely absent in the disease state, a lack of binding of the antibody to the protein or peptide is indicative of subject having the disease state. In cases where glycosylation is present in the disease state but completely absent in the non-disease state, binding of the antibody to the protein or peptide is indicative of the presence of the disease state in the subject. Optionally, the method may further include incubating a second, non-diseased, biological sample with an antibody of the invention, detecting binding of the antibody to a protein or peptide, and comparing antibody binding in the first and second samples.

Additionally, for protein and peptides where glycosylation is present in both the disease state and the non-disease state, but is altered (i.e. increased or decreased) in the disease state, the method may further include quantitating the level of antibody binding in the first sample, quantitating the level of antibody binding in the second, non-diseased sample, and comparing the binding levels. A change in antibody binding in the first sample compared to the non-diseased sample is indicative of the presence of the infection, disease or disorder in the subject.

For preparation of an antibody of the present invention, any technique which provides for the production of antibody molecules by continuous cell lines in culture may be used. For example, the hybridoma technique originally developed by Kohler and Milstein (256 Nature 495-497 (1975)) may be used. See also Ausubel et al., Antibodies: a Laboratory Manual, (Harlow & Lane eds., Cold Spring Harbor Lab. 1988); Current Protocols in Immunology, (Colligan et al., eds., Greene Pub. Assoc. & Wiley Interscience N.Y., 1992-1996).

The present invention also provides for a hybridoma cell line that produces a monoclonal antibody, preferably one that has a high degree of specificity and affinity toward its antigen. The present invention further includes variants and mutants of the hybridoma cell lines. Such cell lines can be produced artificially using known methods and still have the characteristic properties of the starting material. For example, they may remain capable of producing the antibodies according to the invention or derivatives thereof, and secreting them into the surrounding medium. Optionally, the hybridoma cell lines may occur spontaneously. Clones and sub-clones of hybridoma cell lines are to be understood as being hybridomas that are produced from the starting clone by repeated cloning and that still have the main features of the starting clone.

Antibodies can be elicited in an animal host by immunization with the glycolipopeptide of the invention, or can be formed by in vitro immunization (sensitization) of immune cells. The antibodies can also be produced in recombinant systems in which the appropriate cell lines are transformed, transfected, infected or transduced with appropriate antibody-encoding DNA. Alternatively, the antibodies can be constructed by biochemical reconstitution of purified heavy and light chains.

Once an antibody molecule has been produced by an animal, chemically synthesized, or recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. In addition, the antibodies of the present invention or fragments thereof can be fused to heterologous polypeptide sequences known in the art to facilitate purification.

In a preferred embodiment, the monoclonal antibody recognizes and/or binds to an antigen present on the carbohydrate component or the peptide component of the glycolipopeptide of the invention. In a particularly preferred embodiment, the monoclonal antibody binds to an antigen present on a selected feature of the carbohydrate component. An example of a selected feature would include the modification on a glycopeptide such as O-GlcNAc. Other modifications include, but are not limited to, GalNAc and other saccharide modifications.

The term “antibody” is used in the broadest sense and specifically covers monoclonal antibodies (including full length monoclonal antibodies) and antibody fragments so long as they exhibit the desired biological activity. “Antibody fragments” comprise a portion of a full length antibody, generally the antigen binding or variable region thereof. Examples of antibody fragments include, but are not limited to Fab, Fab′, and Fv fragments; diabodies; linear antibodies; and single-chain antibody molecules. The term “monoclonal antibody” as used herein refers to antibodies that are highly specific, being directed against a single antigenic site. The term “antibody” as used herein also includes naturally occurring antibodies as well as non-naturally occurring antibodies, including, for example, single chain antibodies, chimeric, bifunctional and humanized antibodies, as well as antigen-binding fragments thereof. Such non-naturally occurring antibodies can be constructed using solid phase peptide synthesis, can be produced recombinantly or can be obtained, for example, by screening combinatorial libraries consisting of variable heavy chains and variable light chains as described by Huse et al. (Science 246:1275-1281 (1989)). These and other methods of making functional antibodies are well known to those skilled in the art (Winter and Harris, Immunol. Today 14:243-246 (1993); Ward et al., Nature 341:544-546 (1989); Harlow and Lane, supra, 1988); Hilyard et al., Protein Engineering: A practical approach (IRL Press 1992); Borrabeck, Antibody Engineering, 2d ed. (Oxford University Press 1995)).

In all mammalian species, antibody peptides contain constant (i.e., highly conserved) and variable regions, and, within the latter, there are the complementarity determining regions (CDRs) and the so-called “framework regions” made up of amino acid sequences within the variable region of the heavy or light chain but outside the CDRs. Preferably the monoclonal antibody of the present invention has been humanized. As used herein, the term “humanized” antibody refers to antibodies in which non-human (usually from a mouse or a rat) CDRs are transferred from heavy and light variable chains of the non-human immunoglobulin into a variable region designed to contain a number of amino acid residues found within the framework region in human IgG. Similar conversion of mouse/human chimeric antibodies to a humanized antibody has been described before. General techniques for cloning murine immunoglobulin variable domains are described, for example, by the publication of Orlandi et al., Proc. Nat'l Acad. Sci. USA 86: 3833 (1989), which is incorporated by reference in its entirety. Techniques for producing humanized MAbs are described, for example, by Jones et al., Nature 321: 522 (1986), Riechmann et al., Nature 332: 323 (1988), Verhoeyen et al., Science 239: 1534 (1988), and Singer et al., J. Immun. 150: 2844 (1993), each of which is hereby incorporated by reference.

Methods of using the monoclonal antibody that recognizes and/or binds to a component of the glycolipopeptide are also encompassed by the invention. Uses for the monoclonal antibody of the invention include, but are not limited to, diagnostic, therapeutic, and research uses. In a preferred embodiment, the monoclonal antibody can be used for diagnostic purposes. Because O-GlcNAc modifications are associated with a variety of disease states, detection of changes in the levels of O-GlcNAc modifications may be interpreted as early indicators of the onset of such diseases. For example, an increase in O-GlcNAc modifications in skeletal muscle and pancreas glycopeptides correlates with development of Type II Diabetes while a reduction in O-GlcNAc modifications in neural glycopeptides correlates with the onset of Alzheimer's disease (Dias and Hart, Mol. BioSyst. 3:766-772 (2007); Lefebvre et al., Exp. Rev. Proteomics 2(2):265-275 (2005)). Therefore, identifying an increase in the amount of O-GlcNAc in a sample of skeletal muscle tissue relative to a non-disease control sample may be indicative of development of Type II Diabetes.

It should be understood that the monoclonal and polyclonal antibodies of the invention are not limited to those that recognize any particular ligand but include, without limitation and by way of example only, antibodies against any type of tumor associated carbohydrate antigen (TACA) and against any saccharides derived from any microorganism. The antibodies of the invention are broadly useful in diagnostic or therapeutic applications.

Antibodies of the invention can be used to detect the presence or overexpression of a specific protein or a specific modification. Techniques for detection are known to the art and include but are not limited to Western blotting, dot blotting, immunoprecipitation, agglutination, ELISA assays, immunoELISA assays, tissue imaging, mass spectrometry, immunohistochemistry, and flow cytometry on a variety of tissues or bodily fluids, and a variety of sandwich assays. See, for example, U.S. Pat. No. 5,876,949, hereby incorporated by reference.

In order to detect changes in the level of O-GlcNAc modified glycopeptides, monoclonal antibodies of the invention may be labeled covalently or non-covalently with any of a number of known detectable labels, such as fluorescent, radioactive, or enzymatic substances, as is known in the art. Alternatively, a secondary antibody specific for the monoclonal antibody of the invention is labeled with a known detectable label and used to detect the O-GlcNAc-specific antibody in the above techniques.

Preferred detectable labels include chromogenic dyes. Among the most commonly used are 3-amino-9-ethylcarbazole (AEC) and 3,3′-diaminobenzidine tetrahydrochloride (DAB). These can be detected using light microscopy. Also preferred are fluorescent labels. Among the most commonly used fluorescent labeling compounds are fluorescein isothiocyanates (e.g. FITC and TRITC), Idotricarbocyanines (e.g. Cy5 and Cy7), rhodamine, phycoerythrin, phycocyanin, allophycocyanin, o-phthaldehyde and fluorescamine. Chemiluminescent and bioluminescent compounds such as luminol, isoluminol, theromatic acridinium ester, imidazole, acridinium salt, oxalate ester, luciferin, luciferase, and aequorin may also be used. When the fluorescent-labeled antibody is exposed to light of the proper wavelength, its presence can be detected due to its fluorescence. Also preferred are radioactive labels. Radioactive isotopes which are particularly useful for labeling the antibodies of the present invention include ³H, ¹²⁵I, ¹³¹I, ³⁵S, ³²P, and ¹⁴C. The radioactive isotope can be detected by such means as the use of a gamma counter, a scintillation counter, or by autoradiography. Enzymes which can be used to detectably label antibodies and which can be detected, for example, by spectrophotometric, fluorometric, or visual means include, but are not limited to, malate dehydrogenase, staphylococcal nuclease, delta-5-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase, and acetylcholinesterase. Other methods of labeling and detecting antibodies are known in the art and are within the scope of this invention.

A three component immunogenic vaccine of the present invention including a TLR agonist, a T-helper epitope, and a glycosylated MUC1 epitope (BIT-cell epitope) demonstrates many advantages. A glycosylated BIT cell epitope may be more effective than a non-glycosylated epitope. The vaccine elicits a strong cytolytic T cell response elicited, lysing cells expressing MUC1. The secretion of interferon gamma-often by both CD4+ and CD8+ T cells is indicative of the activations of a T cell cellular response. Further, the activation of a B cell response is indicated by Ig class switching and the generation of antibodies effective at inducing ADCC (antibody dependent cell-mediated cytotoxicity) of cells (both tumor cells and YAC cells) expressing MUC1. Thus, a MUC1-based three component immunogenic cancer vaccine dually elicits both a humoral and a cellular immune response, including antibody development, interferon gamma production, and cytolytic activity, yielding superior therapeutic outcomes. In some embodiments, the addition of a second TLR agonist further increase in effectiveness, for example, demonstrating decreased tumor burden, increased IFN-γ production, and increased T cell mediated cytotoxicity.

The present invention includes methods of generating antibody-dependent cell-mediated cytotoxicity (ADCC) in a subject by immunizing the subject with one or more of the immunogenic vaccine constructs described herein. In some aspects, the ADCC is natural killer (NK) cell mediated. In some aspects, the ADCC lyses tumor cells. In some aspects, the tumor cells are breast cancer cells or epithelial cancer cells. In some aspects, the ADCC lyses cells expressing a MUC1 peptide sequence. In some aspects, the MUC1 peptide is aberrantly glycosylated.

The present invention includes methods of treating cancer, reducing tumor burden, preventing tumor recurrence, and/or preventing cancer in a subject by immunizing the subject with one or more of the immunogenic vaccine constructs described herein. In some aspects of the methods of the present invention, the cancer or tumor is breast cancer or epithelial cancer. In some aspects of the methods of the present invention, the cancer or tumor expresses aberrantly glycosylated MUC1.

The present invention include methods of generating a cytotoxic T cell response directed at MUC1 expressing cells, generating anti-MUC1 antibodies, and/or promoting anti-MUC1 antibody class switching in a subject by immunizing the subject with one or more of the immunogenic vaccine constructs described herein. In some aspects, the MUC1 expressing cells are tumor cells. In some aspects of the methods of the present invention, the cancer or tumor expresses aberrantly glycosylated MUC1.

The present invention includes methods of immunizing the subject with a glycolipopeptide including at least one glycosylated MUC1 glycopeptide component including a B-cell epitope; at least one peptide component including a MHC class H restricted helper T-cell epitope; and at least one lipid component. In some aspects, antibodies of the IgG subtype that specifically bind to a MUC1 protein expressed on a tumor cell are induced in the subject. Because it is antigenic and immunogenic, the glycolipopeptide of the invention is well-suited for use in an immunotherapeutic pharmaceutical composition. The invention thus includes pharmaceutical compositions that include a glycolipopeptide of the invention as well as a pharmaceutically acceptable carrier. In a preferred embodiment, the pharmaceutical composition contains liposomes, for example phospholipid-based liposomes, and the glycolipopeptide is incorporated into liposomes as a result of noncovalent interactions such as hydrophobic interactions. Alternatively, the glycolipopeptide can be covalently linked to a component of the liposome. The liposome formulation can include glycolipopeptides that have the same or different B-epitopes; the same or different T-cell epitopes; and/or the same or different lipid components.

The three component immunogenic vaccine of the present invention has covalently linked, at least one carbohydrate component, at least one peptide component, and at least one adjuvant component. The three component immunogenic vaccine contains a B epitope and a T epitope, preferably a helper T epitope. Typically, the carbohydrate component includes a B epitope and the peptide component contains a T epitope. The B epitope may further include T epitopes. However, these epitopes may overlap, and a single glycopeptide, such as MUC-1 glycopeptide, may include both a B epitope and a T epitope.

The glycolipopeptide of the invention is readily formulated as a pharmaceutical composition for veterinary or human use. The pharmaceutical composition optionally includes excipients or diluents that are pharmaceutically acceptable as carriers and compatible with the glycolipopeptide. The term “pharmaceutically acceptable carrier” refers to a carrier(s) that is “acceptable” in the sense of being compatible with the other ingredients of a composition and not deleterious to the recipient thereof or to the glycolipopeptide. Suitable excipients include, for example, water, saline, dextrose, glycerol, ethanol, or the like and combinations thereof. In addition, if desired, the pharmaceutical composition may contain minor amounts of auxiliary substances such as wetting or emulsifying agents, pH buffering agents, salts, and/or adjuvants which enhance the effectiveness of the immune-stimulating composition. For oral administration, the glycolipopeptide can be mixed with proteins or oils of vegetable or animal origin. Methods of making and using such pharmaceutical compositions are also included in the invention.

The pharmaceutical composition of the invention can be administered to any subject including humans and domesticated animals (e.g., cats and dogs). In a preferred embodiment, the pharmaceutical composition is useful as a vaccine and contains an amount of glycolipopeptide effective to induce an immune response in a subject. Dosage amounts, schedules for vaccination and the like for the glycolipopeptide vaccine of the invention are readily determinable by those of skill in the art. The vaccine can be administered to the subject using any convenient method, preferably parenterally (e.g., via intramuscular, intradermal, or subcutaneous injection) or via oral or nasal administration. The useful dosage to be administered will vary, depending on the type of animal to be vaccinated, its age and weight, the immunogenicity of the attenuated virus, and mode of administration.

A three component or two component immunogenic vaccine of the invention can be administered alone or together. Additionally, because the two component vaccine is useful as an adjuvant, it can be administered to augment other cancer therapies, such as chemotherapy, radiation therapy or other types of immunotherapy.

In one method of treatment, at least one TLR ligand is co-administered with the three component immunogenic vaccine and/or the two component immunogenic vaccine of the invention. The co-administered TLR ligand is administered as an additional adjuvant. Exemplary TLR ligands are described herein. Any TLR ligand can be co-administered with the immunogenic vaccine. Preferably, a TLR2 or a TLR9 ligand such as a CpG ODN is co-administered with the immunogenic vaccine. When the immunogenic vaccine contains, as the covalently linked adjuvant component, a TLR ligand, for example a covalently linked TLR2 ligand, it should be understood that the co-administered TLR ligand, for example a co-administered TLR9 ligand, may be different from the covalently linked TLR ligand.

The method of treatment may involve administration of any combination of three component vaccine, two component vaccine, and/or co-administered TLR ligand, as necessitated by the condition to be treated or as indicated by the health care professional.

Inclusion of an adjuvant in the pharmaceutical composition is optional. Adjuvant includes, for example, alum, QS-21, and TLR agonists. TLR agonists include, but not limited to any of the TLR agonists described herein. Preferred TLR agonists include TLR2 agonists, TLR4 agonists, TLR7 agonists, TLR8 agonists, and TLR9 agonists. TLR9 is activated by unmethylated CpG-containing sequences, including those found in bacterial DNA or synthetic oligonucleotides (ODNs). Such unmethylated CpG containing sequences are present at high frequency in bacterial DNA, but are rare in mammalian DNA. Thus, unmethylated CpG sequences distinguish microbial DNA from mammalian DNA. See, for example, Janeway and Medzhitov, 2002, Ann Rev Immunol; 20:197; Barton and Medzhitov, 2002, Curr Top Microbiol Immunol; 270:81; Medzhitov, 2001, Nat Rev Immunol; 1:135; Heine and Lein, 2003, Int Arch Allergy Immunol; 130:180; Modlin, 2002, Ann Allergy Asthma Immunol; 88:543; and Dunne and O'Neill, 2003, Sci. STKE 2003:re3.

A TLR9 agonist may be a preparation of microbial DNA, including, but not limited to, E. coli DNA, endotoxin free E. coli DNA, or endotoxin-free bacterial DNA from E. coli K12. A TLR9 agonist may be isolated from a bacterium, for example, separated from a bacterial source; synthetic, for example, produced by standard methods for chemical synthesis of polynucleotides; produced by standard recombinant methods, then isolated from a bacterial source; or a combination of the foregoing. In many embodiments, a TLR agonist is purified, and is, for example, at least about 70%, at least about 75%, at least about 80%, at least about 85%, at least about 90%, at least about 95%, at least about 98%, at least about 99%, or more, pure.

A TLR9 agonist may be a synthetic oligonucleotide containing unmethylated CpG motifs, also referred to herein as “a CpG-oligodeoxynucleotide,” “CpGODNs,” or “ODN” (see, for example, Hemmi et al. “A Toll-like receptor recognizes bacterial DNA,” Nature 2000; 408: 740-745). At least three types of immunostimulatory CpG-ODNs have been described. Type A (or D) ODNs preferentially activate plasmacytoid dendritic cells (pDC) to produce IFN?, whereas type B (or K) ODNs induce the proliferation of B cells and the secretion of IgM and IL-6. Another type has been generated that combines features of both types A and B termed, and is termed type C. A TLR9 agonist of the present invention may include any of the at least three types of stimulatory ODNs have been described, type A, type B, and type C.

A CpG-oligodeoxynucleotide TLR9 agonist includes a CpG motif. A CpG motif includes two bases to the 5′ and two bases to the 3′ side of the CpG dinucleotide. CpG-oligodeoxynucleotides may be produced by standard methods for chemical synthesis of polynucleotides. CpG-oligodeoxynucleotides may be purchased commercially, for example, from Coley Pharmaceuticals (Wellesley, Mass.), Axxora, LLC (San Diego, Calif.), or InVivogen, (San Diego, Calif.). A CpG-oligodeoxynucleotide TLR9 agonist may includes a wide range of DNA backbones, modifications and substitutions.

In some aspects of the invention, a TLR9 agonist is a nucleic acid that includes the nucleotide sequence 5′ CG 3′. In some aspects of the invention, a TLR9 agonist is a nucleic acid that includes the nucleotide sequence 5′-purine-purine-cytosine-guanine-pyrimidine-pyrimidine-3′. In other aspects of the invention, a TLR9 agonist is a nucleic acid that includes the nucleotide sequence 5′-purine-TCG-pyrimidine-pyrimidine-3′. In some aspects of the invention, a TLR9 agonist is a nucleic acid that includes the nucleotide sequence 5′-(TGC)n-3′. In other aspects of the invention, a TLR9 agonist is a nucleic acid that includes the sequence 5′-TCGNN-3′, where N is any nucleotide.

In some aspects, a TLR9 agonist may have a sequence of from about 5 to about 200, from about 10 to about 100, from about 12 to about 50, from about 15 to about 25, from about 5 to about 15, from about 5 to about 10, or from about 5 to about 7 nucleotides in length. In some aspects, a TLR9 agonist may be less than about 15, less than about 12, less than about 10, or less than about 8 nucleotides in length.

A TLR9 agonist includes, but is not limited to, any of those described in U.S. Pat. Nos. 6,194,388; 6,207,646; 6,239,116; 6,339,068; and 6,406,705, 6,426,334 and 6,476,000, and published US Patent Applications US 2002/0086295, US 2003/0212028, and US 2004/0248837.

In some aspects, a TLR agonist may be part of a larger nucleotide construct (for example, a plasmid vector, a viral vector, or other such construct). A wide variety of plasmid and viral vector are known in the art, and need not be elaborated upon here. A large number of such vectors have been described in various publications. See, for example, Current Protocols in Molecular Biology, (F. M. Ausubel, et al., Eds. 1987, and updates). Many such vectors are commercially available.

An immunogenic vaccine of the present invention may be administered with one or more additional therapeutic agents. Additional therapeutic treatments include, but are not limited to, surgical resection, radiation therapy, chemotherapy, hormone therapy, anti-tumor vaccines, antibody based therapies, whole body irradiation, bone marrow transplantation, peripheral blood stem cell transplantation, and the administration of chemotherapeutic agents (also referred to herein as “antineoplastic chemotherapy agent”). Antineoplastic chemotherapy agents include, but are not limited to, cyclophosphamide, methotrexate, 5-fluorouracil, doxorubicin, vincristine, ifosfamide, cisplatin, gemcitabine, busulfan (also known as 1,4-butanediol dimethanesulfonate or BU), ara-C (also known as 1-beta-D-arabinofuranosylcytosine or cytarabine), adriamycin, mitomycin, cytoxan, methotrexate, and combinations thereof. The administration of a TLR agonist may take place before, during, and/or after the administration of an additional chemotherapeutic agent. Additional therapeutic agents include, for example, one or more cytokines, an antibiotic, antimicrobial agents, antiviral agents, such as AZT, ddI or ddC, and combinations thereof. The cytokines used include, but are not limited to, IL-1α, IL-1β, IL-2, IL-3, IL-4, IL-6, IL-8, IL-9, IL-10, IL-12, IL-18, IL-19, IL-20, IFN-α, IFN-β, IFN-γ, tumor necrosis factor (TNF), transforming growth factor-beta (TGF-β), granulocyte colony stimulating factor (G-CSF), macrophage colony stimulating factor (M-CSF), granulocyte-macrophage colony stimulating factor (GM-CSF)) (U.S. Pat. Nos. 5,478,556, 5,837,231, and 5,861,159), or Flt-3 ligand (Shurin et al., Cell Immunol. 1997; 179:174-184). Antitumor vaccines include, but are not limited to, peptide vaccines, whole cell vaccines, genetically modified whole cell vaccines, recombinant protein vaccines or vaccines based on expression of tumor associated antigens by recombinant viral vectors. An additional therapeutic agent may be an immune modulator, such as, for example, a TLR4 agonist, a TLR 8 agonist, a TLR9 agonist, a COX-2 inhibitor, GM-CSF, an inhibitor of indoleamine 2, 3-dioxygenase (IDO), a chemotherapy agent, or a combinations thereof.

As noted, the pharmaceutical composition is useful as a vaccine. The vaccine can be a prophylactic or protective vaccine. Likewise, the vaccine can be a therapeutic vaccine, administered after the development of a disease or disorder such as cancer. Thus vaccines that include a glycolipopeptide as described herein, including antimicrobial (e.g., anti-viral or anti-bacterial) and anti-cancer vaccines, are encompassed by the present invention.

Cancers that can be effectively treated or prevented include, but are not limited to, prostate cancer, bladder cancer, colon cancer, breast cancer, melanoma, pancreatic cancer, lung cancer, leukemia, lymphoma, sarcoma, ovarian cancer, Kaposi's sarcoma, Hodgkin's Disease, Non-Hodgkin's Lymphoma, multiple myeloma, neuroblastoma, rhabdomyosarcoma, primary thrombocytosis, primary macroglobulinemia, small-cell lung tumors, primary brain tumors, stomach cancer, malignant pancreatic insulinoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, and cancers of epithelial cell origin. As used herein, “tumor” refers to all types of cancers, neoplasms, or malignant tumors found in mammals.

The efficacy of treatment of a tumor may be assessed by any of various parameters well known in the art. This includes, but is not limited to, determinations of a reduction in tumor size, determinations of the inhibition of the growth, spread, invasiveness, vascularization, angiogenesis, and/or metastasis of a tumor, determinations of the inhibition of the growth, spread, invasiveness and/or vascularization of any metastatic lesions, and/or determinations of an increased delayed type hypersensitivity reaction to tumor antigen. The efficacy of treatment may also be assessed by the determination of a delay in relapse or a delay in tumor progression in the subject or by a determination of survival rate of the subject, for example, an increased survival rate at one or five years post treatment. As used herein, a relapse is the return of a tumor or neoplasm after its apparent cessation, for example, such as the return of leukemia.

The glycolipopeptide of the invention can also be used in passive immunization methods. For example, the glycolipopeptide can be administered to a host animal such as a rabbit, mouse, rat, chicken or goat to generate antibody production in the host animal. Protocols for raising polyclonal antibodies in host animals are well known. The T-epitope or T-epitopes included in the glycolipopeptide optionally are selected to be the same as or similar to the corresponding T-epitope of the host animal in which the antibody is raised. The antibodies are isolated from the animal, then administered to a mammalian subject, preferably a human subject, prophylactically or therapeutically to treat or prevent disease or infection. Monoclonal antibodies against the glycolipopeptide of the invention can be isolated from hybridomas prepared in accordance with standard laboratory protocols; they can also be produced using recombinant techniques such as phage display. Such antibodies are also useful for passive immunization. Optionally, the anti-glycolipopeptide monoclonal antibodies are human antibodies or humanized antibodies. The B-epitope or B-epitopes included in the glycolipopeptide used to create the polyclonal or monoclonal antibodies is selected with reference to the intended purpose of treatment. The invention encompasses polyclonal and monoclonal anti-glycolipopeptide antibodies, as well as methods for making and using them.

Accordingly, also provided by the invention is a pharmaceutical composition that includes the monoclonal or polyclonal antibody of the invention as well as a pharmaceutically acceptable carrier. Preferably the monoclonal antibody is a humanized antibody. Humanized antibodies are more preferable for use in therapies of human diseases or disorders because the humanized antibodies are much less likely to induce an immune response, particularly an allergic response, when introduced into a human host. As noted, the pharmaceutical composition optionally includes excipients or diluents that are pharmaceutically acceptable as carriers and are compatible with the monoclonal antibody and can be administered to any subject including humans and domesticated animals (e.g. cats and dogs). Methods of making and using such a pharmaceutical composition are also included in this invention.

A common feature of oncogenic transformed cells is the over-expression of oligosaccharides, such as Globo-H, Lewis^{Y}, and Tn antigens. Optionally, the pharmaceutical composition of the invention that includes the monoclonal or polyclonal antibody of the invention as well as a pharmaceutically acceptable carrier may be useful in targeting a tumor comprising oncogenic transformed cells over-expressing such oligosaccharides. For example, an antibody conjugated to a chemotherapeutic molecule may be used to deliver the chemotherapeutic molecule to the tumor.

Another pharmaceutical composition of the invention may include a compound (e.g. an antibody, ligand, small molecule, or peptide) that can affect the activity of a protein as well as a pharmaceutically acceptable carrier. The effect of the compound on the protein may include, without limitation, agonizing, antagonizing, inhibiting, or enhancing the normal biological process of the protein. Preferably, the compound is an antibody than binds to an epitope on the protein that includes an O-glycosylation site. Preferably, the O-glycosylation site is an O-GlcNAc site. Numerous studies have shown that this abnormal glycosylation can promote metastasis and hence it is strongly correlated with poor survival rates of cancer patients. Thus, the ability to affect the activity of an abnormally glycosylated protein may enable the prevention of the abnormal activity.

Therapeutically effective concentrations and amounts may be determined for each application described herein empirically by testing the compounds in known in vitro and in vivo systems, including, but not limited to, any of those described herein, dosages for humans or other animals may then be extrapolated therefrom. The efficacy of treatment may be assessed by any of various parameters well known in the art. This includes, but is not limited to, a decrease in tumor size, an increase in CD8⁺ T cell activity, and/or increased survival time.

As noted elsewhere herein, it has been surprisingly found that covalent attachment of a Toll-like receptor (TLR) ligand to a glycopeptide comprising a carbohydrate component (containing a B epitope) and a peptide component (containing a T-epitope) enhances uptake and internalization of the glycopeptide by a target cell (see Example 3). TLR ligands thus identified that are characterized as lipids are preferred lipid components for use in the glycolipopeptide of the invention. The invention thus further provides a method for identifying TLR ligands, preferably lipid ligands, that includes contacting a candidate compound with a target cell containing a Toll-like receptor (TLR), and determining whether the candidate compound binds to the TLR (i.e., is a TLR ligand). Preferably, the candidate compound is internalized by the target cell through the TLR. Lipid-containing TLR ligands identified by binding to a TLR and, optionally, by internalization into the target cell are expected to be immunogenic and are well-suited for use as the lipid component of the glycolipopeptide of the invention. The invention therefore also encompasses glycolipopeptides which include, as the lipid component(s), one or more lipid-containing TLR ligands identified using the method of the invention.

The present invention also includes a diagnostic kit. The kit provided by the invention can contain an antibody of the invention, preferably a monoclonal antibody, and a suitable buffer (such as Tris, phosphate, carbonate, etc.), thus enabling the kit user to identify O-GlcNAc modifications. The user can then detectably label the antibodies as desired. Alternatively, the kit provided by the invention can contain the antibody in solution, preferably frozen in a quenching buffer, or in powder form (as by lyophilization). The antibody, which may be conjugated to a detectable label, or unconjugated, is included in the kit with buffers that may optionally also include stabilizers, biocides, inert proteins, e.g., serum albumin, or the like. Generally, these materials will be present in less than 5% wt. based on the amount of active antibody, and usually present in total amount of at least about 0.001% wt. based again on the antibody concentration. Optionally, the kit may include an inert extender or excipient to dilute the active ingredients, where the excipient may be present in from about 1% to 99% wt. of the total composition. In a preferred embodiment, the antibody provided by the kit is detectably labeled such that bound antibody is detectable. The detectable label can be a radioactive label, an enzymatic label, a fluorescent label, or the like. Optionally, the kit may contain an unconjugated monoclonal antibody of the invention and further contain a secondary antibody capable of binding to the primary antibody. Where a secondary antibody capable of binding to the primary antibody is employed in an assay, this will usually be present in a separate vial. The secondary antibody is typically conjugated to a detectable label and formulated in an analogous manner with the antibody formulations described above. The kit will generally also include packaging and a set of instructions for use.

As used herein, the term “subject” includes, but is not limited to, humans and non-human vertebrates. Non-human vertebrates include livestock animals, companion animals, and laboratory animals. Non-human subjects also include non-human primates as well as rodents, such as, but not limited to, a rat or a mouse. Non-human subjects also include, without limitation, chickens, horses, cows, pigs, goats, dogs, cats, guinea pigs, hamsters, mink, and rabbits. As used herein, the terms “subject,” “individual,” “patient,” and “host” are used interchangeably. In preferred embodiments, a subject is a mammal, particularly a human.

As used herein “in vitro” is in cell culture and “in vivo” is within the body of a subject.

As used herein, “treatment” or “treating” include both therapeutic and prophylactic treatment. To treat a disease or condition shall mean to intervene in such disease or condition so as to prevent or slow the development of, prevent or slow the progression of, halt the progression of, or eliminate the disease or condition.

As used herein, the term “pharmaceutically acceptable carrier” refers to one or more compatible solid or liquid filler, diluents or encapsulating substances which are suitable for administration to a human or other vertebrate animal.

As used herein, the term “isolated” as used to describe a compound shall mean removed from the natural environment in which the compound occurs in nature. In one embodiment isolated means removed from non-nucleic acid molecules of a cell. Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

In some embodiments, an “effective amount” is an amount that results in a reduction of at least one pathological parameter. Thus, for example, an amount that is effective to achieve a reduction of at least about 10%, at least about 15%, at least about 20%, or at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, compared to the expected reduction in the parameter in an individual not receiving treatment.

### EXAMPLES

The present invention is illustrated by the following examples. It is to be understood that the particular examples, materials, amounts, and procedures are to be interpreted broadly in accordance with the scope and spirit of the invention as set forth herein.

### Example 1

### Towards a Fully Synthetic Carbohydrate-Based Anti-Cancer Vaccine

### Synthesis and Immunological Evaluation of a Lipidated Glycopeptide Containing the Tumor-Associated Tn-Antigen

In this Example, a fully synthetic candidate cancer vaccine, composed of a tumor associated Tn-antigen, a peptide T-epitope and the lipopeptide Pam₃Cys was prepared by a combination of polymer-supported and solution phase chemistry. Incorporation of the glycolipopeptide into liposomes gave a formulation that was able to elicit a T-cell dependent antibody response in mice.

A common feature of oncogenic transformed cells is the over-expression of oligosaccharides, such as Globo-H, Lewis^{Y}, and Tn antigens (Lloyd, Am. J Clin. Pathol. 1987, 87, 129; Feizi et al., Trends in Biochem. Sci. 1985, 10, 24-29; Springer, J. Mol. Med. 1997, 75, 594-602; Hakomori, Acta Anat. 1998, 161, 79-90). Numerous studies have shown that this abnormal glycosylation can promote metastasis (Sanders et al., Mol. Pathol. 1999, 52, 174-178) and hence its expression is strongly correlated with poor survival rates of cancer patients.

Several elegant studies have exploited the differential expression of tumor-associated carbohydrates for the development of cancer vaccines (Ragupathi, Cancer Immunol. 1996, 43, 152-157; Musselli et al., J Cancer Res. Clin. Oncol. 2001, 127, R20-R26). The inability of carbohydrates to activate helper T-lymphocytes has complicated, however, their use as vaccines (Kuberan et al., Current Organic Chemistry 2000, 4, 653-677). For most immunogens, including carbohydrates, antibody production depends on the cooperative interaction of two types of lymphocytes, B-cells and helper T-cells (Jennings et al., Neoglycoconjugates, preparation and application, Academic, San Diego, 1994). Saccharides alone cannot activate helper T-cells and therefore have a limited immunogenicity. The formation of low affinity IgM antibodies and the absence of IgG antibodies manifest this limited immunogenicity.

In order to overcome the T-cell independent properties of carbohydrates, past research has focused on the conjugation of saccharides to a foreign carrier protein (e.g. Keyhole Limpet Hemocyanin (KLH) detoxified tetanus toxoid). In this approach, the carrier protein enhances the presentation of the carbohydrate to the immune system and provides T-epitopes (peptide fragments of 12-15 amino acids) that can activate T-helper cells.

However, the conjugation of carbohydrates to a carrier protein poses several problems. In general, the conjugation chemistry is difficult to control, resulting in conjugates with ambiguities in composition and structure, which may affect the reproducibility of an immune response (Anderson et al., J. Immunol. 1989, 142, 2464-2468). In addition, the foreign carrier protein can elicit a strong B-cell response, which may lead to the suppression of an antibody response against the carbohydrate epitope. The latter is a greater problem when self-antigens are employed such as tumor-associated carbohydrates. Also linkers for the conjugation of carbohydrates to proteins can be immunogenic, leading to epitope suppression (Buskas et al., Chem. Eur. J. 2004, 10, 3517-3523). Not surprisingly, several clinical trials with carbohydrate-protein conjugate cancer vaccines failed to induce sufficiently strong helper T-cell responses in all patients (Sabbatini et al., Int. J. Cancer 2000, 87, 79-85). Therefore, alternative strategies need to be developed for the presentation of tumor associated carbohydrate epitopes that will result in a more efficient class switch to IgG antibodies (Keil et al., Angew. Chem. Int. Ed. 2001, 40, 366-369; Angew. Chem. 2001, 113, 379-382; Toyokuni et al., Bioorg. & Med. Chem. 1994, 2, 1119-1132; Lo-Man et al., Cancer Res. 2004, 64, 4987-4994; Kagan et al., Cancer Immunol. Immunother. 2005, 54, 424-430; Reichel et al., Chem. Commun. 1997, 21, 2087-2088).

Here we report the synthesis and immunological evaluation of a structurally well-defined fully synthetic anti-cancer vaccine candidate (compound 9) that constitutes the minimal structural features required for a focused and effective T-cell dependent immune response. The vaccine candidate is composed of the tumor-associated Tn-antigen, the peptide T-epitope YAFKYARHANVGRNAFELFL (YAF) (SEQ ID NO:2), and the lipopeptide S—[(R)-2,3-dipalmitoyloxy-propyl]-N-palmitoyl-(R)-cysteine (Pam₃Cys). The Tn-antigen, which will serve as a B-epitope, is over-expressed on the surface of human epithelial tumor-cells of breast, colon, and prostate. This antigen is not present on normal cells, and thus rendering it an excellent target for immunotherapy. To overcome the T-cell independent properties of the carbohydrate antigen, the YAF peptide was incorporated. This 20 amino acid peptide sequence is derived from an outer-membrane protein of Neisseria meningitides and has been identified as a MHC class II restricted site for human T-cells (Wiertz et al., J. Exp. Med. 1992, 176, 79-88). It was envisaged that this helper T-cell epitope would induce a T-cell dependent immune response resulting in the production of IgG antibodies against the Tn-antigen. The combined B-cell and helper T-cell epitope lacks the ability to provide appropriate “danger signals” (Medzhitov et al., Science 2002, 296, 298-300) for dendritic cell (DC) maturation. Therefore, the lipopeptide Pam₃Cys, which is derived from the immunologically active N-terminal sequence of the principal lipoprotein of Escherichia coli (Braun, Biochim. Biophys. Acta 1975, 415, 335-377), was incorporated. This lipopeptide has been recognized as a powerful immunoadjuvant (Weismuller et al., Physiol. Chem. 1983, 364, 593) and recent studies have shown that it exerts its activity through the interaction with Toll-like receptor-2 (TLR-2) (Aliprantis et al., Science 1999, 285, 736-73). This interaction results in the production of pro-inflammatory cytokines and chemokines, which, in turn, stimulates antigen-presenting cells (APCs), and thus, initiating helper T cell development and activation (Werling et al., Vet. Immunol. Immunopathol. 2003, 91, 1-12). The lipopeptide also facilitates the incorporation of the antigen into liposomes. Liposomes have attracted interest as vectors in vaccine design (Kersten et al., Biochim. Biophys. Acta 1995, 1241, 117-138) due to their low intrinsic immunogenicity, thus, avoiding undesirable carrier-induced immune responses.

The synthesis of target compound 9 requires a highly convergent synthetic strategy employing chemical manipulations that are compatible with the presence of a carbohydrate, peptide and lipid moiety. It was envisaged that 9 could be prepared from spacer containing Tn-antigen 7, polymer-bound peptide 1, and S-[2,3-bis(palmitoyloxy)propyl]-N-Fmoc-Cys (Pam₂FmocCys, 2, (Metzger et al., Int. J. Peptide Protein Res. 1991, 38, 545-554)). The resin-bound peptide 1 was assembled by automated solid-phase peptide synthesis using Fmoc protected amino acids in combination with the hyper acid-sensitive HMPB-MBHA resin and 2-(1H-benzotriazole-1-yl)-oxy-1,1,3,3-tetramethyluronium hexafluorophosphate/1-hydroxybenzotriazole (HBTU/HOBt) (Knorr et al., Tetrahedron Lett. 1989, 30, 1927-1930) as the activation cocktail (Scheme 10). The HMPB-MBHA resin was selected because it allows the cleavage of a compound from the resin without concomitant removal of side-chain protecting groups. This feature was important because side-chain functional groups of aspartic acid, glutamic acid and lysine would otherwise interfere with the incorporation of the Tn-antigen derivative 7. Next, the Pam₂FmocCys derivative 2 was manually coupled to the N-terminal amine of peptide 1 using PyBOP (Martinez et al., J. Med. Chem. 1988, 28, 1874-1879) and HOBt in the presence of DIPEA in a mixture of DMF and dichloromethane to give the resin-bound lipopeptide 3. The Fmoc group of 3 was removed under standard conditions and the free amine of the resulting compound 4 was coupled with palmitic acid in the presence of PyBOP and HOBt to give the fully protected and resin-bound lipopeptide 5. The amine of the Pam₂Cys moiety was palmitoylated after coupling with 1 to avoid racemization of the cysteine moiety. Cleavage of compound 5 from the resin was achieved with 2% TFA in dichloromethane followed by the immediate neutralization with 5% pyridine in methanol. After purification by LH-20 size exclusion chromatography, the C-terminal carboxylic acid of lipopeptide 6 was coupled with the amine of Tn-derivative 7, employing DIC/HOAt/DIPEA (Carpino, J. Am. Chem. Soc 1993, 115, 4397-4398) as coupling reagents to give, after purification by Sephadex LH-20 size-exclusion chromatography, fully protected lipidated glycopeptide 8 in a yield of 79%. Mass spectrometric analysis by MALDI-TOF showed signals at m/z 5239.6 and 5263.0, corresponding to [M+H]⁺ and [M+Na]⁺, respectively. Finally, the side-chain protecting groups of 8 were removed by treatment with 95% TFA in water using 1,2-ethanedithiol (EDT) as a scavenger. It was found that the alternative use of triisopropyl silane (TIS) resulted in the formation of unidentified by-products. The target compound 9 was purified by size-exclusion chromatography followed by RP-HPLC using a Synchropak C4 column. MALDI mass analysis of 9 showed a signal at m/z 3760.3 corresponding to [M+Na]⁺.

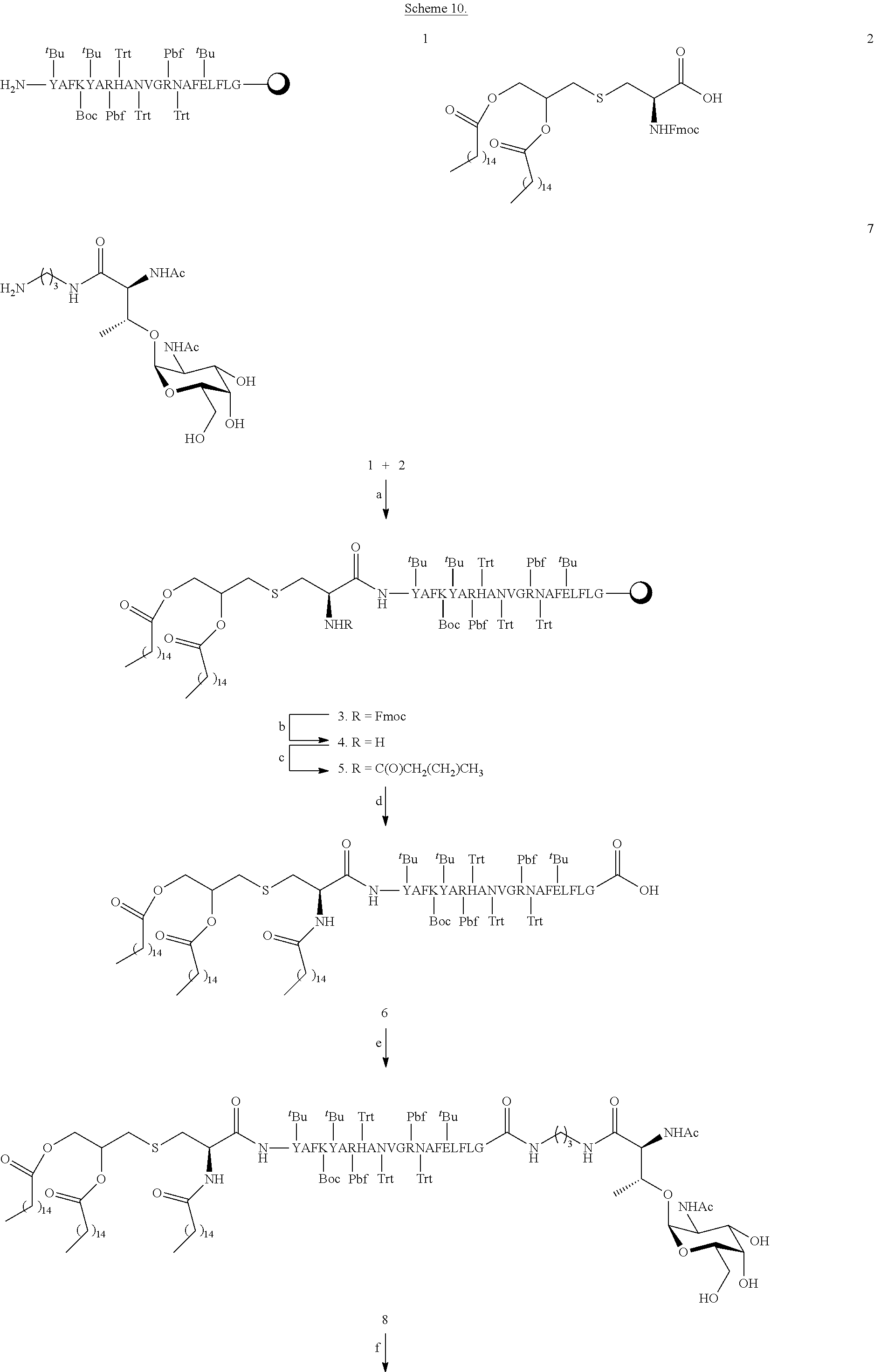

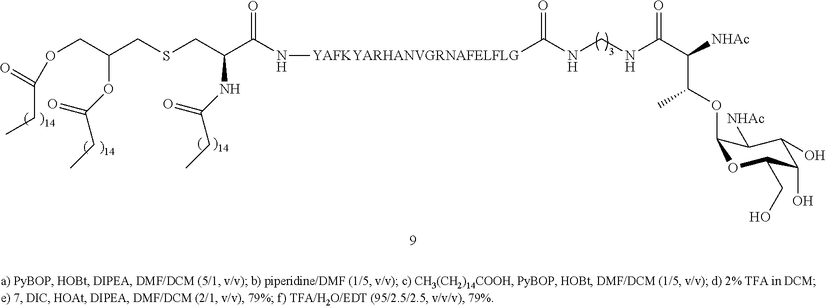

Next, the compound 9 was incorporated into phospholipid-based liposomes. Thus, after hydration of a lipid-film containing 9, cholesterol, phosphatidylcholine and phosphatidylethanolamine, small uni-lamellar vesicles (SUVs) were prepared by extrusion through 100 nm Nuclepore® polycarbonate membranes. Transmission electron microscopy (TEM) by negative stain confirmed that the liposomes were uniformly sized with an expected diameter of approximately 100 nm (see FIG. 1 of Buskas et al., Angew. Chem. Int. Ed. 2005, 44, 5985-5988). The liposome preparations were analyzed for N-acetyl galactosamine content by hydrolysis with TFA followed by quantification by high pH anion exchange chromatography. Concentrations of approximately 30 μg/mL of GalNAc were determined, which corresponded to an incorporation of approximately 10% of the starting compound 9.

Groups of five female BALB/c mice were immunized subcutaneously at weekly intervals with freshly prepared liposomes containing 0.6 μg carbohydrate. To explore the adjuvant properties of the built-in lipopeptide Pam₃Cys, the antigen-containing liposomes were administered with or without the potent saponin immuno-adjuvant QS-21 (Antigenics Inc., Lexington, Mass.). Anti-Tn antibody titers were determined by coating microtiter plates with a BSA-Tn conjugate and detection was accomplished with anti-mouse IgM or IgG antibodies labeled with alkaline phosphatase. As can be seen in Table 1, the mice immunized with the liposome preparations elicited IgM and IgG antibodies against the Tn-antigen (Table 1, entries 1 and 2). The presence of IgG antibodies indicated that the helper T-epitope peptide of 9 had activated helper T-lymphocytes. Furthermore, the observation that IgG antibodies were raised by mice which were only immunized with liposomes (group 1) indicated that the built-in adjuvant Pam₃Cys had triggered appropriate signals for the maturation of DCs and their subsequent activation of helper T-cells. However, the mice which received the liposomes in combination with QS-21 (group 2), elicited higher titers of anti Tn-antibodies. This stronger immune response may be due to a shift from a mixed Th1/Th2 to a Th1 response (Moore et al., Vaccine 1999, 17, 2517-2527).

TABLE 1
ELISA anti-Tn antibody titers^{[a]} after 4 immunizations
with the glycolipopeptide/liposome formulation.

| Entry | Group | IgM Titers | IgG Titers |
|---|---|---|---|
| 1. | 1. Pam₃Cys-YAF-Tn | 250 | 1410 |
| 2. | 2. Pam₃Cys-YAF-Tn + QS-21 | 170 | 2675 |

^{[a]}ELISA plates were coated with a BSA-BrAc-Tn conjugate. All titers are means for a group of five mice. Titers were determined by regression analysis, plotting log₁₀ dilution vs. absorbance. The titers were calculated to be the highest dilution that gave 0.1 or higher than the absorbance of normal saline mouse sera diluted 1:100.

The results presented herein provide, for the first time, a proof-of-principle for the use of lipidated glycopeptides as a minimal subunit vaccine. It is to be expected that several improvements can be made. For example, it has been found that a clustered presentation of the Tn-antigen is a more appropriate mimetic of mucins, and hence antibodies raised against this structure recognize better Tn-antigens expressed on cancer cells (Nakada et al., J. Biol. Chem. 1991, 266, 12402-12405; Nakada et al., Proc. Natl. Acad. Sci. USA 1993, 90, 2495-2499; Reddish et al., Glycoconj. J. 1997, 14, 549-560; Reis et al., Glycoconj. J. 1998, 15, 51-62). The Th-epitope employed in this study is known to be a MHC class II restricted epitope for humans. Thus, a more efficient class-switch to IgG antibodies may be expected when a murine Th-epitope is employed. On the other hand, compound 9 is a more appropriate vaccine candidate for use in humans. A recent report indicated that Pam₂Cys is a more potent immunoadjuvant than Pam₃Cys (Jackson et al., Proc. Nat. Acad. Sci. USA 2004, 101, 15440-15445). It has also been suggested that the Pam₂Cys adjuvant has improved solubility properties (Zeng et al., J. Immunol. 2002, 169, 4905-4912), which is a problematic feature of compound 9. Studies addressing these issues are ongoing.

This work is reported in Buskas et al., Angew. Chem. Int. Ed. 2005, 44, 5985-5988.

### Supporting Information

Reagents and General Experimental Procedures.

Amino acids and resins were obtained from Applied Biosystems and NovaBiochem; DMF from EM science; and NMP from Applied Biosystems. Phosphatidylethanolamine (PE), cholesterol, phosphatidylcholine (PC; egg yolk), and phosphatidylglycerol (PG; egg yolk) were from purchased from Sigma-Aldrich and Fluka. All other chemicals were purchased from Aldrich, Acros, and Fluka and used without further purification. All solvents employed were of reagent grade and dried by refluxing over appropriate drying agents. TLC was performed using Kieselgel 60 F₂₅₄ (Merck) plates, with detection by UV light (254 nm) and/or by charring with 8% sulfuric acid in ethanol or by ninhydrine. Column chromatography was performed on silica gel (Merck, mesh 70-230). Size exclusion column chromatography was performed on Sephadex LH-20. Extracts were concentrated under reduced pressure at ≦40° C. (water bath). An Agilent 1100 series HPLC system equipped with an autosampler, UV-detector and fraction-collector and a Synchropak C4 column 100×4 6 mm RP with a flow rate of 1 mL/min was used for analysis and purifications. Positive ion matrix assisted laser desorption ionization time of flight (MALDI-TOF) mass spectra were recorded using an HP-MALDI instrument using gentisic acid as a matrix. ¹H NMR and ¹³C NMR spectra were recorded on a Varian Inova300 spectrometer, a Varian Inova500 spectrometer, and a Varian Inova600 spectrometer all equipped with Sun workstations. ¹H spectra recorded in CDCl₃ were referenced to residue CHCl₃ at 7.26 ppm or TMS, and ¹³C spectra to the central peak of CDCl₃ at 77.0 ppm. Assignments were made using standard 1D experiments and gCOSY/DQCOSY, gHSQC and TOCSY 2D experiments.

Lipopeptide 6.

Compound 1 was synthesized on HMPB-MBHA resin (maximum loading, 0.1 mmol). The synthesis of peptide 1 was carried out on an ABI 433A peptide synthesizer equipped with a UV-detector using Fmoc-protected amino acids and 2-(1H-benzotriazole-1-yl)-oxy-1,1,3,3-tetramethyluronium hexafluorophosphate (HBTU)/1-hydroxybenzotriazole (HOBt) as the coupling reagents. Single coupling steps were performed with conditional capping as needed. After completion of the synthesis of peptide 1, the remaining steps were performed manually. N-Fluorenylmethoxycarbonyl-S-(2,3-bis(palmitoyloxy)-(2R-propyl)-(R)-cysteine 2 (120 mg, 0.13 mmol) was dissolved in DMF (5 mL) and PyBOP (0.13 mmol), HOBt (0.13 mmol), and DIPEA (0.27 mmol) were added. After premixing for 2 min., DCM (1 mL) was added and the mixture was added to the resin. The coupling step was performed twice. Upon completion of the coupling, as determined by the Kaiser test, the N-Fmoc group was cleaved using 20% piperidine in DMF (5 mL). Palmitic acid (77 mg, 0.3 mmol) was coupled to the free amine as described above using PyBop (0.3 mmol), HOBt (0.3 mmol) and DIPEA (0.6 mmol) in DMF. The resin was thoroughly washed with DMF and DCM and dried under vacuum for 4 h. The fully protected lipopeptide 6 was released from the resin by treatment with 2% trifluoroacetic acid in DCM (2.5 mL) for 2 min. The mixture was filtered into 5% pyridine in methanol solution (5 mL). The procedure was repeated and fractions containing the lipopeptide were pooled and concentrated to dryness. The crude product was purified by size-exclusion chromatography (LH-20, DCM/MeOH, 1:1) to give lipo-peptide 6 (275 mg, 0.057 mmol) as a white solid: R_{f}=0.57 (DCM/MeOH 9:1); selected NMR data (CDCl₃/CD₃OD 1/1 v/v 600 MHz): ¹H, δ 0.48-0.90 (m, 27H, Pam CH₃, Leu CH₃, Val CH₃), 0.96-1.61 (m, Leu CH₂, Leu CH, Lys CH₂, ^{t}Bu CH₃, Boc CH₃, Ala CH₃, Arg CH₂), 1.18 (br s, 72H, Pam CH₂), 1.95, 1.99 (s, 4×3H, Pbf CH₃C), 2.36, 2.41, 2.44 (s, 6×3H, Pbf CH₃), 2.48 (s, 2×2H, Pbf CH₂) 2.65-2.73 (m, 6H, S—CH₂-glyceryl, His CH₂, Cys^{β}), 3.47 (m, 2H, Gly^{α}), 3.57 (m, 2H, Gly^{α}), 4.06 (m, 1H, S-glyceryl-CH₂^{b}O), 4.32 (m, 1H, S-glyceryl-CH₂^{a}O), 3.65-4.39 (m, 17H, Phe^{α}, Ala^{α}, His^{α}, Lys^{α}, Val^{α}, Asn^{α}, Glu^{α}, Tyr^{α}, Arg^{α}), 4.45 (m, 1H, Cys^{α}), 5.06 (m, 1H, S-glyceryl-CH), 6.72-7.39 (m, 70H, His CH, Tyr aromat, Phe aromat, Trt aromat), 7.48-8.29 (m, NH). MALDI-MS calcd for C₂₆₉H₃₇₃N₃₃O₄₂S₃ [M+Na] m/z=4860.22: found 4860.31.

Protected Glycolipopeptide 8.

A solution of lipopeptide 6 (22 mg, 4.6 μmol), HOAt (6.3 mg, 46 μmol), and DIC (7 μL, 46 μmol) in DCM/DMF (2/1 v/v, 1.5 mL) was stirred under argon atm. at ambient temperature for 15 min. Compound 7 (8 mg, 19 μmol) and DIPEA (14 μL, 92 μmol) in DMF (1.5 mL) was added to the stirred mixture of lipopeptide and the reaction was kept at room temperature for 18 h. The mixture was diluted with toluene and concentrated to dryness under reduced pressure. Purification of the residue by size-exclusion chromatography (LH-20, DCM/MeOH 1:1) gave compound 8 (19 mg, 79%) as a white solid: selected NMR data (CDCl₃/CD₃OD 1/1 v/v 600 MHz): ¹H, δ□ 0.60-0.90 (m, 27H, Pam CH₃, Leu CH₃, Val CH₃), 0.96-1.61 (m, Leu CH₂, Leu CH, Lys CH₂, ^{t}Bu CH₃, Boc CH₃, Ala CH₃, Arg CH₂), 1.18 (br s, 72H, Pam CH₂), 1.94, 1.98, 1.99, 2.00 (s, 6×3H, Pbf CH₃C, HNAc CH₃), 2.36, 2.41, 2.45 (s, 6×3H, Pbf CH₃), 2.48 (s, 2×2H, Pbf CH₂), 3.42-4.31 (m, Phe^{α}, Ala, Lys, Val, Asp, Glu, Tyr, Arg, Gly, Leu, His, Asn CH₂, Tyr CH₂, Phe CH₂, Arg CH₂), 3.71 (H-3), 3.88 (H-4) 4.06 (S-glyceryl-CH₂^{β}O), 4.20 (t, 1H, H-2), 4.32 (m, 1H, S-glyceryl-CH₂^{α}O), 4.42 (m, 1H, Cys^{α}), 4.82 (d, 1H, H-1, J=3.68 Hz), 5.06 (m, 1H, S-glyceryl-CH), 6.72-7.39 (m, 70H, His CH, Tyr aromat, Phe aromat, Trt aromat), 7.48-8.29 (m, NH). MALDI-MS calcd for C₂₈₆H₄₀₃N₃₇O₄₉S₃ [M+Na] m/z=5262.67: found 5262.99.

Glycolipopeptide 9.

Compound 8 (12 mg, 2.3 μmol) in a deprotection cocktail of TFA/H₂O/ethane-1,2-dithiol (95:2.5:2.5, 3 mL) was stirred at room temperature for 1 h. The solvents were removed under reduced pressure and the crude compound was first purified by a short size-exclusion LH-20 column (DCM/MeOH 1:1) and the then by HPLC using a gradient of 0-100% acetonitrile in H₂O (0.1% TFA) to give, after lyophilization, compound 9 (6.8 mg, 79%) as a white solid: selected NMR data (CDCl₃/CD₃OD 600 MHz): ¹H, δ 0.74-0.96 (m, 27H, Pam CH₃, Leu CH₃, Val CH₃), 1.11-2.35 (Leu CH₂, Leu CH, sp CH₂, Lys CH₂, Glu CH₂, Ala CH₃, Val CH, Asp CH₂), 1.29 (br S, 72H, Pam CH₂), 2.43-3.87 (Ala^{α}, Gly^{α}, S-glyceryl-OCH₂, Cys^{β}, H-2, H-3, H-4, H-5, H-6), 4.05-4.73 (m, Cys^{α}, Phe^{α}, Tyr^{α}, His^{α}, Leu^{α}, Lys^{α}, Asp^{α}, Val^{α}, Arg^{α}, Glu^{α}, H-1), 5.12 (m, 1H, S-glyceryl-CH), 6.64-6.71 (dd+dd, 2H, His CH, NH), 6.86-7.12 (dd+dd 2H, His CH, NH) 7.16-8.23 (m, Tyr aromat, Phe aromat, NH). HR-MALDI-MS calcd for C₁₃₆H₂₉₇N₃₇O₄₁S [M+Na] m/z=3760.1911: found 3760.3384.

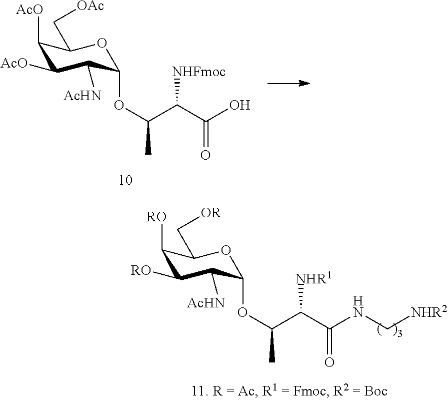

Tn Derivative 11.

Compound 10 was dissolved in DMF (10 mL) and di-isopropylcarbodiimide (DIC) (82 μL, 0.53 mmol) and HOAt (216 mg, 1.58 mmol) were added. After stirring for 15 min., 3-(N-(tert.butyloxycarbonyl)-amino)propanol (111 mg, 0.63 mmol) was added and the reaction was kept at ambient temperature for 15 h. The mixture was concentrated to dryness under reduced pressure and the residue was purified by silica gel column chromatography (0-5% MeOH in DCM) and LH-20 size-exclusion chromatography (DCM/MeOH 1:1) to give compound 11 (363 mg, 83%). R_{f}=0.63 (DCM/MeOH 9:1); [α]_{D}+4.4 (c 1.0 mg/mL, CH₂Cl₂); NMR data (CDCl₃, 500 MHz): ¹H, δ 1.27 (d, 3H, CH₃ Thr), 1.43 (s, 9H, ^{t}Bu CH₃), 1.46-1.61 (m, 2H, CH₂), 1.99 (s, 3H, CH₃ Ac), 2.05 (s, 6H, CH₃Ac), 2.06 (s, 3H, CH₃Ac), 2.17 (s, 3H, CH₃Ac), 3.17-3.27 (m, 3H, CH₂, CH₂ₐ), 3.48-3.50 (m, 1H, CH_{2b}), 4.07-4.28 (m, 6H, H-6, H-5, Thr^{α}, Thr^{β}, CH Fmoc), 4.43-4.51 (m, 2H, CH₂ Fmoc), 4.62 (dd, 1H, H-2), 4.89 (br t, 1H, NH), 5.04-5.11 (m, 2H, H-1, H-3), 5.41 (d, 1H, H-4), 5.75 (br d, 1H, NH T), 6.81 (br d, 1H, NH GalNAc), 7.17-7.79 (m, 8H, aromatic H); ¹³C (CDCl₃, 75 MHz) □δ17.19, 20.92, 20.99, 21.09, 23.30, 28.55, 30.69, 35.87, 36.92, 47.43, 47.77, 58.57, 62.36, 67.47, 68.68, 77.46, 80.08, 99.88, 120.25, 125.34, 127.35, 128.00, 128.76, 129.13, 141.55, 143.94, 144.01, 156.51, 157.52, 169.68, 170.66, 170.94, 170.99.

HR-MALDI-MS calcd for C₄₁H₅₄N₄O₁₄ [M+Na] m/z=849.3535: found 849.3391.

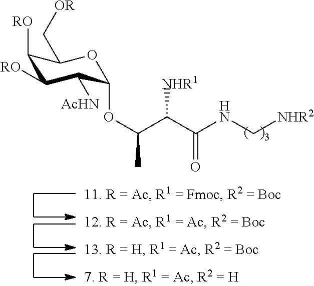

Tn Derivative 7.

A solution of compound 11 (194 mg, 0.24 mmol) in 20% piperidine in DMF (5 mL) was stirred at ambient temperature for 1 h. The mixture was concentrated to dryness and the residue was treated with pyridine/acetic anhydride (3:1, 5 mL) for 2 h. The reaction mixture was diluted with toluene and concentrated to dryness. The residue was dissolved in dichloromethane and washed with 1M HCl and sat. aq. NaHCO₃, dried with MgSO₄, filtered and concentrated. Purification of the residue by size-exclusion chromatography (LH-20, DCM/MeOH 1:1) furnished compound 12 (167 mg, 91%): NMR data (CDCl₃, 300 MHz): ¹H, δ 1.24 (d, 1H, Thr CH₃), 1.42 (s, 9H, ^{t}Bu CH₃), 1.55-1.59 (m, 2H, NHCH₂CH₂CH₂NH), 1.95, 2.02, 2.03, 2.12, 2.14 (s, 15H, CH₃Ac), 3.13-3.23 (m, 3H, CH₂+CH₂ₐ), 3.36-3.41 (m, 1H, CH_{2b}), 4.03-4.12 (m, 2H), 4.19-4.23 (m, 2H, Thr^{β}), 4.54-4.61 (m, H-2, Thr^{α}), 4.88 (m, 1H, NH), 4.96 (s, 1H, J=3.57 Hz, H-1), 5.07 (dd, 1H, H-3), 5.35 (d, 1H, H-4), 6.43 (br S, 1H, NH), 6.72 (br S, 1H, NH). MALDI-MS calcd for C₂₈H₄₆N₄O₁₃ [M+Na] m/z=669.296: found 669.323. Compound 12 was deprotected by stirring with 5% hydrazine-hydrate in methanol (5 mL) at room temperature for 35 min. The reaction mixture was diluted with toluene and concentrated. The residue was co-evaporated twice with toluene. Purification by silica gel column chromatography (DCM/MeOH 5:1) yielded 13 (119 mg, 89%): NMR data (CD₃OD, 300 MHz): ¹H, δ 1.26 (d, 3H, Thr CH₃), 1.43 (s, 9H, ^{t}Bu CH₃), 1.57-1.63 (m, 2H, NHCH₂CH₂CH₂NH), 2.06, 2.10 (s, 2×3H NHAc), 2.12-3.09 (m, 2H, CH₂), 3.15 (m, 2H, CH₂), 3.31 (br s, 2H, H-6), 3.68-3.76 (m, 2H, H-3, H-5), 3.88 (d, 1H, H-4), 4.22-4.26 (m, 2H, H-2, Thr^{β}), 4.46 (m, 1H, Thr^{α}), 4.84 (d, 1H, H-1), 6.60 (br m, 1H, NH), 7.50 (br d, 1H, NH). MALDI-MS calcd. for C₂₂H₄₀N₄O₁₀ [M+Na] m/z=543.264: found 543.301. A solution of 13 in trifluoro acetic acid (4 mL) was stirred under an argon atmosphere at ambient temperature for 45 min. The reaction mixture was then diluted with DCM and concentrated to dryness. The crude product was purified by column chromatography (Iatro beads, EtOAc/MeOH/H₂O 2:2:1→MeOH/H₂O 1:1). After concentration of the pooled fractions, the solid was lyophilized from H₂O to give compound 7 (91 mg, 0.21 mmol, 95%) as a white powder. R_{f}=0.17 (EtOAc/MeOH/H₂O 6:3:1); [α]_{D}-37 (c 1.0 mg/mL, H₂O); NMR data (D₂O, 300 MHz): ¹H, δ 1.15 (d, 3H, J=6.3 Hz, Thr CH₃), 1.73-1.77 (m, 2H, CH₂), 1.95 (s, 3H, NHAc), 2.04 (s, 3H, NHAc), 2.82-2.87 (m, 2H, CH₂), 3.11-3.15 (m, 1H, CH₂ₐ), 3.22-3.26 (m, 1H, CH_{2b}), 3.65 (m, 2H, H-6), 3.76 (dd, 1H, J=2.9, 11.2 Hz, H-3), 3.87 (d, 1H, J=2.9 Hz, H-4), 3.92 (t, 1H, H-5), 3.99 (dd, 1H, J=3.41, 11.2 Hz, H-2), 4.28-4.30 (m, 1H, Thr^{β}), 4.32 (d, 1H, J=2.4 Hz, Thr^{α}) 4.78 (d, 1H, J=3.56 Hz, J=3.9 Hz, H-1), 7.97 (br d, 1H, NH), 8.17 (br t, 1H, NH), 8.27 (br d, 1H, NH); ¹³C (D₂O, 75 MHz), δ 18.17 Thr CH₃), 21.93, 22.33 (2×NAc) 26.98 (CH₂), 36.55 (CH₂), 37.22 (CH₂), 49.98 (C-6), 58.30 (C-3), 61.46 (C-4), 67.76 (C-5), 68.65 (C-2), 71.54 (C-Thr^{β}), 74.60 (C-Thr^{α}), 98.60 (C-1), 172.09, 174.37, 175.18 (3×C═O, NHAc). HR-MALDI-MS calcd for C₁₇H₃₂N₄O₈ [M+Na] m/z=443.2118: found 443.2489.

Liposome Preparation.

Liposomes were prepared from PC, PG, cholesterol, and the glycolipopeptide 9 (15 μmol, molar ratio 65:25:50:10). The lipids were dissolved in DCM/MeOH (3/1, v/v) under an atmosphere of argon. The solvent was then removed by passing a stream of dry nitrogen gas, followed by further drying under high vacuum for one hour. The resulting lipid film was suspended in 1 mL 10 mM Hepes buffer, pH 6.5, containing 145 mM NaCl. The solution was vortexed on a shaker (250 rpm), under Ar atmosphere at 41° C. for 3 hours. The liposome suspension was extruded ten-times through 0.6 μm, 0.2 μm and 0.1 μm polycarbonate membranes (Whatman, Nuclepore®, Track-Etch Membrane) at 50° C. to obtain SUV.

Immunizations.

Groups of five mice (female BALB/c, 6 weeks) were immunized subcutaneously on days 0, 7, 14 and 21 with 0.6 μg of carbohydrate-containing liposomes and 10 μg of the adjuvant QS-21 in each boost. The mice were bled on day 28 (leg-vein) and the sera were tested for the presence of antibodies.

ELISA.

96-well plates were coated over night at 4° C. with Tn-BSA, (2.5 μg mL⁻¹) in 0.2 M borate buffer (pH 8.5) containing 75 mM sodium chloride (100 μL) per well). The plates were washed three times with 0.01 M Tris buffer containing 0.5% Tween 20% and 0.02% sodium azide. Blocking was achieved by incubating the plates 1 h at room temperature with 1% BSA in 0.01 M phosphate buffer containing 0.14 M sodium chloride. Next, the plates were washed and then incubated for 2 h at room temperature with serum dilutions in phosphate buffered saline. Excess antibody was removed and the plates were washed three times. The plates were incubated with rabbit anti-mouse IgM and IgG Fey fragment specific alkaline phosphatase conjugated antibodies (Jackson ImmunoResearch Laboratories Inc., West Grove, Pa.) for 2 h at room temperature. Then, after the plates were washed, enzyme substrate (p-nitrophenyl phosphate) was added and allowed to react for 30 min before the enzymatic reaction was quenched by addition of 3 M aqueous sodium hydroxide and the absorbance read at dual wavelengths of 405 and 490 nm. Antibody titers were determined by regression analysis, with log₁₀ dilution plotted against absorbance. The titers were calculated to be the highest dilution that gave two times the absorbance of normal mouse sera diluted 1:120.

### Example 2

### Non-Covalently Linked Diepitope Liposome Preparations

In a first set of experiments, the tumor-related carbohydrate B-epitope and the universal T-epitope peptide were incorporated separately into preformed liposomes to form a diepitopic construct. Additionally, the lipopeptide Pam₃Cys was incorporated into the liposome with the expectation that it would function as a built-in adjuvant, and thus circumvent the necessity of using an additional external adjuvant, such as QS-21.

The liposomes were prepared from lipid anchors carrying two different thiol-reactive functionalities, maleimide and bromoacetyl, at their surface. The Pam₃Cys adjuvant was also incorporated into the preformed liposome and included a maleimide functionality. Conveniently, the maleimide and the bromoacetyl group show a marked difference in their reactivity towards sulfhydryl groups. The maleimide reacts rapidly with a sulfhydryl compound at pH 6.5, whereas the bromoacetyl requires slightly higher pH 8-9 to react efficiently with a thiol compound.

By exploiting this difference in reactivity, a diepitope liposome construct carrying the cancer related Le^{y} tetrasaccharide and the universal T helper peptide QYIKANSKFIGITEL (QYI) (SEQ ID NO:1) was prepared (Scheme 11). For the conjugation to the thiol-reactive anchors, both the oligosaccharide and the peptide were functionalized with a thiol-containing linker. The two-step consecutive conjugation to preformed liposomes has a great advantage: it is a very flexible approach that makes it easy to prepare liposomes carrying an array of different carbohydrate B-epitopes. The yield of conjugation, as based on quantitating the carbohydrate and peptide covalently coupled to the vesicles, was high, 70-80% for the oligosaccharide and 65-70% for the peptide, and the results were highly reproducible.

It is important to note that in these first diepitope liposome constructs, the carbohydrate B-epitope and peptide T-epitope are not themselves joined together by covalent linkages, but rather are held in proximity by their respective lipid anchors to which they are conjugated, and by hydrophobic interactions. It has been shown in several reports in the literature regarding vaccine candidates with pathogen-related peptide B-epitopes that this approach is successful leading to good titers of both IgM and specific IgG antibodies. These studies also indicate that the built-in adjuvant Pam₃Cys is sufficient to induce a proper immune response.

However, in our study with the tumor-related carbohydrate B-epitope Le^{y}, immunizations of mice using the non-covalently linked diepitope liposome preparation described in this Example resulted in only very low titers of IgM antibodies. No IgG anti-Le^{y} antibodies were detected. Even more surprising, co-administering the liposomal vaccine candidate with the powerful external adjuvant, QS-21, did not improve the outcome. Additionally, it was found that mice that had been immunized with an un-coated liposome control, i.e. a liposome that carried nothing but the maleimide and bromoacetyl functional groups on the surface, elicited high titers of IgG antibodies as detected by ELISA. More detailed ELISA studies of the anti-sera from this group of mice using a variety of protein conjugates revealed that the mice had responded to and elicited antibodies towards the maleimide linker. Also the anti-sera from the mice immunized with the liposomes coated with the Le^{y} antigen and the QYI peptide were screened for anti-linker antibodies and it was found that also these mice had elicited IgG antibodies towards the maleimide linker.

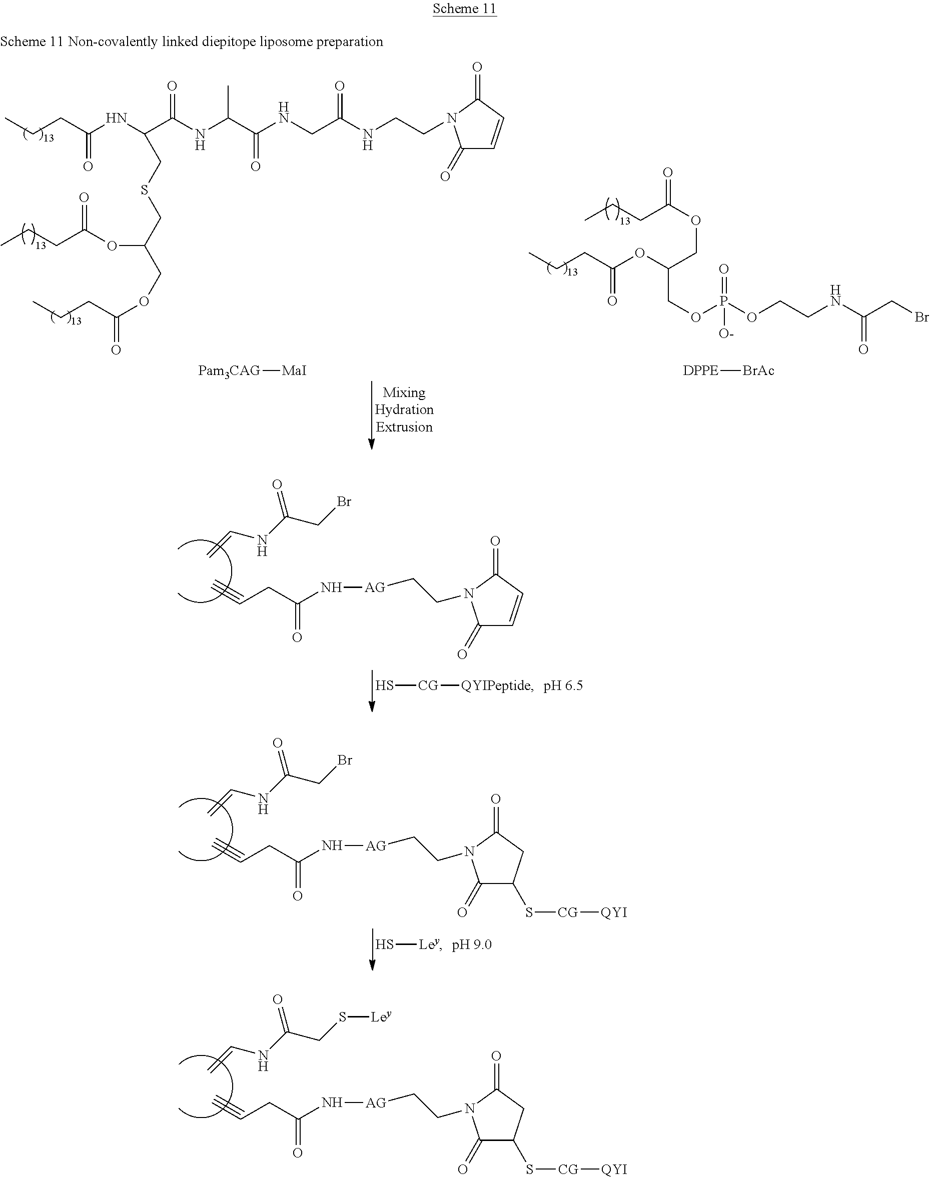

Due to its high reactivity at near neutral pH, the maleimide linker is widely used in conjugation chemistry to reach glyco- and peptide-protein conjugates that are further used in immunization studies. There are commercially available protein conjugation kits (Pierce Endogen Inc.) that utilize the maleimide linker both for the antigenic conjugate and the detection conjugate. Our data show that using these kits can lead to false positive results, especially when working with antigens of low immunogenicity (See T. Buskas, Y. Li and G-J. Boons, Chem. Eur. J., 10:3517-3523, 2004).

To test whether the highly immunogenic maleimide linker suppressed the immune response towards the Le^{y} tetrasaccharide, we prepared the non-covalent diepitope liposome using only the bromoacetyl linker. In this experiment, the thiol-containing Le^{y} tetrasaccharide and the universal T helper peptide were conjugated, in separate reactions, to lipids containing the bromoacetyl linker. The conjugated lipids were then mixed together to form lipid vesicles. Administering this new liposome formulation to mice, with or without the external adjuvant QS-21, raised only low titers of anti-Le^{y} antibodies. Thus, the lack of an effective immune response toward the Le^{y} tetrasaccharide was not due solely to the immunogenic maleimide linker.

Since the tumor-associated Le^{y} tetrasaccharide is known to be only weakly immunogenic, we prepared another diepitope liposomal construct where the more immunogenic Tn(cluster) antigen was used as a target B-epitope. However, the same negative results were obtained with this antigen. Again, immunizations of mice resulted in only very low titers of anti-Tn(c) IgM antibodies. Co-administering with QS-21 as an external adjuvant did nothing to enhance the immune response.

From these results we concluded that the non-covalently linked diepitope liposome approach that has proven successful for a range of peptide antigens failed when a tumor-associated carbohydrate antigen of low immunogenicity was used as a B-epitope. Thus, we reasoned that the tumor-associated carbohydrate B-epitope and the helper T-epitope needed to be presented differently to the immune system to evoke a T-cell dependent immune response.

### Example 3

### Covalently Linked Diepitope Liposome Preparations

We speculated that in order to achieve a better presentation of the carbohydrate B-epitope and peptide T-epitope, perhaps they needed to be covalently linked together. To test this idea we synthesized construct 1 (Scheme 12), a structurally well-defined anti-cancer vaccine candidate containing the structural features needed for a focused and effective T-cell dependent immune response. The vaccine candidate is composed of the tumor-associated Tn-antigen, the peptide T-epitope YAFKYARHANVGRNAFELFL (YAF) (SEQ ID NO:2) (Neisseria meningitides) and the lipopeptide Pam₃Cys. Due to difficulties in the synthesis using the original helper T-epitope peptide QYI, a different universal T-epitope (YAF) that displayed better solubility properties was used in this study.

Compound 1 was synthesized in a highly convergent manner by a combination of solid-phase and solution phase synthesis.

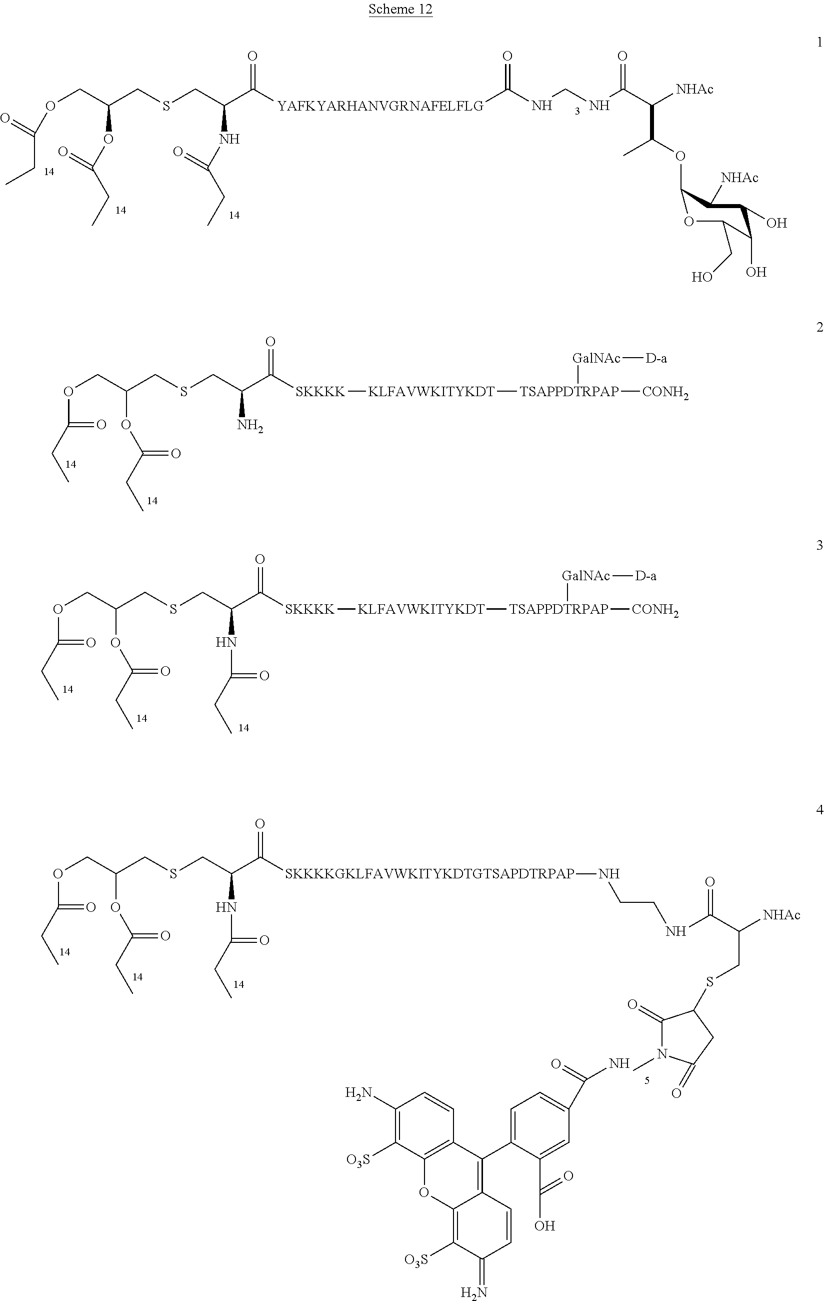

The construct was then incorporated into phospholipid-based liposomes. Compound 1 suffers from low solubility in a range of solvents, which probably is the main reason the incorporation into the liposomes was only 10%.

Mice were immunized with the construct at weekly intervals. To explore the adjuvant properties of the built-in lipopeptide Pam₃Cys, the antigen-containing liposomes were administered with (group 2) or without (group 1) the adjuvant QS-21.

As can be seen in Table 1 (Example 1), the mice immunized with the liposome preparations elicited both IgM and IgG antibodies against the Tn-antigen (Table 1, entries 1 and 2). The presence of IgG antibodies indicated that the helper T-epitope peptide of 1 had activated helper T-lymphocytes. Furthermore, the observation that IgG antibodies were raised by mice which were immunized with liposomes in the absence of the external adjuvant QS-21 (group 1) indicated that the built-in adjuvant Pam₃Cys had triggered appropriate signals for the maturation of DCs and their subsequent activation of helper T-cells. However, the mice which received the liposomes in combination with QS-21 (group 2) elicited higher titers of anti Tn-antibodies. This stronger immune response may be due to a shift from a mixed Th1/Th2 to a Th1 skewed response.

The results provide, for the first time, a proof-of-principle for the use of a lipidated glycopeptide that contains a carbohydrate B-epitope, a helper T-cell epitope and a lipopeptide adjuvant as a minimal, self-contained subunit vaccine. It was also concluded that to evoke a T-cell dependent immune response toward the tumor-associated carbohydrate antigen, it is not enough that the carbohydrate B-epitope and the peptide T-epitope are presented together in a non-covalent manner on the surface of a adjuvant-containing liposome; rather, the entities are preferably covalently joined together. Finally, it was observed that an external adjuvant (QS-21) was not needed when the three components (carbohydrate B-epitope, helper T-cell epitope and lipopeptide) are covalently linked to form the lipidated glycopeptides.

### Alternative Glycolipopeptide Components

Several improvements can be made to compound 1. For example, it has been found that antibodies elicited against the Tn-antigen poorly recognize cancer cells. However, clustering (Nakada et al., Proc. Natl. Acad. Sci. USA 1993, 90, 2495-2499; Reddish et al., 1997, 14, 549-560; Zhang et al., Cancer Res. 1995, 55, 3364-3368; Adluri et al., Cancer Immunol. Immunother 1995, 41, 185-192) or presenting the Tn antigen as part of the MUC-1 glycopeptide elicits antibodies with improved binding characteristics (Snijdewint et al., Int. J. Cancer 2001, 93, 97-106 The T-epitope employed in compound 1 is a MHC class II restricted epitope for humans. Thus, a more efficient class-switch to IgG antibodies may be expected when a murine T-epitope is used. Furthermore, it has been found that the lipopeptide Pam₂Cys or Pam₃CysSK₄ are more potent immunoadjuvants than Pam₃Cys (Spohn et al., Vaccine 2004, 22, 2494-2499). However, it was not known whether attachment of Pam₂Cys or Pam₃CysSK₄ to the T- and B-epitope would affect their efficacies and potencies. Thus, based on these considerations, compounds 2 and 3 (Scheme 12) were designed, which contain the MUC-1 glycopeptide as a B-epitope, the well-documented murine helper T-cell epitope KLFAVWKITYKDT (KLF) (SEQ ID NO:3) derived from Polio virus (Leclerc et al., J. Virol. 1991, 65, 711-718) as the T-epitope, and the lipopeptide Pam₂Cys or Pam₃CysSK₄, respectively.

Glycolipopeptides 2 and 3 were incorporated into phospholipid-based liposomes as described for compound 1. Surprisingly, the solubility problems that plagued compound 1 were not an issue for compounds 2 and 3. Female BALB/c mice were immunized four times at weekly intervals with the liposome formulations with or without the external adjuvant QS-21 (Kensil et al., J. Immunol. 1991, 146, 431-437). Anti-Muc1 antibody titers were determined by coating microtiter plates with CTSAPDT(αGalNAc)RPAP conjugated to BSA and detection was accomplished with anti-mouse IgG antibodies labeled with alkaline phosphatase. The results are summarized in Tables 2 and 3.

TABLE 2
ELISA anti-MUC-1 antibody titers* after 4 immunizations with the
glycolipopeptide/liposome formulations.

| Entry | Group | IgG1 |
|---|---|---|
| 1. | 1. Pam₂Cys-MUC-1 | 24,039 |
| 2. | 2. Pam₂Cys-MUC-1 + QS-21 | 36,906 |
| 3. | 3. Pam₃Cys-MUC-1 | 183,085 |
| 4. | 4. Pam₃Cys-MUC-1 + QS-21 | 450,494 |

*ELISA plates were coated with a BSA-BrAc-MUC-1 conjugate. Anti-MUC1 antibody titers are presented as means of groups of five mice. Titers are defined as the highest dilution yielding an optical density of 0.1 or greater over background of blank mouse sera.

TABLE 3
ELISA anti-MUC-1 antibody titers* after 4 immunizations with the
glycolipopeptide/liposome formulations.

| Entry | Group | IgG1 | IgG2a | IgG2b | IgG3 |
|---|---|---|---|---|---|
| 1. | 1. Pam₂Cys-MUC-1 | 74,104 | 3,599 | 5,515 | 17,437 |
| 2. | 2. Pam₂Cys- | 126,754 | 22,709 | 5,817 | 20,017 |
| | MUC-1 + QS-21 | | | | |
| 3. | 3. Pam₃Cys-MUC-1 | 448,023 | 57,139 | 61,094 | 115,131 |
| 4. | 4. Pam₃Cys- | 653,615 | 450,756 | 70,574 | 305,661 |
| | MUC-1 + QS-21 | | | | |

*ELISA plates were coated with a BSA-BrAc-MUC-1 conjugate. Anti-MUC1 antibody titers are presented as means of groups of five mice. Titers are defined as the highest dilution yielding an optical density of 0.1 or greater over background of blank mouse sera.

As can be seen in Table 2, mice immunized with liposomal preparations of compounds 2 and 3 elicited high titers of anti-MUC-1 IgG antibodies. Surprisingly, mice that were immunized with the Pam₃CysSK₄-based vaccine elicited higher titers of antibodies than mice immunized with Pam₂Cys derivative. These results are contradictory to reports that have compared adjuvancy of Pam₂Cys and Pam₃CysSK₄. Sub-typing of the IgG antibodies (IgG1, IgG2a, IgG2b and IgG3) indicated a bias towards a Th2 immune response (entries 1 and 3, Table 3). Co-administering of the adjuvant QS-21 did not lead to a significant increase of IgG antibody, however, in these cases a mixed Th1/Th2 response was observed (entries 2 and 4, Table 3).

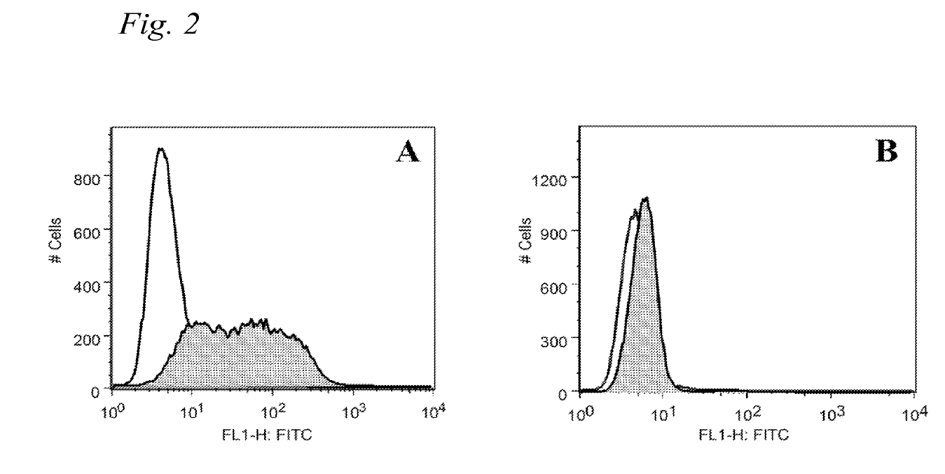

To ensure that the mouse sera were able to recognize native MUC-1 glycopeptide present on cancer cells, the binding of the sera to the MUC-1 expressing MCF-7 human breast cancer cell line was examined. Thus, the cells were treated with a 1:50 diluted sera for 30 minutes after which goat anti-mouse IgG antibodies labeled with FITC was added. The percentage of positive cells and mean fluorescence was determined by flow cytometry analysis. As can be seen in (FIG. 2), the anti-sera reacted strongly with the MUC-1 positive tumor cells whereas no binding was observed for sera obtained from naïve mice. Furthermore, no binding was observed when SK-MEL 28 cell were employed, which do not express the MUC-1 glycopeptide. These results demonstrate that anti-MUC-1 antibodies induced by 3 recognize the native antigen on human cancer cells. Further ELISA studies showed that titers against the T-epitope were very low, showing that no significant epitope suppression had occurred.

The lipopeptide moiety of the three-component vaccine is required for initiating the production of necessary cytokines and chemokines (danger signals) (Bevan, Nat. Rev. Immunol. 2004, 4, 595-602; Eisen et al., Curr. Drug Targets 2004, 5, 89-105; Akira et al., Nat. Immunol. 2001, 2, 675-680; Pasare et al., Immunity 2004, 21, 733-741; Dabbagh et al., Curr. Opin. Infect. Dis. 2003, 16, 199-204; Beutler, Mol. Immunol. 2004, 40, 845-859). The results of recent studies indicate that the lipopeptide initiates innate immune responses by interacting with the Toll-like receptor 2 on the surface of mononuclear phagocytes. After activation, the intracellular domain of TLR-2 recruits the adaptor protein MyD88, resulting in the activation of a cascade of kinases leading to the production of a number of cytokines and chemokines. On the other hand, lipopolysaccharides induce cellular responses by interacting with the Toll-like receptor 4 (TLR4)/MD2, which results in the recruitment of the adaptor proteins MyD88 and TRIF leading to a more complex pattern of cytokine. TNF-α secretion is the prototypical measure for activation of the MyD88-dependent pathway, whereas secretion of IFN-β is commonly used as an indicator of TRIF-dependent cellular activation (Akira et al., Nat. Immunol. 2001, 2, 675-680; Beutler, Mol. Immunol. 2004, 40, 845-859).

To examine whether attachment of a glycopeptide containing a T epitope and a B epitope to the TLR ligand affects cytokine production, the efficacy (EC₅₀) and potency (maximum responsiveness) of TNF-α and IFN-β secretion induced by compounds 1, 2 and 3 was determined and the results compared with those of Pam₂CysSK₄, Pam₃CysSK₄ and LPS. Thus, RAW NO⁻ mouse macrophages were exposed over a wide range of concentrations to compounds 1, 2 and 3, Pam₂CysSK₄, Pam₃CysSK₄ and E. coli 055:B5 LPS. After 5 hours, the supernatants were harvested and examined for mouse TNF-α and IFN-β using commercial or in-house developed capture ELISA assays, respectively.

TABLE 4
EC₅₀ and Eₘₐₓ values of concentration-response curves of E. coli LPS
and synthetic compounds for TNF-α production by mouse macrophages
(RAW γNO(−) cells).

| | EC₅₀ (nM)* | Eₘₐₓ (pg/mL)* |
|---|---|---|
| E. coli LPS | 0.002 | 2585 |
| 1 | 10.230 | 363 |
| Pam₂CysSK₄ | 0.003 | 631 |
| 2 | 0.223 | 622 |
| Pam₃CysSK₄ | 3.543 | 932 |
| 3 | 2.151 | 802 |

*Values of EC50 and Emax are reported as best-fit values according to Prism (GraphPad Software, Inc). Concentration-response data were analyzed using nonlinear least-squares curve fitting in Prism.

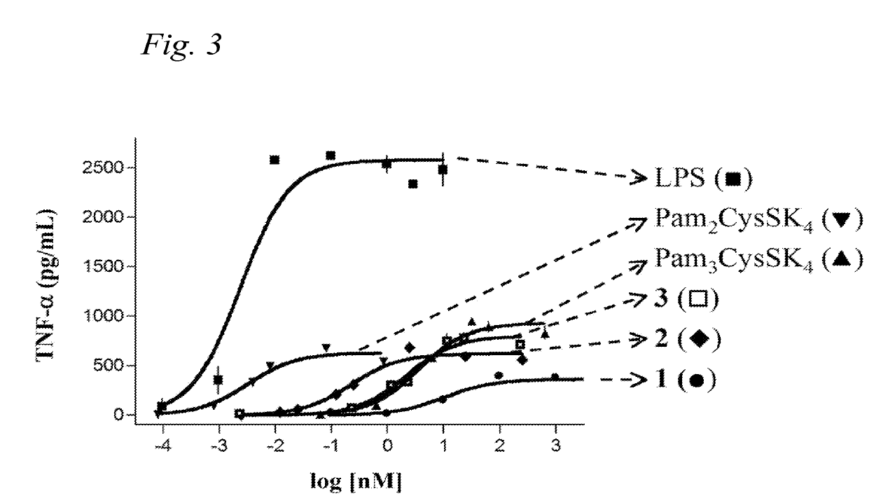

As can be seen in FIG. 3 and Table 4, glycolipopeptide 3 and Pam₃CysSK₄ induced the secretion of TNF-α with similar efficacies and potencies indicating that attachment of the B-epitope and T-epitope had no effect on cytokine and chemokine responses. Surprisingly, attachment of the B-epitope and the T-epitope to Pam₂CysSK₄ led to a significant reduction in potency and thus in this case the attachment of the B-epitope and the T-epitope led to a reduction in activity. Compound 1 which contains the Pam₃Cys moiety is significantly less active than the compounds 2 and 3, which may explain the poor antigenicity of compound 1. Compounds 1, 2 and 3 did not induce the production of TNF-β. Surprisingly, E. coli 055:B5 displayed much larger potencies and efficacies for TNF-a induction compared to compounds 1, 2, 3, and Pam₃CysSK₄. In addition, it was able to stimulate the cells to produce INF-β. E. coli LPS is too active resulting in over-activation of the innate immune system, leading to symptoms of septic shock.

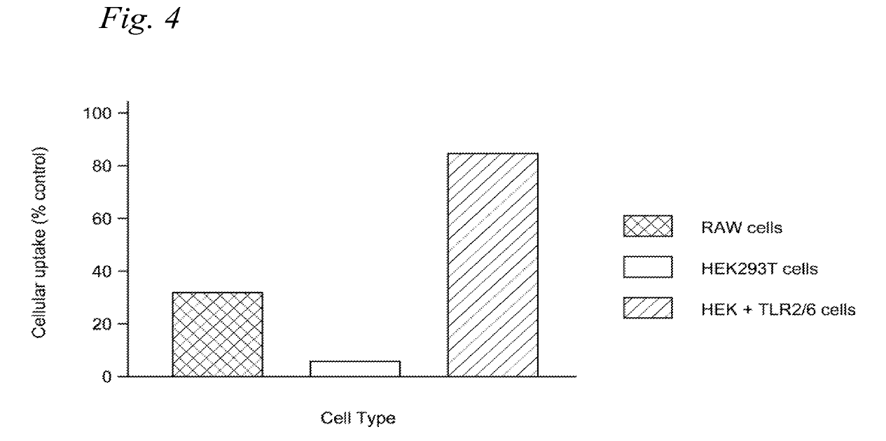

It was speculated that in addition to initiating the production of cytokines and chemokines, the lipopeptide may facilitate selective targeting and uptake by antigen presenting cells in a TLR2 dependent manner. To test this hypothesis, compounds 4, which contains a fluorescence label, was administered to RAW NO⁻ mouse macrophages and after 30 minutes the cells were harvested, lysed and the fluorescence measured. To account for possible cell surface binding without internalization, the cells were also trypsinized before lyses and then examination for fluorescence. As can be seen in FIG. 4, a significant quantity of the 4 was internalized whereas a small amount was attached to the cell surface. To determine whether the uptake was mediated by TLR2, the uptake studies were repeated using native HEK297 cell and HEK297 cell transfected with either TLR2 or TLR4/MD2. Importantly, significant uptake was only observed when the cells were transfected with TLR2 indicating that uptake is mediated by this receptor. These studies show that TLR2 facilitates the uptake of antigen, which is an important step in antigen processing and immune responses.

### Example 4

### Covalent Attachment of the Lipid Component

To establish the importance of covalent attachment of the TLR ligand to the vaccine candidate, compound 5 (Scheme 13) which only contains the B-epitope and the T-epitope was designed and synthesized. Mice were immunized four times at weekly intervals with this compound in the presence of PAM₃CysSK₄. Interestingly, the mixture of glycopeptide 5 and the adjuvant Pam₃CysSK₄ elicited no- or very low titers of IgG antibodies, demonstrating that covalent attachment of Pam₃CysSK₄ to the B-epitope and T-epitope is critical for strong immune responses.

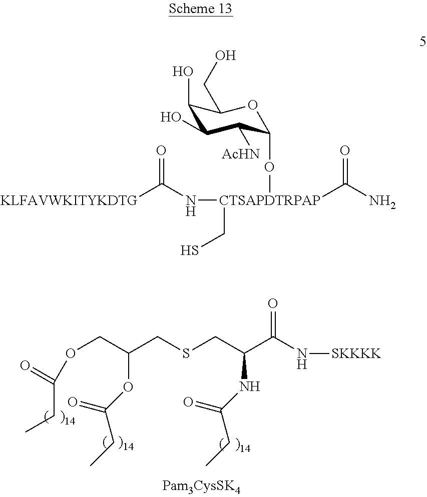

### Example 5

### Lipid Component

To determine the importance of lipidation with a ligand of a Toll like receptor, compound 6 (Scheme 14) was designed and synthesized. This compound is composed of the B-epitope and T-epitope linked to non-immunogenic lipidated amino acids. Mice were immunized with a liposomal preparation of compound 6, similar to the procedure employed for compound 1 and 2. Liposomes containing compound 6 induced titers that were significantly lower than those elicited by compound 3, demonstrating that a TLR ligand of the three-component vaccine is important for optimal immune responses.

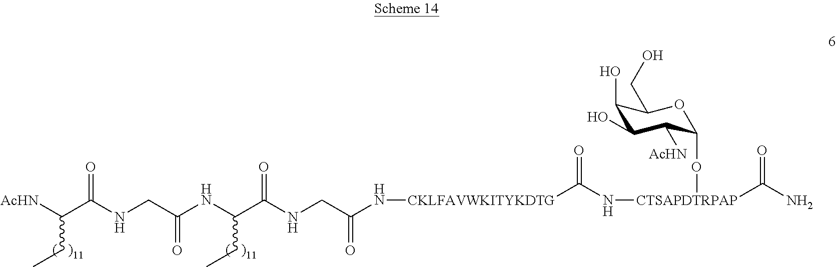

### Conclusions

The three-component carbohydrate-based vaccine has a number of distinctive advantages over a traditional conjugate vaccine. For example, the minimal subunit vaccine does not suffer from epitope suppression, a characteristic of carbohydrate-protein conjugates. Apart from providing danger signals, the lipopeptide Pam₃CysSK₄ also facilitates the incorporation of the antigen into liposomes. A liposomal formulation is attractive because it presents efficiently the antigen to the immune system. A unique feature of the vaccine is that Pam₃CysSK₄ promotes selective targeting and uptake by antigen presenting cells, T-helper cells and B-lymphocytes, which express Toll loll like receptors (Example 3). Finally, a fully synthetic compound has as an advantage that it can be fully characterized, which facilitates its production in a reproducible manner.

### Example 6

### Increasing the Antigenicity of Synthetic Tumor-Associated Carbohydrate Antigens by Targeting Toll-Like Receptors

In this Example, a number of fully synthetic vaccine candidates have been designed, chemically synthesized, and immunologically evaluated to establish strategies to overcome the poor immunogenicity of tumor-associated carbohydrates and glycopeptides and to study in detail the importance of TLR engagement for antigenic responses. Covalent attachment of a TLR2 agonist, a promiscuous peptide T-helper epitope, and a tumor-associated glycopeptide, gives a compound that elicits in mice exceptionally high titers of IgG antibodies which recognize cancer cells expressing the tumor-associated carbohydrate.

The over-expression of oligosaccharides, such as Globo-H, LewisY, and Tn antigens is a common feature of oncogenic transformed cells (Springer, Mol. Med. 1997, 75, 594-602; Hakomori, Acta Anat. 1998, 161, 79-90; Dube, Nat. Rev. Drug Discov. 2005, 4, 477-488). Numerous studies have shown that this abnormal glycosylation can promote metastasis (Sanders, J. Clin. Pathol. Mol. Pathol. 1999, 52, 174-178) and hence the expression of these compounds is strongly correlated with poor survival rates of cancer patients. A broad and expanding body of preclinical and clinical studies demonstrates that naturally acquired, passively administered or actively induced antibodies against carbohydrate-associated tumor antigens are able to eliminate circulating tumor cells and micro-metastases in cancer patients (Livingston, Cancer Immunol. 1997, 45, 10-19; Ragupathi, Cancer Immunol. 1996, 43, 152-157; von Mensdorff-Pouilly, Int. J. Cancer 2000, 86, 702-712; Finn, Nat. Rev. Immunol. 2003, 3, 630-641). Traditional cancer vaccine candidates composed of a tumor-associated carbohydrate (Globo-H, Lewis^{Y}, and Tn) conjugated to a foreign carrier protein (e.g. KLH and BSA) have failed to elicit sufficiently high titers of IgG antibodies in most patients. It appears that the induction of IgG antibodies against tumor-associated carbohydrates is much more difficult than eliciting similar antibodies against viral and bacterial carbohydrates. This observation is not surprising because tumor associated saccharides are self-antigens and consequently tolerated by the immune system. The shedding of antigens by the growing tumor reinforces this tolerance. In addition, a foreign carrier protein such as KLH can elicit a strong B-cell response, which may lead to the suppression of an antibody response against the carbohydrate epitope. The latter is a greater problem when self-antigens such as tumor-associated carbohydrates are employed. Also, linkers that are utilized for the conjugation of carbohydrates to proteins can be immunogenic leading to epitope suppression (Buskas, Chem. Eur. J. 2004, 10, 3517-3524; Ni, Bioconjug. Chem. 2006, 17, 493-500). It is clear that the successful development of a carbohydrate-based cancer vaccine requires novel strategies for the more efficient presentation of tumor-associated carbohydrate epitopes to the immune system, resulting in a more efficient class switch to IgG antibodies (Reichel, J. Chem. Commun. 1997, 21, 2087-2088; Alexander, J. Immunol. 2000, 164, 1625-1633; Kudryashov, Proc. Natl. Acad. Sci. U.S.A. 2001, 98, 3264-3269; Lo-Man, J. Immunol. 2001, 166, 2849-2854; Jiang, Curr. Med. Chem. 2003, 10, 1423-1439; Jackson, Proc. Natl. Acad. Sci. U.S.A. 2004, 101, 15440-5; Lo-Man, Cancer Res. 2004, 64, 4987-4994; Buskas, Angew. Chem. Int. Ed. 2005, 44, 5985-5988 (Example 1); Dziadek, Angew. Chem. Int. Ed. 2005, 44, 7630-7635; Krikorian, Bioconjug. Chem. 2005, 16, 812-819; Pan, J. Med. Chem. 2005, 48, 875-883).

Advances in the knowledge of the cooperation of innate and adaptive immune responses (Pasare, Semin. Immunol. 2004, 16, 23-26; Pashine, Nat. Med. 2005, 11, S63-S68; Akira, Nat. Rev. Immunol. 2004, 4, 499-511; O'Neill, Curr Opin Immunol 2006, 18, 3-9; Lee, Semin Immunol 2007, 19, 48-55; Ghiringhelli, Curr Opin Immunol 2007, 19, 224-31) are offering new avenues for vaccine design for diseases such as cancer, for which traditional vaccine approaches have failed. The innate immune system responds rapidly to families of highly conserved compounds, which are integral parts of pathogens and perceived as danger signals by the host. Recognition of these molecular patterns is mediated by sets of highly conserved receptors, such as Toll-like receptors (TLRs), whose activation results in acute inflammatory responses such as direct local attack against invading pathogens and the production of a diverse set of cytokines. Apart from antimicrobial properties, the cytokines and chemokines also activate and regulate the adaptive component of the immune system (Lin, J Clin Invest 2007, 117, 1175-83). In this respect, cytokines stimulate the expression of a number of co-stimulatory proteins for optimum interaction between T-helper cells and B- and antigen presenting cells (APC). In addition, some cytokines and chemokines are responsible for overcoming suppression mediated by regulatory T-cells. Other cytokines are important for directing the effector T-cell response towards a T-helper-1 (Th-1) or T-helper-2 (Th-2) phenotype (Dabbagh, Curr. Opin. Infect. Dis. 2003, 16, 199-204).

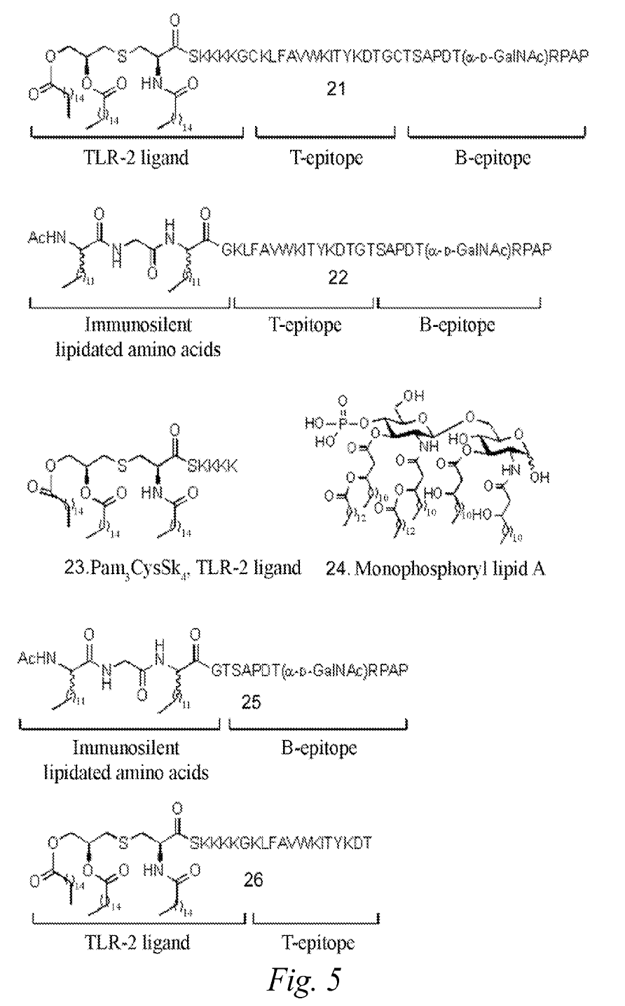

Recently, we described a fully synthetic three-component vaccine candidate (compound 21, FIG. 5) composed of a tumor-associated MUC-1 glycopeptide B-epitope, a promiscuous helper T-cell epitope and a TLR2 ligand (Buskas, Angew. Chem. Int. Ed. 2005, 44, 5985-5988 (Example 1); Ingale, Nat. Chem. Biol. 2007, 3, 663-667; Ingale, J. Org. Lett. 2006, 8, 5785-5788; Bundle, Nat. Chem. Biol. 2007, 3, 604-606). The exceptional antigenic properties of the three-component vaccine were attributed to the absence of any unnecessary features that are antigenic and may induce immune suppression. It contains, however, all the mediators required for eliciting relevant IgG immune responses. Furthermore, attachment of the TLR2 agonist Pam₃CysSK₄ to the B- and T-epitopes ensures that cytokines are produced at the site where the vaccine interacts with immune cells. This leads to a high local concentration of cytokines facilitating maturation of relevant immune cells. Apart from providing danger signals, the lipopeptide Pam₃CysSK₄ facilitates the incorporation of the antigen into liposomes and promotes selective targeting and uptake by antigen presenting cells and B-lymphocytes.

To establish the optimal architecture of a fully synthetic three-component cancer vaccine and to study in detail the importance of TLR engagement for antigenic responses, we have chemically synthesized, and immunologically evaluated a number of fully synthetic vaccine candidates. It has been found that a liposomal preparation of compound 22, which is composed of an immunosilent lipopeptide, a promiscuous peptide T-helper epitope, and a MUC-1 glycopeptide, is significantly less antigenic than compound 21, which is modified with a TLR2 ligand (Pam₃CysSK₄). However, liposomal preparations of compound 22 with Pam₃CysSK₄ (23) or monophosphoryl lipid A (24), which are TLR2 and TLR4 agonists, respectively, elicited titers comparable to compound 21. However, the antisera elicited by mixtures of 22 and 23 or 24 had an impaired ability to recognize cancer cells. Surprisingly, a mixture of compounds 25 and 26, which are composed of a MUC-1 glycopeptide B-epitope linked to lipidated amino acids and the helper T-epitope attached to Pam₃CysSK₄, did not raise antibodies against the MUC-1 glycopeptide. Collectively, the results demonstrate that TLR engagement is not essential but greatly enhanced antigenic responses against the tumor-associated glycopeptide MUC-1. Covalent attachment of the TLR agonist to the B- and helper T-epitope is important for antibody maturation for improved cancer cell recognition.

### Results and Discussion.

### Chemical Synthesis.

Compound 21 (FIG. 5), which contains as B-epitope a tumor-associated glycopeptide derived from MUC-1 (Berzofsky, Nat. Rev. Immunol. 2001, 1, 209-219; Baldus, Crit. Rev. Clin. Lab. Sci. 2004, 41, 189-231; Apostolopoulos, Curr. Opin. Mol. Ther. 1999, 1, 98-103; Hang, Bioorg. Med. Chem. Lett. 2005, 13, 5021-5034), the well-documented murine MHC class II restricted helper T-cell epitope KLFAVWKITYKDT (SEQ ID NO:3) derived from the Polio virus (Leclerc, J. Virol. 1991, 65, 711-718), and the lipopeptide Pam₃CysSK₄ (TLR2 agonist) (Spohn, Vaccine 2004, 22, 2494-2499), was previously shown to elicit exceptionally high titers of IgG antibodies in mice (Ingale, Nat. Chem. Biol. 2007, 3, 663-667). Compound 22 has a similar architecture as 21, however, the TLR2 ligand has been replaced by lipidated amino acids (Toth, Tetrahedron Lett. 1993, 34, 3925-3928). The lipidated amino acids do not induce production of cytokines, however, they enable incorporation of the compound into liposomes. Thus, glycolipopeptide 22 is ideally suited to establish the importance of TLR engagement for antigenic responses against tumor-associated glycopeptides. To determine the importance of covalent attachment of the TLR ligand, liposomal preparations of compound 22 and Pam₃CysSK₄ (23) or monophosphoryl lipid A (24), which are TLR2 and TRL4 agonists, respectively were employed (Spohn, Vaccine 2004, 22, 2494-2499; Chow, J. Biol. Chem. 1999, 274, 10689-10692). Finally, compounds 25 and 26, which are composed of a MUC-1 glycopeptide B-epitope linked to lipidated amino acids and the helper T-epitope attached to Pam₃CysSK₄, were employed to establish the importance of covalent linkage of the B- and helper T-epitope. Compound 21 was prepared as described previously (Ingale, Nat. Chem. Biol. 2007, 3, 663-667; Ingale, Org. Lett. 2006, 8, 5785-5788). Compound 22 was synthesized by SPPS using a Rink amide resin, Fmoc protected amino acids, Fmoc-Thr-(AcO₃-α-D-GalNAc) (Cato, J. Carbohydr. Chem. 2005, 24, 503-516) and Fmoc protected lipidated amino acid (Gibbons, Liebigs Ann. Chem. 1990, 1175-1183; Koppitz, Helv. Chim. Acta 1997, 80, 1280-1300). The standard amino acids were introduced using 2-(1H-bezotriazole-1-yl)-oxy-1,1,3,3-tetramethyl hexafluorophosphate (HBTU)/N-hydroxybenzotriazole (HOBt) (Knorr, Tetrahedron Lett. 1989, 30, 1927-1930) as an activating reagent, the glycosylated amino acid was installed with O-(7-azabenzotriazol-1-yl)-N,N,N′,N′t-tetramethyl-uronium hexafluorophosphate (HATU)/1-hydroxy-7-azabenzotriazole (HOAt), and the lipidated amino acids with benzotriazole-1-yl-oxy-tris-pyrrolidino-phosphonium hexafluorophosphate (PyBOP)/HOBt. After completion of the assembly of the glycolipopeptide, the N-terminal Fmoc protecting group was removed using standard conditions and the resulting amine capped by acetylation with acetic anhydride and diisopropylethyl amine (DIPEA) in N-methylpyrrolidone (NMP). Next, the acetyl esters of the saccharide moiety were cleaved with 60% hydrazine in MeOH and treatment with reagent B (TFA, H₂O, phenol, triethylsilane, 88/5/5/2, v/v/v/v) resulted in removal of the side chain protecting groups and release of the glycopeptide from the solid support.

Pure compound 22 was obtained after purification of the crude product by precipitation with ice-cold diethyl ether followed by HPLC on a C-4 semi-preparative column. A similar protocol was used for the synthesis of compound 25. Derivative 26 was synthesized by SPPS on a Rink amide resin and after assembly of the peptide, the resulting product was coupled manually with N-fluorenylmethoxycarbonyl-R-(2,3-bis(palmitoyloxy)-(2R-propyl)-(R)-cysteine (Fmoc-Pam₂Cys-OH) (Metzger, Int. J. Pept. Protein Res. 1991, 38, 545-554). The N-Fmoc group of the product was removed with 20% piperidine in DMF and the resulting amine was coupled with palmitic acid using and PyBOB, HOBt and DIPEA in DMF. The lipopeptide was treated with reagent B to cleavage it from the resin and to remove side chain protecting groups. The crude product was purified by precipitation with ice-cold diethyl ether followed by HPLC on a C-4 semi-preparative column.

### Immunizations and Immunology.

Compounds 21 and 22 were incorporated into phospholipid-based small uni-lamellar vesicles (SUVs) by hydration of a thin film of egg phosphatidylcholine (PC), phosphatidylglycerol (PG), cholesterol (Chol), and compound 21 or 22 (molar ratios: 65/25/50/10) in a HEPES buffer (10 mM, pH 6.5) containing NaCl (145 mM) followed by extrusion through 100 nm Nuclepore® polycarbonate membrane. Groups of five female BALB/c mice were immunized subcutaneously four times at weekly intervals with liposomes containing 3 μg of saccharide. Furthermore, similar liposomes were prepared of a mixture of glycopeptide 22 with 23 or 24 (molar ratios: PC/PG/Chol/22/23 or 24, 65/25/5/5/5) in HEPES buffer and administered four times at weekly intervals prior to sera harvesting. Finally, mice were immunized with a liposomal preparation of compound 25 and 26 (molar ratios: PC/PG/Chol/25/26, 65/25/5/5/5) employing standard procedures.

Anti-MUC-1 antibody titers of anti-sera were determined by coating microtiter plates with the MUC-1 derived glycopeptide TSAPDT(α-D-GalNAc)RPAP conjugated to BSA and detection was accomplished with anti-mouse IgM or IgG antibodies labeled with alkaline phosphatase. Mice immunized with 21 elicited exceptionally high titers of anti-MUC-1 IgG antibodies (Table 5). Sub-typing of the IgG antibodies (IgG1, IgG2a, IgG2b, and IgG3) indicated a bias towards a Th2 immune response. Furthermore, the observed high IgG3 titer is typical of an anti-carbohydrate response. Immunizations with glycolipopeptide 22, which contains lipidated amino acids instead of a TLR2 ligand, resulted in significantly lower titers of IgG antibodies demonstrating that TLR engagement is very important for optimum antigenic responses. However, liposomal preparations of compound 22 with Pam₃CysSK₄ (23) or monophosphoryl lipid A (24) elicited IgG (total) titers similar to 21. In the case of the mixture of 22 with 23, the immune response was biased towards a Th2 response as evident by high IgG1 and low IgG2a,b titers. On the other hand, the use of monophosphoryl lipid A led to significant IgG1 and IG2a,b responses, and thus this preparation elicited a mixed Th1/Th2 response. Finally, liposomes containing compound 25 and 26 did not induce measurable titers of anti MUC-1 antibodies indicating that the B- and T epitope need to be covalent linked for antigenic responses.

TABLE 5
ELISA anti-MUC1 and anti-T-epitope antibody titers^{a} after
4 immunizations with various preparations.

| Im- | | | | | | | |
|---|---|---|---|---|---|---|---|
| mu- | IgG | | | | | | IgG |
| niza- | total | IgG1 | IgG2a | IgG2b | IgG3 | IgM | total |
| tion^{b} | MUC1 | MUC1 | MUC1 | MUC1 | MUC1 | MUC1 | T-epit. |
| 21 | 177,700 | 398,200 | 49,200 | 37,300 | 116,200 | 7,200 | 23,300 |
| 22 | 13,300 | 44,700 | 300 | 1,800 | 18,600 | 1,300 | 100 |
| 22/23 | 160,500 | 279,800 | 36,200 | 52,500 | 225,600 | 11,000 | 700 |
| 22/24 | 217,400 | 359,700 | 161,900 | 106,000 | 131,700 | 33,400 | 100 |
| 25/26 | 12,800 | 12,700 | 4,800 | 10,100 | 34,400 | 29,000 | 7,600 |

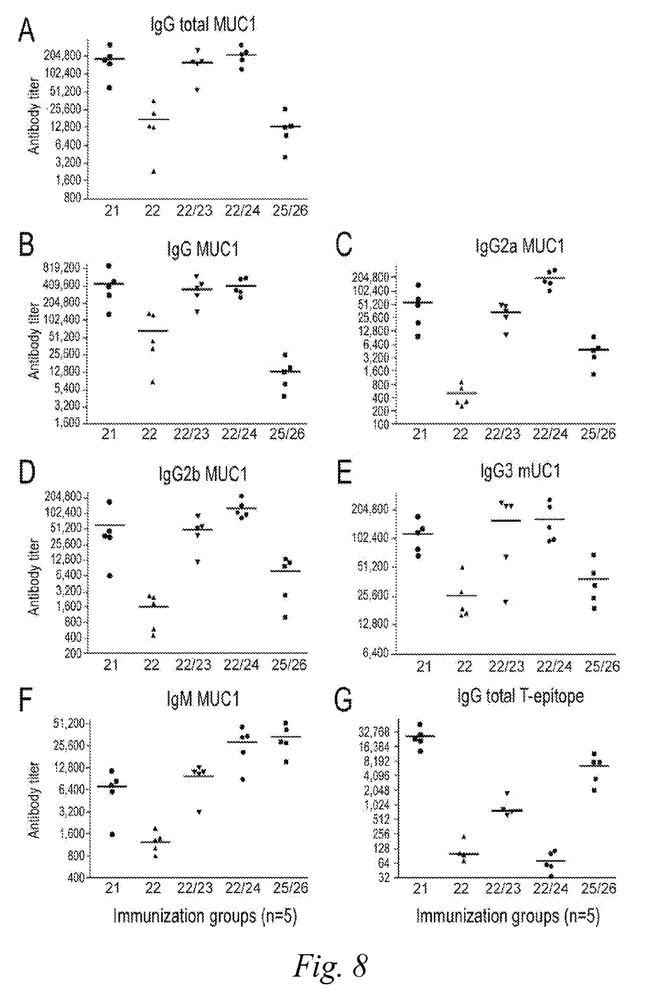

^{a}Anti-MUC1 and anti-T-epitope antibody titers are presented as the median for groups of five mice. ELISA plates were coated with BSA-MI-MUC1 conjugate for anti-MUC1 antibody titers or neutravidin-biotin-T-epitope for anti-T-epitope antibody titers. Titers were determined by linear regression analysis, plotting dilution vs. absorbance. Titers are defined as the highest dilution yielding an optical density of 0.1 or greater over that of normal control mouse sera.
^{b}Liposomal preparations were employed. Individual anti-MUC1 titers for IgG total, IgG1, IgG2a, IgG2b, IgG3 and IgM, and anti-T-epitope for IgG total are reported in FIG. 8.

Next, possible antigenic responses against the helper T-epitope were investigated. Thus, streptavidin coated microtiter plates were treated with the helper T-epitope modified with biotin. After the addition of serial dilutions of sera, detection was accomplished with anti-mouse IgM or IgG antibodies labeled with alkaline phosphatase. Interestingly, compound 21 elicited low whereas mixtures of 22 with 23 or 24 elicited no antibodies against the helper T-epitope.

Pam₃CysSK₄ or monophosphoryl lipid A are employed for initiating the production of cytokines by interacting with TLR2 or TLR4, respectively, on the surface of mononuclear phagocytes (Kawai, Semin. Immunol. 2007, 19, 24-32). After activation with Pam₃CysSK₄, the intracellular domain of TLR2 recruits the adaptor protein MyD88 resulting in the activation of a cascade of kinases leading to the production of a number of cytokines and chemokines. On the other hand, lipopolysaccharides (LPS) and lipid As induce cellular responses by interacting with the TLR4/MD2 complex, which results in the recruitment of the adaptor proteins MyD88 and TRIF leading to the induction of a more complex pattern of cytokine. TNF-α secretion is the prototypical measure for activation of the MyD88-dependent pathway, whereas secretion of IFN-β is commonly used as an indicator of TRIF-dependent cellular activation.

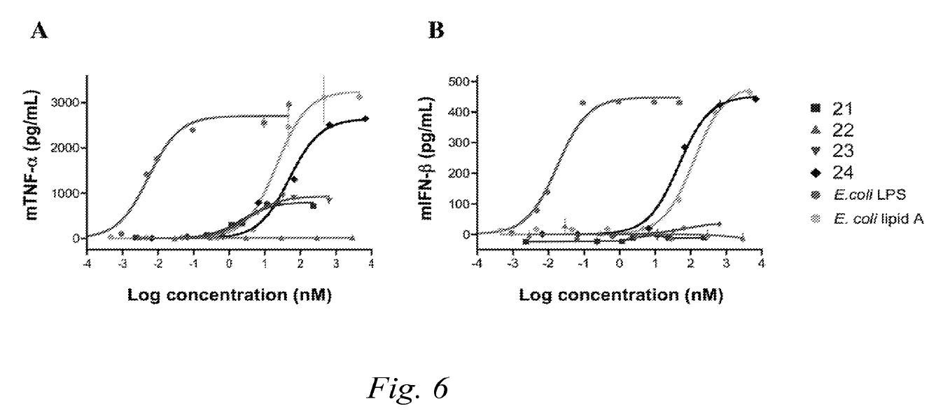

To examine cytokine production, mouse macrophages (RAW γNO(−) cells) were exposed over a wide range of concentrations to compounds 21-24, E. coli 055:B5 LPS and prototypic E. coli bisphosphoryl lipid A (Zhang, J. Am. Chem. Soc. 2007, 129, 5200-5216). After 5.5 h, the supernatants were harvested and examined for mouse TNF-α and IFN-β using commercial or in-house developed capture ELISAs, respectively (FIG. 6). Potencies (EC₅₀, concentration producing 50% activity) and efficacies (maximal level of production) were determined by fitting the dose-response curves to a logistic equation using PRISM software. Glycolipopeptide 21 and Pam₃CysSK₄ (23) induced secretion of TNF-α with similar efficacies and potencies, indicating that attachment of the B- and T-epitopes had no effect on cytokine responses. As expected, none of the compounds induced the production of INF-β. Furthermore, compound 22 did not induce TNF-α and IFN-β secretion, indicating that its lipid moiety is immunosilent. Compound 24 stimulated the cells to produce TNF-α and INF-β but its potency was much smaller than that of E. coli 055:B5 LPS. It displayed a much larger efficacy of TNF-α production compared to compounds 21 and 23. The reduced efficacy of compounds 21 and 23 is probably a beneficial property, because LPS can over-activate the innate immune system leading to symptoms of septic shock.

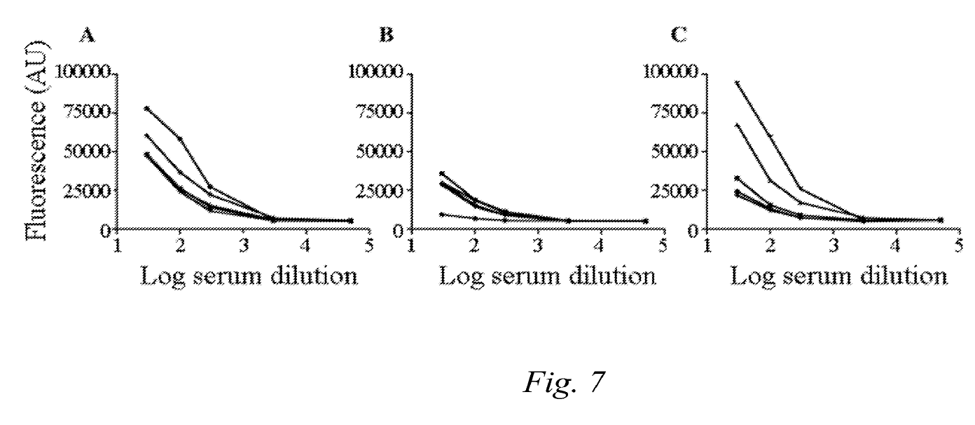

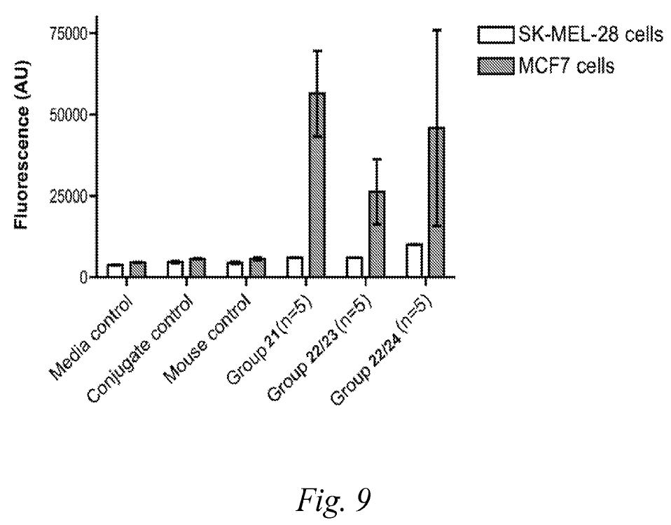

Next, the ability of the mouse antisera to recognize native MUC-1 antigen present on cancer cells was established. Thus, serial dilutions of the serum samples were added to MUC-1 expressing MCF-7 human breast cancer cells (Horwitz, Steroids 1975, 26, 785-95) and recognition was established using a FITC-labeled anti-mouse IgG antibody. As can be seen in FIG. 7, anti-sera obtained from immunizations with the three-component vaccine 1 displayed excellent recognition of MUC-1 tumor cell whereas no binding was observed when SK-MEL 28 cells, which do not express the MUC-1 antigen, were employed (FIG. 9).

Although sera obtained from mice immunizations with a mixture of lipidated T-B epitope (22) and Pam₃CysSK₄ (23) elicited equally high IgG antibody titers as 21 (Table 5), a much-reduced recognition of MCF-7 cells was observed. This result indicates that covalent attachment of the adjuvant PamsCysSK₄ (23) to the B-T epitope is important for proper antibody maturation leading to improved cancer cell recognition. Immunizations with a mixture of compound 22 and monophosphoryl lipid A (24) led to variable results and two mice displayed excellent, and three modest, recognition of MCF-7 cells.

### Discussion

Most efforts aimed at developing carbohydrate-based cancer vaccines have focused on the use of chemically synthesized tumor-associated carbohydrates linked through an artificial linker to a carrier protein (Springer, Mol. Med. 1997, 75, 594-602; Dube, Nat. Rev. Drug Discov. 2005, 4, 477-488; Ouerfelli, Expert Rev. Vaccines 2005, 4, 677-685; Slovin, Immunol. Cell Biol. 2005, 83, 418-428). It has been established that the use of KLH as a carrier protein in combination with the powerful adjuvant QS-21 gives the best results. However, a drawback of this approach is that KLH is a very large and cumbersome protein that can elicit high titers of anti-KLH-antibodies (Cappello, Cancer Immunol Immunother 1999, 48, 483-492), leading to immune suppression of the tumor-associated carbohydrate epitope. Furthermore, the conjugation chemistry is often difficult to control as it results in conjugates with ambiguities in composition and structure, which may affect the reproducibility of immune responses. Also, the linker moiety can elicit strong B-cell responses (Buskas, Chem. Eur. J. 2004, 10, 3517-3524; Ni, Bioconjug. Chem. 2006, 17, 493-500). Not surprisingly, preclinical and clinical studies with carbohydrate-protein conjugates have led to results of mixed merit. For example, mice immunized with a trimeric cluster of Tn-antigens conjugated to KLH (Tn(c)-KLH) in the presence of the adjuvant QS-21 elicited modest titers of IgG antibodies (Kuduk, J. Am. Chem. Soc. 1998, 120, 12474-12485). Examination of the vaccine candidate in a clinical trial of relapsed prostate cancer patients gave low median IgG and IgM antibody titer (Slovin, J. Clin. Oncol. 2003, 21, 4292-4298).

The studies reported herein show that a three-component vaccine, in which a MUC-1 associated glycopeptide B-epitope, a promiscuous murine MHC class II restricted helper T-cell epitope, and a TLR2 agonist (21) are covalently linked, can elicit robust IgG antibody responses. Although covalent attachment of the TLR2 ligand to the T-B glycopeptide epitope was not required for high IgG antibody titers, it was found to be very important for optimal cancer cell recognition. In this respect, liposomes containing compounds 21 or a mixture compound 22 and TLR2 agonist 23 elicited similar high anti-MUC-1 IgG antibody titers. However, antisera obtained from immunizations with 21 recognized MUC-1 expressing cancer cells at much lower sera dilutions than antisera obtained from immunizations with a mixture of 22 and 23. It appears that immunizations with three-component vaccine 21 lead to more efficient antibody maturation resulting in improved cancer cell recognition.

Differences in antigenic responses against the helper T-epitope were also observed. Thus, 21 elicited low titers of IgG antibodies against the helper T-epitope whereas mixtures of 22 with 23 induced no antigenic responses against this part of the candidate vaccine. Thus, the covalent attachment of the TLR2 ligand makes compound 21 more antigenic resulting in low antibody responses against the helper T-epitope.

It was observed that a mixture of compound 22 with 23 or 24 induced similar high titers of total IgG antibodies. However, a bias towards a Th2 response (IgG1) was observed when the TLR2 agonist Pam₃CysSK₄ (23) was employed whereas mixed Th1/Th2 responses (IgG2a,b) was obtained when the TLR4 agonist monophosphoryl lipid A (24) was used. The difference in polarization of helper T-cells is probably due to the induction of different patterns of cytokines by TLR2 or TLR4. In this respect, it was previously observed that Pam₃Cys induces lower levels of Th1 inducing cytokines Il-12(p70) and much higher levels of Th2-inducing IL-10 than E. coli LPS (Dillon, B. J Immunol 2004, 172, 4733-43). The differences are likely due to the ability of TLR4 to recruit the adaptor proteins MyD88 and Trif whereas TLR2 can only recruit MyD88. The results indicate that the immune system can be tailored in a particular direction by proper selection of an adjuvant, which is significant since different IgG isotypes perform different effector functions.

The results described herein also show that compound 22 alone, which contains an immuno-silent lipopeptide, elicits much lower IgG titers compared to compound 21, which is modified by a TLR2 ligand. In particular, the ability of compound 22 to elicit IgG2 antibodies was impaired. Recent studies employing mice deficient in TLR signaling have cast doubt about the importance of these innate immune receptors for adaptive immune responses (Blander, Nature 2006, 440, 808-812; Gavin, Science 2006, 314, 1936-1938; Meyer-Bahlburg, J Exp Med 2007, 204, 3095-101; Pulendran, N Engl J Med 2007, 356, 1776-8). In this respect, studies with MyD88 deficient mice showed that IgM and IgG1 are largely, but not completely, dependent of TLR signaling whereas the IgG2 isotype is entirely TLR-dependent (Blander, Nature 2006, 440, 808-812). These observations, which are in agreement with the results reported here, were attributed to a requirement of TLR signaling for B-cell maturation. However, another study found that MyD88^{-/-}/Trif^{lps/lps} double knockout mice elicited similar titers of antibodies as wild type mice when immunized with trinitrophenol-hemocyanin (TNP-Hy) or TNP-KLH in the presence or absence of several adjuvants (Gavin, Science 2006, 314, 1936-1938). It was concluded that it might be desirable to exclude TLR agonists from adjuvants. It has been noted that the importance of an adjuvant may depend on the antigenicity of the immunogen (Meyer-Bahlburg, J Exp Med 2007, 204, 3095-101; Pulendran, N Engl J Med 2007, 356, 1776-8). In this respect, proteins conjugates of TNP are highly antigenic and may not require an adjuvant for optimal responses. However, self-antigens such as tumor-associated carbohydrates have low intrinsic antigenicity and the results reported here clearly show that much more robust antibody responses are obtained when a TLR ligand is co-administered. In addition, it is demonstrated here that the architecture of a candidate vaccine is very important for optimal antigenic responses and in particular covalent attachment of a TLR ligand to a T-B epitope led to improved cancer cell recognition.

The failure of a mixture of compounds 25 and 26 to elicit anti-MUC-1 glycopeptide antibodies indicates that covalent attachment of the T- to the B-epitope is needed to elicit antigenic responses. In this respect, activation of B-cells by helper T-cells requires a similar type of cell-cell interaction as for helper T-cell activation by antigen presenting cells. Thus, a protein or peptide-containing antigen needs to be internalized by B-cells for transport to endosomal vesicles, where proteases will digest the protein and some of the resulting peptide fragments will be complexed with class II MHC protein. The class II MHC-peptide complex will then be transported to the cell surface of the B-lymphocyte to mediate an interaction with helper T-cell resulting in a class switch from low affinity IgM to high affinity IgG antibody production. Unlike antigen presenting cells, B-cells have poor phagocytic properties and can only internalize molecules that bind to the B-cell receptor. Therefore, it is to be expected that internalization of the helper T-epitope is facilitated by covalent attachment to the B-epitope (MUC-1 glycopeptide) and as a result covalent attachment of the two epitopes will lead to more robust antigenic responses.

In conclusion, it has been demonstrated that antigenic properties of a fully synthetic cancer vaccine can be optimized by structure-activity relationship studies. In this respect, it has been established that a three-component vaccine in which a tumor-associated MUC-1 glycopeptide B-epitope, a promiscuous helper T-cell epitope and a TLR2 ligand are covalently linked can elicit exceptionally high IgG antibody responses, which have an ability to recognize cancer cells. It is very important that the helper T-epitope is covalently linked to the B-epitope, probably since internalization of the helper T-epitope by B-cells requires the presence of a B-epitope. It has also been shown that incorporation of a TLR agonist is important for robust antigenic responses against tumor associated glycopeptide antigens. In this respect, cytokines induced by the TLR2 ligand are important for maturation of immune cells leading to robust antibody responses. A surprising finding was that improved cancer cell recognition was observed when the TLR2 epitope was covalently attached to the glycopeptide T-B epitope. The result presented here provides important information of the optimal constitution of three-component vaccines and will guide successful development of carbohydrate-based cancer vaccines.

### Experimental

Peptide Synthesis:

Peptides were synthesized by established protocols on an ABI 433A peptide synthesizer (Applied Biosystems), equipped with a UV-detector using N^{α}-Fmoc-protected amino acids and 2-(1H-bezotriazole-1-yl)-oxy-1,1,3,3-tetramethyl hexafluorophosphate (HBTU)/N-hydroxybenzotriazole (HOBt) (Knorr, Tetrahedron Lett. 1989, 30, 1927-1930) as the activating reagents. Single coupling steps were performed with conditional capping. The following protected amino acids were used: N^{α}-Fmoc-Arg(Pbf)-OH, N^{α}-Fmoc-Asp(O^{t}Bu)—OH, N^{α}-Fmoc-Asp-Thr(Ψ^{Me,Me}pro)-OH, N^{α}-Fmoc-Ile-Thr(Ψ^{Me,Me}pro)-OH, N^{α}-Fmoc-Lys(Boc)-OH, N^{α}-Fmoc-Ser(^{t}Bu)—OH, N^{α}-Fmoc-Thr(^{t}Bu)—OH, and N^{α}-Fmoc-Tyr(^{t}Bu)—OH. The coupling of glycosylated amino acid N^{α}-Fmoc-Thr-(AcO₃-α-D-GalNAc) 1S (Cato, J. Carbohydr. Chem. 2005, 24, 503-516) was carried out manually using O-(7-azabenzotriazol-1-yl)-N,N,N′,N′-tetramethyl-uronium hexafluorophosphate (HATU)/1-hydroxy-7-azabenzotriazole (HOAt) as a coupling agent. The coupling of N^{α}-Fmoc-lipophilic amino acid (N^{α}-Fmoc-D,L-tetradeconic acid) 2S (Gibbons, Liebigs Ann. Chem. 1990, 1175-1183; Koppitz, Helv. Chim. Acta 1997, 80, 1280-1300) and N^{α}-Fmoc-S-(2,3-bis(palmitoyloxy)-(2R-propyl)-(R)-cysteine 3S (Metzger, Int. J. Pept. Protein Res. 1991, 38, 545-554; Roth, Bioconj. Chem. 2004, 15, 541-553), which was prepared from (R)-glycidol, were carried out using benzotriazole-1-yl-oxy-tris-pyrrolidino-phosphonium hexafluorophosphate (PyBOP)/HOBt as coupling agent (See Supporting Information). Progress of the manual couplings was monitored by standard Kaiser test (Kaiser, Anal. Biochem. 1970, 34, 595).

Liposome Preparation:

Egg phosphatidylcholine (PC), phosphatidylglycerol (PG), cholesterol (Chol) and compound 21 or 22 (15 mmol, molar ratios 65:25:50:10) or PC/PG/Chol/22/23 or 24 (15 mmol, molar ratios 60:25:50:10:5) or PC/PG/Chol/25/26 (15 mmol, molar ratios 65:25:50:5:5) were dissolved in a mixture of trifluoroethanol and MeOH (1:1, v/v, 5 mL). The solvents were removed in vacuo to give a thin lipid film, which was hydrated by shaking in HEPES buffer (10 mM, pH 6.5) containing NaCl (145 mM) (1 mL) under argon atmosphere at 41° C. for 3 h. The vesicle suspension was sonicated for 1 min and then extruded successively through 1.0, 0.4, 0.2, and 0.1 μm polycarbonate membranes (Whatman, Nuclepore® Track-Etch Membrane) at 50° C. to obtain SUVs. The GalNAc content was determined by heating a mixture of SUVs (50 μL) and aqueous TFA (2 M, 200 μL) in a sealed tube for 4 h at 100° C. The solution was then concentrated in vacuo and analyzed by high-pH anion exchange chromatography using a pulsed amperometric detector (HPAEC-PAD; Methrome) and CarboPac columns PA-10 and PA-20 (Dionex).

Dose and Immunization Schedule:

Groups of five mice (female BALB/c, age 8-10 weeks; Jackson Laboratories) were immunized four times at weekly intervals. Each boost included 3 μg of saccharide in the liposome formulation. Serum samples were obtained before immunization (pre-bleed) and one week after the final immunization. The final bleeding was done by cardiac bleed.

Serologic Assays:

Anti-MUC-1 IgG, IgG1, IgG2a, IgG2b, IgG3, and IgM antibody titers were determined by enzyme-linked immunosorbent assay (ELISA), as described previously (Buskas, Chem. Eur. J. 2004, 10, 3517-3524). Briefly, ELISA plates (Thermo Electron Corp.) were coated with a conjugate of the MUC-1 glycopeptide conjugated to BSA through a maleimide linker (BSA-MI-MUC-1). Serial dilutions of the sera were allowed to bind to immobilized MUC-1. Detection was accomplished by the addition of phosphate-conjugated anti-mouse IgG (Jackson ImmunoResearch Laboratories Inc.), IgG1 (Zymed), IgG2a (Zymed), IgG2b (Zymed), IgG3 (BD Biosciences Pharmingen), or IgM (Jackson ImmunoResearch Laboratories Inc.) antibodies. After addition of p-nitrophenyl phosphate (Sigma), the absorbance was measured at 405 nm with wavelength correction set at 490 nm using a microplate reader (BMG Labtech). Antibody titers against the T (polio)-epitope were determined as follows. Reacti-bind NeutrAvidin coated and pre-blocked plates (Pierce) were incubated with biotin-labeled T-epitope (10 μg/mL) for 2 h. Next, serial dilutions of the sera were allowed to bind to immobilized T-epitope. Detection was accomplished as described above. The antibody titer was defined as the highest dilution yielding an optical density of 0.1 or greater over that of normal control mouse sera.

Cell Culture:

RAW 264.7 γNO(−) cells, derived from the RAW 264.7 mouse monocyte/macrophage cell line, were obtained from ATCC. The cells were maintained in RPMI 1640 medium with L-glutamine (2 mM), adjusted to contain sodium bicarbonate (1.5 g L⁻¹), glucose (4.5 g L⁻¹), HEPES (10 mM) and sodium pyruvate (1.0 mM) and supplemented with penicillin (100 u mL⁻¹)/streptomycin (100 μmL⁻¹; Mediatech) and FBS (10%; Hyclone). Human breast adenocarcinoma cells (MCF7) (Horwitz, Steroids 1975, 26, 785-95), obtained from ATCC, were cultured in Eagle's minimum essential medium with L-glutamine (2 mM) and Earle's BSS, modified to contain sodium bicarbonate (1.5 g L⁻¹), non-essential amino acids (0.1 mM) and sodium pyruvate (1 mM) and supplemented with bovine insulin (0.01 mg mL⁻¹; Sigma) and FBS (10%). Human skin malignant melanoma cells (SK-MEL-28) were obtained from ATCC and grown in Eagle's minimum essential medium with L-glutamine (2 mM) and Earle's BSS, adjusted to contain sodium bicarbonate (1.5 g L⁻¹), non-essential amino acids (0.1 mM) and sodium pyruvate (1 mM) and supplemented with FBS (10%). All cells were maintained in a humid 5% CO₂ atmosphere at 37° C.

TNF-α and IFN-β Assays.

RAW 264.7 γNO(−) cells were plated on the day of the exposure assay as 2×10⁵ cells/well in 96-well plates (Nunc) and incubated with different stimuli for 5.5 h in the presence or absence of polymyxin B. Culture supernatants were collected and stored frozen (−80° C.) until assayed for cytokine production. Concentrations of TNF-α were determined using the TNF-α DuoSet ELISA Development kit from R&D Systems. Concentrations of IFN-β were determined as follows. ELISA MaxiSorp plates were coated with rabbit polyclonal antibody against mouse IFN-β (PBL Biomedical Laboratories). IFN-β in standards and samples was allowed to bind to the immobilized antibody. Rat anti-mouse IFN-β antibody (USBiological) was then added, producing an antibody-antigen-antibody “sandwich”. Next, horseradish peroxidase (HRP) conjugated goat anti-rat IgG (H+L) antibody (Pierce) and a chromogenic substrate for HRP 3,3′,5,5′-tetramethylbenzidine (TMB; Pierce) were added. After the reaction was stopped, the absorbance was measured at 450 nm with wavelength correction set to 540 nm. Concentration-response data were analyzed using nonlinear least-squares curve fitting in Prism (GraphPad Software, Inc.). These data were fit with the following four parameter logistic equation: Y=Eₘₐₓ/(1+(EC₅₀/X)^{Hill slope}), where Y is the cytokine response, X is the concentration of the stimulus, Eₘₐₓ is the maximum response and EC₅₀ is the concentration of the stimulus producing 50% stimulation. The Hill slope was set at 1 to be able to compare the EC₅₀ values of the different inducers. All cytokine values are presented as the means±SD of triplicate measurements, with each experiment being repeated three times.

Evaluation of Materials for Contamination by LPS:

To ensure that any increase in cytokine production was not caused by LPS contamination of the solutions containing the various stimuli, avidly binds to the lipid A region of LPS, thereby preventing LPS-induced cytokine production (Tsubery, Biochemistry 2000, 39, 11837-44). TNF-α and IFN-β concentrations in supernatants of cells preincubated with polymyxin B (30 μg mL⁻¹; Bedford Laboratories) for 30 min before incubation with E. coli 055:B5 LPS for 5.5 h showed complete inhibition, whereas preincubation with polymyxin B had no effect on TNF-α synthesis by cells incubated with the synthetic compounds 21 and 23. Therefore, LPS contamination of the latter preparations was inconsequential.

Cell Recognition Analysis by Fluorescence Measurements:

Serial dilutions of pre- and post-immunization sera were incubated with MCF7 and SK-MEL-28 single-cell suspensions for 30 min on ice. Next, the cells were washed and incubated with goat anti-mouse IgG γ-chain specific antibody conjugated to fluorescein isothiocyanate (FITC; Sigma) for 20 min on ice. Following three washes and cell lysis, cell lysates were analyzed for fluorescence intensity (485 ex/520 em) using a microplate reader (BMG Labtech). Data points were collected in triplicate and are representative of three separate experiments.

### Example 7

### Synthesis of Compounds

General Methods:

Fmoc-L-amino acid derivatives and resins were purchased from NovaBioChem and Applied Biosystems; peptide synthesis grade N,N-dimethylformamide (DMF) from EM Science; and N-methylpyrrolidone (NMP) from Applied Biosystems. Egg phosphatidylcholine (PC), phosphatidylglycerol (PG), cholesterol (Chol), and monophosphoryl lipid A (MPL-A) were obtained from Avanti Polar Lipids. EZ-Link® NHS-Biotin reagent (succinimidyl-6-(biotinamido)hexanoate) was obtained from Pierce. All other chemical reagents were purchased form Aldrich, Acros, Alfa Aesar, and Fisher Scientific and used without further purification. All solvents employed were reagent grade. Reversed phase high performance liquid chromatography (RP-HPLC) was performed on an Agilent 1100 series system equipped with an auto-injector, fraction-collector, and UV-detector (detecting at 214 nm) using an Agilent Zorbax Eclipse™ C8 analytical column (5 μm, 4.6×150 mm) at a flow rate of 1 mL/min, Agilent Zorbax Eclipse™ C8 semi preparative column (5 μm, 10×250 mm) at a flow rate of 3 mL/min or Phenomenex Jupiter™ C4 semi preparative column (5 μm, 10×250 mm) at a flow rate of 2 mL/min. All runs were performed using a linear gradient of 0-100% solvent B over 40 min (solvent A=5% acetonitrile, 0.1% trifluoroacetic acid (TFA) in water, solvent B=5% water, 0.1% TFA in acetonitrile). Matrix assisted laser desorption ionization time of flight mass spectrometry (MALDI-ToF) mass spectra were recorded on a ABI 4700 proteomic analyzer.

Synthesis of Glycolipopeptide 22:

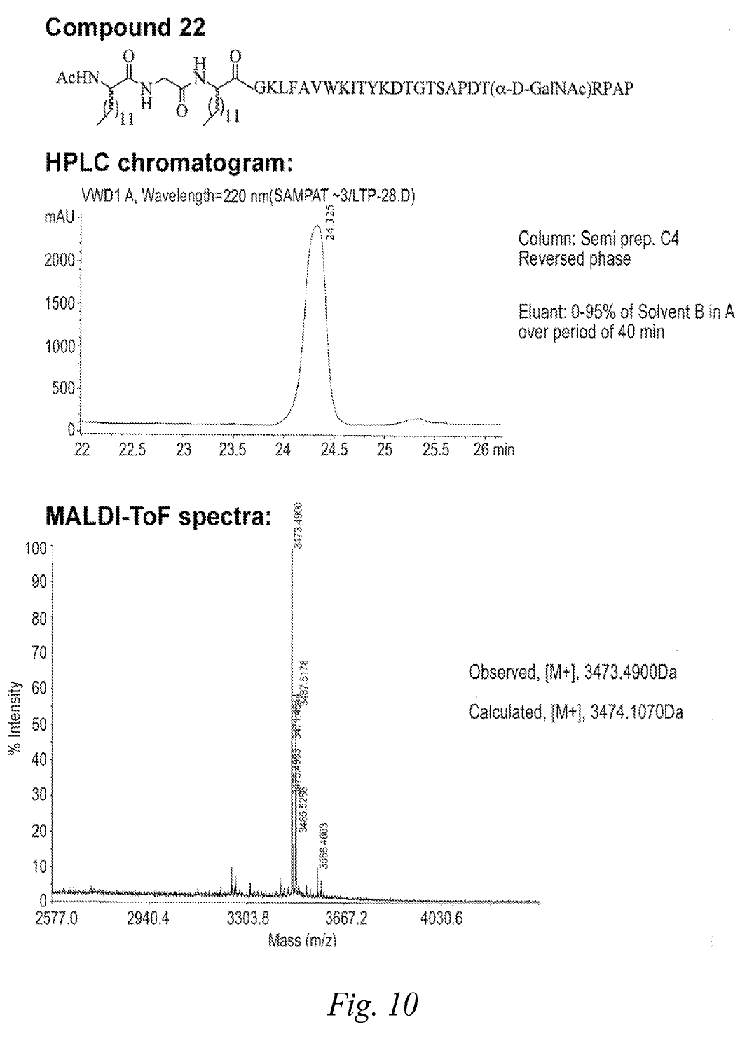

The synthesis 22 was carried out on a Rink amide resin (28, 0.1 mmol) as described under peptide synthesis in the experimental. The first four amino acids, Arg-Pro-Ala-Pro were coupled on the peptide synthesizer using a standard protocol to obtain 29. After the completion of the synthesis, a manual coupling of 1S (0.2 mmol, 134 mg) was carried out. N^{α}-Fmoc-Thr-(AcO₃-α-D-GalNAc) 1S (Cato, J. Carbohydr. Chem. 2005, 24, 503-516) was dissolved in NMP (5 mL) and O-(7-azabenzotriazol-1-yl)-N,N,N′,N′-tetramethyl-uronium hexafluorophosphate (HATU; 0.2 mmol, 76 mg), 1-hydroxy-7-azabenzotriazole (HOAt; 0.2 mmol, 27 mg), and diisopropylethylamine (DIPEA; 0.4 mmol, 70 μL) were added to the solution and the resulting mixture was added to the resin. The coupling reaction was monitored by standard Kaiser test. After 12 h, the resin was washed with NMP (6 mL) and methylene chloride (DCM; 6 mL), and resubjected to the same coupling conditions to ensure complete coupling. The glycopeptide 30 was then elongated on the peptide synthesizer. After the completion of the synthesis, the resin was thoroughly washed with NMP (6 mL), DCM (6 mL) and methanol (MeOH; 6 mL) and dried in vacuo. The resin was then swelled in DCM (5 mL) for 1 h and the rest of the couplings were carried out manually. Next, N^{α}-Fmoc-lipophilic amino acid (N^{α}-Fmoc-D,L-tetradeconic acid) 2S (Gibbons, Liebigs Ann. Chem. 1990, 1175-1183; Koppitz, Helv. Chim. Acta 1997, 80, 1280-1300) (0.3 mmol, 139 mg) dissolved in NMP (5 mL), benzotriazole-1-yl-oxy-tris-pyrrolidino-phosphonium hexafluorophosphate (PyBOP; 0.3 mmol, 156 mg), HOBt (0.3 mmol, 40 mg) and DIPEA (0.4 mmol, 67 μL) were premixed for 2 min., and then added to the resin. The coupling reaction was monitored by the Kaiser test and was complete after standing for 8 h. The N^{α}-Fmoc group was cleaved using piperidine (20%) in DMF (6 mL). N^{α}-Fmoc-Gly-OH (0.3 mmol, 90 mg) dissolved in NMP (5 mL), PyBOP (0.3 mmol, 156 mg), HOBt (0.3 mmol, 40 mg), and DIPEA (0.4 mmol, 67 μL) were premixed for 2 min, and were then added to the resin. The coupling reaction was monitored by Kaiser test and was complete after standing for 4 h. The N^{α}-Fmoc group was cleaved using piperidine (20%) in DMF (6 mL). One more cycle of coupling of 2S (0.3 mmol, 139 mg) was carried out as described above using PyBOP (0.3 mmol, 156 mg), HOBt (0.3 mmol, 40 mg), and DIPEA (0.4 mmol, 67 μL) in NMP (5 mL). Finally, the N^{α}-Fmoc group was cleaved using piperidine (20%) in DMF (6 mL) and the resulting free amino group was acetylated by treatment of the resin with Ac₂O (10%) and DIPEA (5%) in NMP (5 mL) for 10 min. The resin was washed thoroughly with NMP (5 mL×2), DCM (5 mL×2), and MeOH (5 mL×2), and dried in vacuo. The resin was swelled in DCM (5 mL) for 1 h, treated with hydrazine (60%) in MeOH^{4,5} (10 mL) for 2 h, thoroughly washed with NMP (5 mL×2), DCM (5 mL×2), and MeOH (5 mL×2), and dried in vacuo. The resin was swelled in DCM (5 mL) for 1 h and then treated with reagent B (TFA (88%), water (5%), phenol (5%), and TIS (2%), 10 mL) for 2 h. The resin was filtered, washed with neat TFA (2 mL), and the filtrate was then concentrated in vacuo to approximately ⅓ of its original volume. The glycolipopeptide was precipitated using diethyl ether (0° C., 40 mL) and recovered by centrifugation at 3,000 rpm for 15 min. The crude glycolipopeptide was purified by RP-HPLC on a semi preparative C-4 column using a linear gradient of 0-95% solvent B in A over 40 min, and the appropriate fractions were lyophilized to afford 22 (FIG. 10) (57 mg, 16%). C₁₆₅H₂₆₇N₃₇O₄₄, MALDI-ToF MS: observed, [M+] 3473.4900 Da; calculated, [M+] 3473.1070 Da.

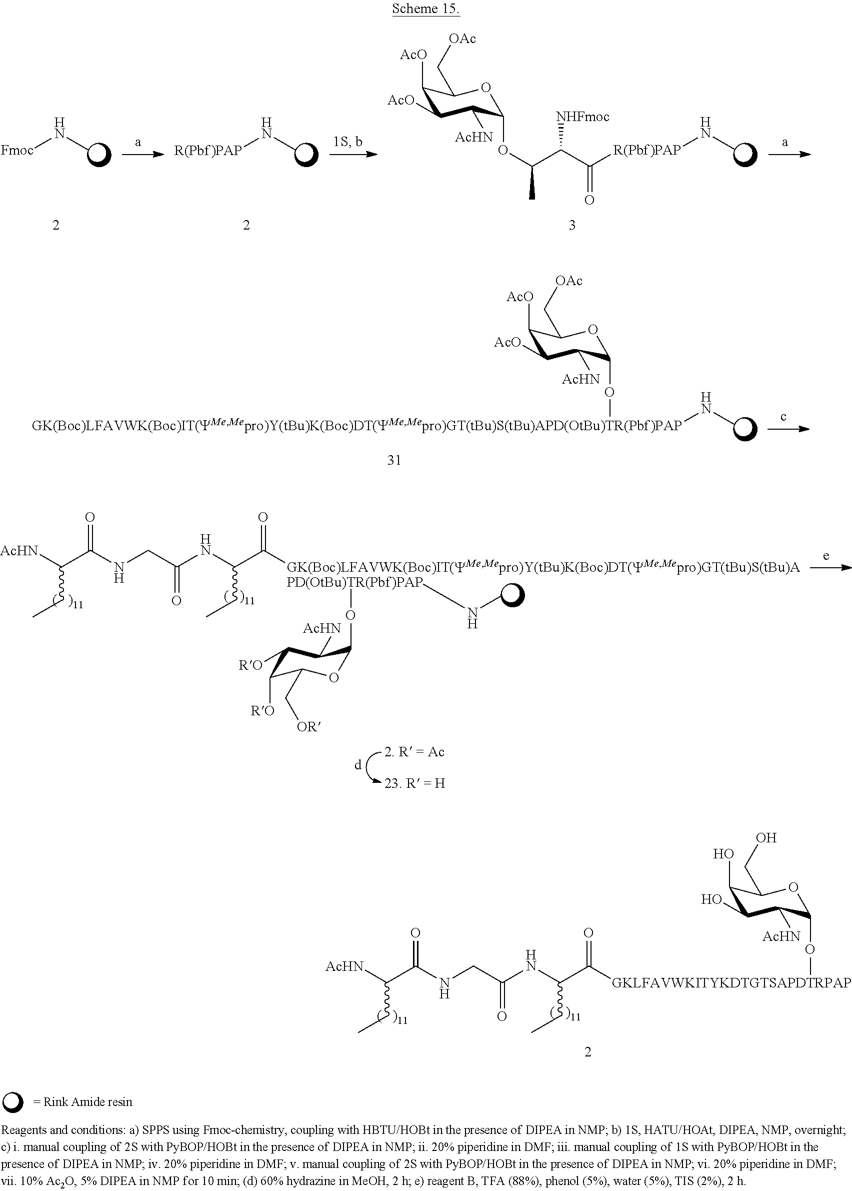

Synthesis of Lipopeptide 23:

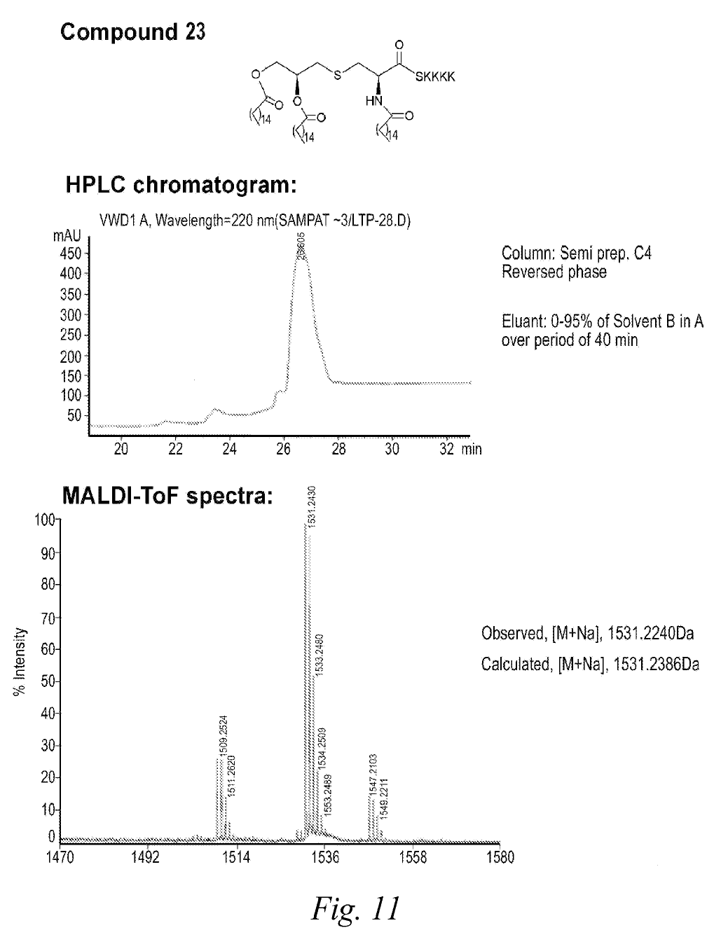

The synthesis of 23 was carried out on a Rink amide resin (28, 0.1 mmol) as described under peptide synthesis in the experimental. After coupling of the first five amino acids, the lipid portion of the molecule was coupled manually. N^{α}-Fmoc-S-(2,3-bis(palmitoyloxy)-(2R-propyl)-(R)-cysteine, 3S (Metzger, Int. J. Pept. Protein Res. 1991, 38, 545-554; Roth, Bioconj. Chem. 2004, 15, 541-553) (0.3 mmol, 267 mg) was dissolved in DMF (5 mL) and PyBOP (0.3 mmol, 156 mg), HOBt (0.3 mmol, 40 mg), and DIPEA (0.4 mmol, 67 μL) were added to the solution. After 2 min the reaction mixture was added to the resin. The coupling reaction was monitored by the Kaiser test and was complete after standing for 12 h. Next, the N^{α}-Fmoc group was cleaved using piperidine (20%) in DMF (6 mL) to obtain 36. Palmitic acid (0.3 mmol, 77 mg) was coupled to the free amine of 36 as described above using PyBOP (0.3 mmol, 156 mg), HOBt (0.3 mmol, 40 mg), and DIPEA (0.4 mmol, 67 μL) in DMF. The resin was washed thoroughly with DMF (5 mL×2), DCM (5 mL×2), and MeOH (5 mL×2) and then dried in vacuo. The resin was swelled in DCM (5 mL) for 1 h and then treated with TFA (95%), water (2.5%), and TIS (2.5%) (10 mL) for 2 h at room temperature. The resin was filtered and washed with neat TFA (2 mL). The filtrate was then concentrated in vacuo to approximately ⅓ of its original volume. The lipopeptide was precipitated using diethyl ether (0° C.; 30 mL) and recovered by centrifugation at 3000 rpm for 15 min. The crude lipopeptide was purified by RP-HPLC on a semi preparative C-4 column using a linear gradient of 0 to 95% solvent B in solvent A over a 40 min period and the appropriate fractions were lyophilized to afford 23 (FIG. 11) (40 mg, 26%). C₈₁H₁₅₆N₁₁O₁₂S, MALDI-ToF MS: observed [M+Na],1531.2240 Da; calculated [M+Na], 1531.1734 Da.

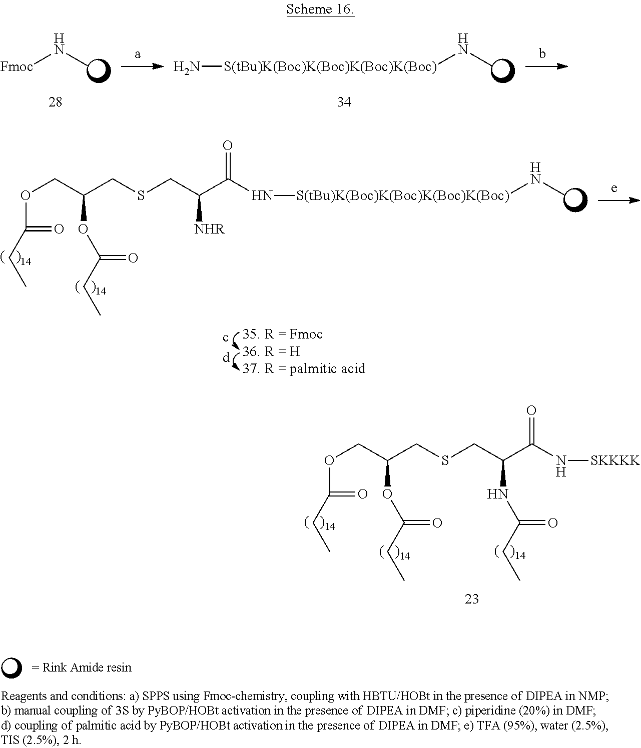

Synthesis of Glycolipopeptide 25:

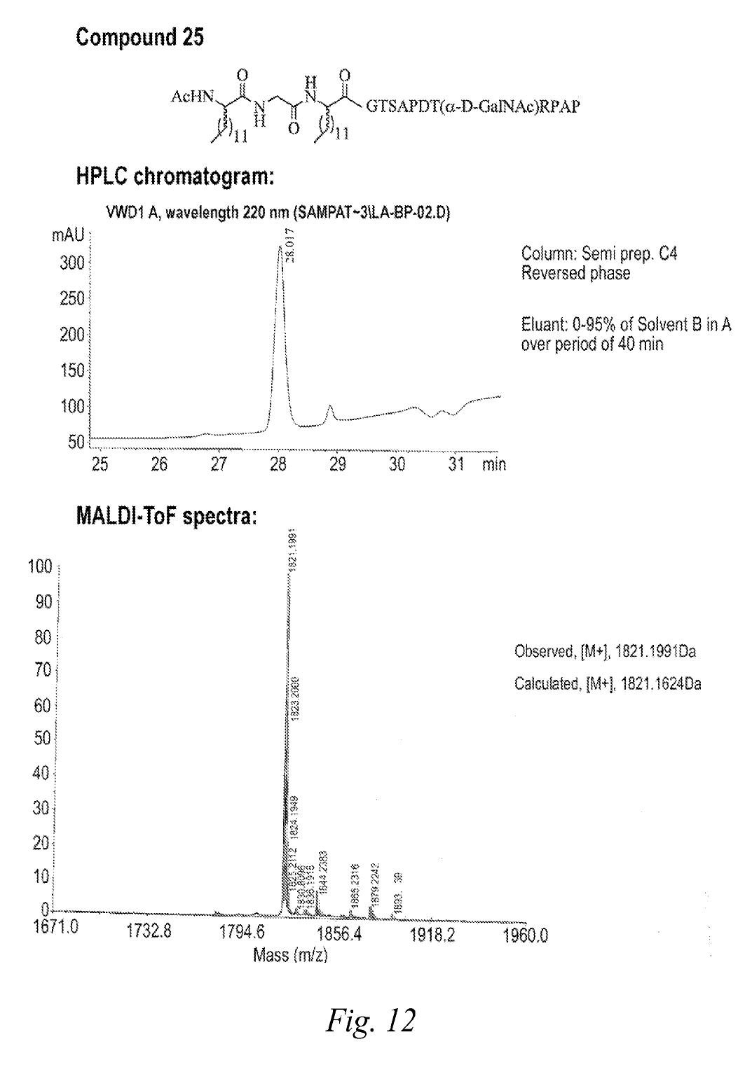

The synthesis 25 was carried out on a Rink amide resin (28, 0.1 mmol) as described under peptide synthesis in the experimental. The first four amino acids, Arg-Pro-Ala-Pro were coupled on the peptide synthesizer using a standard protocol to obtain 29. After the completion of the synthesis, a manual coupling was carried out using 1S (0.2 mmol, 134 mg). 1S was dissolved in NMP (5 mL) and HATU (0.2 mmol, 76 mg), HOAt (0.2 mmol, 27 mg), and DIPEA (0.4 mmol, 70 μL) were added and the resulting mixture was added to the resin. The coupling reaction was monitored by standard Kaiser test. After 12 h, the resin was washed with NMP (6 mL) and DCM (6 mL), and re-subjected to the same coupling conditions to ensure complete coupling. Glycopeptide 30 was then elongated on the peptide synthesizer. After the completion of the synthesis, the resin was thoroughly washed with NMP (6 mL), DCM (6 mL), and MeOH (6 mL) and dried in vacuo. The resin was then swelled in DCM (5 mL) for 1 h and the rest of the peptide sequence was completed manually. 2S (0.3 mmol, 139 mg) was dissolved in NMP (5 mL) and PyBOP (0.3 mmol, 156 mg), HOBt (0.3 mmol, 40 mg), and DIPEA (0.4 mmol, 67 μL) were added to the solution. After 2 min, the mixture was added to the resin. The coupling reaction was monitored by standard Kaiser test and was complete after standing for 8 h. Next, the N^{α}-Fmoc group was cleaved using piperidine (20%) in DMF (6 mL). N^{α}-Fmoc-L-glycine (0.3 mmol, 90 mg) was dissolved in NMP (5 mL) and premixed with PyBOP (0.3 mmol, 156 mg), HOBt (0.3 mmol, 40 mg), and DIPEA (0.4 mmol, 67 μL) for 2 min before the reaction mixture was added to the resin. The coupling reaction was monitored by Kaiser test and was complete after standing for 4 h. The N^{α}-Fmoc group was cleaved using piperidine (20%) in DMF (6 mL). One more cycle of coupling of 2S (0.3 mmol, 139 mg) was carried out as described above using PyBOP (0.3 mmol, 156 mg), HOBt (0.3 mmol, 40 mg), and DIPEA (0.4 mmol, 67 μL) in NMP (5 mL). Finally, the N^{α}-Fmoc group was cleaved using piperidine (20%) in DMF (6 mL) and the resulting free amino group was acetylated using Ac₂O (10%) and DIPEA (5%) in NMP (5 mL) for 10 min. The resin was washed thoroughly with NMP (5 mL×2), DCM (5 mL×2), and MeOH (5 mL×2), and dried in vacuo. The resin was swelled in DCM (5 mL) for 1 h, treated with hydrazine (60%) in MeOH (10 mL) for 2 h, washed thoroughly with NMP (5 mL×2), DCM (5 mL×2) and MeOH (5 mL×2) and dried in vacuo. The resin was swelled in DCM (5 mL) for 1 h after which it was treated with reagent B (TFA (88%), water (5%), phenol (5%), and TIS (2%), 10 mL) for 2 h. The resin was filtered, washed with neat TFA (2 mL) and the filtrate was then concentrated in vacuo to approximately ⅓ of its original volume. The glycolipopeptide was precipitated using diethyl ether (0° C.; 40 mL) and recovered by centrifugation at 3,000 rpm for 15 min. The crude glycolipopeptide was purified by RP-HPLC on a semi preparative C-4 column using a linear gradient of 0-95% solvent B in A over 40 min, and the appropriate fractions were lyophilized to afford 5 (FIG. 12) (35 mg, 19%). C₈₄H₁₄₅N₁₉O₂₅, MALDI-ToF MS: observed, [M+] 1821.1991 Da; calculated, [M+] 1821.1624 Da.

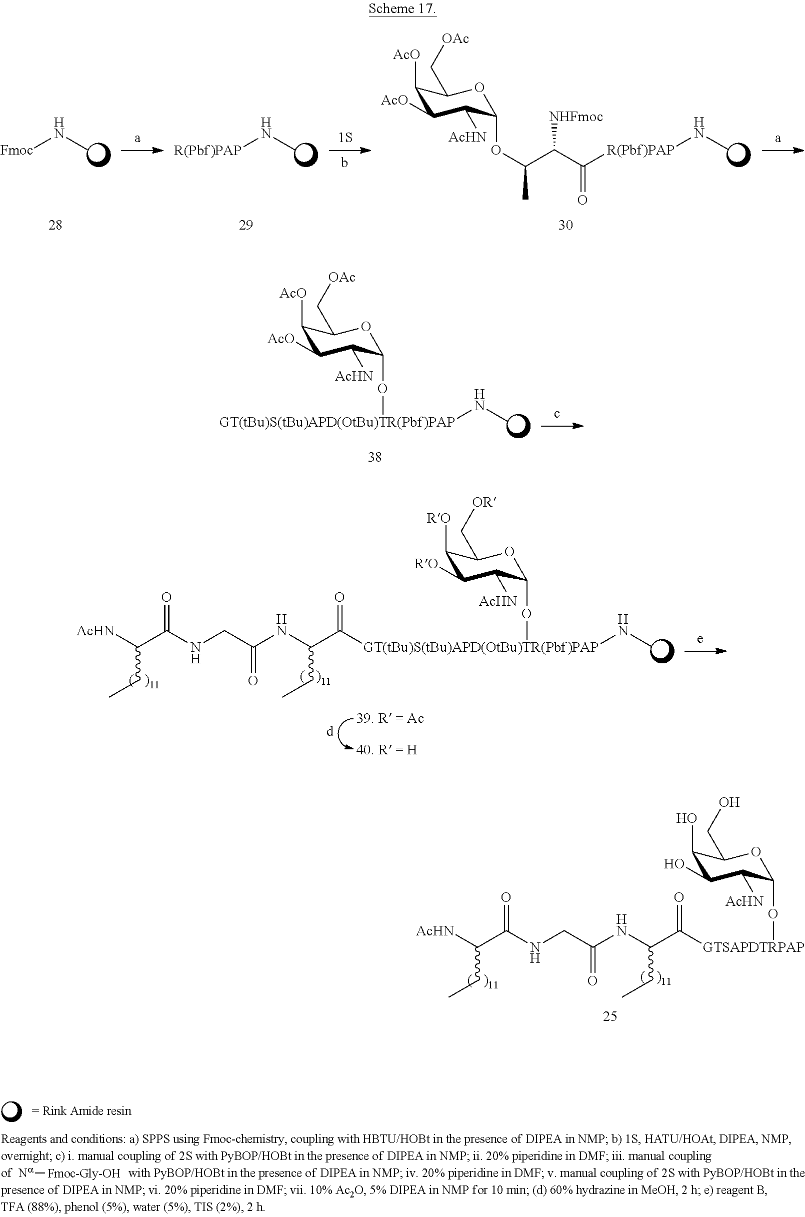

Synthesis of Lipopeptide 26:

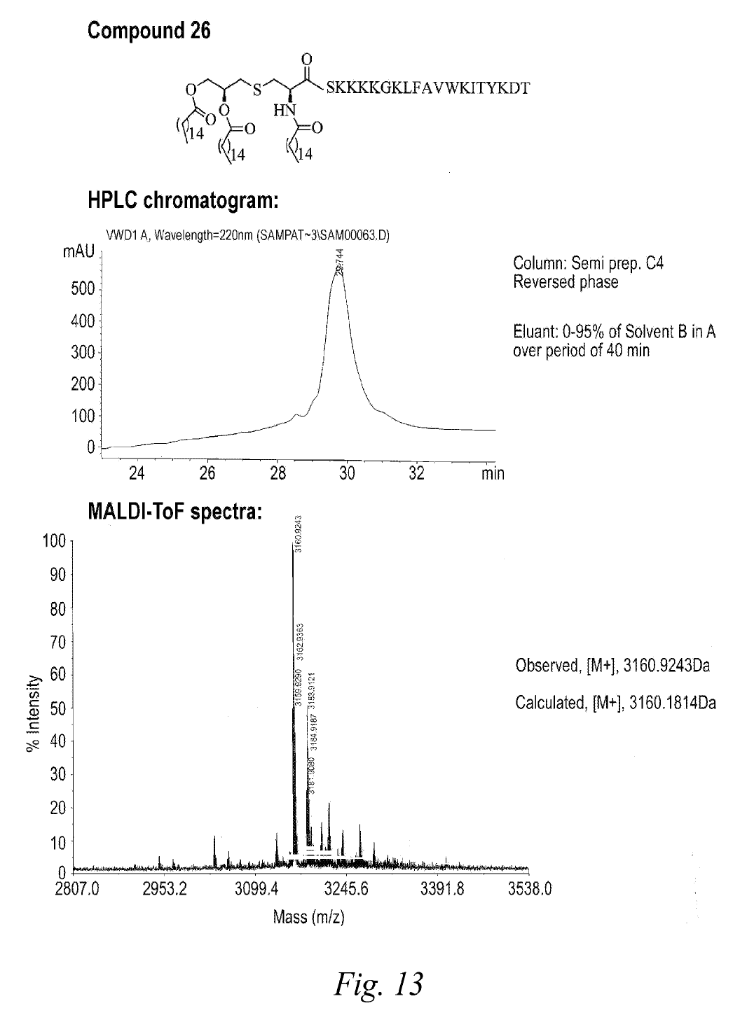

The synthesis of 26 was carried out on a Rink amide resin (28, 0.1 mmol). After the assembly of the peptide by using standard SPPS, the lipid portion of the molecule was coupled manually. 3S (0.3 mmol, 267 mg) was dissolved in DMF (5 mL) and PyBOP (0.3 mmol, 156 mg), HOBt (0.3 mmol, 40 mg), and DIPEA (0.4 mmol, 67 μL) were added to the solution. After activation of 3S for 2 min the reaction mixture was added to the resin. The coupling reaction was monitored by the Kaiser test and was complete after standing for 12 h. The N-Fmoc group was cleaved using piperidine (20%) in DMF (6 mL) to obtain 43. Palmitic acid (77 mg, 0.3 mmol) was coupled to the free amine of 43 as described above using PyBOP (0.3 mmol, 156 mg), HOBt (0.3 mmol, 40 mg), and DIPEA (0.4 mmol, 67 μL) in DMF. The resin was washed thoroughly with DMF (5 mL×2), DCM (5 mL×2), and MeOH (5 mL×2) and then dried in vacuo. The resin was swelled in DCM (5 mL) for 1 h, treated with reagent B (TFA (88%), water (5%), phenol (5%), and TIS (2%), 10 mL) for 2 h, filtered and washed with neat TFA (2 mL). The filtrate was then concentrated in vacuo to approximately ⅓ of its original volume, and the lipopeptide was precipitated using diethyl ether (0° C.; 30 mL) and recovered by centrifugation at 3000 rpm for 15 min. The crude lipopeptide was purified by RP-HPLC on a semi preparative C-4 column using a linear gradient of 0-95% solvent B in A over a 40 min., and the appropriate fractions were lyophilized to afford 26 (FIG. 13) (57 mg, 18%). C₁₆₂H₂₇₈N₂₉O₃₁S, MALDI-ToF MS: observed, [M+] 3160.9423 Da; calculated, [M+] 3160.1814 Da.

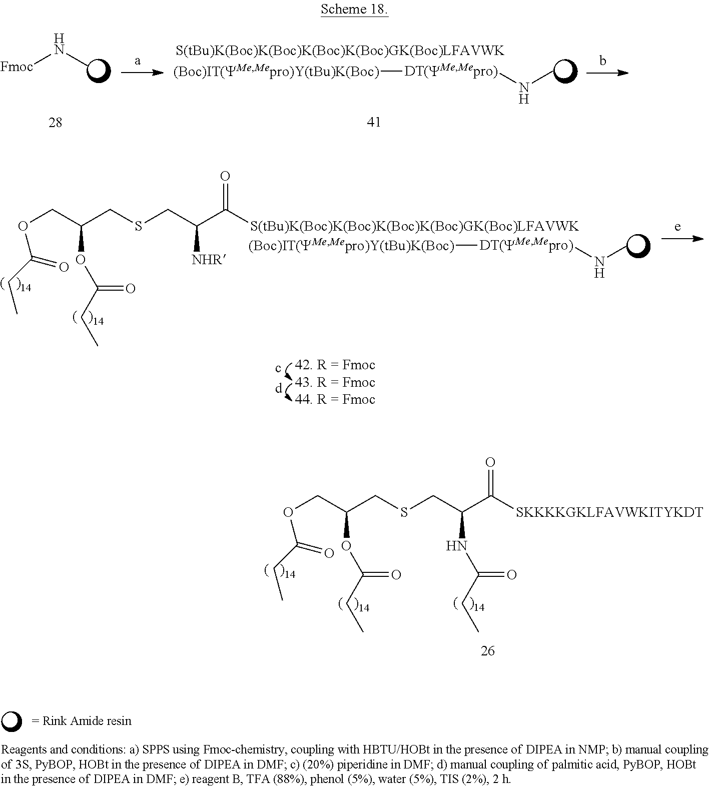

Synthesis of Biotin-T-Epitope Peptide 27:

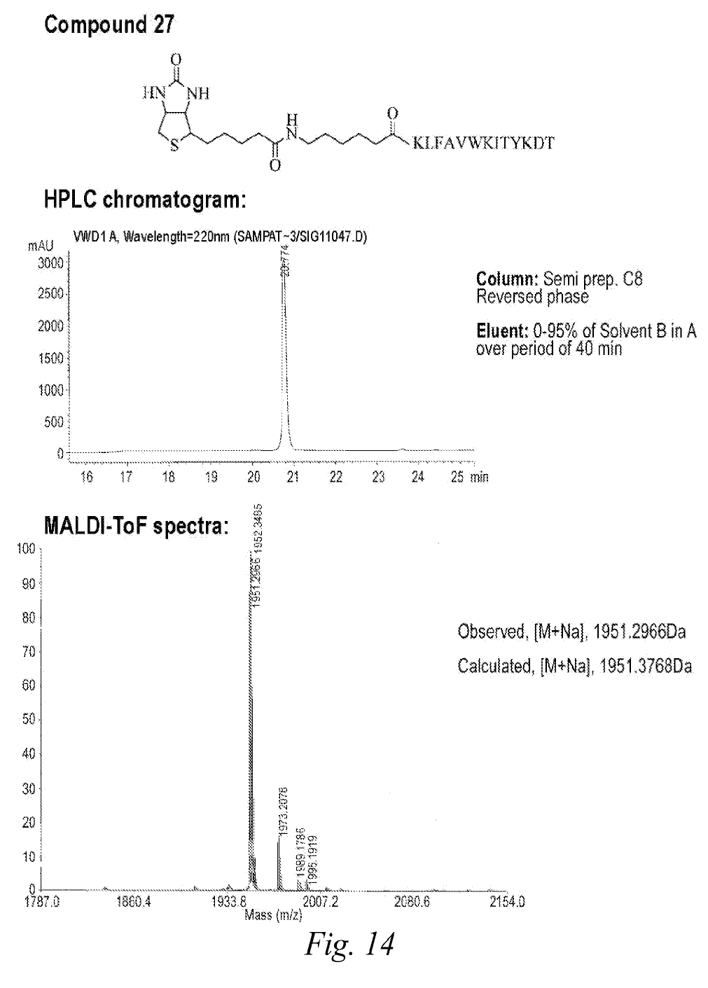

The synthesis of 27 was carried out on a Rink amide resin (28, 0.1 mmol) as described in the general method. After the completion of synthesis the resin was washed thoroughly with DMF (5 mL×2), DCM (5 mL×2), and MeOH (5 mL×2) and then dried in vacuo. The resin was swelled in DCM (5 mL) for 1 h. Next, a mixture of EZ-Link® NHS-Biotin reagent (succinimidyl-6-(biotinamido)hexanoate) (0.2 mmol, 90 mg) and DIPEA (0.2 mmol, 36 μL) in DMF (5 mL) was added to the resin. The coupling was monitored by standard Kaiser test and was complete within 8 h. The resin was washed thoroughly with DMF (5 mL×2), DCM (5 mL×2), and MeOH (5 mL×2) and then dried in vacuo. The resin was swelled in DCM (5 mL) for 1 h and treated with reagent B (TFA (88%), water (5%), phenol (5%), and TIS (2%), 15 mL) for 2 h at room temperature. The resin was filtered and washed with neat TFA (2 mL). The filtrate was concentrated in vacuo to approximately ⅓ of its original volume. The peptide was precipitated using diethyl ether (0° C.; 30 mL) and recovered by centrifugation at 3,000 rpm for 15 min. The crude peptide was purified by RP-HPLC on a semi preparative C-8 column using a linear gradient of 0 to 95% solvent B in solvent A over a 40 min period and the appropriate fractions were lyophilized to afford 27 (FIG. 14) (60% based on resin loading capacity). C₉₅H₁₄₇N₂₁O₂₁S, MALDI-ToF MS: observed [M+], 1951.2966 Da; calculated [M+], 1951.3768 Da.

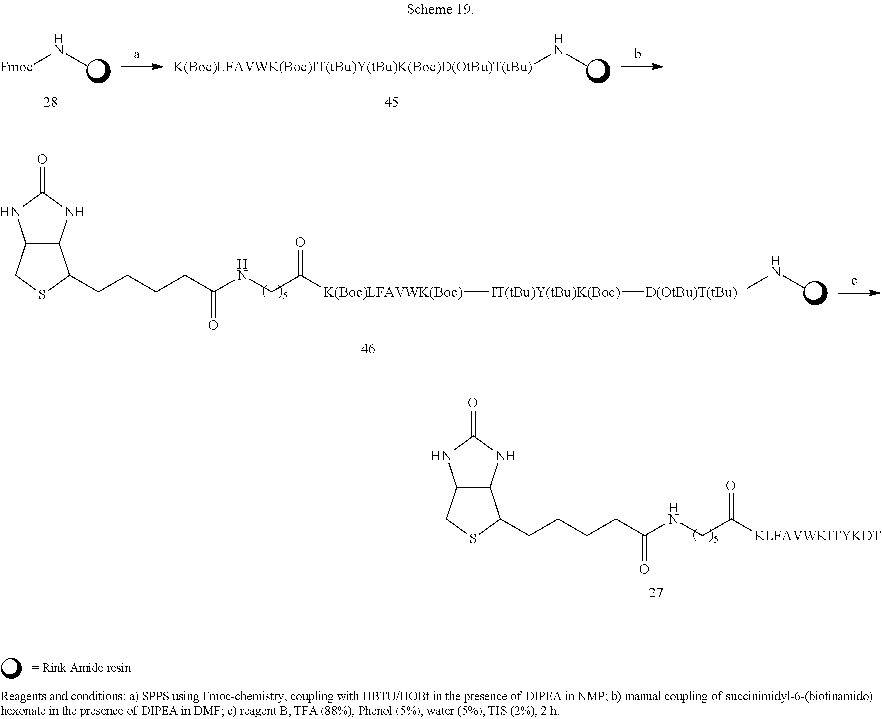

### Example 8

### A Fully Synthetic Multi-Component Cancer Vaccine Elicits Multi-Model Immune Responses

This example demonstrates that a glycosylated MUC1 derived glycopeptide covalently linked to a Toll-like receptor (TLR) agonist can elicit potent humoral and cellular immune responses and is efficacious in reversing tolerance and generating a therapeutic response. The examination of a number of control compounds demonstrate that the therapeutic effect of the three-component vaccine is due to nonspecific antitumor responses elicited by the adjuvant, and specific humoral and cellular immune responses elicited by the MUC1 derived glycopeptide. It has been found that glycosylation of the MUC1 peptide is critical for inducing optimal responses and furthermore, it is essential that the helper T- and B-epitope are covalently attached to the TLR ligand.

### Results

Antigen design and tumor challenge studies. The efficacy of liposomal preparations of compounds 1, 2, 3, a mixture of 4 and 5 and 5 alone (FIG. 16) were examined in a well-established mouse model for mammary cancer (Akporiaye et al., 2007, Vaccine; 25:6965-6974). The multi-component vaccine candidate 1 contains a tumor-associated glycopeptide derived from MUC1 (Baldus et al., 2004, Crit. Rev Clin Lab Sci; 41:189-231; and Springer, 1997, J Mol Med; 75:594-602), the well-documented murine MHC class II restricted helper T-cell epitope KLFAVWKITYKDT (SEQ ID NO:1) derived from polio virus (Leclerc et al., 1991, J Virol; 65:711-718), and the lipopeptide Pam3CysSK4, which is a potent agonist of Toll-like receptors-2 (TLR2) (Spohn et al., 2004, Vaccine; 22:2494-2499). Previously, the MUC1 derived glycopeptide SAPDT(α-GalNAc)RPAP, was identified as the antigenic-dominant domain of the tandem repeat of MUC1 (Baldus et al., 2004, Crit. Rev Clin Lab Sci; 41:189-231; and Springer, 1997, J Mol Med; 75:594-602). Furthermore, this epitope can also be presented in complex with MHC class I (K^{b}) resulting in the activation of cytotoxic T-lymphocytes (CTLs) (Apostolopoulos et al., 2003, Proc Natl Acad Sci USA; 100:15029-15034).

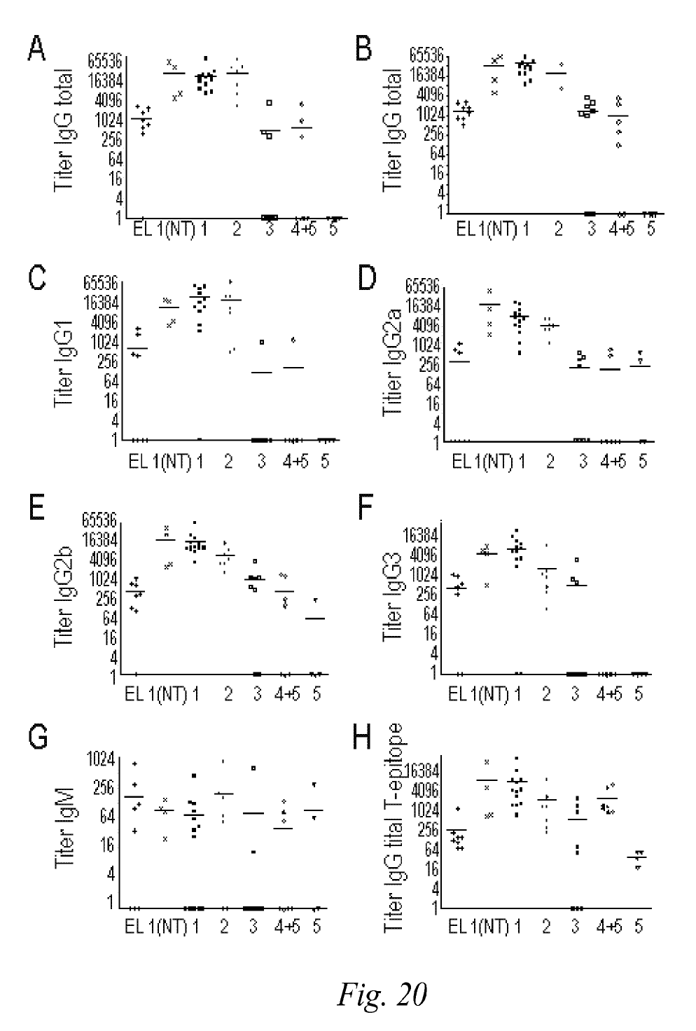

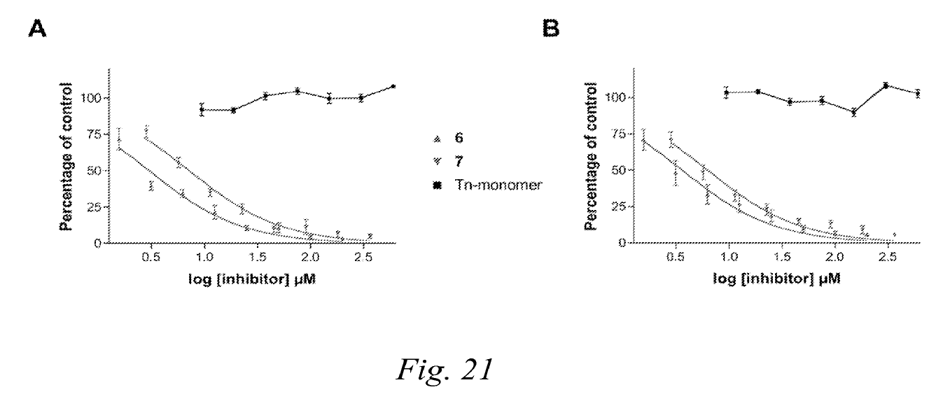

As shown in this example, the MHC class II restricted helper T-cell epitope of 1 induced a class switch from IgM to IgG antibody production (FIG. 20) and facilitated the presentation of exogenous glycopeptides on MHC class 1. Finally, the Pam3CysSK4 moiety of 1 functioned as an inbuilt adjuvant by eliciting relevant cytokines and chemokines (Spohn et al., 2004, Vaccine; 22:2494-2499). To determine the importance of the carbohydrate moiety of 1, construct 2 was examined, which has a similar structure as 1 except that the threonine of the MUC1 peptide is not glycosylated. Compound 3 lacks the MUC1 glycopeptide epitope of 1 and 2 and was examined to account for possible therapeutic effects due immune activation by the adjuvant. Finally, a mixture of the glycopeptide 4 and adjuvant Pam3CysSK4 5 was examined to establish the importance of covalent attachment of the adjuvant to the MUC1 glycopeptide and helper T-epitope.

The multi-component vaccine 1 was prepared by liposome-mediated native chemical ligation (Ingale et al., 2006, Org Lett; 8:5785-5788). Compounds 2, 3, 4 were synthesized by a SPPS protocol using a Rink amide resin, Fmoc protected amino acids, Fmoc-Thr-(AcO₃-α-D-GalNAc). The resulting compounds were incorporated into phospholipid-based small uni-lamellar vesicles (SUVs) by hydration of a thin film of the synthetic compounds, egg phosphatidylcholine, phosphatidylglycerol and cholesterol in a HEPES buffer (10 mM, pH 6.5) containing NaCl (145 mM) followed by extrusion through a 100 nm Nuclepore® polycarbonate membrane. Groups of MUC1.Tg mice (C57BL/6; H-2^{b}) that express human MUC1 were immunized three-times at biweekly intervals with liposomal preparations of compounds 1, 2, 3 a mixture of 4 and 5 and 5 alone. After 35 days, the mice were challenged with MMT mammary tumor cells (positive for MUC1 and Tn) followed by one more boost after one week. Two weeks after the last immunization, the mice were sacrificed and the efficacy of the vaccines determined by tumor weight. Furthermore, the robustness of humoral immune responses was assessed by titers of MUC1-specific antibodies and the ability of the antisera to lyse MUC1-bearing tumor cells. In addition, cellular immune responses were evaluated by determining the number of IFN-γ producing CD8⁺ T-cells and the ability of these cells to lyse tumor cells.

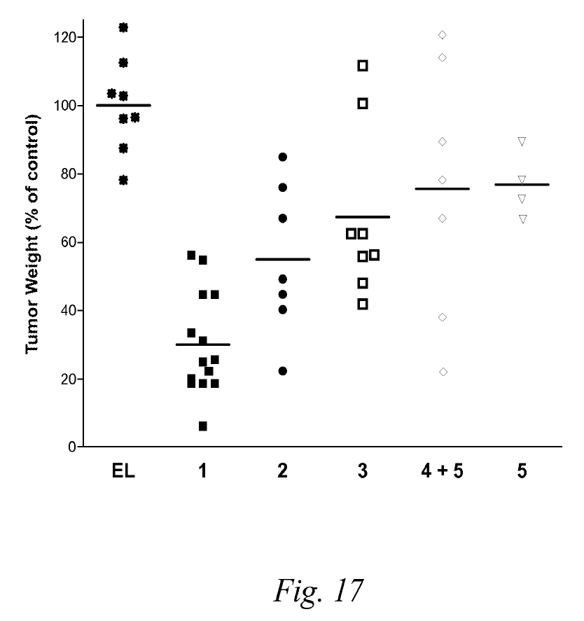

Immunization with multi-component vaccine candidate 1 led to a significant reduction in tumor burden compared to empty liposomes or treatment with compound 3, which does not contain a MUC1 glycopeptide epitope (FIG. 17). Interestingly, immunizations with compound 3 led to somewhat smaller tumors compared to the application of empty liposomes, indicating antitumor properties due to nonspecific adjuvant effects. A glycosylated multi-component vaccine candidate 2 and a mixture of compounds 4 and 5 did not exhibit a significant improvement of anti-cancer properties compared to control immunizations. In these cases, large scatter in tumor weights was observed whereas immunization with compound 1 led to substantial reduction in tumor weight in all mice.

Humoral Immunity. Anti-MUC1 antibody titers were determined by coating microtiter plates with CTSAPDT(α-D-GalNAc)RPAP conjugated to bromoacetyl modified BSA. Compound 1 had elicited robust IgG antibody responses, and subtyping of the antibodies indicated a mixed Th1/Th2 response (Table 6). Mice immunized with 1 but not challenged with MMT tumor cells elicited similar titers of antibodies, indicating that immune suppression by cancer cells was probably reversed. Inhibition ELISA showed that the polyclonal sera had slightly higher affinities for the glycosylated MUC1 epitope (Table 7). Furthermore, low titers of antibodies against the helper T-epitope were measured indicating that the candidate vaccine does not suffer from immune suppression. Although compound 2 does not contain a carbohydrate moiety, the resulting antisera could recognize the CTSAPDT(α-D-GalNAc)RPAP epitope. However, in this case, no IgG3 antibodies were detected. Interestingly, the mixture of compounds 4 and 5 had elicited low titers of antibodies, highlighting the importance of covalent attachment of the Pam3CysSK4 to glycopeptide epitope for robust antigenic responses. As expected, the controls that did not contain a MUC1 derived epitope (3 and 5) did not elicit anti-MUC1 antibody responses.

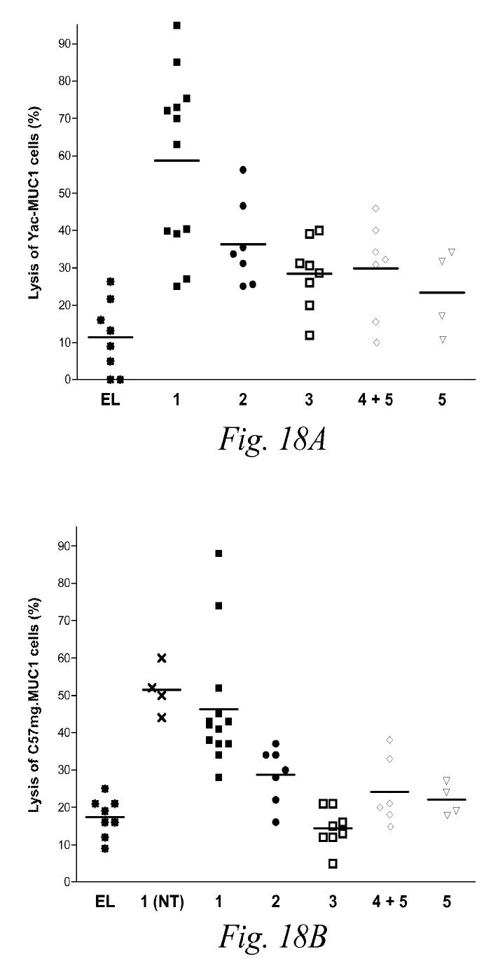

Antibody-dependent cell-mediated cytotoxicity (ADCC) was examined by labeling two MUC1 expressing cancer cell types with ⁵¹Cr, followed by the addition of antisera and cytotoxic effector cells (NK cells) and measurement of released ⁵¹Cr. As can be seen in FIGS. 18A and 18B, the antisera obtained by immunization with 1 was able to significant increase cancer cell lysis compared to the control compound 3. Importantly, antibodies elicited by compound 2 were significantly less efficacious in cell lysis compared to compound 1, highlighting the importance of glycosylation for relevant antigenic responses. As expected, the antisera derived from a mixture of 4 and 5 and the control derivatives lacking the MUC1 glycopeptide did not induce significant cell lysis.

TABLE 6
ELISA anti-MUC1 and anti-T-epitope antibody titers^{[a]} after
4 immunizations with various preparations.

| Im- | | | | | | | |
|---|---|---|---|---|---|---|---|
| mu- | IgG | | | | | | IgG |
| niza- | total | IgG1 | IgG2a | IgG2b | IgG3 | IgM | total |
| tion^{[b]} | MUC1 | MUC1 | MUC1 | MUC1 | MUC1 | MUC1 | T-epit. |
| ELI^{[c]} | 1,500 | 200 | 0 | 300 | 300 | 100 | 100 |
| 1(NT)^{[d]} | 31,900 | 10,600 | 10,000 | 15,500 | 3,900 | 100 | 2,100 |
| 1 | 30,200 | 16,000 | 6,600 | 10,700 | 3,900 | 50 | 3,000 |
| 2 | 12,900 | 10,400 | 4,100 | 4,500 | 700 | 100 | 1000 |
| 3 | 1,300 | 0 | 100 | 900 | 0 | 0 | 50 |
| 4 + 5 | 300 | 0 | 0 | 200 | 0 | 0 | 1,000 |
| 5 | 0 | 0 | 200 | 0 | 0 | 50 | 50 |

^{[a]}Anti-MUC1 and anti-T-epitope antibody titers are presented as median values for groups of four to thirteen mice. ELISA plates were coated with BSA-MI-MUC1(Tn) conjugate for anti-MUC1 antibody titers or neutravidin-biotin-T-epitope for anti-T-epitope antibody titers. Titers were determined by linear regression analysis, with plotting of dilution versus absorbance. Titers are defined as the highest dilution yielding an optical density of 0.1 or greater relative to normal control mouse sera.
^{[b]}Liposomal preparations were employed. MTT tumors were induced between the 3rd and 4th immunization.
^{[c]}EL = empty liposomes.
^{[d]}No tumor induced.

TABLE 7
Competitive inhibition IC₅₀ values for MUC1(Tn) and MUC1
(unglycosylated) of antibody binding to BSA-MI-MUC1(Tn) conjugate
by ELISA^{[a]}.

IC₅₀ inhibitors (μM)

| Immunization | MUC1(Tn) | MUC1 (unglyc) |
|---|---|---|
| 1 | 3.01 | 7.19 |
| | (2.54 to 3.59) | (6.23 to 8.29) |
| 2 | 3.63 | 6.30 |
| | (2.88 to 4.56) | (5.36 to 7.41) |

^{[a]}ELISA plates were coated with BSA-MI-MUC1(Tn) conjugate. Serum samples of groups of 7 mice after immunizations with 1 or 2, diluted to obtain in the absence of an inhibitor an OD of approximately 1 in the ELISA, were first mixed with MUC1(Tn) or unglycosylated MUC1 (0-500 μM final concentration) and then applied to the coated microtiter plate. Optical density values were normalized for the optical density values obtained with serum alone (0 μM inhibitor, 100%). Inhibition data were fit with the following logistic equation: Y = Bottom + (Top − Bottom)/(1 + 10^{(X - Log IC50)}), where Y is the normalized optical density, X is the logarithm of the concentration of the inhibitor and IC₅₀ is the concentration of the inhibitor that reduces the response by half. The IC₅₀ values are reported as best-fit values and as 95% confidence intervals.

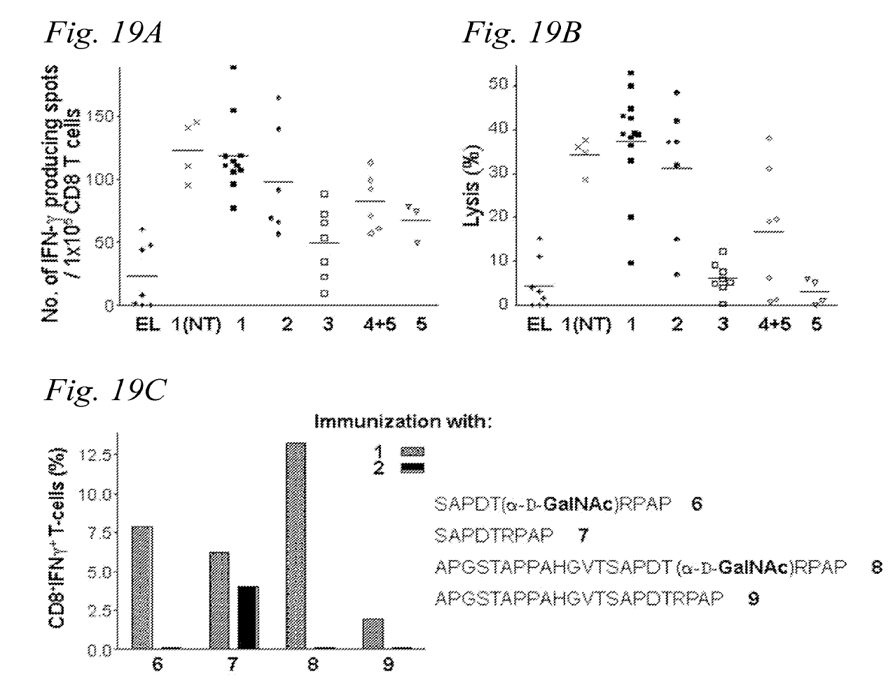

Cellular Immunity. To assess the ability of the vaccine candidates to activate cytotoxic T-lymphocytes, CD8⁺ T-cells from lymph nodes of the mice immunized with the various compounds were isolated by magnetic cell sorting and incubated with irradiated DCs pulsed with the immunizing peptides on ELLISPOT plates. Vaccine candidates 1 and 2 exhibited robust CD8⁺ responses compared to control (FIG. 19A, 1 and 2 vs. 3). Interestingly, a mixture of glycopeptides 4 and adjuvant 5 (Pam3CysSK4) induced the activation of a smaller number of CD8⁺, indicating that covalent attachment of the MUC1 and helper T-epitope to the adjuvant is important for optimal activation of CTLs.

The lytic activity of the isolated CD8+ cells without in-vitro stimulation was examined by a ⁵¹Cr-release assay in which DCs were pulsed with the MUC1-derived glycopeptides SAPDT(Tn)RPAP (SEQ ID NO:26) or with the peptide SAPDTRPAP (SEQ ID NO:20) in case of immunization 2. As can be seen in FIG. 19B, CTLs activated by compounds 1 and 2 exhibited significantly greater cytotoxicity compared to controls. Furthermore, mice immunized with a mixture of 4 and 5 exhibited a reduced lytic activity further demonstrating the importance of covalent attachment of the various epitopes.

To investigate in detail the epitope requirements of the CD8⁺ cells, groups of five MUC1.tg were immunized with liposomal preparations of compounds 1 and 2, followed by harvesting and combining CD8⁺ cells, which were stimulated in-vitro for 1 day by DCs pulsed with the glycopeptide SAPDT(Tn)RPAP (6) (SEQ ID NO:26) and peptide SAPDTRPAP (7) (SEQ ID NO:20), respectively and then allowed to expand for 14 days by culturing with IL-2 and IL-7. The percentage of IFN-γ producing CD8⁺ cells was established after pulsing dendritic cells with MUC1-derived glycopeptides 6-9. Compound 1 had activated a diverse range of CTL that could be activated by glycosylated and nonglycosylated structures, whereas those obtained by immunization with 2 only showed responsiveness with aglycosylated peptide 7. Furthermore, CD8⁺ cells obtained from immunizing with 1 could lyse DCs pulsed with glycosylated and aglycosylated structures (FIG. 20).

These results indicate that CTLs activated by immunizations with 1 recognize a wider range of structures including glycosylated and aglycosylated MUC1-derived peptides whereas CTLs obtained from compound 2 exhibit a strong preference for aglycosylated peptides.

Cytokine induction. The lipopeptide moiety of the three-component vaccine is required for initiating the production of necessary cytokines and chemokines by interacting with TLR2 on the surface of mononuclear phagocytes (Akira et al., 2001, Nat Immunol; 2:675-680; Finlay and Hancock, 2004, Nat Rev Microbiol; 2:497-504; van Amersfoort et al., 2003, Clin Microbiol Rev; 16:379-414; and Spohn et al., 2004, Vaccine; 22:2494-2499). After activation, the intracellular domain of TLR2 recruits the adaptor protein MyD88, resulting in the activation of a cascade of kinases leading to the production of a number of cytokines and chemokines. On the other hand, lipopolysaccharides induce cellular responses by interacting with the TLR4/MD2/CD14 complex, which results in the recruitment of the adaptor proteins MyD88 and TRIF leading to a more complex pattern of cytokine induction. TNF-γ secretion is the prototypical measure for activation of the MyD88-dependent pathway, whereas secretion of IFN-γ is commonly used as an indicator of TRIF-dependent cellular activation (Akira et al., 2001, Nat Immunol; 2:675-680; and van Amersfoort et al., 2003, Clin Microbiol Rev; 16:379-414).

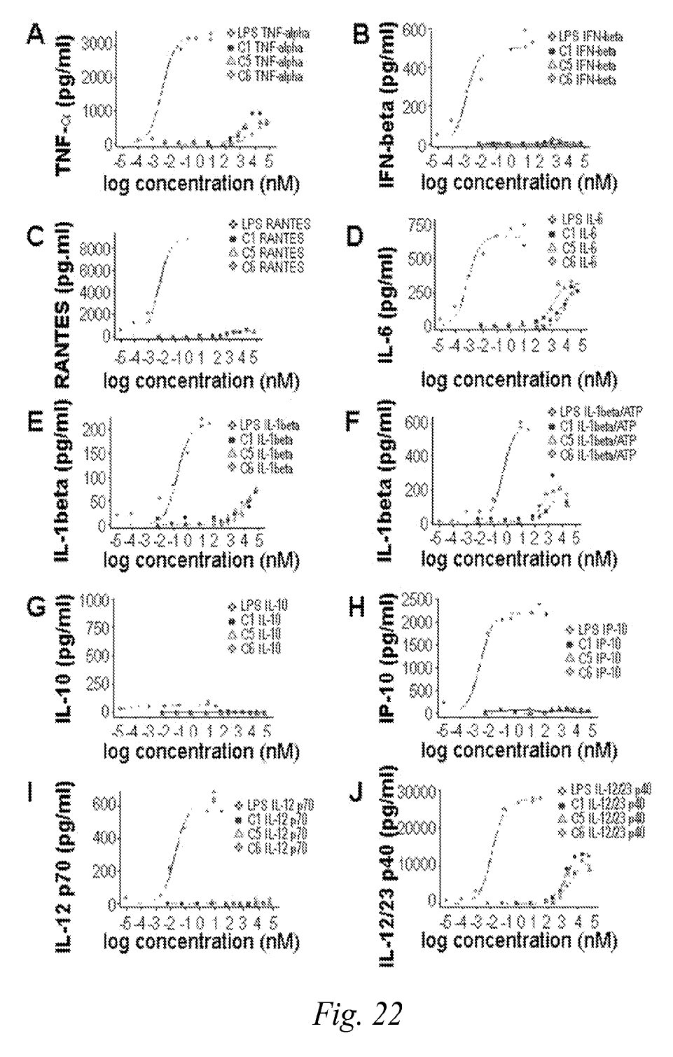

To examine the pattern of cytokine production by the multi-component vaccine 1 and establish whether glycosylation affects responsiveness, the efficacy (EC₅₀) and potency (maximum responsiveness) of secretion of TNF-α, IFN-β, Rantes, IL-6, IL-1, IL-10, IP-10, IL-12p70, and IL-12/23p40 induced by compounds 1, 2 and 5 was examined. Thus, primary dendritic cells obtained by established methods were exposed over a wide range of concentrations to the compounds 1, 2, 5, and E. coli 055:B5 LPS and the supernatants examined for the various mouse cytokines using capture ELISA. Glycolipopeptide 1, lipopeptide 2 and Pam3CysSK4 (5) induced secretion of TNF-α, Rantes, IL-6, IL-1 and IL-12/23p40 with similar efficacies and potencies indicating that attachment of the B- and T-epitopes and glycosylation had no effect on cytokine responses. See FIG. 22, Table 8, and Table 9. As expected, both compounds did not induce the production of IFN-β. Interestingly, E. coli 055:B5 LPS displayed much larger potencies and efficacies for TNF-α induction compared to compounds 1, 2 and 5. In addition, it was able to stimulate the cells to produce IFN-β, IL-10, IP10, and IL-12p70. The reduced potency and efficacy of 1, 2 and 5 is a beneficial property, because it is known that LPS can over-activate the innate immune system, leading to symptoms of septic shock.

To ensure that cytokine production was initiated in a TLR2-dependent manner, compounds 1 and 5 were exposed to HEK 293T cells stably transfected with murine TLR2 and transiently transfected with a plasmid containing the reporter gene pELAM-Luc (NF-B-dependent firefly luciferase reporter vector) and a plasmid containing the control gene pRL-TK (Renilla luciferase control reporter vector). After an incubation time of 4 hours, the activity was measured using a commercial dual-luciferase assay and it was found that compounds 1 and 5 were able to activate NF-B in a TLR2-dependent manner.

TABLE 8
Cytokine plateau values^{[a]} (pg/mL) of dose-response curves of liposome
preparations loaded with compound 1, 2 or 3 and E. coli LPS obtained after
incubation of primary dendritic cells for 24 h.

| Cytokine (pg/mL) | 1 | 2 | 3 | LPS |
|---|---|---|---|---|
| TNF-alpha | 836 ± 103 | 695 ± 50 | 854 ± 67 | 3,265 ± 96 |
| IFN-beta | nd^{[b]} | nd | Nd | 505 ± 34 |
| RANTES | 584 ± 59 | 553 ± 54 | 536 ± 28 | 8,869 ± 416 |
| IL-6 | 298 ± 28 | 316 ± 40 | 401 ± 43 | 668 ± 34 |
| IL-1beta | 60 ± 10 | 84 ± 13 | 77 ± 4 | 209 ± 15 |
| IL-1beta/ATP | 187 ± 50 | 181 ± 26 | 194 ± 14 | 596 ± 24 |
| IL-10 | nd | nd | Nd | 91 ± 6 |
| IL-10 | nd | nd | Nd | 2,196 ± 44 |
| IL-12 p70 | nd | nd | Nd | 623 ± 19 |
| IL-12/23 p40 | 13,668 ± 496 | 10,692 ± 853 | 11,192 ± 382 | 27,679 ± 460 |

^{[a]}Plateau values as reported by Prism as best-fit values ± std error using non-linear least squares curve fitting as picogram of cytokine per μg of total protein.
^{[b]}nd indicates not detected.

TABLE 9
Cytokine log EC₅₀ values^{[a]} (nm) of liposome preparations loaded
with compound 1, 2 or 3 and E. coli LPS in primary dendritic cells.

| Cytokine | | | | |
|---|---|---|---|---|
| (pg/mL) | 1 | 2 | 3 | LPS |
| TNF-alpha | 3.08 ± 0.25 | 2.99 ± 0.14 | 4.17 ± 0.10 | −2.38 ± 0.12 |
| IFN-beta | nd^{[b]} | nd | nd | −3.04 ± 0.24 |
| RANTES | 3.12 ± 0.17 | 2.88 ± 0.19 | 3.66 ± 0.09 | −2.25 ± 0.16 |
| IL-6 | 3.58 ± 0.16 | 2.88 ± 0.23 | 4.05 ± 0.14 | −3.15 ± 0.18 |
| IL-1beta | 3.52 ± 0.28 | 3.99 ± 0.21 | 4.01 ± 0.08 | −0.80 ± 0.22 |
| IL-1beta/ATP | 2.48 ± 0.48 | 2.44 ± 0.31 | 3.06 ± 0.13 | −0.37 ± 0.12 |
| IL-10 | nd | nd | nd | nd |
| IP-10 | nd | nd | nd | −2.59 ± 0.09 |
| IL-12 p70 | nd | nd | nd | −1.67 ± 0.14 |
| IL-12/23 p40 | 3.15 ± 0.07 | 3.10 ± 0.16 | 3.51 ± 0.06 | −1.89 ± 0.06 |

^{[a]}Log EC₅₀ values as reported by Prism as best-fit values ± std error using non-linear least squares curve fitting.
^{[b]}nd indicates not detected at levels for accurate EC₅₀ determination.

### Discussion

Evidence is emerging that successful cancer vaccine development requires a multimodal treatment that affects several aspects of the immune system at once. Although cellular and humoral immune responses against MUC1 have been observed in some cancer patients, it has been difficult to design cancer vaccine candidates that can elicit both these responses. This example demonstrates that a multi-component vaccine composed of a glycopeptides derived from MUC1, a promiscuous peptide helper T-epitope and a TLR2 agonist can elicits IgG antibodies that can lyse MUC1 expressing cancer cell and stimulate cytotoxic T-lymphocytes cellular thereby reversing tolerance and generating a therapeutic response in a mouse model of mammary cancer.

Careful analysis of control compounds revealed that reduction in tumor burden mediated by the multi-component vaccine was caused by specific immunity against MUC1 and by nonspecific adjuvant effects mediated by the in-built TLR2 agonist. Evidence is emerging that TLRs are widely expressed by tumor cells and their activation can result in inhibition or promotion of tumorigenicity. Furthermore, cytokines and chemokines, which are produced following the activation of the TLRs, can stimulate the expression of a number of co-stimulatory proteins for optimum interactions between T-helper cells and B- and antigen presenting cells. A recent study indicates that TLR1/2 agonists have a unique ability to reduce the suppressive function of Foxp3⁺ regulatory T cells (Tregs) and enhance the cytotoxicity of tumor-specific CTL in vitro and in vivo and potentially have more favorable antitumor effects than other TLR agonists.

This example also demonstrates that covalent attachment of the TLR2 agonist to the glycolipoptide epitope is critical for eliciting antibodies and optimal CTL function. Lipidation with the TLR2 agonist makes it possible to formulate the multicomponent vaccine in a liposomal preparation, which probably will enhance its circulation time. Furthermore, a liposomal preparation presents the glycopeptide epitopes in a multivalent manner, thereby providing an opportunity for efficient clustering of B-cell epitopes, which is required to initiate B-cell signaling and antibody production. As shown in the previous examples, the covalent attachment of the TLR2 agonist Pam3CysSK4 facilitates selective internalization by TLR2-expressing immune cells such B- and antigen presenting cells (APC). Uptake and processing of antigen and subsequent presentation of the T-epitope as a complex with MHC class I or II on the cell surface of APCs, is critical for eliciting IgG antibodies. Over the past decade, numerous studies have shown that selective targeting of antigens to APCs will result in improved immune responses. For example, oxidized mannan, heat shock proteins, bacterial toxins, and antibodies targeting cell surface receptors of dendritic cells have been attached to antigens to increase uptake by dendritic cells. Although these uptake strategies are attractive, they have as a disadvantage that the targeting device is antigenic, which may result in immune suppression of tumor-associated carbohydrates. The attractiveness of Pam3CysSK4 for facilitating uptake by APCs lies in its low intrinsic immunity. Thus, the three-component vaccine will facilitate uptake without suffering immune suppression.

Finally, the present example demonstrates that glycosylation of the MUC1 epitope was critical for optimal reduction in tumor burden. The mechanistic studies provided a rationale for these observations and it was found that immunization with compound 1 led to somewhat higher titers of antibodies that were significantly more lytic compared to the use of compound 2 that lacks the Tn-antigen. Conformational studies by NMR complemented by light scattering measurements have indicated that deglycosylation of MUC1 results in a less extended and more globular structure. Similar studies using MUC1 related O-glycopeptides have shown that the carbohydrate moieties exert conformational effects, which may provide a rationale for differences in immune responses. Also, the use of glycosylated 1 led to the efficient activation of CTLs, which were able to recognize glycosylated and unglycosylated structures, with the former ones being preferred. On the other hand, immunizations with non-glycosylated compound 2 led to CTL that mainly recognize unglycosylated structures. It is known short O-linked glycans such as the Tn and STn on MUC1 tandem repeats remain intact during DC processing in the MHC class II pathway and thus it is possible to elicit glycopeptide selective CTL responses. Moreover, there is evidence that MUC1 glycopeptides can bind more strongly to the MHC class I mouse allele H2K^{b} compared with the corresponding unglycosylated peptide. Also the progression of carcinomas is not only associated with the modification of MUC1 with truncated saccharides such as the Tn antigen but these structures are present at much higher densities and thus effective immunotherapy needs to elicit responses that are directed to such structures.

### Experimental Section

General methods for automated solid-phase peptide synthesis (SPPS): Peptides were synthesized by established protocols on an Applied Biosystems, ABI 433A peptide synthesizer equipped with a UV detector using N^{α}-Fmoc-protected amino acids and 2-(1H-benzotriazole-1-yl)-1,1,3,3-tetramethyluronium hexafluorophosphate (HBTU)/1-hydroxybenzotriazole (HOBt) as the activating reagents. Single coupling steps were performed with conditional capping.

General methods for liposome preparation for native chemical ligation: A pH 7.8 200 mM sodium phosphate buffer containing 2 mM tris(2-carboxyethyl)phosphine (TCEP) and 0.3% EDTA was prepared. The buffer was degassed for 1 h. The cysteine-containing peptide (1 eq.), thioester (2 eq.), and dodecylposphocholine (13 eq.) were dissolved in 1:1 CHCl3:trifluoroethanol and the solvents were removed. The lipid/peptide film was then hydrated in an incubator at 41° C. for 4 h. The mixture was sonicated and the peptide/lipid suspension was extruded through 1.0 μm polycarbonate membranes (Whatman, Nucleopore, Track-Etch Membrane) at 50° C. to obtain uniform vesicles.

Synthesis of glycosylated three-component vaccine candidate 1: Peptide thioester X (1.1 mg, 0.674 μmol), peptide X (1.0 mg, 0.337 μmol), and dodecylphosphocholine (1.5 mg, 4.38 μmol) were dissolved in a mixture of 1:1 CHCl3:trifluoroethanol (5 mL). The solvents were removed under reduced pressure to give a lipid/peptide film. The lipid/peptide film was hydrated for 4 h at 41° C. using a 200 mM sodium phosphate buffer containing 2 mM TCEP and 0.3% EDTA. The mixture was sonicated and the peptide/lipid suspension was extruded through 1.0 μm polycarbonate membranes (Whatman, Nucleopore, Track-Etch Membrane) at 50° C. to obtain uniform vesicles. To the vesicle suspension was added sodium 2-mercaptoethane sulfonate (1 mM) to initiate the reaction (1.5 mM final peptide concentration). After 20 min, the reaction mixture was purified by RP-HPLC on an analytical C-4 reversed phase column using a gradient of 0-100% B in A over a period of 40 min. Lyophilization of the appropriate fractions afforded 1 (1.2 mg, 80%). C₂₁₇H₃₆₇N₄₅O₅₃S₂ HR MALDI-ToF MS: observed; calculated 4515.685 [M+].

General methods for liposome preparation for immunizations: Each glycolipopeptide was incorporated into phospholipid-based small unilamillar vesicles (SUVs) by hydration of a thin film of the synthetic compounds, egg phosphatidylcholine, phosphatidylglycerol, and cholesterol in a HEPES buffer (10 mM, pH 7.4) containing NaCl (145 mM) followed by extrusion through a 0.1 μm Nucleopore® polycarbonate membrane.

Immunizations: Eight to 12-week-old MUC1.Tg mice (C57BL/6; H-2b) that express human MUC1 were immunized three-times at biweekly intervals at the base of the tail intradermally with liposomal preparations of three-component vaccine constructs (25 μg containing 3 μg of carbohydrate) and the respective controls which lack the tumor-associated MUC1 epitope. After 35 days, the mice were challenged with MMT mammary tumor cells (1 10⁶ cells), which express MUC1 and Tn. On day 42, one week after tumor cell injection, one more immunization was given. On day 49, one week after the last immunization, the mice were sacrificed and the efficacy of the vaccine was determined.

Tumor palpation: MUC1.Tg mice were injected 7 days after the third immunization subcutaneously in the left flank with 1 10⁶ cancer cells in 100 μL PBS. Palpable tumors were measured by calipers, and tumor weight was calculated according to the formula: grams=[(length)×(width) 2]/2, where length and width are measured in centimeters. At the end point tumors were surgically removed and tumor weight was determined.

⁵¹Chromium (Cr) release assay: Cytolytic activity was determined by a standard ⁵¹Cr release method using CD8⁺ T-cells from TDLNs without any in vitro stimulation as effector cells and ⁵¹Cr labeled DCs pulsed with respective peptide as target cells at a 100:1 ratio for 6 h. Target cells were loaded with 100 μCi ⁵¹Cr (Amersham Biosciences) per 10⁶ target cells for 2 h before incubation with effectors. Radioactive ⁵¹Cr release was determined using the Topcount Microscintillation Counter (Packard Biosciences) and specific lysis was calculated: (experimental cpms−spontaneous cpms/complete cpms−spontaneous cpms)×100. Spontaneous lysis was <15% of total lysis.

Determination of antibody-dependent cell-mediated cytotoxicity (ADCC): Tumor cells (Yac-MUC1 or C57 mg.MUC1) were labeled with 100 μCi ⁵¹Cr for 2 h at 37° C., washed and incubated with control antibody (mouse IgG) at 5 μg/mL, or with serum (1 in 25 dilutions) obtained from the vaccinated mice for 30 min at 37° C. NK cells (KY-1 clone, a generous gift from Dr. Wayne M. Yokoyama, Washington University, St. Louis) which have high expression of CD16 receptor were used as effectors. These cells were stimulated with IL-2 (200 units/mL) for 24 h prior to assay. Effector cells were seeded with the antibody-labeled tumor cells in 96-well culture plates (Costar high binding plates) at an effector:target cell ratio of 50:1 for 4 h. The release of ⁵¹Cr in the supernatant was determined by the Top Count. Spontaneous and maximum release of ⁵¹Cr was determined and was below 20%. The percentage of specific release was determined: (release−spontaneous release/maximal release−spontaneous release)×100.

IFN-γ ELISPOT assay: At time of sacrifice, MAC sorted CD4⁺ and CD8⁺ T-cells from TDLNs were isolated from treated MUC1.Tg mice and used as responders in an IFN-γ ELISPOT assay. Spot numbers were determined using computer-assisted video image analysis by ZellNet Consulting, Inc. (Fort Lee, N.J.). Splenocytes from C57BL/6 mice stimulated with Concavalin A were used as a positive control.

Serologic assays: Anti-MUC-1 IgG, IgG1, IgG2a, IgG2b, IgG3, and IgM antibody titers were determined by enzyme-linked immunosorbent assay (ELISA), as described previously (Buskas et al., 2004, Chemistry, 10(14):3517-24). Briefly, ELISA plates (Thermo Electron Corp.) were coated with a conjugate of the MUC-1 glycopeptide conjugated to BSA through a maleimide linker (BSA-MI-MUC-1). Serial dilutions of the sera were allowed to bind to immobilized MUC-1. Detection was accomplished by the addition of phosphate-conjugated anti-mouse IgG (Jackson ImmunoResearch Laboratories Inc.), IgG1 (Zymed), IgG2a (Zymed), IgG2b (Zymed), IgG3 (BD Biosciences Pharmingen), or IgM (Jackson ImmunoResearch Laboratories Inc.) antibodies. After addition of p-nitrophenyl phosphate (Sigma), the absorbance was measured at 405 nm with wavelength correction set at 490 nm using a microplate reader (BMG Labtech). Antibody titers against the T (polio)-epitope were determined as follows. Reacti-bind NeutrAvidin coated and pre-blocked plates (Pierce) were incubated with biotin-labeled T-epitope (10 μg/mL; 100 μL/well) for 2 h. Next, serial dilutions of the sera were allowed to bind to immobilized T-epitope. Detection was accomplished as described above. The antibody titer was defined as the highest dilution yielding an optical density of 0.1 or greater over that of normal control mouse sera.

Inhibition ELISAs: To explore competitive inhibition of the binding of MAbs to MUC(Tn) by the corresponding glycopeptide, peptide and sugar, serum samples were diluted in diluent buffer in such a way that, without inhibitor, expected final optical density values were approximately 1. For each well 60 μL of the diluted serum samples were mixed in an uncoated microtiter plate with 60 μL diluent buffer, glycopeptide 6 (MUC(Tn)), peptide 7 (unglycosylated MUC1) or Tn-monomer in diluent buffer with a final concentration of 0-500 μM. After incubation at room temperature for 30 min, 100 μl of the mixtures were transferred to a plate coated with BSA-MI-MUC1(Tn). The microtiter plates were incubated and developed as described above using an alkaline phosphatase-conjugated detection antibody for IgG total. Optical density values were normalized for the optical density values obtained with monoclonal antibody alone (0 μM inhibitor, 100%).

Dendritic cell (DC) preparation: DCs were prepared from mouse bone marrow cultures as previously described (Inaba et al., 1992, J Exp Med; 176(6):1693-702 and Mukherjee, 2003, J Immunother; 26:47-62).

Cytokine assays: On the day of the exposure assay mature DCs were plated as 4 10⁶ cells/well in 1.8 mL in 24-well tissue culture plates. Cells were then incubated with different stimuli (200 μL, 10×) for 24 h in a final volume of 2 mL/well. Stimuli were given at a wide concentration range (corresponding to final concentrations of 0.1 ng/mL to 100 μg/mL PAM₃CysSK₄ for 1, 5, or 6 in liposomes and 0.001 ng/mL to 10 μg/mL for E. coli LPS). Supernatants were collected. For estimation of the effect of ATP on IL-1β secretion, DCs were re-incubated for 30 min in the same volume of medium containing ATP (5 mM; Sigma), after which supernatants were harvested. All collected culture supernatants were stored frozen (minus 80° C.).

Cytokine ELISAs were performed in 96-well MaxiSorp plates (Nalge Nunc International). Cytokine DuoSet ELISA Development Kits (R&D Systems) were used for the cytokine quantification of mouse TNF-α, RANTES, IL-6, IL-1β, IL-10, IP-10, IL-12 p70 and IL-12/23 p40 according to the manufacturer's instructions. The absorbance was measured at 450 nm with wavelength correction set to 540 nm using a microplate reader (BMG Labtech). Concentrations of mouse IFN-β in culture supernatants were determined as follows. Plates were coated with rabbit polyclonal antibody against mouse IFN-β (PBL Biomedical Laboratories). IFN-β in standards (PBL Biomedical Laboratories) and samples was allowed to bind to the immobilized antibody. Rat anti-mouse IFN-β antibody (USBiological) was then added. Next, HRP-conjugated goat anti-rat IgG (H+L) antibody (Pierce) and a chromogenic substrate for HRP 3,3′,5,5′-tetramethylbenzidine (Pierce) were added. After the reaction was stopped, the absorbance was measured at 450 nm with wavelength correction set to 540 nm. Cytokine values are expressed as pg cytokine/mL. Concentration-response data were analyzed using nonlinear least-squares curve fitting in Prism (GraphPad Software, Inc.). These data were fit with the following four parameter logistic equation: Y=Eₘₐₓ/(1+(EC₅₀/X)^{Hill slope}), where Y is the cytokine response, X is the concentration of the stimulus, Eₘₐₓ is the maximum response (plateau value) and EC₅₀ is the concentration of the stimulus producing 50% stimulation. The Hill slope was set at 1 to be able to compare the EC₅₀ values of the different inducers.

Statistical Analysis: Multiple comparisons were performed using one-way analysis of variance (ANOVA) with Bonferroni's multiple comparison test. Differences were considered significant when P <0.05. For comparisons between two groups, the data were analyzed using the two-tailed Student t-test with 95% confidence interval. A P-value <0.05 was regarded as statistically significant.

### Example 9

### Addition of a Second TLR Agonist

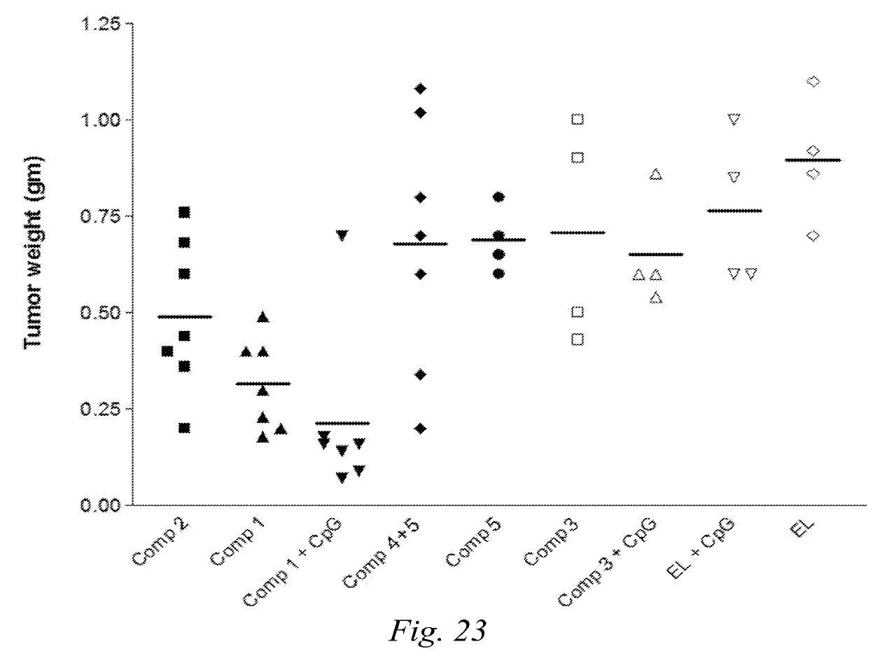

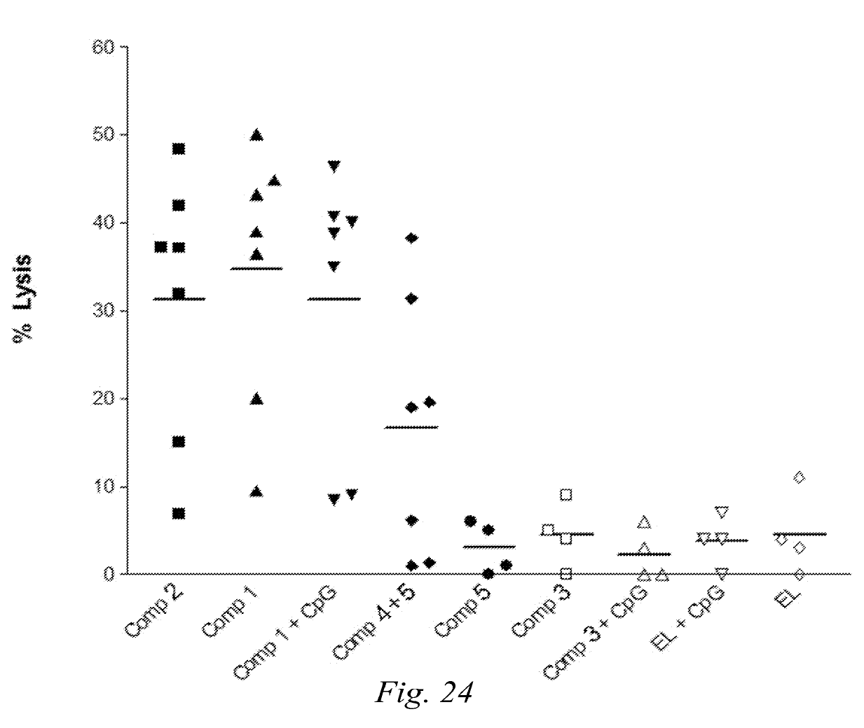

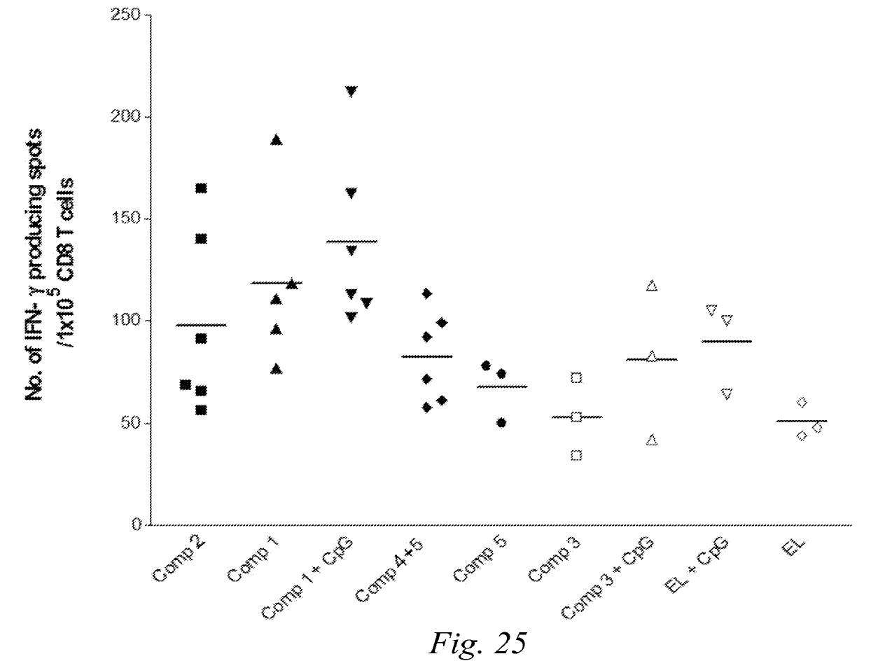

This example determined that effect of the addition of a second TLR agonist, CpG on the effectiveness of immunization. Following procedures described in more detail in Example 8, old MUC1.Tg mice (C57BL/6; H-2b) that express human MUC1 were immunized with preparations of Compound 2 (Pam₃CysSK₄-T helper ep. (Polio)-MUC1 (unglycosylated)); Compound 1 (Pam₃CysSK₄ T helper ep. (polio)-MUC1(Tn)); Compound 1 plus CpG (CpG oligodeoxynucleotides (CpG ODN))); Compound 5 (Pam₃CysSK₄) plus Compound 4 (T helper ep. (Polio)-MUC1(Tn)); Compound 5; Compound 3 (Pam₃CysSK₄-T helper ep. (Polio)); Compound 3 plus CpG; EL (empty liposomes) plus CpG; or EL. The structures of Compounds 1, 2, 3, 4, and 5 are shown in FIG. 16. Compounds were co-administered with the TLR9 agonist CpG using a standard immunization schedule. As shown in FIGS. 23-25, the administration of a combination of the TLR9 ligand CpG further improved the anti-tumor properties of three-component vaccine 1. Specifically, the addition of a second agonist led to a significant further reduction in tumor weight (FIG. 23), and induced more potent immune responses (FIGS. 24 and 25).

### Example 10

### Three-Component MUC1 Glycopeptide Vaccine Induced Both Humoral and Cellular Immune Responses in MUC1.Tg Mice with MMT Tumors

Effective immunotherapy for cancer depends on both cellular and humoral immune responses to tumor antigens. MUC1, which is expressed at increased levels on breast cancer, also exhibits altered glycosylation that contributes to the formation of novel antigens. The identification of MHC class I and II binding peptides derived from tumor-associated MUC1 has facilitated the development of MUC1 based cancer vaccines. MHC class I binding epitopes from MUC1 tandem repeat, when given as emulsification with adjuvants, result in strong cellular response with no antibody response. It is possible that better immunogenicity would be obtained using glycopeptides more representative of the novel forms of MUC1 as seen in cancer to which individuals may be less tolerant and that direct linking of the vaccine components would result in a superior immune response than delivering them as a cocktail. This example shows that a three-component vaccine composed of a TLR2 agonist, a helper epitope and a T cell epitope which is also a B cell epitope derived from the MUC1 can break tolerance and elicit both humoral and cellular immune response. Immunization with the MUC1 glycopeptide vaccine led to a significant reduction in tumor burden compared to mice treated with adjuvants only and empty liposomes. The three-component vaccine activated MUC1 glycopeptide-specific cytotoxic CD8+ T cells and elicited robust titers of IgG antibodies that mediated lysis of relevant tumor cells by ADCC.

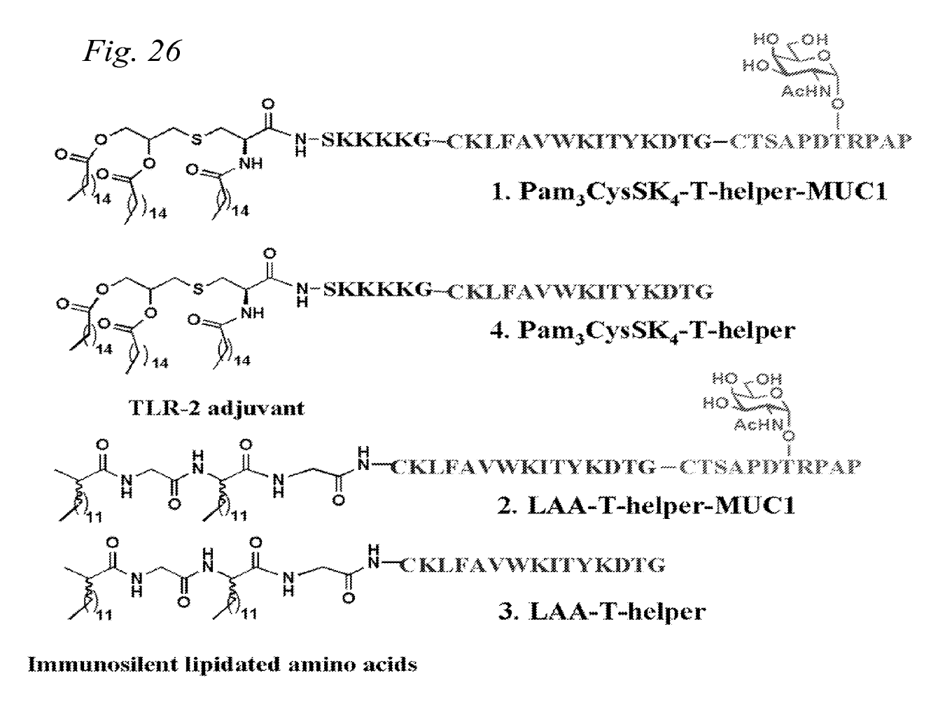

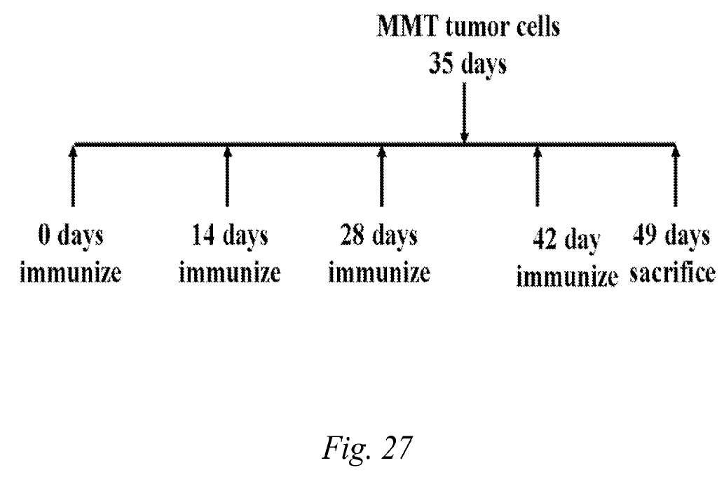

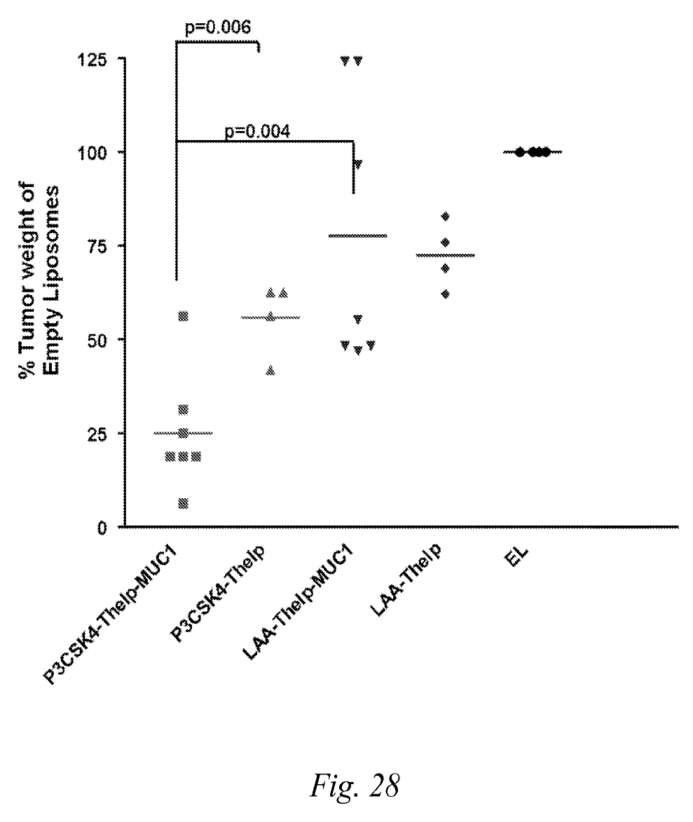

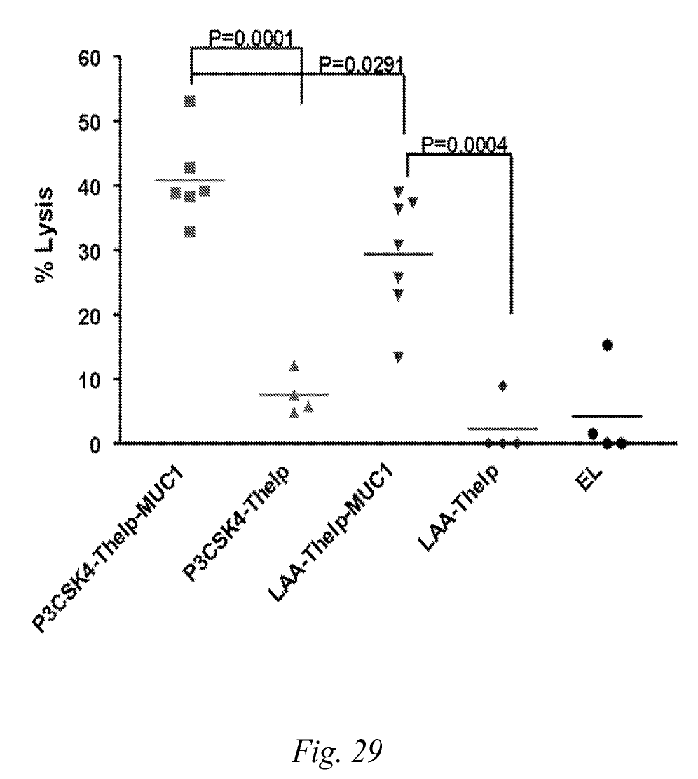

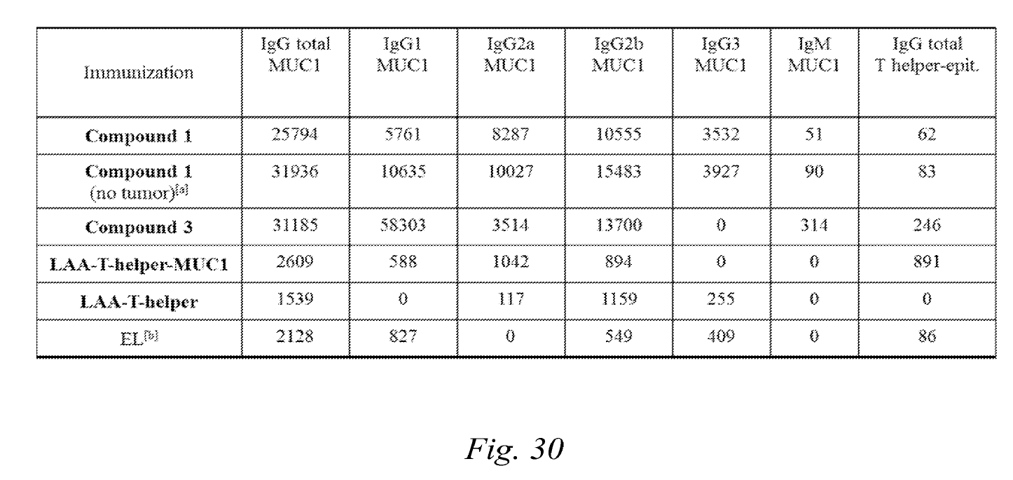

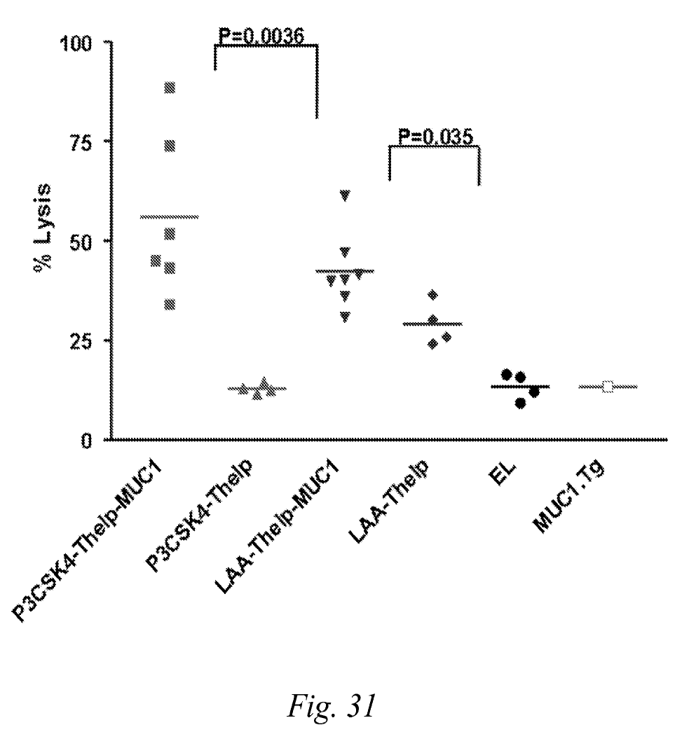

MUC1.Tg mice (C57BL/6; H-2b) that express human MUC1 were immunized three-times at biweekly intervals with liposomal preparations of the three-component vaccine Compound 1 (Pam₃CysSK₄-T-helper-MUC1) and LAA-T-helper-MUC1 (which contains immunosilent lipids) and as controls, Compound 2 ((Pam₃CysSK₄-T-helper) and LAA-T-helper (both of which lack the tumor-associated MUC1 epitope). The structure of the compounds is shown in FIG. 26. After 35 days, the mice were challenged with MMT mammary tumor cells, which express MUC1 and Tn. The immunization schedule is shown in FIG. 27. Three weeks after the last immunization, the mice were sacrificed and the efficacy of the vaccines determined by tumor burden; cell mediated immune response; and antibody mediated immune response. Immunization with three-component glycosylated vaccine Compound 1 led to a significant reduction in tumor mass compared to LAA-T-helper-MUC1 (a compound lacking the TLR2 agonist) and the respective controls, Compound 2 (TLR2 agonist-T helper epitope) and empty liposomes (see FIG. 28). Three-component glycosylated vaccine Compound 1 elicited robust titers of IgG antibodies that mediated lysis of relevant tumor cells by ADCC (see FIGS. 30 and 31). The lytic potential of the sorted CD8+ T cells from immunized MMT tumor-bearing mice as determined by the chromium release assay showed that immunization with the three component glycosylated vaccine showed significantly greater lysis as compared to the respective controls, Compound 2 ((Pam₃CysSK₄-T-helper) and EL as well as the LAA-T-helper-MUC1 compound that lacked the TLR2 ligand (see FIG. 29). This is the first vaccine preparation to elicit both a cellular and humoral response.

### Example 11

### Synthetic Three Component Constructs Utilizing Human MUC1 T-Helper Sequences

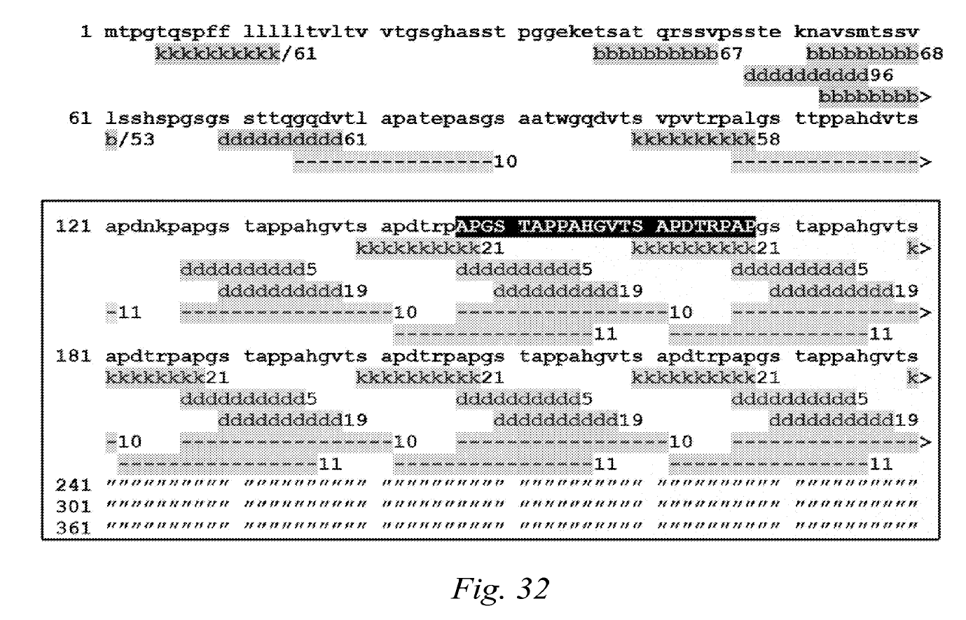

The Rankpe (Harvard, Mass.) Position Specific Scoring Matrices (PSSM) program is primarily polled for prediction of 1-A^{b}, H2-K^{b} and H2-D^{b} binding epitopes. A second program, SYPEITHI (Institute for Cell Biology, Heidelberg, Germany) is counter polled to cross-validated H2-K^{b} and H2-D^{b} binding epitopes. FIG. 32 displays the analysis for the binding of human MUC1-derived peptides to mouse I-A^{b} 15mers as well as to H2-D^{b} and H2-Kb 9mers. Many encouraging predictions are apparent. The dashed line shows 15mers showing RANKPEP score for binding to I-A^{b}. 9mers showing RANKPEP score for binding to H2-D^{b} (dddd) or H2-K^{b} (kkkk) or promiscuous binding to both (bbbb) are designated.

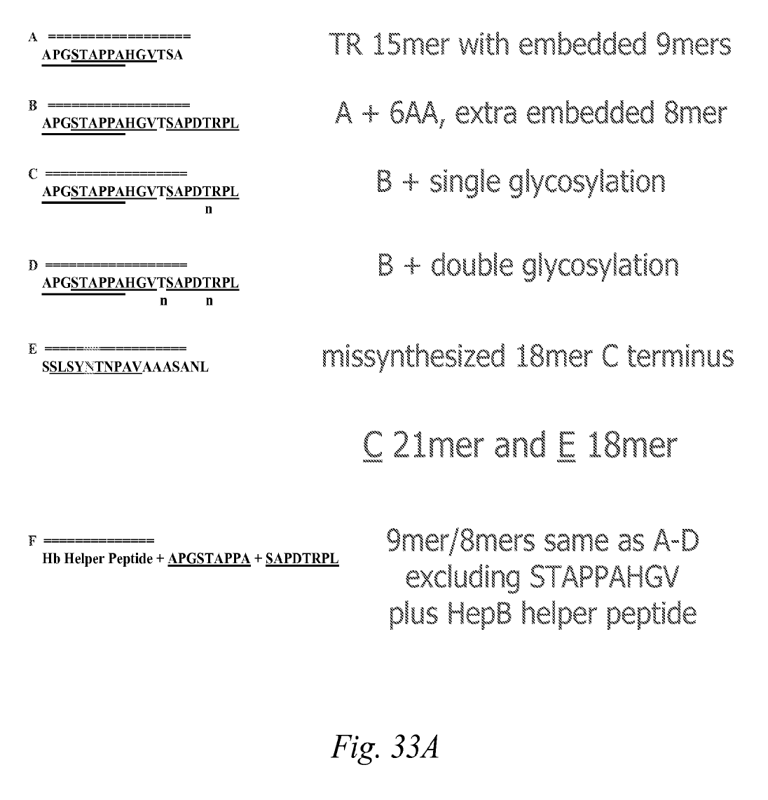

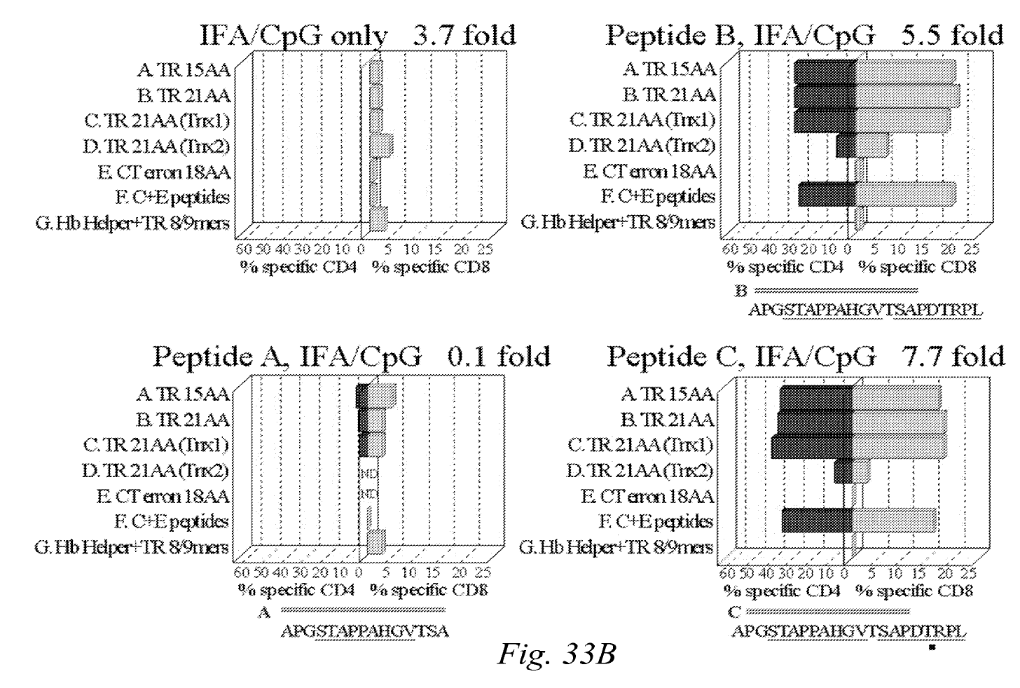

Compounds identified by this analysis were tested for induction of interferon γ production by CD4 and CD8 cells. Mice were immunized with the peptides described in FIG. 33A and lymph node-derived T-cells expressing low levels of CD62L were obtained by cell sorting and cultured for 14 days in the presence of DCs pulsed with the immunizing peptide. The resulting cells were analyzed by intracellular cytokine for the presence of CD4⁺IFNγ⁺ and CD8⁺IFNγ⁺ T-cells after exposure of the DCs pulsed with the peptides listed on the y-axis (FIG. 33B). Immunization with the glycosylated 21mer (peptide C) elicited a strong specific CD4⁺ and CD8⁺ response to itself as well as to the non glycosylated 15mer (peptide A) and 21mer (peptide B).

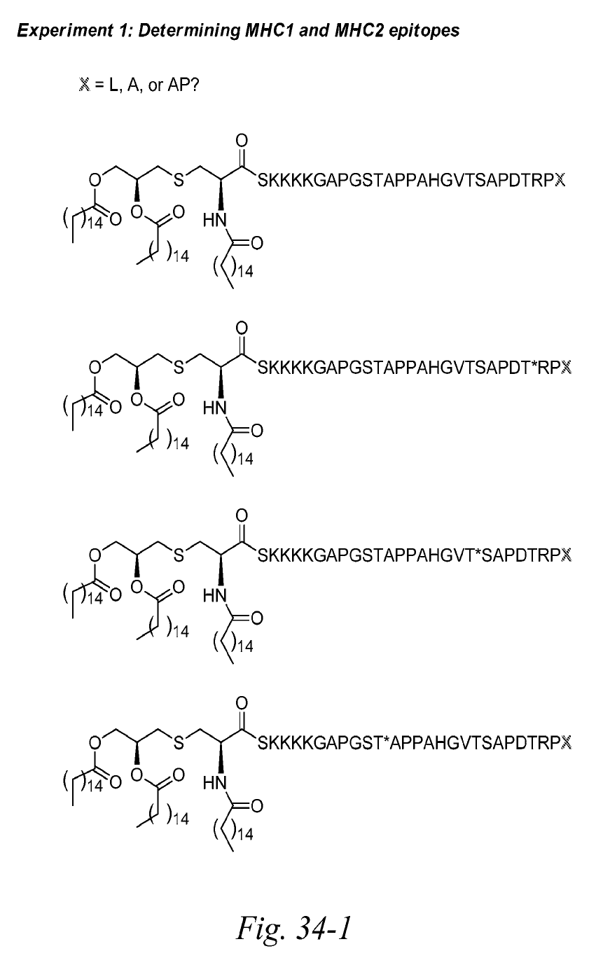

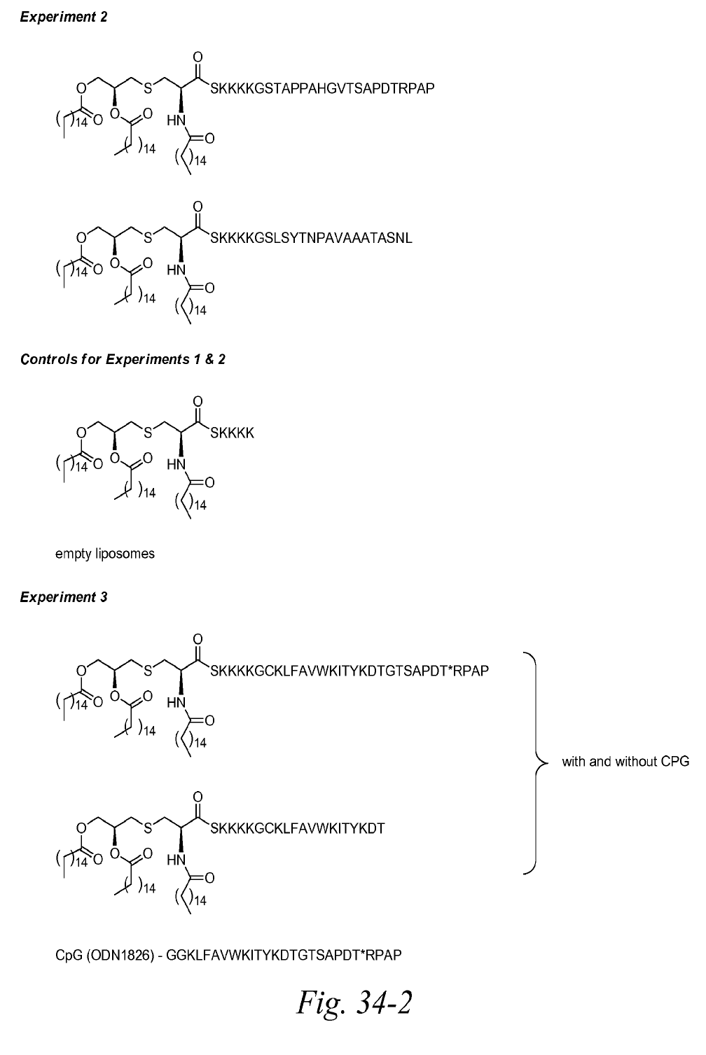

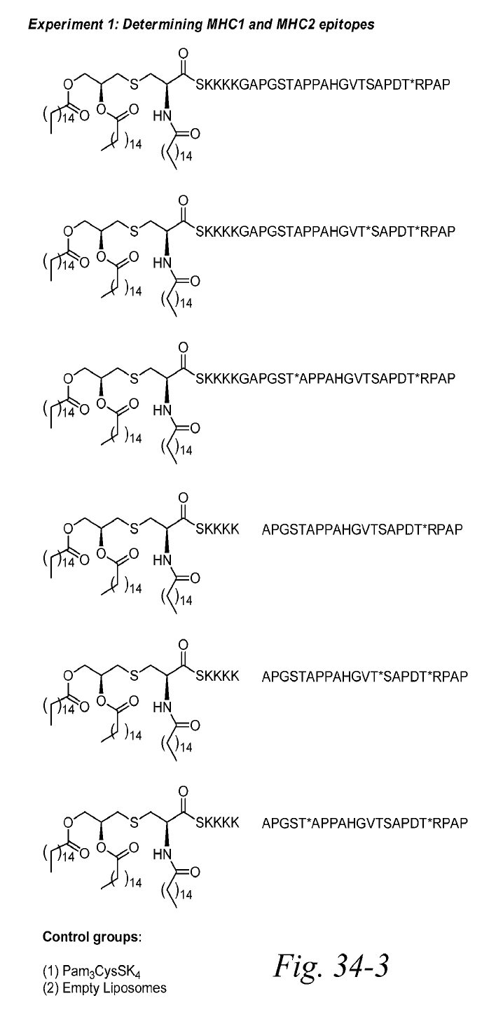

The various synthetic constructs utilizing human MUC1 T-helper sequences shown in FIG. 34 have been produced. Following procedures described in more detail in Example 8-10, MUC1.Tg mice (C57BL/6; H-2^{b}) that express human MUC1 will be immunized with the constructs shown in FIGS. 33 and 34. The effectiveness of the constructs in reducing in tumor mass, eliciting IgG antibodies, mediating lysis of tumor cells by ADCC, eliciting CD8+ cytotoxic activity, and producing IFN-γ and other cytokines will be determined following procedures described in more detail in Examples 8-10.

### Example 12

### Monoclonal Antibodies Against Carbohydrates and Glycopeptides by Using Fully Synthetic Three-Component Immunogens

Glycoconjugates are the most functionally and structurally diverse molecules in nature and it is now well established that protein- and lipid-bound saccharides play essential roles in many molecular processes impacting eukaryotic biology and disease. Examples of such processes include fertilization, embryogenesis, neuronal development, hormone activities, the proliferation of cells and their organization into specific tissues. Remarkable changes in the cell-surface carbohydrates occur with tumor progression, which appears to be intimately associated with metastasis. Furthermore, carbohydrates are capable of inducing a protective antibody response and this immunological reaction is a major contributor to the survival of the organism during infection.

The inability of saccharides to activate helper T-lymphocytes has complicated their development as vaccines. For most immunogens, including carbohydrates, antibody production depends on the cooperative interaction of two types of lymphocytes, the B-cells and helper T-cells (Jennings, Neoglyconjugates: Preparation and Applications 325-371 (Academic Press, Inc., 1994); Kuberan, Curr. Org. Chem. 2000, 4, 653-677). Saccharides alone cannot activate helper T-cells and therefore have a limited immunogenicity as manifested by low affinity IgM antibodies and the absence of IgG antibodies. In order to overcome the T-cell independent properties of carbohydrates, past research has focused on the conjugation of saccharides to a foreign carrier protein (e.g. Keyhole Limpet Hemocyanin (KLH) detoxified tetanus toxoid) (Jennings, Neoglyconjugates: Preparation and Applications 325-371 (Academic Press, Inc., 1994); Kuberan, Curr. Org. Chem. 2000, 4, 653-677; Jones, An. Acad. Bras. Cienc. 2005, 77, 293-324). In this approach, the carrier protein enhances the presentation of the carbohydrate to the immune system and provides T-epitopes (peptide fragments of 12-15 amino acids) that can activate T-helper cells. As a result, a class switch from low affinity IgM to high affinity IgG antibodies is accomplished. This approach has been successfully applied for the development of a conjugate vaccine to prevent infections with Haemophilus influenzae.

Carbohydrate-protein conjugate candidate vaccines composed of more demanding carbohydrate antigens, such as tumor associated carbohydrate and glycopeptides, have failed to elicit high titers of IgG antibodies. These results are not surprising because tumor-associated saccharides are of low antigenicity, because they are self-antigens and consequently tolerated by the immune system. The shedding of antigens by the growing tumor reinforces this tolerance. In addition, foreign carrier proteins such as KLH and BSA and the linker that attach the saccharides to the carrier protein can elicit strong B-cell responses, which may lead to the suppression of antibody responses against the carbohydrate epitope (Buskas, Chem. Eur. J. 2004, 10, 3517-3524; Ni, Bioconjug. Chem. 2006, 17, 493-500). It is clear that the successful development of carbohydrate-based cancer vaccines requires novel strategies for the more efficient presentation of tumor-associated carbohydrate epitopes to the immune system, resulting in a more efficient class switch to IgG antibodies (Reichel, Chem. Commun. 1997, 21, 2087-2088; Alexander, J. Immunol. 2000, 164, 1625-1633; Kudryashov, Proc. Natl. Acad. Sci. U.S.A. 2001, 98, 3264-3269; Lo-Man, J. Immunol. 2001, 166, 2849-2854; Jiang, Curr. Med. Chem. 2003, 10, 1423-1439; Jackson, Proc. Natl. Acad. Sci. U.S.A. 2004, 101, 15440-5; Lo-Man, Cancer Res. 2004, 64, 4987-4994; Buskas, Angew. Chem. Int. Ed. 2005, 44, 5985-5988 (Example I); Dziadek, Angew. Chem. Int. Ed. 2005, 44, 7624-7630; Krikorian, Bioconjug. Chem. 2005, 16, 812-819; Pan, J. Med. Chem. 2005, 48, 875-883).

As shown in the previous examples, a three-component vaccine composed of a TLR2 agonist, a promiscuous peptide T-helper epitope and a tumor-associated glycopeptide, can elicit in mice exceptionally high titers of IgG antibodies that can recognize cancer cells expressing the tumor-associated carbohydrate (see compound 21, FIG. 5, Example 6 and compound 51, FIG. 15) (Ingale, Nat. Chem. Biol. 2007, 3, 663-667). The superior properties of the vaccine candidate are attributed to the local production of cytokines, upregulation of co-stimulatory proteins, enhanced uptake by macrophages and dendritic cells and avoidance of epitope suppression.

The three-component immunogen technology of the invention can be used to generate monoclonal antibodies (MAbs) for poorly antigenic carbohydrates and glycopeptides. We have initially focused on MAbs against β-N-acetylglucosamine (β-O-GlcNAc) modified peptides (Wells, Science 2001, 291, 2376-2378; Whelan, Methods Enzymol. 2006, 415, 113-133; Zachara, Biochim. Biophys. Acta, 2006, 1761, 599-617; Dias and Hart, Mol. Biosyst. 2007, 3, 766-772; Hart, Nature 2007, 446, 1017-1022; Lefebvre, Exp. Rev. Proteomics 2005, 2, 265-275). Myriad nuclear and cytoplasmic proteins in metazoans are modified on Ser and Thr residues by the monosaccharide β-O-GlcNAc. The rapid and dynamic change in O-GlcNAc levels in response to extracellular stimuli suggests a key role for O-GlcNAc in signal transduction pathways. Modulation of O-GlcNAc levels has profound effects on the functioning of cells, in part mediated through a complex interplay between O-GlcNAc and O-phosphate. Recently, O-GlcNAc has been implicated in the etiology of type II diabetes, the regulation of stress response pathways and in the regulation of the proteasome. Progress in this exciting field of research is seriously hampered by the lack of reagents such as appropriate MAbs. In this respect, only one poorly performing IgM MAb with relative broad specificity (Corner, Anal. Biochem. 2001, 293, 169-177) is commercially available (Covance Research Products Inc).

We have designed and synthesized compound 52 (FIG. 15), which contains as a B-epitope a β-GlcNAc modified glycopeptide derived from casein kinase II (CKII) (Kreppel, J. Biol. Chem. 1999, 274, 32015-32022), the well-documented murine MHC class II restricted helper T-cell epitope KLFAVWKITYKDT (SEQ ID NO:3) derived from the polio virus and the inbuilt adjuvant Pam₃CysSK₄. In addition, compound 53 was prepared which has an artificial thio-linked GlcNAc moiety, which was expected to have better metabolic stability. Compounds 52 and 53 were incorporated into phospholipid-based small uni-lamellar vesicles (SUVs) by hydration of a thin film of the synthetic compounds, egg phosphatidylcholine, phosphatidylglycerol and cholesterol in a HEPES buffer (10 mM, pH 6.5) containing NaCl (145 mM) followed by extrusion through a 100 nm Nuclepore® polycarbonate membrane. Groups of five female BALB/c mice were immunized intra-peritoneal four times at weekly intervals with liposomes containing 3 μg of saccharide.

Anti-glycopeptide antibody titers were determined by coating microtiter plates with CGSTPVS(β-O-GlcNAc)SANM conjugated to maleimide (MI) modified BSA and detection was accomplished with anti-mouse IgG antibodies labeled with alkaline phosphatase. As can be seen in Table 10, compounds 52 and 53 elicited excellent titers of anti-MUC1 IgG antibodies. Furthermore, no significant difference in titer was observed between the O- and S-linked saccharide derivatives.

TABLE 10
ELISA anti-GSTPVS(β-O-GlcNAc) SANM (68) titers^{a}
after 4 immunizations two different preparations

| Immunization^{b} | IgG total | IgG1 | IgG2a | IgG2b | IgG3 | IgM |
|---|---|---|---|---|---|---|
| O-GlcNAc 52^{c} | 76,500 | 61,400 | 33,200 | 12,500 | 69,400 | 81,900 |
| S-GlcNAc 53^{d} | 151,600 | 111,800 | 55,600 | 21,300 | 111,700 | 21,900 |

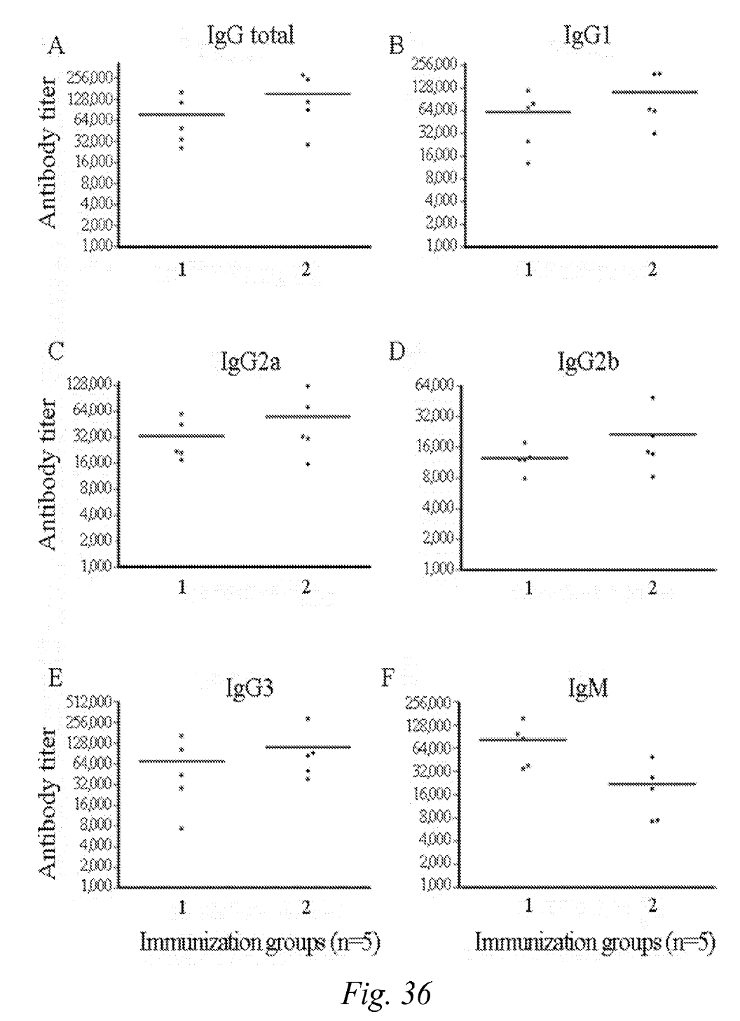

^{a}Anti-GSTPVS(β-O-GleNAc)SANM (68) antibody titers are presented as the mean of groups of five mice. ELISA plates were coated with BSA-MI-GSTPVS((β-O-GleNAc)SANM (BSA-MI-66) conjugate and titers were determined by linear regression analysis, plotting dilution vs. absorbance. Titers are defined as the highest dilution yielding an optical density of 0.1 or greater over that of normal control mouse sera.
^{b}Liposomal preparations were employed.
^{c}O-GlcNAc 52; Pam₃CysSK₄G-C-KLEAVWKITYKDT-G-GSTPVS (β-O-GluNAc) SANM.
^{d}S-GlcNAc 53; Pam₃CysSK₄G-C-KLFAVWKITYKDT-G-GSTPVS (β-S-GluNAc) SANM, A statistically significant difference was observed between 52 versus 53 for IgM titers (P = 0.0327). Individual titers for IgG total, IgGl, IgG2a, IgG2b, IgG3 and IgM are reported in FIG. 36.

Next, spleens of two mice immunized with the O-linked glycolipopeptide 52 were harvested and standard hybridoma culture technology gave seven IgG1, seven IgG2a, two IgG2b and fourteen IgG3 producing hybridoma cell lines. The ligand specificity of the resulting MAbs was investigated using ELISA and inhibition ELISA. All MAbs recognized CGSTPVS(β-O-GlcNAc)SANM linked to BSA whereas only a small number recognized the peptide CGSTPVSSANM (SEQ ID NO:12) conjugated to BSA. Furthermore, the interaction of nineteen MAbs with BSA-MI-CGSTPVS(β-O-GlcNAc)SANM could be inhibited with the glycopeptide GSTPVS(β-O-GlcNAc)SANM.

Hybridoma cell lines 1F5.D6, 9D1.E4 and 18B10.C7, as described in more detail in WO 2010/002478 (“Glycopeptide and Uses Thereof”) were deposited with the American Type Culture Collection (ATCC), 10801 University Blvd., Manassas, Va., 20110-2209, USA, on Jul. 1, 2008, and assigned ATCC deposit numbers PTA-9339, PTA-9340, and PTA-9341, respectively. It is nonetheless to be understood that the written description herein is considered sufficient to enable one skilled in the art to fully practice the present invention. Moreover, the deposited embodiment is intended as a single illustration of one aspect of the invention and is not to be construed as limiting the scope of the claims in any way.

Following similar procedures, polyblocnal and monoclonal antibodies with specificities for any of the MUC1 constructs described herein may be made.

### Example 13

### Generation of O-GlcNAc Specific Monoclonal Antibodies Using a Novel Synthetic Immunogen

Combining a fully synthetic three-component immunogen with hybridoma technology led to the generation of O-GlcNAc-specific IgG MAbs having a broad spectrum of binding targets. Large-scale shotgun proteomics led to the identification of 254 mammalian O-GlcNAc modified proteins, including a large number of novel glycoproteins. The data imply a role of O-GlcNAc in transcriptional/translational regulation, signal transduction, the ubiquitin pathway, anterograde trafficking of intracellular vesicles and post-translational modification.

O-glycosylation of serine and threonine of nuclear and cytoplasmic proteins by a single β-N-acetyl-D-glucosamine moiety (β-GlcNAc) is a ubiquitous post-translational modification that is highly dynamic and fluctuates in response to cellular stimuli through the action of the cycling enzymes, O-linked GlcNAc transferase (OGT) and O-GlcNAcase (OGA). This type of glycosylation has been implicated in many cellular processes, frequently via interplay with phosphorylation that can occur on the same amino acid residue 1. Importantly, alteration of O-GlcNAc levels has been linked to the etiology of prevalent human diseases including type II diabetes and Alzheimer's disease (Hart et al., 2007 Nature 446, 1017-1022).

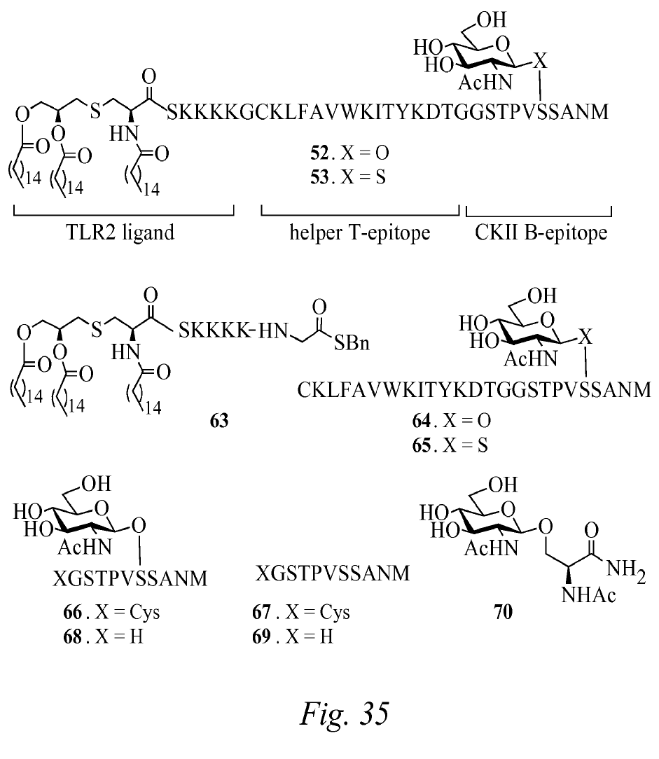

Unlike phosphorylation for which a wide range of pan- and site-specific phospho-antibodies are available, studies of O-GlcNAc modification are hampered by a lack of effective tools for its detection, quantification, and site localization. In particular, only two pan-O-GlcNAc specific antibodies have been described: an IgM pan-O-GlcNAc antibody (CTD 110.6; Corner et al., 2001 Anal. Biochem. 293, 169-177), and an IgG antibody raised against O-GlcNAc modified components of the nuclear pore (RL-2; Snow et al., 1987 J. Cell Biol. 104, 1143-1156) that shows restricted cross-reactivity with O-GlcNAc modified proteins. In fact, multiple studies have shown that O-GlcNAc modified glycoconjugates do not elicit relevant IgG isotype antibodies and thus, the challenge to elicit O-GlcNAc specific IgG antibodies is considerable. We reasoned that O-GlcNAc-specific antibodies can be elicited by employing a three-component immunogen (compound 52, FIG. 35) composed of an O-GlcNAc containing peptide, which in this study is derived from casein kinase II (CKII) α subunit, (Kreppel and Hart, 1999 J. Biol. Chem. 274, 32015-32022) a well-documented murine MHC class II restricted helper T-cell epitope and a Toll-like receptor-2 (TLR2) agonist as an in-built adjuvant. Such a compound is expected to circumvent immune suppression caused by a carrier protein or linker region of a classical conjugate vaccine; yet it contains all mediators required for eliciting a strong and relevant IgG immune response (Ingale et al., 2007 Nat. Chem. Biol. 3, 663-667). In addition, compound 53 was prepared that has an artificial thio-linked GlcNAc moiety, which has an improved metabolic stability compared to its O-linked counter-part thereby providing additional opportunities to eliciting O-GlcNAc specific antibodies.

Compounds 52 and 53 were readily obtained by liposome-mediated native chemical ligations (Ingale et al., 2006 Org. Lett. 8, 5785-5788) of C-terminal lipopeptide thioester 63 with glycopeptides 64 and 65, respectively (FIG. 35). The starting thioester 63 was assembled on a sulfonamide “safety-catch” linker followed by release by alkylation with iodoacetonitrile and treatment with benzyl mercaptan to give a compound that was deprotected using standard conditions. Compounds 64 and 65 were prepared employing a Rink amide resin, Fmoc protected amino acids and Fmoc-Ser-(AcO3-α-D-GluNAc) or Fmoc-Ser-(1-thio-AcO3-α-D-GluNAc), respectively. After completion of the assembly, the acetyl esters were cleaved by treatment with 60% hydrazine in MeOH and the resulting compounds were cleaved from the resin by treatment with reagent K and purified by reverse phase HPLC. Compounds 52 and 53 were incorporated into phospholipid based small unilamellar vesicles (SUVs) followed by extrusion through a 100 nm Nuclepore® polycarbonate membrane. Groups of five female BALB/c mice were immunized intra-peritoneal four times at two-weekly intervals with liposomes containing 3 μg of saccharide. Antiglycopeptide antibody titers were determined by coating microtiter plates with CGSTPVS(β-O-GlcNAc) SANM (66) conjugated to maleimide (MI) modified BSA and detection was accomplished with anti-mouse IgG antibodies labeled with alkaline phosphatase. Compounds 52 and 53 elicited excellent titers of IgG antibodies (Table 10; FIG. 36). Furthermore, no significant difference in IgG titers was observed between the O- and S-linked saccharide derivatives, and therefore further attention was focused on mice immunized with 52.

Spleens of two mice immunized with 52 were harvested and standard hybridoma culture technology gave seven IgG1, seven IgG2a, two IgG2b and fourteen IgG3 producing hybridoma cell lines. The ligand specificity of the resulting MAbs was investigated by ELISA. All MAbs recognized CGSTPVS(β-O-GlcNAc)SANM linked to BSA (BSA-MI-66) whereas only a small number recognized the peptide CGSTPVSSANM (SEQ ID NO:12) conjugated to BSA (BSA-MI-67). Furthermore, the interaction of twenty MAbs could be inhibited with the glycopeptide GSTPVS(β-O-GlcNAc)SANM (68), but not with peptide GSTPVSSANM (SEQ ID NO: 13) (69) or β-O-GlcNAc-Ser (70) demonstrating glycopeptide specificity.

Three hybridomas (18B10.C7(3), 9D1.E4(10), 1F5.D6(14)) were cultured at a one-liter scale and the resulting antibodies purified by saturated ammonium sulfate precipitation followed by Protein G column chromatography to yield, in each case, approximately 10 mg of IgG. Inhibition ELISA confirmed that the MAbs require carbohydrate and peptide (glycopeptide) for binding.

In conclusion, the three-component immunogen methodology has been successfully employed to generate a panel of pan-GlcNAc specific MAbs, which offer powerful new tools for exploring the biological implications of this type of protein glycosylation. The newly identified O-GlcNAc modified proteins open new avenues to explore the importance of this type of posttranslational for a variety of biological processes. It is to be expected that the three-component immunization technology will find wide application for the generation of MAbs for other forms of protein glycosylation.

### Methods

Reagents and General Procedures for Synthesis.

Fmoc-L-Amino acid derivatives and resins were purchased from NovaBioChem and Applied Biosystems, peptide synthesis grade N,N-dimethylformamide (DMF) from EM Science and N-methylpyrrolidone (NMP) from Applied Biosystems. Egg phosphatidylcholine (PC), egg phosphatidylglycerol (PG), cholesterol, monophosphoryl lipid A (MPL-A) and dodecyl phosphocholine (DPC) were obtained from Avanti Polar Lipids. All other chemical reagents were purchased from Aldrich, Acros, Alfa Aesar and Fischer and used without further purification. All solvents employed were reagent grade. Reversed phase high performance liquid chromatography (RP-HPLC) was performed on an Agilent 1100 series system equipped with an auto-injector, fraction-collector and UV-detector (detecting at 214 nm) using an Agilent ZorbaxEclipse™ C8 analytical column (5 μm, 4.6×150 mm) at a flow rate of 1 ml min⁻¹, Agilent Zorbax Eclipse™ C8 semi preparative column (5 μm, 10×250 mm) at a flow rate of 3 ml min⁻¹ or Phenomenex Jupiter™ C4 semi preparative column (5 μm, 10×250 mm) at a flow rate of 2 ml min⁻¹. All runs were performed using a linear gradients of 0 to 100% solvent B over 40 min. (solvent A=5% acetonitrile, 0.1% trifluoroacetic acid (TFA) in water, solvent B=5% water, 0.1% TFA in acetonitrile). Matrix assisted laser desorption ionization time of flight mass spectrometry (MALDI-ToF) mass spectra were recorded on an ABI 4700 proteomic analyzer.

General Methods for Solid-Phase Peptide Synthesis (SPPS).

Peptides were synthesized by established protocols on an ABI 433A peptide synthesizer (Applied Biosystems) equipped with UV-detector using N^{α}-Fmoc-protected amino acids and 2-(1H-bezotriazole-1-yl)-oxy-1,1,3,3-tetraethyl hexafluorophosphate (HBTU)/1-hydroxybenzotriazole (HOBt; Knorr et al., 1989 Tetrahedron Lett. 30, 1927-1930) as the activating reagents. Single coupling steps were performed with conditional capping. The following protected amino acids were used: N^{o}-Fmoc-Arg(Pbf)-OH, N^{α}-Fmoc-Asp(O^{T}Bu)—OH, N^{α}-Fmoc-Asp-Thr(Ψ^{Me,Me}pro)-OH, N^{α}-Fmoc-Ile-Thr(Ψ^{Me},Mepro)-OH, N^{α}-Fmoc-Lys(Boc)—OH, N^{α}-Fmoc-Ser(^{t}Bu)-OH, N^{α}-Fmoc-Thr(^{t}Bu)-OH, N^{α}-Fmoc-Tyr(^{t}Bu)—OH. The coupling of the glycosylated amino acid N^{α}-FmocSer-(AcO3-α-D-O-GlcNAc)OH, N^{α}-FmocSer-(AcO3-α-D-S-GlcNAc)OH, was carried out manually using O-(7-azabenzotriazol-1-yl)-N,N,N′,N′-tetramethyl-uronium hexafluorophosphate (HATU)/1-hydroxy-7-azabenzotriazole (HOAt) as a coupling agent. The coupling of N^{α}-Fmoc-S-(2,3-bis(palmitoyloxy)-(2R-propyl)-(R)-cysteine (Metzger et al., 1991 hit. J. Pept. Protein Res. 38, 545-554; Roth et al., 2004 Bioconjugate Chem. 15, 541-553) which was prepared from (R)-glycidol were carried out using benzotriazole-1-yl-oxy-tris-pyrrolidino-phosphonium hexafluorophosphate (PyBOP)/HOBt as coupling agent. Progress of the manual couplings was monitored by standard Kaiser test (Kaiser et al., 1970 Anal. Biochem. 34, 595).

Synthesis of lipopeptide 63.

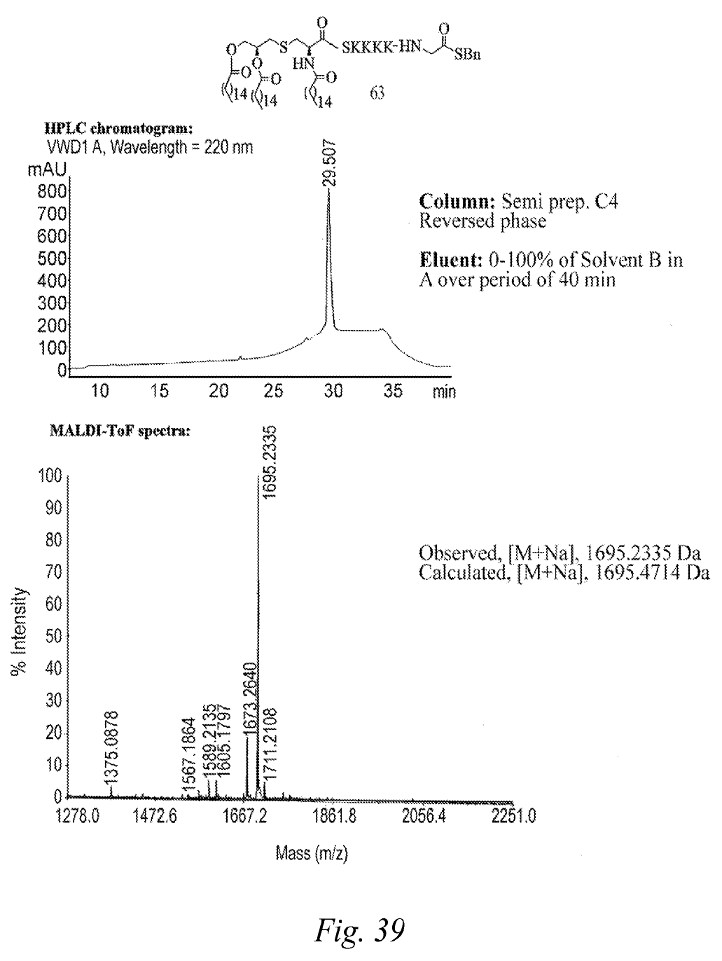

The synthesis of 63 was carried out on a H-Gly-sulfamylbutyryl Novasyn TG resin as described in the general method section for peptide synthesis. After coupling of the first five amino acids, the remaining steps were performed manually. N-α-Fmoc-S-(2,3-bis(palmitoyloxy)-(2R-propyl)-(R)-cysteine (267 mg, 0.3 mmol) was dissolved in DMF (5 ml) and PyBOP (156.12 mg, 0.3 mmol), HOBt (40 mg, 0.3 mmol) and DIPEA (67 μl, 0.4 mmol) were premixed for 2 min, and was added to the resin. The coupling reaction was monitored by the Kaiser test and was complete after standing for 12 h. Upon completion of the coupling, the N-Fmoc group of was cleaved using 20% piperidine in DMF (6 ml) and palmitic acid (77 mg, 0.3 mmol) was coupled to the free amine of as described above using PyBOP (156.12 mg, 0.3 mmol), HOBt (40 mg, 0.3 mmol) and DIPEA (67 μl, 0.4 mmol) in DMF. The resin was thoroughly washed with DMF (10 ml), DCM (10 ml) and MeOH (10 ml) and then dried in vacuo. The resin was swelled in DCM (5 ml) for 1 h and treated with DIPEA (0.5 ml, 3 mmol), iodoacetonitrile (0.36 ml, 5 mmol) in NMP (6 ml). It is important to note that the iodoacetonitrile was filtered through a plug of basic alumina before addition to the resin. The resin was agitated under the exclusion of light for 24 h, filtered and washed with NMP (5 ml×4), DCM (5 ml×4) and THF (5 ml×4). The activated N-acyl sulfonamide resin was swollen in DCM (5 ml) for 1 h, drained and transferred to a 50 ml round bottom flask. To the resin-containing flask was added THF (4 ml), benzyl mercaptan (0.64 ml, 5 mmol) and sodium thiophenate (27 mg, 0.2 mmol). After agitation for 24 h, the resin was filtered and washed with hexane (5 ml×2). The combined filtrate and washings were collected and concentrated in vacuo to approximately ⅓ of its original volume. The crude product was then precipitated by the addition of tert-butyl methyl ether (0° C.; 60 ml) and recovered by centrifugation at 3000 rpm for 15 min, and after the decanting of the ether the peptide precipitate was dissolved in mixture DCM and MeOH (1.5 ml/1.5 ml). The thiol impurity present in the peptide precipitate was removed by passing it through a LH-20 size exclusion column. The fractions containing product were collected and solvents removed to give the fully protected peptide thioester. The protected peptide was treated with a reagent B (TFA 88%, phenol 5%, H₂O 2O 5%, TIS 2%; 5 ml) for 4 h at room temperature. The TFA solution was then added dropwise to a screw cap centrifuge tube containing ice cold tert-butyl methyl ether (40 ml) and the resulting suspension was left overnight at 4° C., after which the precipitate was collected by centrifugation at 3000 rpm (20 min), and after the decanting of the ether the peptide precipitate was re-suspended in ice cold tert-butyl methyl ether (40 ml) and the process of washing was repeated twice. The crude peptide was purified by HPLC on a semi preparative C-4 reversed phase column using a linear gradient of 0 to 100% solvent B in A over a 40 min, and the appropriate fractions were lyophilized to afford 63 (110 mg, 65%). C₉₀H₁₆₅N₁₁O₁₃S₂, MALDI-ToF MS: observed, [M+Na] 1695.2335 Da; calculated, [M+Na] 1695.4714 Da (FIG. 39).

Synthesis of Glycopeptide 64.

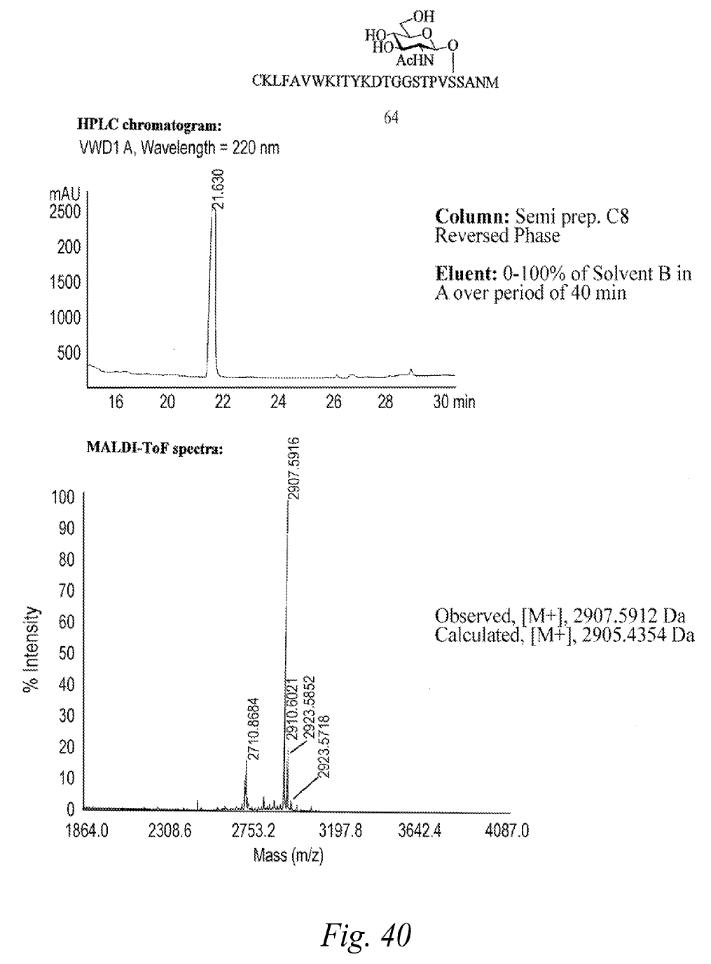

SPPS was performed on Rink amide resin (0.1 mmol) as described in the general procedures. The first four amino acids, Ser-Ala-Asn-Met, were coupled on the peptide synthesizer using a standard protocol. After the completion of the synthesis, a manual coupling was carried out using Nα-FmocSer-(AcO₃-α-D-O-GlcNAc)OH (0.2 mmol, 131 mg), with O-(7-azabenzotriazol-1-yl)-N,N,N′,N′-tetramethyl-uronium hexafluorophosphate (HATU; 0.2 mmol, 76 mg), 1-hydroxy-7-azabenzotriazole (HOAt; 0.2 mmol, 27 mg) and diisopropylethylamine (DIPEA; 0.4 mmol, 70 μl) in NMP (5 ml) for 12 h. The coupling reaction was monitored by standard Kaiser test. The resin was then washed with NMP (6 ml) and methylene chloride (DCM; 6 ml), and resubjected to the same coupling conditions to ensure completion of the coupling. The glycopeptide was then elongated on the peptide synthesizer after which the resin was thoroughly washed with NMP (6 ml), DCM (6 ml) and MeOH (6 ml) and dried in vacuo. The resin was swelled in DCM (5 ml) for 1 h and then treated with hydrazine (60%) in MeOH (10 ml) for 2 h and washed thoroughly with NMP (5 ml×2), DCM (5 ml×2) and MeOH (5 ml×2) and dried in vacuo. The resin was swelled in DCM (5 ml) for 1 h, after which it was treated with reagent K (TFA (81.5%), phenol (5%), thioanisole (5%), water (5%), EDT (2.5%), TIS (1%)) (30 ml) for 2 h at room temperature. The resin was filtered and washed with neat TFA (2 ml). The filtrate was then concentrated in vacuo to approximately ⅓ of its original volume. The peptide was precipitated using diethyl ether (0° C.) (30 ml) and recovered by centrifugation at 3000 rpm for 15 min. The crude peptide was purified by RP-HPLC on a semi preparative C-8 column using a linear gradient of 0 to 100% solvent B in solvent A over a 40 min period and the appropriate fractions were lyophilized to afford 64 (118 mg, 40%). C₁₂₉H₂₀₄N₃₂O₄₀S₂, MALDI-ToF MS: observed 2907.5916 Da; calculated [M+], 2905.4354 Da (FIG. 40).

Synthesis of Glycopeptide 65.

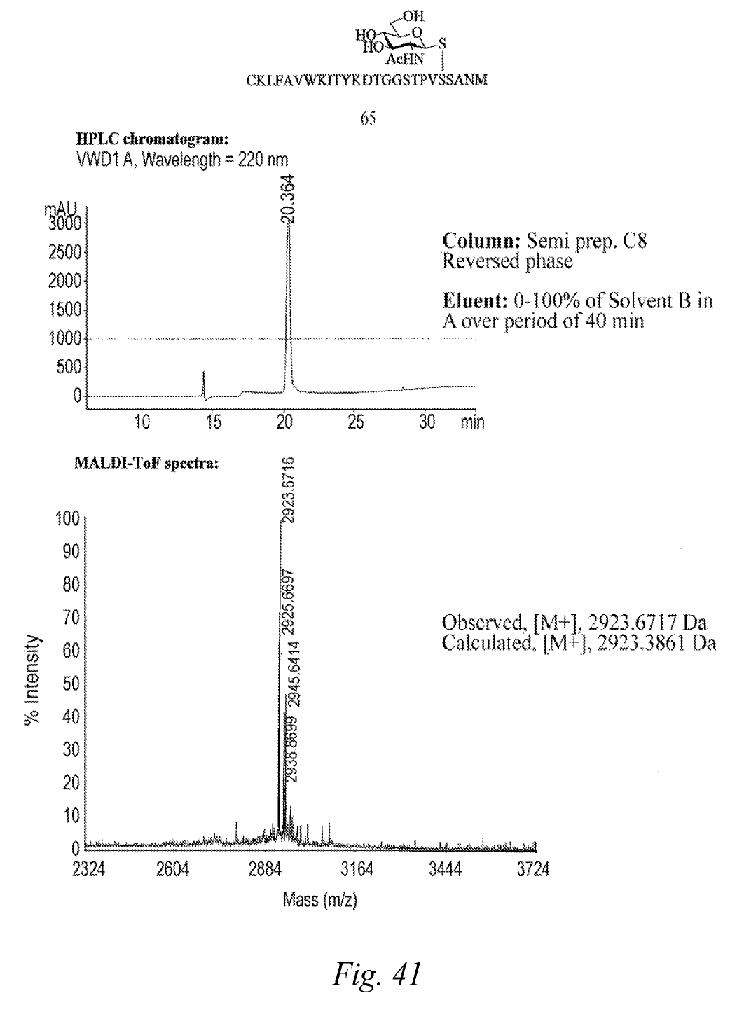

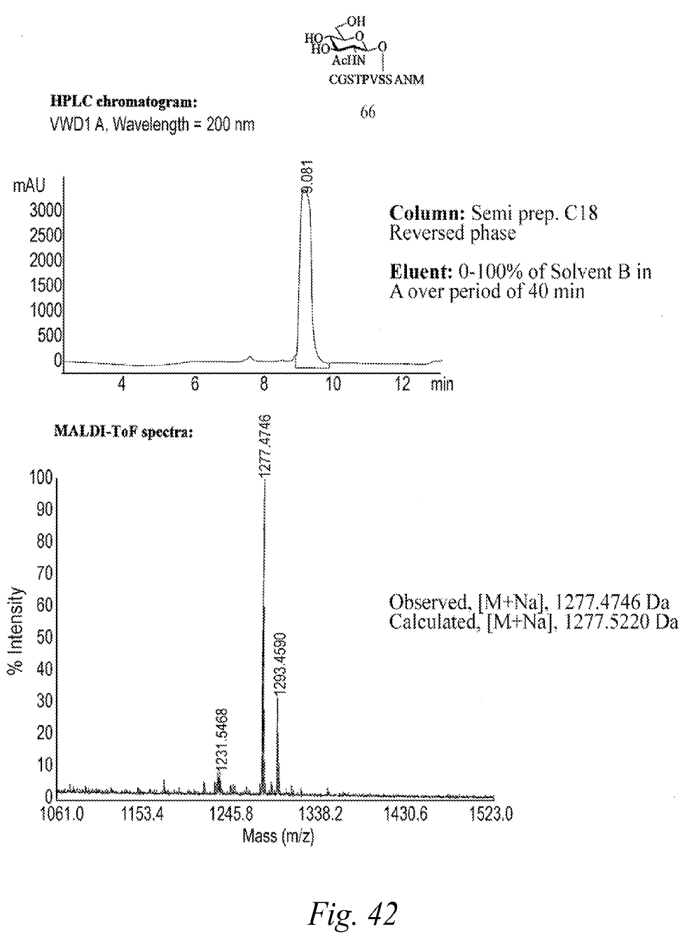

SPPS was performed on Rink amide resin (0.1 mmol) as described in the general procedures. The first four amino acids, Ser-Ala-Asn-Met, were coupled on the peptide synthesizer using a standard protocol. After the completion of the synthesis, a manual coupling was carried out using Nα-FmocSer-(AcO₃-α-D-S-GlcNAc)OH (0.2 mmol, 134 mg),with 0-(7-azabenzotriazol-1-yl)-N,N,N′,N′-tetramethyl-uronium hexafluorophosphate (HATU; 0.2 mmol, 76 mg), 1-hydroxy-7-azabenzotriazole (HOAt; 0.2 mmol, 27 mg) and diisopropylethylamine (DIPEA; 0.4 mmol, 70 μl) in NMP (5 ml) for 12 h. The coupling reaction was monitored by standard Kaiser test. The resin was then washed with NMP (6 ml) and methylene chloride (DCM; 6 ml), and resubjected to the same coupling conditions to ensure complete coupling. The resulting glycopeptide was then elongated on the peptide synthesizer. After the completion of the synthesis, the resin was thoroughly washed with NMP (6 ml), DCM (6 ml) and MeOH (6 ml) and dried in vacuo. The resin was swelled in DCM (5 ml) for 1 h and then treated with hydrazine (60%) in MeOH (10 ml) for 2 h and washed thoroughly with NMP (5 ml×2), DCM (5 ml×2) and MeOH (5 ml×2) and dried in vacuo. The resin was swelled in DCM (5 ml) for 1 h, after which it was treated with TFA (81.5%), phenol (5%), thioanisole (5%), water (5%), EDT (2.5%), TIS (1%) (30 ml) for 2 h at room temperature. The resin was filtered and washed with neat TFA (2 ml). The filtrate was then concentrated in vacuo to approximately ⅓ of its original volume. The peptide was precipitated using diethyl ether (30 ml, 0° C.) and recovered by centrifugation at 3000 rpm for 15 min. The crude peptide was purified by RP-HPLC on a semi preparative C-8 column using a linear gradient of 0 to 100% solvent B in solvent A over a 40 min period and the appropriate fractions were lyophilized to afford 65 (95 mg, 34%). C₁₂₉H₂₀₄N₃₂O₃₉S₃, MALDI-ToF MS: observed [M+], 2923.6716 Da; calculated [M+], 2923.3861 Da (FIG. 41).

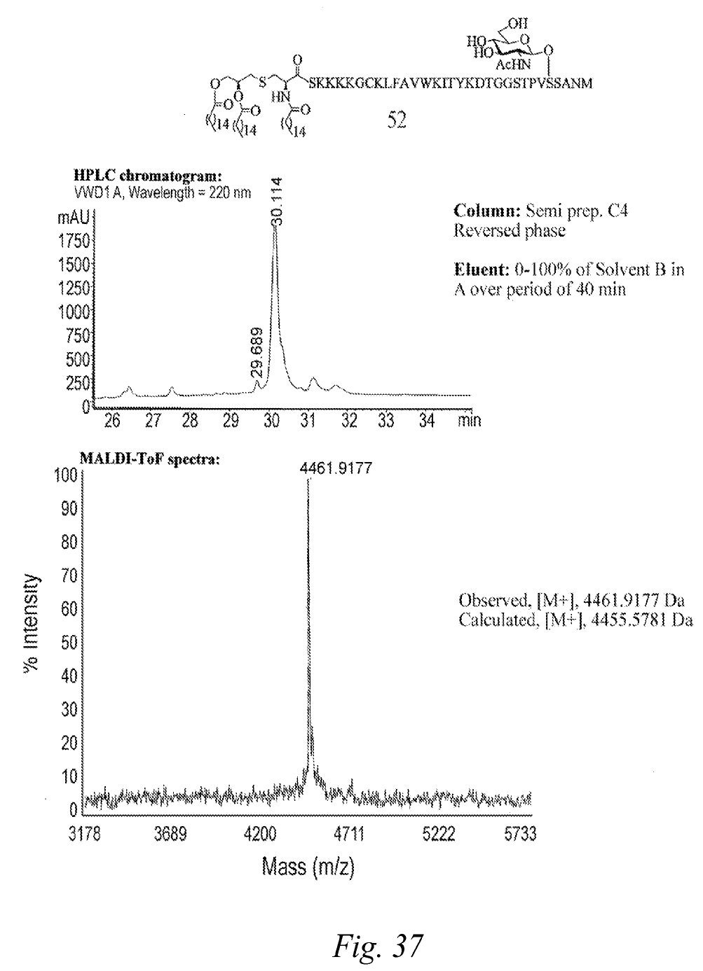

Synthesis of Glycolipopeptide 52. The lipopeptide thioester 63 (4.3 mg, 2.5 μmol), glycopeptide 64 (5.0 mg, 1.7 μmol) and dodecyl phosphocholine (6.0 mg, 17.0 μmol) were dissolved in a mixture of trifluoroethanol and CHCl₃ (2.5 ml/2.5 ml). The solvents were removed under reduced pressure to give a lipid/peptide film, which was hydrated for 4 h at 37° C. using 200 mM phosphate buffer (pH 7.5, 3 ml) in the presence of tris(carboxyethyl)phosphine (2% w/v, 40.0 μg) and EDTA (0.1% w/v, 20.0 μg). The mixture was ultrasonicated for 1 min. To the vesicle suspension was added sodium 2-mercaptoethane sulfonate (2% w/v, 40.0 μg) to initiate the ligation reaction. The reaction was carried out in an incubator at 37° C. and the progress of the reaction was periodically monitored by MALDI-ToF, which showed disappearance of glycopeptide 64 within 2 h. The reaction was then diluted with 2-mercaptoethanol (20%) in ligation buffer (2 ml) and the crude peptide was purified by semi preparative C-4 reversed phase column using a linear gradient of 0 to 100% solvent B in A over a 40 min, and lyophilization of the appropriate fractions afforded 52 (4.3 mg, 57%). C₂₁₂H₃₆₀N₄₃O₅₃S₃, MALDI-ToF MS: observed, 4461.9177 Da, calculated, 4455.578 Da (FIG. 37).

Synthesis of Glycolipopeptide 53.

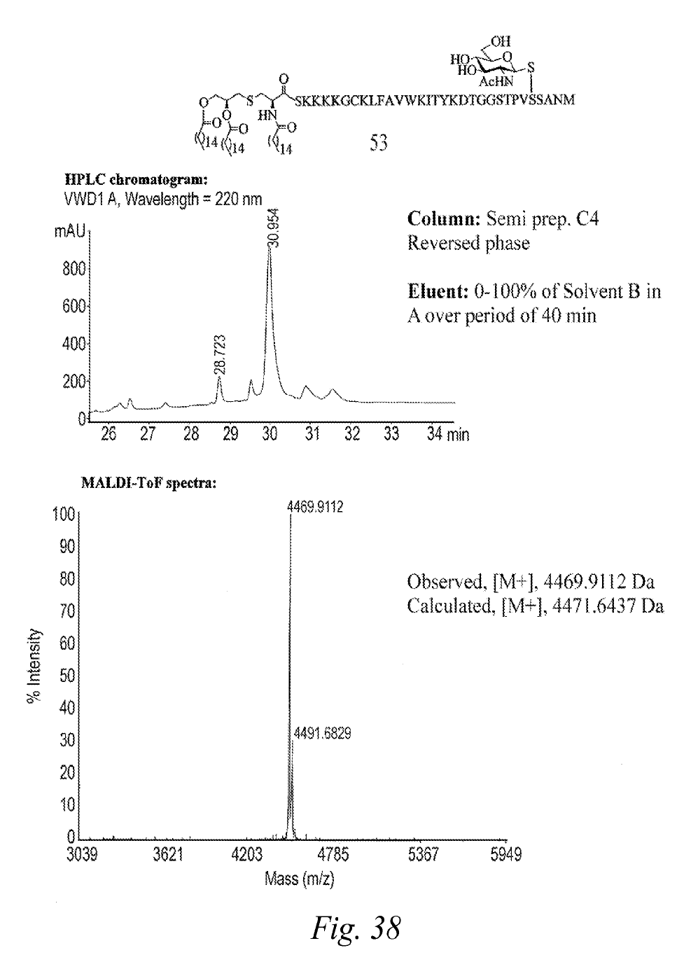

Lipopeptide thioester 63 (2.5 mg, 1.5 mop, glycopeptide 65 (3.0 mg, 1.0 μmol) and dodecyl phosphocholine (3.5 mg, 10 μmol) were dissolved in a mixture of trifluoroethanol and CHCl₃ (2.5 ml/2.5 ml). The solvents were removed under reduced pressure to give a lipid/peptide film, which hydrated for 4 h at 37° C. using 200 mM phosphate buffer (pH 7.5, 2 ml) in the presence of tris(carboxyethyl)phosphine (2% w/v, 40.0 μg) and EDTA (0.1% w/v, 20.0 μg). The mixture was ultrasonicated for 1 min. To the vesicle suspension was added sodium 2-mercaptoethane sulfonate (2% w/v, 40.0 μg) to initiate the ligation reaction. The reaction was carried out in an incubator at 37° C. and the progress of the reaction was periodically monitored by MALDI-ToF, which showed disappearance of glycopeptide within 2 h. The reaction was then diluted with 2-mercaptoethanol (20%) in ligation buffer (2 ml). The crude peptide was purified by semi preparative C-4 reversed phase column using a linear gradient of 0 to 100% solvent B in A over a 40 min, and lyophilization of the appropriate fractions afforded 53 (2.8 mg, 64%). C₂₁₂H₃₆₀N₄₃O₅₂S₄, MALDI-ToF MS: observed, 4469.9112 Da, calculated, 4471.6437 Da (FIG. 38).

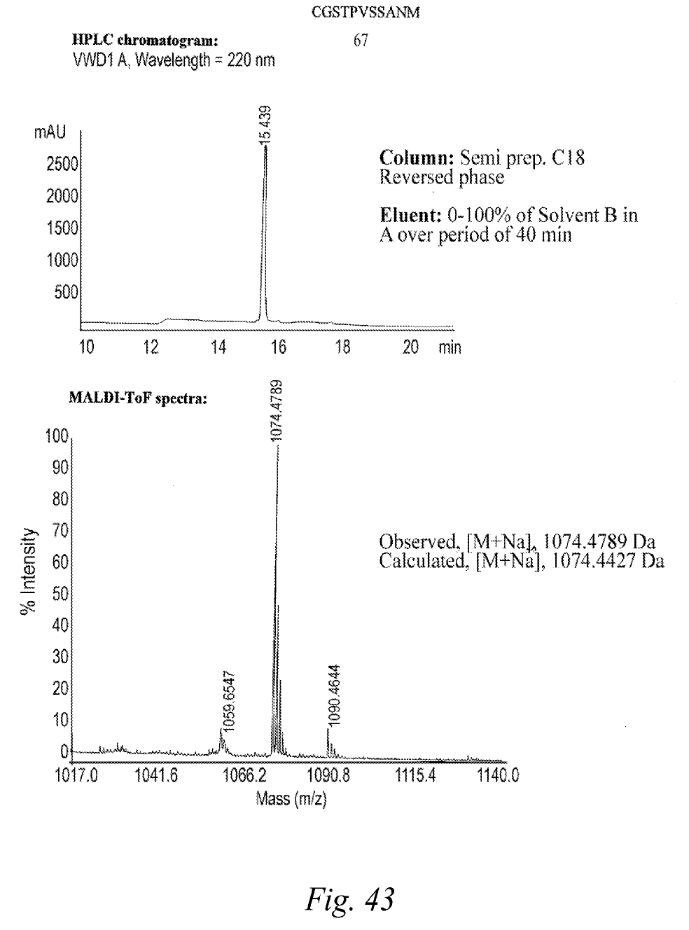

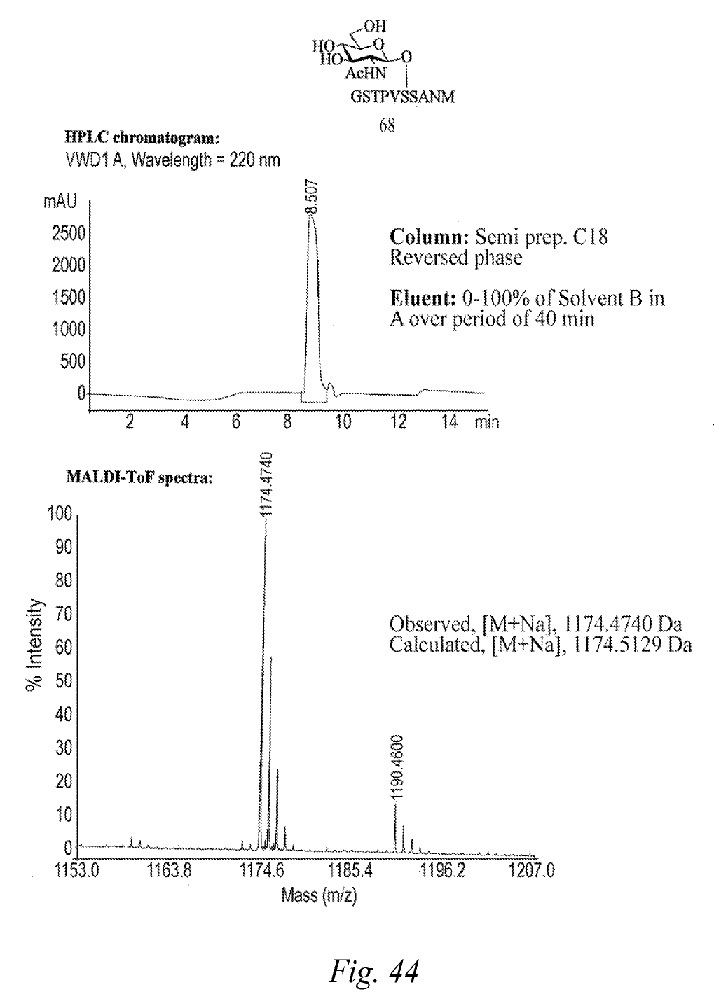

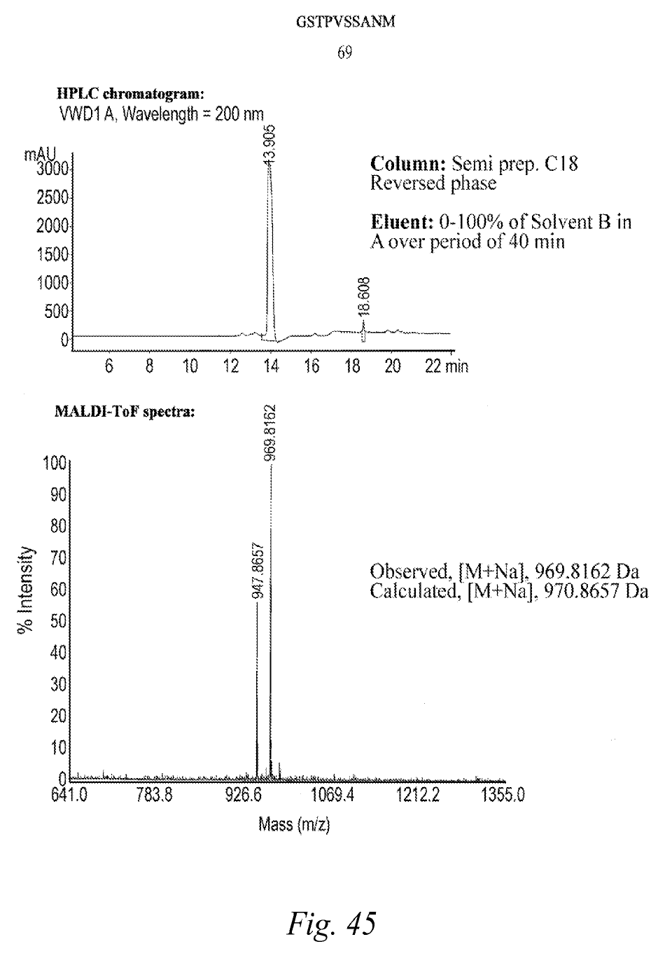

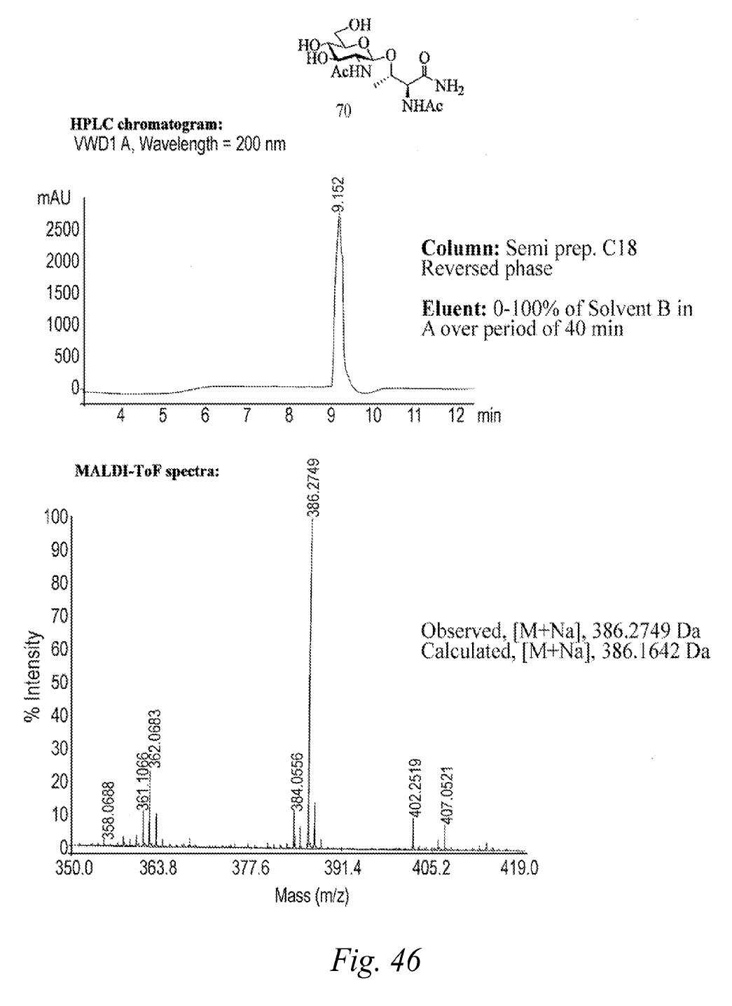

Compounds 66-70 were prepared as described in the standard procedures section on Rink amide resin (0.1 mmol). Glycopeptide 66 (78 mg, 61%); C₄₈H₈₂N₁₄^{O}₂₁S₂, MALDI-ToF MS: observed [M+Na], 1277.4746 Da; calculated [M+Na], 1277.5220 Da (FIG. 43). Peptide 67 (89 mg, 83%); C₄₀H₆₉N₁₃O₁₆S₂, MALDI-ToF MS: observed [M+Na], 1074.4789 Da; calculated [M+Na], 1074.4427 Da (FIG. 44). Glycopeptide 68 (57 mg, 48%); C₄₅H₇₇N₁₃O₂₀S, MALDI-ToF MS: observed [M+Na], 1174.4740 Da; calculated [M+Na], 1174.5129 Da (FIG. 45). Peptide 69 (76 mg, 78%). C₃₇H₆₄N₁₂O₁₅S, MALDI-ToF MS: observed [M+Na], 969.8162 Da; calculated [M+Na], 970.8657 Da (FIG. 46). Glycosylated amino acid 70 (12 mg, 33%), C₁₄H₂₅N₃O₈, MALDI-ToF MS: observed [M+Na], 386.2749 Da; calculated [M+Na] 386.3636 Da (FIG. 46).

General Procedure for the Conjugation to BSA-MI.

The conjugations were performed as instructed by Pierce Endogen Inc. In short, the purified (glyco)peptide 66 or 67 (2.5 equiv. excess to available MI-groups on BSA) was dissolved in the conjugation buffer (sodium phosphate, pH 7.2 containing EDTA and sodium azide; 100 μl) and added to a solution of maleimide activated BSA (2.4 mg) in the conjugation buffer (200 μl). The mixture was incubated at room temperature for 2 h and then purified by a D-Salt™ dextran de-salting column (Pierce Endogen, Inc.), equilibrated and eluted with sodium phosphate buffer, pH 7.4 containing 0.15 M sodium chloride. Fractions containing the conjugate were identified using the BCA protein assay. Carbohydrate content was determined by quantitative monosaccharide analysis by HPAEC/PAD.

General Procedure for the Preparation of Liposomes.

Egg PC, egg PG, cholesterol, MPL-A and compound 52 or 53 (15 μmol, molar ratios 60:25:50:5:10) were dissolved in a mixture of trifluoroethanol and MeOH (1:1, v/v, 5 ml). The solvents were removed in vacuo to produce a thin lipid film, which was hydrated by suspending in HEPES buffer (10 mM, pH 6.5) containing NaCl (145 mM; 1 ml) under argon atmosphere at 41° C. for 3 h. The vesicle suspension was sonicated for 1 min and then extruded successively through 1.0, 0.6, 0.4, 0.2 and 0.1 μm polycarbonate membranes (Whatman, Nucleopore Track-Etch Membrane) at 50° C. to obtain SUVs. The sugar content of liposomes was determined by heating a mixture of SUVs (50 μl) and aqueous TFA (2 M, 200 μl) in a sealed tube for 4 h at 100° C. The solution was then concentrated in vacuo and analyzed by high-pH anion exchange chromatography using a pulsed ampherometric detector (HPAEC-PAD; Methrome) and CarboPac columns PA-10 and PA-20 (Dionex).

Dose and Immunization Schedule.

Groups of five mice (female BALB/c, age 8-10 weeks, from Jackson Laboratories) were immunized four times at two-week intervals. Each boost included 3 μg of saccharide in the liposome formulation. Serum samples were obtained before immunization (pre-bleed) and 1 week after the final immunization. The final bleeding was done by cardiac bleed.

Hybridoma Culture and Antibody Production.

Spleens of two mice immunized with 52 were harvested and standard hybridoma culture technology gave 30 IgG producing hybridoma cell lines. Three hybridomas (18B10.C7(3), 9D1.E4(10), 1F5.D6(14)) were cultured at a one-liter scale and the resulting antibodies were purified by saturated ammonium sulfate precipitation followed by Protein G column chromatography to yield, in each case, approximately 10 mg of IgG.

Reagents for Biological Experiments.

Protease inhibitor cocktail was obtained from Roche (Indianapolis, Ind.). PUGNAc O-(2-acetamido-2-deoxy-D-glucopyranosylidene)amino N-phenyl carbamate was ordered from Toronto Research Chemicals, Inc (Ontario, Canada). Mouse IgM anti-O-GlcNAc (CTD110.6; Comer et al., 2001 Anal. Biochem. 293, 169-177) and rabbit polyclonal anti-OGT (AL28) antibodies were previously generated in Dr. Gerald W. Hart's laboratory (Johns Hopkins University School of Medicine, Baltimore, Md.). Rabbit polyclonal anti-OGA antibody was a kind gift from Dr. Sidney W. Whiteheart (University of Kentucky College of Medicine). Rabbit polyclonal anti-CKII alpha antibodies (NB 100-377 for immunoblotting and NB 100-378 for immunoprecipitation) were purchased from Novus Biologicals (Littleton, Colo.). Mouse monoclonal antibody against α-tubulin and anti-Mouse IgM (g chain)-agarose was obtained from Sigma (St. Louis, Mo.). Normal rabbit IgG agarose, normal rabbit IgG agarose and Protein A/G PLUS agarose were ordered from Santa Cruz Biotechnology, Inc. (Santa Cruz, Calif.).

Serologic Assays.

Anti-GSTPVS(β-O-GlcNAc)SANM (68) IgG, IgG1, IgG2a, IgG2b, IgG3 and IgM antibody titers were determined by enzyme-linked immunosorbent assay (ELISA), as described previously (Buskas and Boons, 2004 Chem. Eur. J. 10, 3517-3524; Ingale et al., 2007 Nat. Chem. Biol. 3, 663-66). Briefly, Immulon II-HB flat bottom 96-well microtiter plates (Thermo Electron Corp.) were coated overnight at 4° C. with 100 μl per well of a conjugate of the glycopeptide conjugated to BSA through a maleimide linker (BSA-MI-GSTPVS(β-O-GlcNAc) SANM; BSA-MI-66) at a concentration of 2.5 μg ml-1 in coating buffer (0.2 M borate buffer, pH 8.5 containing 75 mM sodium chloride). Serial dilutions of the sera or MAb containing cell supernatants were allowed to bind to immobilized GSTPVS(β-O-GlcNAc)SANM for 2 h at room temperature. Detection was accomplished by the addition of alkaline phosphatase-conjugated anti-mouse IgG (Jackson ImmunoResearch Laboratories Inc.), IgG1 (Zymed), IgG2a (Zymed), IgG2b (Zymed), IgG3 (BD Biosciences Pharmingen) or IgM (Jacksons ImmunoResearch Laboratories) antibodies. After addition of p-nitrophenyl phosphate (Sigma), the absorbance was measured at 405 nm with wavelength correction set at 490 nm using a microplate reader (BMG Labtech). The antibody titer was defined as the highest dilution yielding an optical density of 0.1 or greater over that of background.

To explore competitive inhibition of the binding of MAbs to GSTPVS(β-O-GlcNAc)SANM (68) by the corresponding glycopeptide, peptide and sugar, MAbs were diluted in diluent buffer in such a way that, without inhibitor, expected final OD values were approximately 1. For each well 60 p. 1 of the diluted MAbs were mixed in an uncoated microtiter plate with 60 μl diluent buffer, glycopeptide 68 (GSTPVS(β-O-GlcNAc)SANM), peptide 69 (GSTPVSSANM; SEQ ID NO: 11) or sugar 70 (β-O-GlcNAc-Ser) in diluent buffer with a final concentration of 0-500 μM. After incubation at room temperature for 30 min, 100 μl of the mixtures were transferred to a plate coated with BSA-MICGSTPVS(β-O-GlcNAc)SANM (BSA-MI-66). The microtiter plates were incubated and developed as described above using the appropriate alkaline phosphatase-conjugated detection antibody.

Plasmids Construction.

The human OGT and OGA cDNA were PCR amplified in a two-step manner to introduce an attB1 site and a HA epitope at the 5′ end as well as an attB2 site at the 3′ end to facilitate Gateway cloning strategy (Invitrogen, Carlsbad, Calif.). The primers include (1) Sense primer for first PCR to incorporate HA epitope into ogt after the start codon: 5′-CCCCATGTATCCATATGACGTCCCAGACTATGCCGCGTCTTCCGTGGGCAACGT-3′ (SEQ ID NO: 13); (2) Sense primer containing an attB1 site for using HA-ogt PCR product as the template: 5′-GGGGACAAGTTTGTACAAAAAAGCAGGCTGGATGATGTATCCATATGACGTCC CAGACTATGCCGCGTCTTCCG-3′ (SEQ ID NO: 14); (3) Antisense primer with 3′ attB2 site for both ogt PCR: 5′-GGGGACCACTTTGTACAAGAAAGCTGGGTTCTATGCTGACTCAGTGACTTCAA CGGGCTTAATCATGTGG-3′ (SEQ ID NO: 15); (4) Sense primer for first PCR to incorporate HA epitope into oga after the start codon: 5′-CCCCATGTATCCATATGACGTCCCAGACTATGCCGTGCAGAAGGA GAGTCAAGC-3′ (SEQ ID NO: 16); (5) Sense primer containing an attB1 site for using HA-oga PCR product as the template: 5′-GGGGACAAGTTTGTACAAAAAAGCAGGCTGGATGATGTATCCATATG ACGTCCCAGACTATGCCGTGCAGAAGG-3′ (SEQ ID NO: 17); (6) Antisense primer with 3′ attB2 site for both oga PCR: GGGGACCACTTTGTACAAGAAAGCTGGGTTCACAGGCTCCGACCAA GTAT-3′ (SEQ ID NO: 18). The purified DNA fragments were then subjected to Gateway cloning according to manufacturer's instruction yielding final expression constructs, pDEST26/HA-OGT and pDEST26/HA-OGA.

Cell Culture, Transfection and Treatment.

HEK 293T cells were obtained from ATCC (Manassas, Va.) and maintained in Dulbecco's modified Eagle's medium (4.5 g l-1 glucose, Cellgro/Mediatech, Inc., Herndon, Va.) supplemented with 10% fetal bovine serum (GIBCO/Invitrogen, Carlsbad, Calif.) in 37° C. incubator humidified with 5% CO₂. Transfection was performed with 8 μg of DNA and Lipofectamine 2000 reagent (Invitrogen Carlsbad, Calif.) per 10 cm plate of cells according to manufacturer's instruction. Mock transfection was performed in the absence of DNA. Cells were harvested 48 h post-transfection. For immunoprecipitation experiments, cells were washed of the plates with ice-cold PBS and store as a pellet at −80° C. until used. For immunoblotting experiments, cells were washed twice with ice-cold PBS and scraped in lysis buffer (10 mM Tris-HCl, pH 7.5, 150 mM NaCl, 1% Igepal CA-630, 0.1% SDS, 4 mM EDTA, 1 mM DTT, 0.1 mM PUGNAc, Protease inhibitor cocktail) and the lysates were clarified in a microfuge with 16,000 g, for 25 min at 4° C. The protein concentration was quantified with Bradford protein assay with standard procedure (Bio-Rad, Hercules, Calif.) and boiled in sample buffer for 5 min. For mass spectrometry experiment, 2×15 cm plates of 293T cells were treated with 50 μM of PUGNAc for 24 h, cells were pellet and stored as above.

Immunoprecipitation and Western Blotting.

To prepare the nucleocytosolic fraction for CKII immunoprecipitation, HEK293T cell pellets with mock or OGT transfection were resuspended in 4 volumes of hypotonic buffer (5 mM Tris-HCl, pH 7.5, Protease inhibitor cocktail) and transferred into a 2 ml homogenizer. After incubating on ice for 10 min, the cell suspension was subjected to dounce homogenization followed by another 5 Min incubation on ice. One volume of hypertonic buffer (0.1 M Tris-HCl, pH 7.5, 2 M NaCl, 5 mM EDTA, 5 mM DTT, Protease inhibitor cocktail) was then added to the lysate. The lysate was incubated on ice for 5 min followed by another round of dounce homogenization. The resulting lysates were transferred to microfuge tubes containing PUGNAc (final concentration 10 μM) and centrifuged at 18,000 g for 25 min at 4° C. Protein concentration was determined using Bradford protein assay (Bio-Rad, Hercules, Calif.). Prior to IP, the lysates were supplemented with 1% Igepal CA-630 and 0.1% SDS, and precleared with a mixture of normal rabbit or mouse IgG AC and protein A/G PLUS agarose at 4° C. for 30 min. Following clarification, the precleared supernatant was incubated at 4° C. in the presence of antibodies of interested for 4 at 4° C. After adding protein A/G PLUS agarose, the samples were incubated for another 2 h at 4° C. and extensively washed with IP wash buffer (10 mM Tris-HCl, pH 7.5, 150 mM NaCl, 1% Igepal CA-630, 0.1% SDS). Finally, SDSPAGE sample buffer was added into the IP complex and boiled for 3 min. Supernatant was resolved by a 10% or 4-15% Tris-HCl precast minigel (Bio-Rad, Hercules, Calif.), and transferred to Immobilon-P transfer membrane (Millipore, Bedford, Mass.). The membranes were blocked with either 3% BSA (O-GlcNAc blots) or 5% milk (protein blots) in TBST (TBS with 0.1% Tween 20), and probed with each antibody (1:1000 dilution for O-GlcNAc blots, 1:8000 dilution for CKII, OGT and OGA blots, and 1:10,000 dilution for α-tubulin blot) at 4° C. for overnight followed by incubating with secondary antibodies conjugated to HRP at room temperature for 2 h. The final detection of HRP activity was performed using SuperSignal chemiluminescent substrates (Thermo Scientific, Rockford, EL) as followed: MAbs 18B10.C7(3), 9D1.E4(10) and 1F5.D6(14) used Femto; CKII, OGT, OGA and tubulin used PICO. The films were exposed to CL-XPosure film (Thermo Scientific, Rockford, Ill.). After developing the image on the film, the blot was then stripped with 0.1 M glycine (pH 2.5) at room temperature for 1 h, wash with ddH2O and reprobed for loading control (CKII or α-tubulin) as described above.

Conjugation of MAbs to Agarose and Sample Preparation for LC-MS/MS Analysis.

MAbs 18B10.C7(3), 9D1.E4(10) and 1F5.D6(14) or CTD110.6 were covalently conjugated to Protein A/G PULS agarose or anti-Mouse IgM agarose via disuccinimidyl substrate (DSS, Thermo Scientific, Rockford, Ill.) according to manufacturer's instruction. PUGNAc treated HEK293T nucleocytosolic fraction was prepared as above in larger scale, incubated with antibody conjugated agarose, and washed as above. To elute proteins off the agarose, 0.1 M of glycine (pH 2.5) was added and the eluates were immediately neutralized with 1 M Tris-HCl (pH 8.0). The samples were then reduced and alkylated as previously described 8 and subjected to LysC digestion at 37° C. for overnight. After digestion, the samples were processes as previously described (Lim et al., 2008 J. Proteome Res. 7, 1251-1263).

Mass Spectrometry.

The samples were resuspended with 19.5 μl of 0.1% formic acid (in water) and 0.5 μl of 80% acetonitrile/0.1% formic acid (in water) and filtered with 0.2 μm filters (Nanosep, PALL). Samples were then loaded off-line onto a nanospray C18 column and separated with a 160-min linear gradient as previously described (Lim et al., 2008 J. Proteome Res. 7, 1251-1263) using Finnigan LTQ/XL mass spectrometer (ThermoFisher, San Jose, Calif.). Each sample was subjected to 3 runs with different settings: (1) ETD (electron transferred dissociation) mode, where a full MS spectrum was collected followed by 6 MS/MS spectra following ETD (enabled supplemental activation) of the most intense peaks. The dynamic exclusion was set at 1 for 30 sec of duration. (2) CID-NL (collision induced dissociation-pseudo neutral loss) mode, where a full MS spectrum was collected followed by 8 MS/MS spectra following CID of the most intense peaks. Upon encountering a pseudo neutral loss event (a loss of GlcNAc, 203.08), a MS8 spectrum will be created based of the MS/MS spectrum. The dynamic exclusion has the same setting as ETD method. (3) DDNL-ETD (Data dependent neutral loss MS8 under OD followed by ETD activation upon every neutral loss event), where MS/MS spectra from top 5 peaks of each full MS scan were collected with CID (35% normalized collision energy) and monitored for a neutral loss of 203.08 during which a MS8 spectrum will be created. A repeat scan event with neutral loss will be performed using ETD enabled with supplemental activation. The dynamic exclusion was also set the same as above.

Data Analysis and Validation.

MS spectra were searched against the human (Homo sapiens, 32876 entries, Aug. 13, 2007 released) forward and reverse databases extracted from the Swiss-Prot human proteome database using the TurboSequest algorithm (Bioworks 3.3, Thermo Finnigan). The DTA files were generated for spectra with a threshold of 15 ions and a TIC of 1e3. Dynamic mass increases of 15.99, 57.02 and 203.08 Da were considered for oxidized methione, alkylated cysteine and O-GlcNAc modified serine/threonine respectively. The resulting OUT files each samples obtained forward and reversed databases searched were further parsed with ProtoeIQ (Bioinquire) and filtered with 1% FDR (metric used: F-value) and starting peptide coverage for ProFDR at 5.

Statistical Analysis.

Statistical significance between groups was determined by two-tailed, unpaired Student's t test. Differences were considered significant when P <0.05.

The complete disclosure of all patents, patent applications, and publications, and electronically available material (including, for example, nucleotide sequence submissions in, e.g., GenBank and RefSeq, and amino acid sequence submissions in, e.g., SwissProt, PIR, PRP, PDB, and translations from annotated coding regions in GenBank and RefSeq) cited herein are incorporated by reference. The foregoing description and examples have been given for clarity of understanding only. No unnecessary limitations are to be understood therefrom. The invention is not limited to the exact details shown and described, for variations obvious to one skilled in the art will be included within the invention defined by the claims.

1. A method of generating antibody-dependent cell-mediated cytotoxicity (ADCC) in a subject, the method comprising immunizing the subject with a glycolipopeptide
of claim 49.

2-5. (canceled)

6. A method of treating cancer in a subject, the method comprising immunizing the subject with a glycolipopeptide
of claim 49.

7-9. (canceled)

10. The method of claim 6, wherein the cancer or tumor is breast cancer or epithelial cancer.

11. The method of claim 6, wherein the cancer or tumor expresses aberrantly glycosylated MUC1.

12. A method of generating a cytotoxic T cell response directed at MUC1 expressing cells in a subject, the method comprising immunizing the subject with a glycolipopeptide
of claim 49.

13-32. (canceled)

33. The method of claim 1, wherein the peptide component comprising a MHC class II restricted helper T-cell epitope comprises the polio viruses sequence KLFAVWKITYKDT (SEQ ID NO:3) comprises the T cell pan DR epitope PADRE sequence AKFVAAWTLKAAA (SEQ ID NO:24) or FVAAWTLKAAA (SEQ ID NO:25).

34-48. (canceled)

49. A glycolipopeptide comprising:
at least one glycosylated MUC1 glycopeptide component comprising a B-cell epitope;
at least one peptide component comprising a MUC1-derived MHC class II
restricted helper T-cell epitope; and
at least one lipid component.

50. The glycolipopeptide of claim 49, wherein the glycosylated MUC1 glycopeptide component comprising a B-cell epitope comprises glycosylation at one or more serine and/or threonine residues.

51. The glycolipopeptide of claim 50, wherein the glycosylated MUC1 glycopeptide component comprising a B-cell epitope comprises glycosylation with a sugar residue selected from the group consisting of GAlNAc, GlcNAc, Gal, NANA, NGNA, fucose, mannose, and glucose.

52. The glycolipeptide of claim 49, wherein the glycosylated MUC1 glycopeptide component comprising a B-cell epitope is a class I MHC restricted epitope.

53. The glycolipeptide of claim 49, wherein the glycosylated MUC1 glycopeptide component comprising a B-cell epitope and/or the peptide component comprising a MHC class II restricted helper T-cell epitope comprise a human MUC1 peptide sequence.

54. The glycolipopeptide of claim 49, wherein the glycolipopeptide comprising a B-cell epitope and/or the peptide component comprising a MHC class II restricted helper T-cell epitope comprise about 5 to 30 amino acids of a MUC1 protein sequence, the MUC1 protein sequence comprising an extracellular region of the MUC1 protein and comprising one or more serine or threonine residues that are glycosylated.

55. The glycolipopeptide of claim 49, wherein the glycosylated MUC1 glycopeptide component comprising a B-cell epitope comprises an amino acid sequence with at least about 50% sequence identity to SAPDTRPAP (SEQ ID NO:20), TSAPDTRPAP (SEQ ID NO:21), SAPDTRPL (SEQ ID NO:22), or TSAPDTRPL (SEQ ID NO:23).

56. The glycolipopeptide of claim 49, wherein the glycosylated MUC1 glycopeptide component comprising a B-cell epitope comprises SAPDTRPAP (SEQ ID NO:20), TSAPDTRPAP (SEQ ID NO:21), SAPDTRPL (SEQ ID NO:22), or TSAPDTRPL (SEQ ID NO:23).

57. (canceled)

58. The glycolipopeptide of claim 49, wherein the lipid component comprises one or more lipid chains, one or more cysteine residues and one or more lysine residues.

59. The glycolipopeptide of claim 49, wherein the lipid component comprises a Toll-like receptor (TLR) ligand and/or comprises a lipidic adjuvant.

60. The glycolipopeptide of claim 59, wherein the Toll-like receptor (TLR) ligand comprises a TLR2 ligand.

61. The glycolipopeptide of claim 60, wherein the TLR2 ligand comprises Pam₃CysSK₄.

62. (canceled)

63. The glycolipopeptide of claim 59, wherein the lipidic adjuvant comprises a lipidated amino acid (LAA).

64. The glycolipopeptide of claim 49, wherein the MUC1-derived B-cell peptide epitope and the MUC1-derived MHC class II restricted helper T-cell peptide epitope comprise a contiguous amino acid sequence.

65. The glycolipopeptide of claim 64, wherein the contiguous amino acid sequence comprises a sequence with at least 50% sequence identity to the amino acid sequence APGSTAPPAHGVTSA (SEQ ID NO:26), APGSTAPPAHGVTSAPDTRPL (SEQ ID NO:27), APGSTAPPAHGVTSAPDTRPL (SEQ ID NO:28), or APGSTAPPAHGVTSAPDTRPL (SEQ ID NO:29).

66. The glycolipopeptide of claim 64 wherein the contiguous amino acid sequence comprises the amino acid sequence APGSTAPPAHGVTSA (SEQ ID NO:26), APGSTAPPAHGVTSAPDTRPL (SEQ ID NO:27), APGSTAPPAHGVTSAPDTRPL (SEQ ID NO:28), or APGSTAPPAHGVTSAPDTRPL (SEQ ID NO:29).

67. (canceled)

68. The glycolipopeptide of claim 49, further comprising a covalently linked immune modulator.

69. The glycolipopeptide of claim 68, wherein the immune modulator is selected from the group consisting of a TLR9 agonist, a COX-2 inhibitor, GM-CSF, an inhibitor of indoleamine 2, 3-dioxygenase (IDO), a chemotherapy agent, and combinations thereof.

70. A pharmaceutical composition comprising:
a glycolipopeptide according to claim 49; and
a pharmaceutically acceptable carrier.

71-72. (canceled)

73. A composition of claim 70 further comprising an immune modulator.

74-80. (canceled)


SEQUENCE LISTING (39 sequences)

SEQ ID NO: 1 - protein, 15 aa, unknown
  peptide derived from tetanus toxin
QYIKANSKFIGITEL

SEQ ID NO: 2 - protein, 20 aa, Neisseria meningitides
YAFKYARHANVGRNAFELFL

SEQ ID NO: 3 - protein, 13 aa, polio virus
KLFAVWKITYKDT

SEQ ID NO: 4 - protein, 15 aa, unknown
  peptide derived from tetanus toxin
VSIDKFRIFCKANPK

SEQ ID NO: 5 - protein, 16 aa, unknown
  peptide derived from tetanus toxin
LKFIIKRYTPNNEIDS

SEQ ID NO: 6 - protein, 16 aa, unknown
  peptide derived from tetanus toxin
IREDNNITLKLDRCNN

SEQ ID NO: 7 - protein, 21 aa, unknown
  peptide derived from tetanus toxin
FNNFTVSFWLRVPKVSASHLE

SEQ ID NO: 8 - protein, 20 aa, Neisseria meningitidis
YAFKYARHANVGRNAFELFL

SEQ ID NO: 9 - protein, 18 aa, Pseudomonas falsiparum
EKKIAKMEKASSVFNVNN

SEQ ID NO: 10 - protein, 5 aa, artificial
  peptide sequence modified by O-GlcNAc
TPVSS

SEQ ID NO: 11 - protein, 10 aa, artificial
  peptide sequence modified by O-GlcNAc
GSTPVSSANM

SEQ ID NO: 12 - protein, 11 aa, artificial
  peptide sequence modified by O-GlcNAc
CGSTPVSSANM

SEQ ID NO: 13 - DNA, 54 bp, artificial
  synthetic oligonucleotide primer
ccccatgtatccatatgacgtcccagactatgccgcgtcttccgtgggcaacgt

SEQ ID NO: 14 - DNA, 74 bp, artificial
  synthetic oligonucleotide primer
ggggacaagtttgtacaaaaaagcaggctggatgatgtatccatatgacgtcccagactatgccgcgtcttccg

SEQ ID NO: 15 - DNA, 70 bp, artificial
  synthetic oligonucleotide primer
ggggaccactttgtacaagaaagctgggttctatgctgactcagtgacttcaacgggcttaatcatgtgg

SEQ ID NO: 16 - DNA, 54 bp, artificial
  synthetic oligonucleotide primer
ccccatgtatccatatgacgtcccagactatgccgtgcagaaggagagtcaagc

SEQ ID NO: 17 - DNA, 74 bp, artificial
  synthetic oligonucleotide primer
ggggacaagtttgtacaaaaaagcaggctggatgatgtatccatatgacgtcccagactatgccgtgcagaagg

SEQ ID NO: 18 - DNA, 50 bp, artificial
  synthetic oligonucleotide primer
ggggaccactttgtacaagaaagctgggttcacaggctccgaccaagtat

SEQ ID NO: 19 - skipped

SEQ ID NO: 20 - protein, 9 aa, artificial
  B-cell peptide epitope
SAPDTRPAP

SEQ ID NO: 21 - protein, 10 aa, artificial
  B-cell peptide epitope
TSAPDTRPAP

SEQ ID NO: 22 - protein, 8 aa, artificial
  B-cell peptide epitope
SAPDTRPL

SEQ ID NO: 23 - protein, 9 aa, artificial
  B-cell peptide epitope
TSAPDTRPL

SEQ ID NO: 24 - protein, 13 aa, artificial
  T-cell pan DR epitope PADRE sequence
AKFVAAWTLKAAA

SEQ ID NO: 25 - protein, 11 aa, artificial
  T-cell pan DR epitope PADRE sequence
FVAAWTLKAAA

SEQ ID NO: 26 - protein, 15 aa, artificial
  contiguous 15-mer amino acid sequence of MUC1 epitope
APGSTAPPAHGVTSA

SEQ ID NO: 27 - protein, 21 aa, artificial
  contiguous 21-mer amino acid sequence of MUC1 epitope
APGSTAPPAHGVTSAPDTRPL

SEQ ID NO: 28 - protein, 21 aa, artificial
  contiguous 21-mer amino acid sequence of MUC1 epitope with single glycosylation site
APGSTAPPAHGVTSAPDTRPL

SEQ ID NO: 29 - protein, 21 aa, artificial
  contiguous 21-mer amino acid sequence of MUC1 epitope with double glycosylation sites
APGSTAPPAHGVTSAPDTRPL

SEQ ID NO: 30 - protein, 27 aa, artificial
  MUC1 epitope
  misc_feature (27)..(27): Xaa can be any naturally occurring amino acid
SKKKKGAPGSTAPPAHGVTSAPDTRPX

SEQ ID NO: 31 - protein, 25 aa, artificial
  MUC1 epitope
SKKKKGSTAPPAHGVTSAPDTRPAP

SEQ ID NO: 32 - protein, 23 aa, artificial
  MUC1 epitope
SKKKKGSLSYTNPAVAAATASNL

SEQ ID NO: 33 - protein, 31 aa, artificial
  MUC1 epitope
SKKKKGCKLFAVWKITYKDTGTSAPDTRPAP

SEQ ID NO: 34 - protein, 20 aa, artificial
  MUC1 epitope
SKKKKGCKLFAVWKITYKDT

SEQ ID NO: 35 - protein, 26 aa, artificial
  MUC1 epitope
GGKLFAVWKITYKDTGTSAPDTRPAP

SEQ ID NO: 36 - DNA, 20 bp, artificial
  CpG immunostimulatory oliodeoxynucleotide
tccatgacgttcctgacgtt

SEQ ID NO: 37 - protein, 22 aa, artificial
  MUC1 epitope
APGSTAPPAHGVTSAPDTRPAP

SEQ ID NO: 38 - protein, 240 aa, Homo sapiens
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTS

SEQ ID NO: 39 - protein, 18 aa, artificial
  missynthesized 18mer C terminus control peptide
SSLSYNTNPAVAAASANL